SEQUENCE LISTING (13 sequences)

SEQ ID NO: 1 - protein, 222 aa, Homo sapiens
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVWALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 2 - protein, 5 aa, synthetic construct
NFGMH

SEQ ID NO: 3 - protein, 17 aa, synthetic construct
FISSGSSSIYYADTVKG

SEQ ID NO: 4 - protein, 10 aa, synthetic construct
MRLDYYAMDY

SEQ ID NO: 5 - protein, 11 aa, synthetic construct
RASKSISKYLA

SEQ ID NO: 6 - protein, 7 aa, synthetic construct
SGSTLQS

SEQ ID NO: 7 - protein, 9 aa, synthetic construct
QQHNEYPWT

SEQ ID NO: 8 - protein, 119 aa, synthetic construct
EVQLQQSGPELVKPGASVKISCKTSGYTFTEYTMHWVKQSHGKNLEWIGGINPNNGGTSYNQKFKGRATLTVDKSSSTAYMELRSLTSDDSAVYYCARPGWYNYAMDYWGQGTSVTVSS

SEQ ID NO: 9 - protein, 107 aa, synthetic construct
DVQITQSPSYLAASPGETITINCRASKSISKYLAWYQEKPGKTNKLLIYSGSTLQSGIPSRFSGSGSGTDFTLTISSLEPEDFAMYYCQQHNEYPWTFGGGTKLEIK

SEQ ID NO: 10 - protein, 119 aa, synthetic construct
EVQLVESGGGLVQPGGSLRLSCAASGFTFSNFGMHWVRQAPGKGLEWVAFISSGSSSIYYADTVKGRFTISRDNAKNSLYLQMNSLRAEDTAVYYCARMRLDYYAMDYWGQGTMVTVSS

SEQ ID NO: 11 - protein, 107 aa, synthetic construct
DIQMTQSPSSLSASVGDRVTITCRASKSISKYLAWYQQKPGKAPKLLIYSGSTLQSGVPSRFSGSGSGTDFTLTISSLQPEDFATYYCQQHNEYPWTFGGGTKVEIK

SEQ ID NO: 12 - protein, 447 aa, synthetic construct
EVQLVESGGGLVQPGGSLRLSCAASGFTFSNFGMHWVRQAPGKGLEWVAFISSGSSSIYYADTVKGRFTISRDNAKNSLYLQMNSLRAEDTAVYYCARMRLDYYAMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCWVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 213 aa, synthetic construct
DIQMTQSPSSLSASVGDRVTITCRASKSISKYLAWYQQKPGKAPKLLIYSGSTLQSGVPSRFSGSGSGTDFTLTISSLQPEDFATYYCQQHNEYPWTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0179176 A1
(43) Pub. Date: Jun. 5, 2025

(54) ANTI-TIGIT ANTIBODIES AND USES OF THE SAME

(71) Applicant: ARCUS BIOSCIENCES, INC., Hayward, CA (US)

(72) Inventors: Paul G. FOSTER, Novato, CA (US); Kelsey GAUTHIER, Alameda, CA (US); Juan Carlos JAEN, Burlingame, CA (US); Lisa C. SEITZ, Oakland, CA (US); Nigel WALKER, Burlingame, CA (US); Matthew J. WALTERS, Pacifica, CA (US)

(21) Appl. No.: 18/862,061

(22) PCT Filed: May 1, 2023
(86) PCT No.: PCT/US2023/066451

§ 371 Date: Oct. 31, 2024

Related U.S. Application Data
(60) Provisional application No. 63/337,471, filed on May 2, 2022.
(60) Provisional application No. 63/427,688, filed on Nov. 23, 2022.
(60) Provisional application No. 63/433,390, filed on Dec. 16, 2022.

(51) Int. Cl. C07K 16/28; A61K 39/00; A61K 45/06

(52) U.S. Cl. CPC C07K 16/2803; A61K 45/06; A61K 2039/505; A61K 2039/545; C07K 2317/565; C07K 2317/71

(57) ABSTRACT

Described herein are treatments and preventions for cancer using antibodies that bind to TIGIT, and uses of an anti-TIGIT antibody in the manufacture of a medicament for the treatment or prevention of cancer. Also described herein are methods of blocking binding of TIGIT to CD155 without altering certain immune parameters by administering an antibody that binds to TIGIT.

### CROSS-REFERENCE TO RELATED APPLICATIONS

This application claims the benefit under 35 U.S.C. § 119(e) of U.S. Provisional Application No. 63/337,471, filed May 2, 2022, U.S. Provisional Application No. 63/427,688, filed Nov. 23, 2022, and U.S. Provisional Application No. 63/433,390, filed Dec. 16, 2022, each of which is hereby incorporated by reference in its entirety.

### FIELD

Described herein are treatments and preventions for cancer using antibodies that bind to TIGIT, and uses of an anti-TIGIT antibody (e.g., domvanalimab) in the manufacture of a medicament for the treatment or prevention of cancer. Also described herein are methods of blocking binding of TIGIT to CD155 without altering certain immune parameters by administering an antibody that binds to TIGIT (e.g., domvanalimab).

### BACKGROUND

The following discussion is provided to aid the reader in understanding the disclosure and is not admitted to describe or constitute prior art thereto.

The antigen-specific immune response is a complex and often unpredictable biological process that is controlled by multiple layers of positive and negative regulators. T cells are initially stimulated through the T cell receptor (TCR) by the recognition of their cognate peptide antigen presented by major histocompatibility complex (MHC) molecules on antigen-presenting cells. Optimal T cell activation requires a “second signal” provided by costimulatory molecules such as CD28. The immune response is further regulated positively by costimulatory molecules, such as OX40, GITR, and 4-1BB that belong to the TNF receptor super-family, and negatively regulated by checkpoint molecules such as PD-1 and CTLA-4. The function of checkpoint molecules is to prevent undesired over-reaction of the immune system in the body; however, they also restrict the ability of the immune system to effectively fight against cancer and infectious disease. Blocking the function of PD-1 or CTLA-4 by an antagonistic monoclonal IgG antibody has been reported to be effective for immunotherapy of cancer in humans (for review, see Pardoll, Nat. Rev. Cancer, 12:252-264, 2012; Mahoney et al., Nat. Rev. Drug Discov. 14:561-584, 2015; Shin et al., Curr. Opin. Immunol. 33:23-35, 2015; Marquez-Rodas et al. Ann. Transl. Med. 3:267, 2015).

Other checkpoint molecules such as TIM-3, LAG-3, TIGIT, BTLA, and VISTA have been reported (Mercier et al., Front. Immunol. 6:418, 2015). TIGIT (T cell immunoreceptor with lg and ITIM domains), a member of the immunoglobulin superfamily with an immunoreceptor tyrosine-based inhibitory motif (ITIM) in the cytoplasmic tail, is expressed on subsets of activated T cells and natural killer (NK) cells (Yu et al., Nat. Immunol. 10:48-57, 2009). TIGIT is known to interact with CD155 (also called PVR and necl-5), CD112 (also called PVRL2 and nectin-2), and possibly CD113 (also called PVRL3 and nectin-3) (Mercier et al., supra; Martinet et al., Nat. Rev. Immunol. 15:243-254, 2015). Binding of TIGIT with a high affinity ligand CD155, which is expressed on antigen-presenting cells, has been reported to suppress the function of T cells and NK cells (Mercier et al., supra; Jailer et al., J. Immunol. 186: 1338-1342, 2011; Stanietsky et al., Eur. J. Immunol. 43:2138-2150, 2013; Li et al., J. Biol. Chem. 289:17647-17657, 2014; Zhang et al. Cancer Immunol. Immunother. Epub on Feb. 3, 2016). TIGIT has also been reported to inhibit T cells indirectly by modulating cytokine production by dendritic cells (Yu et al., supra).

Tumors constitute highly suppressive microenvironments where infiltrating T cells can be exhausted and NK cells are silenced by checkpoint molecules such as PD-1 and TIGIT to evade immune responses (Johnston et al., Cancer Cell. 26:926-937, 2014; Chauvin et al., J. Clin. Invest. 125:2046-2058, 2015; Inozume et al., J. Invest. Dermatol. Epub on Oct. 12, 2015). A high-level expression of TIGIT on CD8+ T cells has been reported to correlate with poor clinical outcomes of AML subjects (Kong et al., Clin. Cancer Res. Epub on Jan. 13, 2016). The functional defects of exhausted TIGIT+ CD8+ T cells from AML subjects were reported to be reversed by the siRNA-mediated knockdown of TIGIT expression (Kong et al., supra). It has also been reported that effector CD8+ T cells during HIV infection in blood and SIV infection in lymphoid tissue exhibit higher levels of TIGIT (Chew et al., PLOS Pathogens, 12:e1005349, 2016). In addition, an ex vivo antibody blockade of TIGIT was reported to restore viral-specific CD8+ T cell effector responses.

Outside the tumor microenvironment, immune cells circulating through the periphery and other tissues also express certain immune checkpoints, particularly TIGIT. The vast majority of these cells are not involved in anti-tumor immune control. Thus, while it may be beneficial to destroy TIGIT-expressing immune cells within tumors, the destruction of TIGIT-expressing immune cells outside the tumors (e.g., in circulation and other tissues/organs) is not expected to contribute to anti-tumor control and may, in fact, lead to unwanted side effects, such as the appearance of systemic auto-immunity (as may result from depletion of circulating TIGIT+ regulatory T cells (Treg)) or decreased systemic anti-viral protection (as may result from depletion of circulating TIGIT+ effector T cells (Teff)).

There remains a need for effective treatment regimens for anti-TIGIT antibodies with improved safety.

### SUMMARY

Described herein are antibodies that bind to TIGIT, and methods of using the same. In particular, the present disclosure describes treating cancer by administering to a human subject in need thereof an anti-TIGIT antibody (e.g., domvanalimab), wherein the anti-TIGIT antibody has reduced binding to one or more FcγRs (e.g., activating FcγRs) as compared to wild type (WT) IgG1. In some embodiments, the anti-TIGIT antibody may have an inability to bind one or more FcγRs (e.g., activating FcγRs). As a result of this reduced or lacking binding, the anti-TIGIT antibody has reduced or lacking Fc effector function, and treatment with such an anti-TIGIT antibody can result in reduced or negligible depletion of peripheral lymphocyte populations, in regulatory and CD+ T cells and/or a reduction in number and/or severity of one or more immune-related adverse event as compared to an Fc-enabled anti-TIGIT antibody. Improvements in immune-mediated adverse events have numerous benefits for patients, which may include, for example, fewer treatment disruptions, fewer dose reductions, fewer discontinuations, or any combination thereof.

In one aspect, the present disclosure provides methods for treating cancer in a human subject in need thereof, comprising administering to the subject an anti-TIGIT antibody that has reduced binding to one or more activating human FcγRs as compared to wild type (WT) human IgG1, wherein treatment results in a reduction of one or more adverse event as compared to a similar treatment comprising an Fc-enabled anti-TIGIT antibody.

In another aspect, the present disclosure provides methods for treating cancer in a human subject in need thereof, comprising administering to the subject an anti-TIGIT antibody that has reduced binding to one or more activating human FcγRs as compared to wild type (WT) human IgG1, wherein the subject has a reduced likelihood of experiencing one or more adverse event as compared to treatment with an Fc-enabled anti-TIGIT antibody.

In another aspect, the present disclosure provides an anti-TIGIT antibody that has reduced binding to one or more activating human FcγRs as compared to wild type (WT) human IgG1, for use in treating cancer in a human subject in need thereof. In some embodiments, treatment results in a reduction of one or more adverse event as compared to a similar treatment comprising an Fc-enabled anti-TIGIT antibody. In some embodiments, the subject has a reduced likelihood of experiencing one or more adverse event as compared to treatment with an Fc-enabled anti-TIGIT antibody.

In another aspect, the present disclosure provides a pharmaceutical composition comprising an anti-TIGIT antibody that has reduced binding to one or more activating human FcγRs as compared to wild type (WT) human IgG1, for use in treating cancer in a human subject in need thereof. In some embodiments, treatment results in a reduction of one or more adverse event as compared to a similar treatment comprising an Fc-enabled anti-TIGIT antibody. In some embodiments, the subject has a reduced likelihood of experiencing one or more adverse event as compared to treatment with an Fc-enabled anti-TIGIT antibody.

In another aspect, the present disclosure provides for use of an anti-TIGIT antibody that has reduced binding to one or more activating human FcγRs as compared to wild type (WT) human IgG1 for the manufacture of a medicament for treating cancer in a human subject in need thereof. In some embodiments, treatment results in a reduction of one or more adverse event as compared to a similar treatment comprising an Fc-enabled anti-TIGIT antibody. In some embodiments, the subject has a reduced likelihood of experiencing one or more adverse event as compared to treatment with an Fc-enabled anti-TIGIT antibody.

In some embodiments of the foregoing aspects, the Fc-enabled anti-TIGIT antibody is selected from AB308, BMS-986207, tiragolumab, vibostolimab, etigilimab, ociperlimab, EOS-448, SEA-TGT, AGEN1777, AGEN1327, JS006, ralzapastotug, and an Fc-enabled version of domvanalimab comprising a wild-type IgG1 Fc region.

In another aspect, the present disclosure provides methods for treating cancer in a human subject in need thereof without significantly increasing the likelihood of adverse event as compared to a standard of care, comprising administering to the subject an anti-TIGIT antibody that has reduced binding to one or more activating human FcγRs as compared to wild type (WT) human IgG1 in combination with one or more additional therapy. In some embodiments, the one or more additional therapy is a standard of care.

In another aspect, the present disclosure provides an anti-TIGIT antibody that has reduced binding to one or more activating human FcγRs as compared to wild type (WT) human IgG1 and one or more additional therapy, for use in treating cancer in a human subject in need thereof without significantly increasing the likelihood of adverse event as compared to a standard of care. In some embodiments, the one or more additional therapy is a standard of care.

In another aspect, the present disclosure provides a pharmaceutical composition comprising an anti-TIGIT antibody that has reduced binding to one or more activating human FcγRs as compared to wild type (WT) human IgG1, wherein said anti-TIGIT antibody is used in combination with one or more additional therapy for treating cancer in a human subject in need thereof without significantly increasing the likelihood of adverse event as compared to a standard of care. In some embodiments, the one or more additional therapy is a standard of care.

In another aspect, the present disclosure provides methods for reducing one or more adverse event experienced by a human subject being treated for cancer with an anti-TIGIT antibody, comprising administering to the subject an anti-TIGIT antibody that has reduced binding to one or more activating human FcγRs as compared to wild type (WT) human IgG1.

In another aspect, the present disclosure provides an anti-TIGIT antibody that has reduced binding to one or more activating human FcγRs as compared to wild type (WT) human IgG1, for use in reducing one or more adverse event experienced by a human subject being treated for cancer with an anti-TIGIT antibody.

In another aspect, the present disclosure provides a pharmaceutical composition comprising an anti-TIGIT antibody that has reduced binding to one or more activating human FcγRs as compared to wild type (WT) human IgG1, for use in reducing one or more adverse event experienced by a human subject being treated for cancer with an anti-TIGIT antibody.

In another aspect, the present disclosure provides methods for reducing one or more adverse event experienced by a human subject being treated for cancer with an anti-TIGIT antibody and an additional immunotherapeutic agent, comprising administering to the subject an anti-TIGIT antibody that has reduced binding to one or more activating human FcγRs as compared to wild type (WT) human IgG1 and an additional immunotherapeutic agent. In some embodiments, the immunotherapeutic agent is a checkpoint inhibitor, optionally selected from ipilimumab, nivolumab, pembrolizumab, cemiplimab, avelumab, durvalumab, atezolizumab, and zimberelimab.

In another aspect, the present disclosure provides an anti-TIGIT antibody that has reduced binding to one or more activating human FcγRs as compared to wild type (WT) human IgG1 and an additional immunotherapeutic agent, for use in reducing one or more adverse event experienced by a human subject being treated for cancer with an anti-TIGIT antibody and the additional immunotherapeutic agent. In some embodiments, the immunotherapeutic agent is a checkpoint inhibitor, optionally selected from ipilimumab, nivolumab, pembrolizumab, cemiplimab, avelumab, durvalumab, atezolizumab, and zimberelimab.

In another aspect, the present disclosure provides a pharmaceutical composition comprising an anti-TIGIT antibody that has reduced binding to one or more activating human FcγRs as compared to wild type (WT) human IgG1, wherein said anti-TIGIT antibody is used in combination with an additional immunotherapeutic agent, for use in reducing one or more adverse event experienced by a human subject being treated for cancer with an anti-TIGIT antibody and the additional immunotherapeutic agent. In some embodiments, the immunotherapeutic agent is a checkpoint inhibitor, optionally selected from ipilimumab, nivolumab, pembrolizumab, cemiplimab, avelumab, durvalumab, atezolizumab, and zimberelimab.

In some embodiments of the foregoing aspects, the adverse event is a treatment-related adverse event, an immune-related adverse event, or a treatment-related immune-related adverse event. In some embodiments, the adverse event is infusion-related reaction, rash, rash maculo-papular, pyrexia, myocarditis, pneumonitis, immune-mediated lung disease, interstitial lung disease, immune-mediated hepatitis, and/or immune-mediated enterocolitis. In some embodiments the immune-related adverse event is selected from: (i) a skin or subcutaneous tissue disorder, a gastrointestinal disorder, a hepatobiliary disorder, an endocrine disorder, or a respiratory, thoracic or mediastinal disorder; (ii) a skin or subcutaneous tissue disorder; (iii) rash, oral mucositis, dry mouth, colitis, diarrhea, hepatitis, pneumonitis, endocrinopathies, hypophysitis, hypothyroidism, hyperthyroidism, adrenal insufficiency, diabetes, or a combination thereof; (iv) arthritis, hepatitis, hypothyroidism, hyperthyroidism, infusion-related reaction, maculo-papular rash, pneumonitis, pruritis, psoriasis, rash, swelling face, or a combination thereof; or (v) infusion-related reaction, maculo-papular rash, pruritis, psoriasis, rash, or a combination thereof.

In some embodiments of the foregoing aspects, the adverse event is an immune-related adverse event or an infusion-related adverse event.

In another aspect, the present disclosure provides methods for reducing one or more dose reduction, temporary treatment disruption, or treatment discontinuation experienced by a human subject being treated for cancer with an anti-TIGIT antibody, comprising administering to the subject an anti-TIGIT antibody that has reduced binding to one or more activating human FcγRs as compared to wild type (WT) human IgG1.

In another aspect, the present disclosure provides an anti-TIGIT antibody that has reduced binding to one or more activating human FcγRs as compared to wild type (WT) human IgG1, for use in reducing one or more dose reduction, temporary treatment disruption, or treatment discontinuation experienced by a human subject being treated for cancer with an anti-TIGIT antibody.

In another aspect, the present disclosure provides a pharmaceutical composition comprising an anti-TIGIT antibody that has reduced binding to one or more activating human FcγRs as compared to wild type (WT) human IgG1, for use in reducing one or more dose reduction, temporary treatment disruption, or treatment discontinuation experienced by a human subject being treated for cancer with an anti-TIGIT antibody.

In another aspect, the present disclosure provides methods for reducing one or more dose reduction, temporary treatment disruption, or treatment discontinuation experienced by a human subject being treated for cancer with an anti-TIGIT antibody and an additional immunotherapeutic agent, comprising administering to the subject an anti-TIGIT antibody that has reduced binding to one or more activating human FcγRs as compared to wild type (WT) human IgG1. In some embodiments, the immunotherapeutic agent is a checkpoint inhibitor, optionally selected from ipilimumab, nivolumab, pembrolizumab, cemiplimab, avelumab, durvalumab, atezolizumab, and zimberelimab.

In another aspect, the present disclosure provides an anti-TIGIT antibody that has reduced binding to one or more activating human FcγRs as compared to wild type (WT) human IgG1 and an additional immunotherapeutic agent, for use in reducing one or more dose reduction, temporary treatment disruption, or treatment discontinuation experienced by a human subject being treated for cancer with an anti-TIGIT antibody and an additional immunotherapeutic agent. In some embodiments, the immunotherapeutic agent is a checkpoint inhibitor, optionally selected from ipilimumab, nivolumab, pembrolizumab, cemiplimab, avelumab, durvalumab, atezolizumab, and zimberelimab.

In another aspect, the present disclosure provides a pharmaceutical composition comprising an anti-TIGIT antibody that has reduced binding to one or more activating human FcγRs as compared to wild type (WT) human IgG1, wherein said anti-TIGIT antibody is used in combination with an additional immunotherapeutic agent, for use in reducing one or more dose reduction, temporary treatment disruption, or treatment discontinuation experienced by a human subject being treated for cancer with an anti-TIGIT antibody and an additional immunotherapeutic agent. In some embodiments, the immunotherapeutic agent is a checkpoint inhibitor, optionally selected from ipilimumab, nivolumab, pembrolizumab, cemiplimab, avelumab, durvalumab, atezolizumab, and zimberelimab.

In some embodiments of the foregoing aspects, the method may further comprise a reduction in one or more immune-related adverse event, as compared to a similar treatment comprising an Fc-enabled anti-TIGIT antibody. In some embodiments, the immune-related adverse event is selected from: (i) a skin or subcutaneous tissue disorder, a gastrointestinal disorder, a hepatobiliary disorder, an endocrine disorder, or a respiratory, thoracic or mediastinal disorder; (ii) a skin or subcutaneous tissue disorder; (iii) rash, oral mucositis, dry mouth, colitis, diarrhea, hepatitis, pneumonitis, endocrinopathies, hypophysitis, hypothyroidism, hyperthyroidism, adrenal insufficiency, diabetes, or a combination thereof; (iv) arthritis, hepatitis, hypothyroidism, hyperthyroidism, infusion-related reactions, maculo-papular rash, pneumonitis, pruritis, psoriasis, rash, swelling face, or a combination thereof; or (v) infusion-related reactions, maculo-papular rash, pruritis, psoriasis, rash; or a combination thereof.

In some embodiments of the foregoing aspects and embodiments, administering comprises one or more dosing cycles. In some embodiments, a dosing cycle comprises administering the anti-TIGIT antibody to the subject once every 2 weeks, once every 3 weeks, or once every 4 weeks.

In some embodiments of the foregoing aspects and embodiments, the anti-TIGIT antibody is administered to the subject at a dose of about 5 mg/kg, about 10 mg/kg, about 15 mg/kg, about 20 mg/kg, or about 25 mg/kg. In some embodiments of the foregoing aspects, the anti-TIGIT antibody is administered to the subject at a dose of about 500 mg to about 2000 mg, about 600 mg to about 800 mg, about 800 mg to about 1200 mg, about 900 mg to about 1200 mg, about 1200 mg to about 1600 mg, or about 1200 mg to about 1500 mg. In some embodiments, a dosing cycle comprises administering the anti-TIGIT antibody to the subject at a dose of about 800 mg once every three weeks, about 1200 mg once every three weeks or at a dose of about 1600 mg once every four weeks.

In some embodiments of the foregoing aspects and embodiments, the methods may further comprise administration of one or more additional therapeutic agent, wherein one or more additional therapeutic agent is, optionally, an immunotherapeutic agent, a chemotherapeutic agent, a chemotherapeutic regimen, or a combination thereof. In some embodiments, the immunotherapeutic agent is a checkpoint inhibitor, optionally selected from ipilimumab, nivolumab, pembrolizumab, cemiplimab, avelumab, durvalumab, atezolizumab, and zimberelimab; or an ATP-adenosine axis-targeting agent, optionally selected from an A₂ₐR antagonist, an A_{2b}R antagonist, an A₂ₐR and A_{2b}R antagonist, a CD73 inhibitor, and a CD39 inhibitor. In some embodiments, the chemotherapeutic regimen is a fluoropyrimidine-containing chemotherapy (e.g., fluorouracil, capecitabine, floxuridine) or a platinum-containing chemotherapy (e.g., carboplatin, cisplatin, or oxaliplatin). In some embodiments, the chemotherapeutic regimen is FOLFOX, CAPOX, cisplatin and pemetrexed, carboplatin and pemetrexed, carboplatin and paclitaxel, or carboplatin and nab-paclitaxel.

In some embodiments of the foregoing aspects and embodiments, the methods may further comprise administration of one or more additional therapeutic agent, wherein one or more additional therapeutic agent is zimberelimab. In some embodiments, a therapeutically effective amount of zimberelimab is about 360 mg intravenously administered every three weeks, or about 480 mg intravenously administered every four weeks. In some embodiments, a therapeutically effective amount of zimberelimab is about 720 mg intravenously administered every six weeks, about 760 mg intravenously administered every six weeks, about 960 mg intravenously administered every six weeks, or about 720 mg to about 960 mg intravenously administered every six weeks.

In some embodiments of the foregoing aspects and embodiments, the methods may further comprise administration of one or more additional therapeutic agent, wherein one or more additional therapeutic agent is etrumadenant. In some embodiments, a therapeutically effective amount of etrumadenant is about 50 mg to about 250 mg per day administered orally, about 50 mg to about 225 mg per day administered orally, about 50 mg to about 150 mg per day administered orally, or about 100 mg to about 250 mg per day administered orally. In some embodiments, a therapeutically effective amount of etrumadenant is about 50 mg, about 75 mg, about 100 mg, about 150 mg, about 175 mg, about 200 mg, about 225 mg, or about 250 mg per day administered orally.

In some embodiments of the foregoing aspects and embodiments, the methods may further comprise administration of one or more additional therapeutic agent, wherein at least one additional therapeutic agent is zimberelimab and at least one additional therapeutic agent is etrumadenant. In some embodiments, a therapeutically effective amount of zimberelimab is about 360 mg intravenously administered every three weeks, or about 480 mg intravenously administered every four weeks, and a therapeutically effective amount of etrumadenant is about 50 mg to about 250 mg per day administered orally or about 50 mg to about 150 mg per day administered orally. In some embodiments, a therapeutically effective amount of zimberelimab is about 720 mg intravenously administered every six weeks, about 760 mg intravenously administered every six weeks, about 960 mg intravenously administered every six weeks, or about 720 mg to about 960 mg intravenously administered every six weeks, and a therapeutically effective amount of etrumadenant is about 50 mg to about 250 mg per day administered orally or about 50 mg to about 150 mg per day administered orally.

In some embodiments of the foregoing aspects and embodiments, the methods may further comprise administration of one or more additional therapeutic agent, wherein one or more additional therapeutic agent is quemliclustat. In some embodiments, a therapeutically effective amount of quemliclustat is about 100 mg to about 200 mg intravenously administered every two weeks, or about 300 mg intravenously administered every three weeks.

In some embodiments of the foregoing aspects and embodiments, the methods may further comprise administration of one or more additional therapeutic agent, wherein at least one additional therapeutic agent is zimberelimab and at least one additional therapeutic agent is quemliclustat. In some embodiments, a therapeutically effective amount of zimberelimab is about 360 mg intravenously administered every three weeks, or about 480 mg intravenously administered every four weeks, and a therapeutically effective amount of quemliclustat is about 300 mg intravenously administered every three weeks. In some embodiments, a therapeutically effective amount of zimberelimab is about 720 mg intravenously administered every six weeks, about 760 mg intravenously administered every six weeks, about 960 mg intravenously administered every six weeks, or about 760 mg to about 960 mg intravenously administered every six weeks, and a therapeutically effective amount of quemliclustat is about 300 mg intravenously administered every three weeks.

In some embodiments of the foregoing aspects and embodiments, the anti-TIGIT antibody that has reduced binding to one or more activating human FcγR is an IgG4 or an IgG1 with reduced ability to bind one or more activating human FcγR.

In some embodiments of the foregoing aspects and embodiments, the anti-TIGIT antibody that has reduced binding to one or more activating human FcγR does not bind to one or more activating human FcγR.

In some embodiments of the foregoing aspects and embodiments, the anti-TIGIT antibody comprises a heavy chain CDR1 comprising the amino acid sequence of SEQ ID NO: 2, a heavy chain CDR2 comprising the amino acid sequence of SEQ ID NO: 3, a heavy chain CDR3 comprising the amino acid sequence of SEQ ID NO: 4, a light chain CDR1 comprising the amino acid sequence of SEQ ID NO: 5, a light chain CDR2 comprising the amino acid sequence of SEQ ID NO: 6, and a light chain CDR3 comprising the amino acid sequence of SEQ ID NO: 7.

In some embodiments of the foregoing aspects and embodiments, the anti-TIGIT antibody comprises a heavy chain variable region with at least 90% sequence identity to SEQ ID NO: 8 or 10 and a light chain variable region with at least 90% sequence identity to SEQ ID NO: 9 or 11.

In some embodiments of the foregoing aspects and embodiments, the anti-TIGIT antibody comprises a heavy chain with at least 90% sequence identity to SEQ ID NO: 12 and a light chain with at least 90% sequence identity to SEQ ID NO: 13.

In some embodiments of the foregoing aspects and embodiments, the anti-TIGIT antibody binds to the same epitope of TIGIT as domvanalimab or the anti-TIGIT antibody competitively inhibits binding of domvanalimab to human TIGIT by at least 50%.

In some embodiments of the foregoing aspects and embodiments, the cancer is a solid tumor, optionally wherein the tumor is a locally advanced and/or unresectable tumor; a metastatic tumor; a recurrent tumor; a tumor no longer responding to a treatment optionally wherein the treatment is a standard of care, a checkpoint inhibitor, a PD-1 antagonist, or a PD-L1 antagonist; or any combination thereof. In some embodiments, the cancer is lung cancer, genitourinary cancer, or gastrointestinal cancer. In some embodiments, the cancer is lung cancer, such as non-small cell lung cancer (NSCLC), optionally wherein the cancer is (i) locally advanced, unresectable, locally advanced unresectable, or metastatic, (ii) no longer responding to a treatment, optionally wherein the treatment is a standard of care, a checkpoint inhibitor, a PD-1 antagonist, or a PD-L1 antagonist, or (iii) a combination of (i) and (ii). In some embodiments, the lung cancer is squamous or non-squamous, unresectable locally advanced disease or metastatic disease. In some embodiments, the cancer is gastrointestinal cancer, such as esophageal cancer, gastric cancer, colorectal cancer, pancreatic cancer or liver cancer, optionally wherein the cancer is (i) locally advanced, unresectable, locally advanced unresectable, or metastatic and/or (ii) no longer responding to a treatment, such as a standard of care, a checkpoint inhibitor, a PD-1 antagonist, or a PD-L1 antagonist. In some embodiments, the gastrointestinal cancer is esophageal cancer or gastric cancer, optionally wherein the cancer is (i) locally advanced, unresectable, locally advanced unresectable, or metastatic and/or (ii) no longer responding to a treatment, such as a standard of care. In some embodiments, the gastrointestinal cancer is esophageal adenocarcinoma, esophageal squamous cell carcinoma, gastroesophageal junction adenocarcinoma, or gastric adenocarcinoma, optionally wherein the cancer is (i) locally advanced, unresectable, locally advanced unresectable, or metastatic and/or (ii) no longer responding to a treatment, such as a standard of care. In some embodiments, the cancer is NSCLC, head and neck squamous cell carcinoma (HNSCC), renal cell carcinoma (RCC), breast cancer, colorectal cancer (CRC), melanoma, bladder cancer, ovarian cancer, endometrial cancer, Merkel Cell, or gastroesophageal cancer. In some embodiments, the subject is check point inhibitor (CPI) naïve. Alternatively, in some embodiments, the subject is CPI experienced.

In some embodiments of the foregoing aspects and embodiments, the cancer may be PD-L1 positive as measured by a clinically validated PD-L1 IHC assay or FDA-approved test. Cut-offs for PD-L1 positivity vary depending upon the test and the tumor.

In some embodiments of the foregoing aspects and embodiments, PD-L1 expression of the cancer is Tumor Proportion Score (TPS) ≥50%, as measured by a clinically validated PD-L1 IHC assay or FDA-approved test. In some embodiments of the foregoing aspects and embodiments, PD-L1 expression of the cancer is % TC ≥50%, as measured by a clinically validated PD-L1 IHC assay or FDA-approved test.

In some embodiments of the foregoing aspects and embodiments, the PD-L1 expression of the cancer corresponds to TPS <50% as measured by a clinically validated PD-L1 IHC assay or FDA-approved test. In some embodiments, PD-L1 expression of the cancer is about 1-10%, about 10%-20%, about 20-30%, about 30-40%, about 40-49%, about 1-49%, about 1-25%, or about 25-49%, as measured by a clinically validated PD-L1 IHC assay or FDA-approved test. In some embodiments, PD-L1 expression of the cancer is <1%, as measured by a clinically validated PD-L1 IHC assay or FDA-approved test.

In some embodiments of the foregoing aspects and embodiments, PD-L1 expression of the cancer is CPS ≥1, CPS ≥5, or CPS ≥10, as measured by a clinically validated PD-L1 IHC assay or FDA-approved test.

In some embodiments of the foregoing aspects and embodiments, PD-L1 expression of the cancer is TAP ≥1%, TAP ≥5%, or TAP ≥10%, as measured by a clinically validated PD-L1 IHC assay or FDA-approved test.

In some embodiments of the foregoing aspects and embodiments, the cancer is tumor mutational burden-high (TMB-H; ≥10 mutations/megabase (mut/Mb), as determined by an FDA-approved test.

In some embodiments of the foregoing aspects and embodiments, the cancer is not TMB-H.

In some embodiments of the foregoing aspects and embodiments, the cancer does not have an actionable mutation in an oncogene, e.g., a mutation for which a targeted therapy is approved by local health authority and available for use. In some embodiments of the foregoing aspects and embodiments, the cancer does not have an actionable oncogenic mutation in ALK, EGFR, ROS, BRAF, or NTRK and/or has wildtype ALK, EGFR, ROS, BRAF, and/or NTRK.

In some embodiments of the foregoing aspects and embodiments, the cancer expresses or overexpresses one or more biomarker selected CD73, DNAM-1, PVR, TIGIT, and CD8-Ki67.

In some embodiments of the foregoing aspects and embodiments, treatment results in a reduction in tumor size, a reduction in tumor number, a reduction in metastasis, stable disease, partial response, complete response, or a combination thereof.

In some embodiments of the foregoing aspects and embodiments, the treatment results in an improvement in overall survival, progression-free survival, disease control rate, overall response rate, or a combination thereof compared to placebo or a standard of care.

In some embodiments of the foregoing aspects and embodiments, the treatment results in an increased time to progression, increased disease-free survival, increased duration of response, an increase in duration of clinical benefit, an increase in time to treatment failure, a decrease in time to initial response, or any combination thereof, compared to placebo or a standard of care.

In some embodiments of the foregoing aspects and embodiments, the anti-TIGIT antibody that has reduced binding to one or more activating human FcγRs as compared to wild type (WT) human IgG1 is formulated for dilution as an aqueous solution comprising about 10 mg/mL to about 100 mg/mL antibody or about 20 mg/mL to about 60 mg/mL antibody, a buffer containing about 15 to about 30 mM histidine (His)/histidine-Cl (His-Cl), about 4% to about 10% (weight/volume) of an excipient selected from the group consisting of sucrose, dextrose, trehalose, sorbitol, and mannitol, about 0 mg/mL to about 10 mg/mL NaCl, and about 0.05 mg/mL to about 0.6 mg/mL polysorbate 80.

In some embodiments of the foregoing aspects and embodiments, the anti-TIGIT antibody that has reduced binding to one or more activating human FcγRs as compared to wild type (WT) human IgG1 is formulated for dilution as an aqueous solution comprising about 10 mg/mL to about 100 mg/mL antibody or about 20 mg/mL to about 60 mg/mL antibody, a buffer containing about 15 to about 25 mM His/His-Cl, about 5% to about 10% (weight/volume) of an excipient selected from the group consisting of sucrose, dextrose, and mannitol, about 0 mg/mL to about 10 mg/mL NaCl, and about 0.1 mg/mL to about 0.3 mg/mL polysorbate 80.

The foregoing general description and following detailed description are exemplary and explanatory and are intended to provide further explanation of the disclosure as claimed. Other objects, advantages, and features will be apparent to those skilled in the art from the following brief description of the drawings and detailed description of the disclosure.

### BRIEF DESCRIPTION OF THE DRAWINGS

FIG. 1A, FIG. 1B, FIG. 1C, FIG. 1D, and FIG. 1E show results of an ELISA assay to illustrate that domvanalimab does not bind to human Fcγ receptors (FcγR), specifically FcγR 1 (FIG. 1A), FcγR 2A (FIG. 1B), FcγR 2B (FIG. 1C), FcγR 3A (FIG. 1D), and FcγR 3B (FIG. 1E). In each figure, circles=human IgG1, squares=domvanalimab, and triangles=buffer only.

FIG. 2 shows NK-mediated ADCC against T_{reg} or CD8⁺ target cells isolated from peripheral blood mononuclear cell suspensions with representative histograms showing TIGIT expression. Paired symbols indicate treatment with human IgG isotype (square) or anti-TIGIT antibodies (circle) tiragolumab (“Tira”) or domvanalamib (“Dom”) for each NK-T cell donor pair. ****denotes P<0.0001.

FIG. 3A is an illustration showing the principle of a receptor occupancy (RO) assay.

FIG. 3B is exemplary flow cytometry data showing the principle of an RO assay.

FIG. 4A, FIG. 4B, and FIG. 4C shows the evaluation of TIGIT, CD226 and CD155 expression in the tumor microenvironment. FIG. 4A shows CD155 and PD-L1 ligand expression on CD14⁺ monocytes (CD45⁺CD3⁻CD16⁻CD56⁻CD14⁺) and cancer/stromal cells (CD45⁻) and co-expression of PD-1, TIGIT, and CD226 on broad T cell populations. FIG. 4B depicts a contour plot (NSCLC subject) of PD-1 expression on CD8⁺ T cells encompassing various states of activation and differentiation. Each population was further characterized into tissue resident (CD103⁺) and circulating (CD103⁻) CD8⁺ populations, resulting in six subsets. FIG. 4C shows co-expression of TIGIT and CD226 on the six CD8⁺ T cell populations from (B) and co-expression of PD-1, TIGIT, and CD226 on probable tumor-specific CD39⁺CD103⁺CD8⁺ T cells. Lines connect populations from the same subject. Each symbol represents a unique subject while bars and error denote median ±range.

FIG. 5A shows the percent change from baseline for measurable target lesions per RECIST 1.1 over time for two participants in a study for safety and tolerability of domvanalimab in combination with zimberelimab (Example 5).

FIG. 5B and FIG. 5C are images of scans demonstrating a decrease in target lesion size in the two partial responders from FIG. 5A. Scans show one of two target lesions with each participant having lesions not documented in the scans shown here.

FIG. 6 shows T cell absolute counts by individual (top row) or by cohort (middle row) or by study (bottom row) for 11 patients administered domvanalimab in combination with zimberelimab. Time is on the x-axis (D1=day 1, etc.). Treatment was initiated on DL. “D1 pre” are samples obtained on day 1 prior to the start of treatment and “D1 post” are samples obtained on day 1 after administration of the combination treatment. Percent change from baseline in the number of cells per 1 whole blood (WB) is on the y-axis. Patient dosing is indicated by the triangles on the x-axis. Whole blood samples were obtained at the times indicated and absolute cell counts measured as described in the Examples. The rectangular grey shading identifies the range measured in six healthy donors. AB 154=domvanalimab; AB122=zimberelimab.

FIG. 7A is an illustration depicting a representative gating strategy.

FIG. 7B shows T cell and NK cell frequencies measured in whole blood samples from two partial responders in the Example 5 trial. One patient had esophageal cancer and one patient had gastroesophageal junction (GEJ) cancer. Both patients were treated Q3W with domvanalimab (10 mg/kg) and zimberelimab (360 mg). Time is on the x-axis (C1D1=cycle 1 day 1, etc.). Treatment was initiated on D1. “D1 pre” are samples obtained on day 1 prior to the start of treatment and “C1D1 4 hr” are samples obtained on day 1 approximately 4 hours after administration of the combination treatment. Fold change from baseline is on the y-axis. Whole blood samples were obtained at the times indicated and cell counts measured as described in the Examples.

FIG. 8 shows T cell expansion was observed in peripheral blood samples obtained from patients treated with domvanalimab alone or in combination with zimberelimab or zimberelimab and etrumadenant.

FIG. 9A, FIG. 9B, and FIG. 9C show lymphoid cell expansion in patients treated with zimberelimab, zimberelimab and domvanalimab, or zimberelimab, domvanalimab and etrumadenant that were enrolled in a clinical study described in Example 7. The lymphoid cells are CD8⁺Ki67⁺ T cells (FIG. 9A), CD56⁺Ki67⁺NK cells (FIG. 9B), and memory CD39⁺CD103⁺CD8⁺ T cells (FIG. 9C). Time is on the x-axis, measured in days (D1=day 1, etc.). The treatment cycle is also identified on the x-axis (CID1=cycle 1 day 1, etc.). Treatment was initiated on D1. “C1D1 pre” are samples obtained on day 1 prior to the start of treatment and “C1D1 4 hr” are samples obtained on day 1, approximately 4 hours after administration of the treatment. Whole blood samples were obtained at the times indicated and cell counts measured as described in the Examples. Each line is an individual patient.

FIG. 10 depicts a swimmers plot including all intent to treat (ITT)-13 patients: Patients treated in Arm 1, zimberelimab (“Z”); Patients treated in Arm 2, domvanalimab and zimberelimab (“DZ”); Patients treated in Arm 3, etrumadenant, domvanalimab and zimberelimab (“EDZ”). 31 patients with a partial response (PR) remain on treatment and another 14 patients with stable disease (SD) remain on active treatment. Across all treatment arms, the time to initial response ranged from 1.2 to 14.6 months-scored with orange triangle for PR and purple box for SD. There are additional delayed responses in the domvanalimab-containing arms—observed as late as 14 months after initiating therapy. The median duration of response was not yet reached in any arm.

FIG. 11 depicts Kaplan Meier curves estimating progression free survival (PFS) across the 3 arms for ITT-13 patients. There was early separation of the curves at the first scan, as demonstrated by 6-month progression-free survival rates of 43% in Arm 1, 65% in Arm 2, and 63% in Arm 3. The median PFS was 5.4 months in Arm 1, 12 months in Arm 2, and 10.9 months in Arm 3. There was a reduction in probability of progression or death of 45% and 35% for Arms 2 and 3 respectively, relative to zimberelimab monotherapy.

FIG. 12 depicts a spider plot of 12 patients from Arm 1 that had crossed over to treatment with EDZ (Arm 3). At the time of the ITT-13 data cut, 5 patients remained on crossover treatment.

### DETAILED DESCRIPTION

Described herein are antibodies that bind to TIGIT (e.g., domvanalimab), and methods of using such antibodies for the treatment of a disease, such as cancer, in a subject. Provided herein is use of an anti-TIGIT antibody of the disclosure for the manufacture of a medicament for treating a disease, such as cancer. Also provided herein are treatments for cancer comprising administering to a human subject in need thereof an anti-TIGIT antibody (e.g., domvanalimab). For the purposes of this disclosure, the anti-TIGIT antibody is lacking an Fc effector function or has a reduced Fc effector function, for example due to an absence or reduction of binding to one or more human Fc gamma receptors (FcγRs) as compared to wild type (WT) human IgG1. In general, the one or more human FcγRs will be activating human FcγRs (e.g., FcγRI, FcγRIIA, FcγRIIIA). Fc effector functions associated with binding to activating FcγRs include antibody dependent-cellular cytotoxicity (ADCC), complement-dependent cytotoxicity (CDC), antibody-dependent cellular phagocytosis (ADCP), induction of cytokines/chemokines, and endocytosis of opsonized targets.

Treatment with such an anti-TIGIT antibody (e.g., domvanalimab) may not significantly reduce one or more peripheral lymphocyte populations, including for example regulatory T cells and/or CD8+ T cells. For example, any reduction may be within a normal range seen in healthy subjects, or not significantly different therefrom. Alternatively, or in addition, any reduction may be less than a reduction that occurs with an Fc-enabled anti-TIGIT antibody. Treatment with such an anti-TIGIT antibody (e.g., domvanalimab) may also result in a reduction of an adverse event, for example an immune-related adverse event, as compared to an Fc-enabled anti-TIGIT antibody whilst still achieving efficacy. Non-limiting examples of Fc-enabled anti-TIGIT antibodies include AB308, BMS-986207, tiragolumab, vibostolimab, etigilimab, ociperlimab, EOS-448, SEA-TGT, AGEN1777, AGEN1327, ralzapastotug, and JS006. In some individuals immune-related adverse events may be entirely avoided. Non-limiting examples of immune-related adverse events include rash, pruritis, maculo-papular rash, infusion related reactions, arthritis, psoriasis, swelling face, oral mucositis, dry mouth, colitis, diarrhea, immune-mediated hepatitis, pneumonitis, endocrinopathies, hypophysitis, hypothyroidism, hyperthyroidism, adrenal insufficiency, diabetes, and combinations thereof. Alternatively, or in addition, treatment with such an anti-TIGIT antibody may result in fewer treatment disruptions (including fewer dose reductions) and/or fewer treatment discontinuations for a patient or group of patients, as compared to an Fe-enabled anti-TIGIT antibody.

As described in more detail below, the present treatments and the positive outcomes they provide both in terms of efficacy and safety were unexpected and represent a divergence from the understanding in the art that human FcγR engagement and Fc effector function were required for an anti-TIGIT antibody to have clinical utility in humans. In contrast with that expectation, the present disclosure highlights that not only are anti-TIGIT antibodies with a reduced or abolished capacity to bind to one or more human FcγRs (e.g., activating human FcγRs) effective, they may be significantly safer than Fc-enabled anti-TIGIT antibodies. In addition to providing efficacy and safety benefits to patients, this therapeutic profile, which is described in more detail below, may facilitate additional clinical utility including, but not limited to, clinical setting, patient populations, combination therapies, and dosing regimen.

### I. Definitions

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein. Where ranges are provided, they are inclusive of the boundary values and individual values within the range. For instance, a range of 1 to 5 is inclusive of the boundary values 1 and 5, as well as the values 2, 3, and 4.

As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

Also as used herein, “and/or” refers to and encompasses any and all possible combinations of one or more of the associated listed items, as well as the lack of combinations when interpreted in the alternative (“or”).

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

The term “antibody,” as used herein, is used in the broadest sense and encompasses various antibody and antibody-like structures that specifically bind to a single antigen or to multiple antigens (e.g., monospecific antibodies, multispecific antibodies, polyepitopic antibodies, etc.), including but not limited to full-length antibodies, antigen-binding fragments, heavy chain antibodies, single-chain antibodies, and higher order variants of single-chain antibodies. Thus, any reference to an antibody should be understood to refer to the antibody in intact form or an antigen-binding fragment unless the context requires otherwise. Preferably, but not necessarily, antibodies useful herein are isolated and can be produced recombinantly.

The terms “full-length antibody,” “intact antibody,” and “whole antibody” are used herein interchangeably to refer to an antibody having a structure substantially similar to a native antibody structure or having heavy chains that contain an Fc region.

“Native antibodies” are naturally occurring immunoglobulin molecules with varying structures. For example, native IgG antibodies are heterotetrameric glycoproteins of about 150,000 daltons, composed of two identical light chains (each about 25 kDa) and two identical heavy chains (each about 50-70 kDa) that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (x) and lambda (k), based on the amino acid sequence of its constant domain. Heavy chains are classified as gamma, mu, alpha, delta, or epsilon, and define the antibody's isotype as IgG, IgM, IgA, IgD and IgE, respectively. The amino-terminal portion of each light and heavy chain includes a variable region of about 100 to 110 or more amino acid sequences primarily responsible for antigen recognition (VL and VH, respectively). The carboxy-terminal portion of each chain defines a constant region primarily responsible for effector function. Within light and heavy chains, the variable and constant regions are joined by a “J” region of about 12 or more amino acid sequences, with the heavy chain also including a “D” region of about 10 more amino acid sequences.

The term “variable region” or “variable domain” refers to the domain of an antibody heavy or light chain that is involved in binding the antibody to antigen. The variable domains of the heavy chain and light chain of an antibody generally have similar structures, with each domain comprising four conserved framework regions (FRs) and three hypervariable regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

“Framework region” or “FR” refers to variable domain residues other than hypervariable region residues. The FR of a variable domain generally consists of four FR domains: FR1, FR2, FR3, and FR4. Accordingly, the CDR and FR sequences generally appear in the following sequence: FR1-CDR1-FR2-CDR2-FR3-CDR3-FR4. The FR domains of a heavy chain and a light chain may differ, as is known in the art.

As used herein, the terms “hypervariable region” or “HVR”, also commonly referred to as “complementarity determining region” or “CDR”, are used interchangeably and refer to each of the regions of a variable domain which are hypervariable in sequence and/or form structurally defined loops (“hypervariable loops”) and/or contain the antigen-contacting residues (“antigen contacts”). Generally, antibodies comprise six CDRs: three in the VH (Hi, H2, H3), and three in the VL (L1, L2, L3). As used herein, “a CDR derived from a variable region” refers to a CDR that has no more than two amino acid substitutions, as compared to the corresponding CDR from the original variable region. Exemplary CDRs herein include: (a) hypervariable loops occurring at amino acid residues 26-32 (Li), 50-52 (L2), 91-96 (L3), 26-32 (Hi), 53-55 (H2), and 96-101 (H3) (Chothia and Lesk, J. Mol. Biol. 196:901-917 (1987)); (b) CDRs occurring at amino acid residues 24-34 (Li), 50-56 (L2), 89-97 (L3), 31-35b (H1), 50-65 (112), and 95-102 (113) (Kabat et al., Sequences of Proteins of Immunological Interest, 5^{th} Ed. Public Health Service, National Institutes of Health, Bethesda, MD (1991)); (c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and (d) combinations of (a), (b), and/or (c), as defined below for various antibodies of this disclosure. Unless otherwise indicated, CDR residues and other residues in the variable domain (e.g., FR residues) are Eu numbered herein according to Kabat et al., supra.

The term “isolated antibody” refers to an antibody that has been separated from a component of its natural environment. In some embodiments, an isolated antibody is purified to greater than 95% or 99% purity as determined by, for example, electrophoresis or chromatography (e.g., ion exchange or reverse phase HPLC).

The term “chimeric” antibody refers to an antibody in which a portion of the heavy and/or light chain is derived from a particular source or species, while the remainder of the heavy and/or light chain is derived from a different source or species.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter. J. Mol. Biol. 227:381, 1991; Marks et al. J. Mol. Biol. 222:581, 1991. Also available for the preparation of human monoclonal antibodies are methods described in Cole et al. Monoclonal Antibodies and Cancer Therapy, Alan R. Liss, p. 77 (1985); Boerner et al. J. Immunol., 147(1):86-95, 1991. See also van Dijk and van de Winkel. Curr. Opin. Pharmacol. 5:368-74, 2001.

A “humanized” antibody refers to an antibody comprising amino acid residues from non-human CDRs and amino acid residues from human FRs. In certain aspects, a humanized antibody will comprise variable domains, in which all or substantially all of the CDRs correspond to those of a non-human antibody, and all or substantially all of the FRs correspond to those of a human antibody. In certain aspects in which all or substantially all of the FRs of a humanized antibody correspond to those of a human antibody, any of the FRs of the humanized antibody may contain one or more amino acid residues from non-human FR(s), for example at one or more Vernier position residues of FRs, and/or at one or more other chosen residue. A humanized antibody optionally may comprise at least a portion of an antibody constant region derived from a human antibody. A “humanized form” of an antibody, e.g., a non-human antibody, refers to an antibody that has undergone humanization. A humanized antibody retains similar binding specificity and affinity as the starting non-human antibody.

The term “monoclonal antibody” refers to an antibody that is derived from a single copy or clone, including e.g., any eukaryotic, prokaryotic, or phage clone. The term “monoclonal antibody” is not limited to antibodies produced through hybridoma technology. Monoclonal antibodies can be produced using hybridoma techniques well known in the art, as well as recombinant technologies, phage display technologies, synthetic technologies or combinations of such technologies and other technologies readily known in the art.

The term “epitope” refers to the particular site on an antigen to which an antibody binds. The particular site on an antigen to which an antibody binds can be determined, for example, by crystallography. Methods such as hydroxyl radical protein footprinting and alanine scanning mutagenesis can also be used but may provide less resolution.

The term “Fc region” is used herein to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the Eu numbering system) of the Fc region may be removed, for example, during production or purification of the antibody, or by recombinantly engineering the nucleic acid encoding a heavy chain of the antibody. Accordingly, a composition of intact antibodies may comprise antibody populations with all Lys447 residues removed, antibody populations with no Lys447 residues removed, and antibody populations having a mixture of antibodies with and without the Lys447 residue.

A “functional Fc region” possesses an effector function of a native sequence Fc region. Exemplary effector functions include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding domain (e.g., an antibody variable domain) and can be assessed using various assays disclosed herein or otherwise known in the art. A functional Fc region may possess effector function substantially similar to a wild-type IgG, reduced effector function compared to a wild-type IgG, or enhanced effector function compared to a wild-type IgG. For antibodies comprising a human Fc region, the comparison is typically to a wild-type human IgG1.

As used herein, the term “Fc-enabled antibody” refers to an antibody that has similar or enhanced binding to human FcγR as compared to wild-type (WT) human IgG1 and triggers Fc effector functions. Fc effector functions include but are not limited to ADCC, CDC and ADCP.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification (e.g., from about one to about ten amino acid modifications, and in some embodiments from about one to about five amino acid modifications), preferably one or more amino acid substitution(s). The variant Fc region herein will preferably possess at least about 80% homology with a native sequence Fc region and/or with an Fc region of a parent polypeptide, preferably at least about 90% homology therewith, or preferably at least about 95% homology therewith. In some embodiments, variant Fc regions may possess reduced or enhanced effector function, as compared to a wild-type IgG. For antibodies comprising a human Fc region, the comparison is typically to a wild-type human IgG1.

“Fc component” as used herein refers to a hinge region, a CH2 domain or a CH3 domain of an Fc region.

“Hinge region” is generally defined as stretching from about residue 216 to 230 of an IgG (Eu numbering), from about residue 226 to 243 of an IgG (Kabat numbering), or from about residue 1 to 15 of an IgG (IMGT unique numbering).

The term “antibody fragment” refers to a molecule other than an intact antibody that comprises a portion of an intact antibody that binds the antigen to which the intact antibody binds. Examples of antigen-binding fragment include, without limitation, a diabody, a Fab, a Fab′, a F(ab′)₂, a F(ab)_{c}, an Fv fragment, a disulfide stabilized Fv fragment (dsFv), a (dsFv)₂, a bispecific dsFv (dsFv-dsFv′), a disulfide stabilized diabody (ds diabody), a triabody, a tetrabody, a single-chain antibody, an scFv, an scFv dimer, a single domain antibody, a single-domain antibody, and a multivalent domain antibody. Typically, binding fragments compete with the intact antibody from which they were derived for specific binding. Binding fragments can be produced by recombinant DNA techniques, or by enzymatic or chemical separation of intact immunoglobulins.

The term “Fab” refers to that portion of an antibody consisting of a single light chain (both variable and constant regions) bound to the variable region and first constant region of a single heavy chain by a disulfide bond.

The term “Fab′” refers to a Fab fragment that includes a portion of the hinge region.

The term “F(ab′)₂” refers to a dimer of Fab′. F(ab′)2 antibody fragments originally were produced as pairs of Fab′ fragments which have hinge cysteines between them. Other chemical couplings of antibody fragments are also known.

The term “Fv” refers to the smallest fragment of an antibody to bear the complete antigen binding site. An Fv fragment consists of the variable region of a single light chain bound to the variable region of a single heavy chain.

The term “single-chain antibody” refers to an antibody consisting of a heavy chain variable region and a light chain variable region connected by a linker. In most instances, but not all, the linker may be a peptide. The length of the linker varies depending upon the type of single-chain antibody. Covalently or non-covalently linking two or more single-chain antibodies together results in higher order forms. Single-chain antibodies, and their higher order forms, may include, but are not limited to, single-domain antibodies, multivalent domain antibodies, single chain variant fragments (scFvs), divalent scFvs (di-scFvs), trivalent scFvs (tri-scFvs), tetravalent scFvs (tetra-scFvs), diabodies, and triabodies and tetrabodies.

The terms “single-chain Fv antibody” and “scFv” are used herein interchangeably to refer to a single-chain antibody consisting of heavy variable region and a light chain variable region connected by a linker. In most instances, but not all, the linker may be a peptide. The linker peptide is preferably from about 5 to 30 amino acids in length, or from about 10 to 25 amino acids in length. Typically, the linker allows for stabilization of the variable domains without interfering with the proper folding and creation of an active binding site. In preferred embodiments, a linker peptide is rich in glycine, as well as serine or threonine. Covalently or non-covalently linking two or more scFvs together results in higher order forms di-scFvs, tri-scFvs, tetra-scFvs, etc. The antigen-binding sites of each scFv in a higher order form can target the same or different antigen or epitope.

The term “single-chain Fv-Fc antibody” or “scFv-Fc” refers to a full-length antibody consisting of a scFv connected to an Fc region.

A “diabody” is a higher order variant of a single-chain antibody consisting of two single-chain antibodies. For each single-chain antibody, a linker is used that is too short to allow pairing between the two domains on the same chain, forcing the domains to pair with the complementary domains of another chain, thereby creating two antigen-binding sites. In most instances, but not all, the linker may be a peptide. The antigen-binding sites can target the same or different antigens or epitopes. Triabodies (three single chain antibodies assembled to form three antigen-binding sites), tetrabodies (four single chain antibodies assembled to form four antigen-binding sites), and higher order variants can similarly be produced. See, for example, Holliger P. et al., Proc Natl Acad Sci USA. July 15; 90(14):6444-8 (1993); EP404097; WO93/11161.

A “single-domain antibody” refers to an antibody fragment containing only the variable region of a heavy chain or the variable region of a light chain. In certain instances, two or more V_{H} domains are covalently joined with a peptide linker to create a multivalent domain antibody. The two or more V_{H} domains of a multivalent domain antibody can target the same or different antigens or epitopes.

The term “heavy chain antibody” refers to an antibody that consists of two heavy chains. A heavy chain antibody may be an IgG-like antibody from camels, llamas, alpacas, sharks, etc., or an IgNAR from a cartilaginous fish. See, for example, Riechmann L. and Muyldermans S., J Immunol Methods. December 10; 231(1-2): 25-38 (1999); Muyldermans S., J Biotechnol. June; 74(4):277-302 (2001); WO94/04678; WO94/25591; or U.S. Pat. No. 6,005,079. Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V. K. et al. “Heavy-chain antibodies in Camelidae; a case of evolutionary innovation,” Immunogenetics. April; 54(1):39-47 (2002); Nguyen V. K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody that consists of a VHH domain from a heavy chain antibody and two constant domains, CH2 and CH3.

“Percent (%) identity” with respect to a reference amino acid sequence is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, or CLUSTAL software. Those skilled in the art can determine appropriate parameters for aligning sequences, including any algorithms needed to achieve maximal alignment over the full length of the sequences being compared. Generally speaking, the % sequence identity of a given amino acid sequence A to, with, or against a given amino acid sequence B is calculated as follows: 100 times the fraction X/Y where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

As used herein, the phrase “therapeutically effective amount” with reference to an anti-TIGIT antibody means a dose regimen (i.e., amount and interval) of the antibody that provides the specific pharmacological effect for which the drug is administered in a subject in need of such treatment. For prophylactic use, a therapeutically effective amount may be effective to eliminate or reduce the risk, lessen the severity, or delay the onset of the disease, including biochemical, histological and/or behavioral signs or symptoms of the disease. For treatment, a therapeutically effective amount may be effective to reduce, ameliorate, or eliminate one or more signs or symptoms associated with a disease, delay disease progression, prolong survival, decrease the dose of other medication(s) required to treat the disease, or a combination thereof. With respect to cancer specifically, a therapeutically effective amount may, for example, result in the killing of cancer cells, reduce cancer cell counts, reduce tumor burden, eliminate tumors or metastasis, or reduce metastatic spread. It is emphasized that a therapeutically effective amount of an anti-TIGIT antibody will not always be effective in treating cancer in every individual subject, even though such dose is deemed to be a therapeutically effective amount by those of skill in the art. A therapeutically effective amount may vary based on, for example, the age and weight of the subject, and/or the subject's overall health, the stage of the subject's cancer, the route of administration, and prior or concomitant treatments.

The terms “treat,” “treatment,” or “treating” as used herein with reference to cancer refer to a course of action that eliminates, reduces, suppresses, mitigates, ameliorates, or prevents the worsening of, either temporarily or permanently, a disease, disorder or condition to which the term applies, or at least one of the symptoms associated therewith. Treatment includes alleviation of symptoms, diminishment of extent of disease, inhibiting (e.g., arresting the development or further development of the disease, disorder or condition or clinical symptoms association therewith) an active disease, delaying or slowing of disease progression, improving the quality of life, and/or prolonging survival of a subject as compared to expected survival if not receiving treatment or as compared to a published standard of care therapy for a particular disease.

The term “in need of treatment” as used herein refers to a judgment made by a physician or other caregiver that a subject requires or will benefit from treatment. For example, “treating cancer in a subject in need of treatment”, sometimes phrased as “treating cancer in a subject in need thereof”, refers to treating a subject that a physician or other caregiver has judged requires or will benefit from treatment. This judgment is made based on a variety of factors that are in the realm of the physician's or caregiver's expertise, which may include a positive diagnosis of a disease, disorder or condition. In some examples, a physician or other caregiver may determine a subject requires or will benefit from treatment with an anti-TIGIT antibody. In these instances, an additional decision is still required for effective treatment, namely which anti-TIGIT antibody or group of anti-TIGIT antibodies are suitable.

The terms “prevent”, “preventing”, “prevention”, “prophylaxis” and the like refer to a course of action initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state. Prevention also refers to a course of action initiated in a subject after the subject has been treated for a disease, disorder, condition or a symptom associated therewith in order to prevent relapse of that disease, disorder, condition or symptom.

The term “in need of prevention” as used herein refers to a judgment made by a physician or other caregiver that a subject requires or will benefit from preventative care. This judgment is made based on a variety of factors that are in the realm of a physician's or caregiver's expertise.

The terms “individual,” “subject,” and “patient” are used interchangeably herein, and refer to any individual human.

### II. TIGIT and Tregs

TIGIT (also called T cell immunoreceptor with Ig and ITIM domains) is an immune receptor present on some T cells and natural killer cells (NK). TIGIT is considered an immune checkpoint. Human TIGIT has a sequence according to SEQ ID NO: 1 below.

(SEQ ID NO: 1)
MMTGTIETTGNISAEKGGSIILQCHLSSTTAQVTQVNWEQQDQLLAICN
ADLGWHISPSFKDRVAPGPGLGLTLQSLTVNDTGEYFCIYHTYPDGTYT
GRIFLEVLESSVAEHGARFQIPLLGAMAATLVVICTAVIVWALTRKKKA
LRIHSVEGDLRRKSAGQEEWSPSAPSPPGSCVQAEAAPAGLCGEQRGED
CAELHDYFNVLSYRSLGNCSFFTETG.

Regulatory T cells (Tregs) are a specialized subpopulation of T cells that act to suppress the immune response, thereby maintaining homeostasis and self-tolerance. Tregs are immunosuppressive, and generally suppress or downregulate induction and proliferation of effector T cells. Many subtypes of Tregs exist, with the most well-understood being those that express CD4, CD25, and FOXP3 (CD4⁺CD25⁺FOXP3⁺Tregs).

### III. Anti-TIGIT Antibodies

Target-binding specificity, imparted by an antibody's variable region, is well-known to be necessary for the primary functional activities of a given antibody. For anti-TIGIT antibodies, several recent studies have also suggested the importance of FcγR co-engagement by anti-TIGIT antibodies to promote immune activation and tumor control. In particular, it was believed that depletion of Treg cells by antibody-dependent cell cytotoxicity (ADCC) through FcγR co-engagement by anti-TIGIT antibodies is required for meaningful efficacy (see, e.g., Chen et al. Front. Immunol, 2019, 10: 292; Ward et al., BMJ, 2020, 8(Suppl. 3), A253; Johnston et al., Ann Rev. Cancer Biol., 2021, 5:203-19). Indeed, Chen et al. (Front. Immunol, 2019, 10: 292) states, “for targets such as CTLA-4, TIGIT, and VISTA, competent Fc is required for optimal anti-tumor immune responses in various mouse models.” Ward et al. (BMJ, 2020, 8(Suppl. 3), A253) similarly reported “[b]lockade of FcγRIIIA or depletion of CD14+ and CD56+ cells reduced the functional activity of the Fc-enhanced anti-TIGIT antibody, confirming the requirement for FcγR co-engagement to maximize T cell responses.” Finally, Johnson et al. (Ann Rev. Cancer Biol., 2021, 5:203-19) reported “[t]here is an emerging consensus that Fc effector function also plays a role in TIGIT antibody activity.”

As a result, most anti-TIGIT antibodies currently in development are Fc-enabled meaning the antibody possesses wild-type or Fc-enhanced IgG1 constant regions that bind human FcγRI, human FcγRIIA, and human FcγRIIIA with moderate to high affinity and trigger Fc effector functions such as ADCC, CDC and ADCP. These antibodies include, but are not limited to tiragolumab, vibostolimab, etigilimab, ociperlimab, EOS-448, SEA-TGT, AGEN1777, AGEN1327, and JS006. Evidence of Treg depletion after administration has been shown for at least two of these antibodies—EOS-448 (Van den Mooter et al. “Preliminary data from Phase I first LGO—in-human study of EOS884448, a novel potent anti-TIGIT antibody, monotherapy shows favorable tolerability profile and early signs of clinical activity in immune-resistant advanced cancers;” Poster presented at: AACR Annual Meeting 2021: Apr. 10-15, 2021) and SEA-TGT (Smith A, et al., “SEA-TGT is an empowered anti-TIGIT antibody that displays superior combinatorial activity with several therapeutic agents. Poster presented at: AACR Annual Meeting 2021; Apr. 10-15, 2021). Despite the perspective in the art that Fc-binding is needed for anti-TIGIT antibodies to have clinical utility, the present disclosure relies on the use of anti-TIGIT antibodies with reduced or abolished Fc function, in particular reduced or abolished ADCC, CDC and/or ADCP.

Therapeutic antibodies as described herein, for the purposes of the disclosed methods and pharmaceutical compositions, are antibodies or fragments thereof that bind to human TIGIT and inhibit binding of CD155 to TIGIT, but the specific anti-TIGIT antibody is not limited to any one antibody's antigen binding domain. Domvanalimab is a preferred anti-TIGIT antibody, but the antigen binding domain from other anti-TIGIT antibodies (e.g., tiragolumab, vibostolimab, etigilimab, ociperlimab, EOS-448, SEA-TGT, AGEN1777, AGEN1327, JS006, AB308, etc.), or an antigen binding domain comprising at least the 6 CDRs of these anti-TIGIT antibodies, can be used as well. A therapeutic antibody suitable for use in the disclosed methods and pharmaceutical uses may be human, humanized, or chimeric, and it may be a full-length antibody or an antibody fragment. In some embodiments, a therapeutic antibody suitable for use in the disclosed methods and pharmaceutical uses comprises an Fc region. Those antibodies comprising an Fc region may be an IgA, IgG (i.e., IgG1, IgG2, IgG3, and IgG4), IgD, IgE, or IgM antibody or variant thereof, though in all instances the Fc region has reduced binding to one or more FcγRs (e.g., activating FcγRs) as compared to WT IgG1 or an inability to bind one or more FcγRs (e.g., activating FcγRs).

Anti-TIGIT antibodies of the present disclosure have an equilibrium dissociation constant (KD) of 10⁻⁸ M or lower for human TIGIT, measured by surface plasmon resonance (SPR). For example, the anti-TIGIT antibodies of the present disclosure may have a KD for TIGIT of 10⁻⁸ M or lower, 10⁻⁹ M or lower, 10⁻¹⁰ M or lower, 10⁻¹¹ M or lower, 10⁻¹² M or lower, or 10⁻¹³ M or lower, measured by SPR. In various embodiments, the anti-TIGIT antibodies of the present disclosure have a KD for TIGIT in the range of about 1×10⁻⁹ M to about 1×10⁻³ M, or about 1×10⁻⁹ M to about 1×10⁻¹² M, or about 1×10⁻¹⁰ M to about 1×10⁻¹³ M, or about 1×10⁻¹⁰ M to about 1×10⁻¹² M, or about 1×10⁻¹¹ M to about 1×10⁻¹³ M, or about 1×10⁻¹⁰ M to about 1×10⁻¹¹ M, or about 1×10⁻¹¹ M to about 1×10⁻¹² M.

In one embodiment, an anti-TIGIT antibody of the present disclosure comprises a heavy chain variable domain comprising CDRH1, CDRH2 and CDRH3 of domvanalimab, and a light chain variable domain comprising CDRL1, CDRL2 and CDRL3 of domvanalimab. Domvanalimab comprises a CDRH1 comprising the amino acid sequence of NFGMH (SEQ ID NO. 2), a CDRH2 comprising the amino acid sequence of FISSGSSSIYYADTVKG (SEQ ID NO: 3), a CDRH3 comprising the amino acid sequence of MRLDYYAMDY (SEQ ID NO: 4), a CDRL1 comprising the amino acid sequence of RASKSISKYLA (SEQ ID NO: 5), a CDRL2 comprising the amino acid sequence of SGSTLQS (SEQ ID NO: 6), and a CDRL3 of QQHNEYPWT (SEQ ID NO: 7).

In another embodiment, an anti-TIGIT antibody of the present disclosure comprises a heavy chain variable domain comprising the heavy chain variable domain of domvanalimab and a light chain variable domain comprising the light chain variable domain of domvanalimab. Domvanalimab comprises a variable heavy chain (VH) domain comprising the amino acid sequence of EVQLVESGGGLVQPGGSLRLSCAASGFTFSNFGMHWVRQAPGKGLEWVAFISSGSSSIY YADTVKGRFTISRDNAKNSLYLQMNSLRAEDTAVYYCARMRLDY YAMDYWGQGTMV TVSS (SEQ ID NO: 10) and a variable light chain (VL) domain comprising the amino acid sequence of DIQMTQSPSSLSASVGDRVTITCRASKSISKYLAWYQQKPGKAPKLLIYSGSTLQSGVPS RFSGSGSGTDFTLTISSLQPEDFATYYCQQHNEYPWTFGGGTKVEIK (SEQ ID NO: 11). These VH and VL domains are humanized variants of EVQLQQSGPELVKPGASVKISCKTSGYTFTEYTMHWVKQSHGKNLEWIGGINPNNGGTS YNQKFKGRATLTVDKSSSTAYMELRSLTSDDSAVYYCARPGWYNYAMDYWGQGTSV TVSS (SEQ ID NO: 8) and DVQITQSPSYLAASPGETITINCRASKSISKYLAWYQEKPGKTNKLLIYSGSTLQSGIPSRF SGSGSGTDFTLTISSLEPEDFAMYYCQQHNEYPWTFGGGTKLEIK (SEQ ID NO: 9), respectively.

In another embodiment, an anti-TIGIT antibody of the present disclosure is domvanalimab. The mature gamma heavy chain amino acid sequence of domvanalimab is:

(SEQ ID NO: 12)
EVQLVESGGGLVQPGGSLRLSCAASGFTFSNFGMHWVRQAPGKGLEWVA
FISSGSSSIYYADTVKGRFTISRDNAKNSLYLQMNSLRAEDTAVYYCAR
MRLDYYAMDYWGQGTMVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPSVFLF
PPKPKDTLMISRTPEVTCWVDVSHEDPEVKFNWYVDGVEVHNAKTKPRE
EQYNSTYRWSVLTVLHQDWLNGKEYKCKVSNKALPAPIEKTISKAKGQP
REPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSL
SLSPGK;

and the mature kappa light chain amino acid sequence of domvanalimab is:

(SEQ ID NO: 13)
DIQMTQSPSSLSASVGDRVTITCRASKSISKYLAWYQQKPGKAPKLLIY
SGSTLQSGVPSRFSGSGSGTDFTLTISSLQPEDFATYYCQQHNEYPWTF
GGGTKVEIKRTVAAPSVFIFPPSDEQLKSGTASWCLLNNFYPREAKVQW
KVDNALQSGNSQESVTEQDSKDSTYSLSSTLTLSKADYEKHKVYACEVT
HQGLSSPVTKSFNRGEC.

In some embodiments, an anti-TIGIT antibody of the present disclosure is a variant of domvanalimab. For the purposes of this disclosure “variant antibodies” or “variant” of domvanalimab may include, but are not limited to: (i) antibodies with heavy chains comprising at least 55%, at least 65%, at least 70%, at least 75%, at least 80%, at least 85%, at least 90%, at least 95%, at least 97%, at least 98%, at least 99%, or 100% amino acid sequence identity to domvanalimab's heavy chain sequence, (ii) antibodies with light chains comprising at least 55%, at least 65%, at least 70%, at least 75%, at least 80%, at least 85%, at least 90%, at least 95%, at least 97%, at least 98%, at least 99%, or 100% amino acid sequence identity to domvanalimab's light chain sequence, (iii) antibodies with variable regions comprising at least 55%, at least 65%, at least 70%, at least 75%, at least 80%, at least 85%, at least 90%, at least 95%, at least 97%, at least 98%, at least 99%, or 100% amino acid sequence identity to domvanalimab's variable region sequences, (iv) antibodies with CDRs comprising at least 55%, at least 65%, at least 70%, at least 75%, at least 80%, at least 85%, at least 90%, at least 95%, at least 97%, at least 98%, at least 99%, or 100% amino acid sequence identity to domvanalimab's CDR sequences, and (v) combinations thereof. For example, suitable variants include immunoglobulins or immunoglobulin-like molecules with the same or substantially similar heavy and light chain amino acid sequences as domvanalimab. Other suitable therapeutic antibodies may bind to the same isoform of TIGIT as domvanalimab (e.g., TIGITv3), optionally the same epitope of TIGIT, block or neutralize TIGIT, or combinations thereof. Additional exemplary therapeutic antibodies are described, for example, in U.S. Pat. No. 10,537,633.

In another embodiment, an anti-TIGIT antibody of the present disclosure comprises a light chain variable domain and heavy chain variable domain of an anti-TIGIT antibody disclosed in WO2017053748, WO2016028656, WO2016106302, WO2016191643, WO2018102536, WO2019129261, WO2019023504, WO2020144178, WO2018033798, WO2018160704, WO2020041541, WO2020020281, WO2019154415, WO2018234793, WO2021008523, WO2019168382, WO2020098734, WO2017059095, WO2020251187, WO2019129221, WO2021043206, WO2018128939, WO2020242919, WO2021216468, or WO2017152088. In some embodiments, the anti-TIGIT antibody of the present disclosure comprises a light chain variable domain and heavy chain variable domain of an anti-TIGIT antibody disclosed in WO2017152088.

In another embodiment, an anti-TIGIT antibody of the present disclosure comprises a light chain variable domain and a heavy chain variable domain, wherein the amino acid sequences of the light chain variable domain and the heavy chain variable domain each have about 90%, 91%, 92%, 93%, 94%, 95%, 96%, 97%, 98%, 99% sequence identity to the light chain variable domain and the heavy chain variable domain amino acid sequences of an anti-TIGIT antibody selected from the group consisting of tiragolumab, vibostolimab, etigilimab, ociperlimab, EOS-448, SEA-TGT, AGEN1777, AGEN1327, JS006, COM902, IBI939, BGB-A1217, ASP8374, and M6223.

In another embodiment, an anti-TIGIT antibody of the present disclosure comprises a light chain variable domain and a heavy chain variable domain of an anti-TIGIT antibody selected from the group consisting of tiragolumab, vibostolimab, etigilimab, and ociperlimab, EOS-448, SEA-TGT, AGEN1777, AGEN1327, JS006, COM902, IB1I939, BGB-A1217, ASP8374, and M6223.

For the purposes of the disclosed treatments, methods, and uses, the epitope specificity with respect to binding TIGIT may vary. For example, in some embodiments, the anti-TIGIT antibody can bind to a TIGIT polypeptide on one or more amino acid residues comprising D51, wherein the TIGIT polypeptide has an amino acid sequence corresponding to SEQ ID NO: 1. In various embodiments of the present disclosure, an anti-TIGIT antibody may competitively inhibit binding of a reference antibody to human TIGIT, the reference antibody selected from the group consisting of domvanalimab, tiragolumab, vibostolimab, etigilimab, ociperlimab, EOS-448, SEA-TGT, AGEN1777, AGEN1327, and JS006, or selected from the group consisting of domvanalimab, tiragolumab, vibostolimab, etigilimab, and ociperlimab. An antibody is said to competitively inhibit binding of a reference antibody to human TIGIT if the antibody preferentially binds to that epitope to the extent that it blocks binding of the reference antibody to TIGIT by at least 50%, at least 60%, at least 70%, at least 80%, or at least 90%. Competitive inhibition can be determined by any method known in the art, for example, a competition flow assay. Briefly, a reference antibody and an isotype control can be directly conjugated to a fluorophore per manufacturer instructions. Cells expressing human TIGIT can be plated at a suitable density in a multi-well plate, and a dose response curve prepared by serial dilution of each antibody, incubation of each antibody concentration with the cells, and then mean fluorescence intensity of the fluorophore for the live single cell population by flow cytometry. EC₅₀ and EC₉₅ values can calculated using standard 4-parameter non-linear regression analysis. Competition can then be evaluated by using an unlabeled test antibody with the labeled reference antibody and measuring any change in mean fluorescence intensity.

As stated above, anti-TIGIT antibodies of the present disclosure have reduced binding to one or more FcγRs (e.g., activating FcγRs) as compared to WT IgG1 or an inability to bind one or more FcγRs (e.g., activating FcγRs). Binding to FcγRs can be directly measured by the methods detailed in Example 1. Alternatively, or in addition, reduced binding to an activating FcγR can be demonstrated by showing an antibody has reduced Fc effector function, such as CDC, ADCC, and/or ADCP compared to WT IgG1.

In some embodiments, the anti-TIGIT antibody used in the disclosed methods and treatments is an IgG, such as an IgG1 (e.g., an Fc-modified IgG1) or an IgG4. The four subclasses of IgG-IgG1, IgG2, IgG3, and IgG4—are highly conserved. The amino acid sequence of the constant regions of these peptides are known in the art, e.g., see Rutishauser, U. et al. (1968) “Amino acid sequence of the Fc region of a human gamma G-immunoglobulin” PNAS 61(4):1414-1421; Shinoda et al. (1981) “Complete amino acid sequence of the Fc region of a human delta chain” PNAS 78(2):785-789; and Robinson et al. (1980) “Complete amino acid sequence of a mouse immunoglobulin alpha chain (MOPC 511)” PNAS 77(8):4909-4913.

Domvanalimab is an example of an anti-TIGIT antibody with a reduced or abolished capacity to bind FcγRs, particularly activating FcγRs like FcγRI, FcγRIIA, and FcγRIIIA. Domvanalimab, for example, has an engineered IgG1 Fe and reduced binding to one or more FcγRs (e.g., activating FcγRs) as compared to WT IgG1. Domvanalimab was tested by enzyme-linked immunosorbent assay for binding to FcγR isotypes I, IIA, IIB, IIIA, and IIIB. When compared with a wild-type IgG1 control antibody, no significant binding was observed with domvanalimab for any FcγR isotype when tested up to a maximum concentration of 1 μM. Domvanalimab was also tested in an FcγR-IIIA (V158 high-affinity variant) effector reporter bioassay and found to be inactive at concentrations up to 1 μM. A CDC assay was performed with human complement and a Jurkat cell line stably overexpressing human TIGIT in the presence of domvanalimab at increasing concentrations up to 33 nM. No cytotoxicity was seen for domvanalimab at any concentration tested.

Other antibodies that bind to TIGIT and possess either reduced or abolished capacity to bind FcγRs (e.g., activating FcγRs) can include, but are not limited to, IgG4 antibodies or IgG1 antibodies with a modified Fc region. For example, anti-TIGIT antibodies of the present disclosure may be engineered by introduction of constant regions with mutation(s) that result in reduced Fc effector functions, such as CDC and ADCC or ADCP, compared with the same antibody without the mutation(s). Preferably, each or a combination of these Fc effector functions are reduced at least 50%, 75%, 90% or 95% compared with antibodies without the mutation. Measuring CDC may be accomplished according to U.S. Pat. No. 10,537,633. Other assays for measuring CDC are described by Shields et al, 2001 J. Biol. Chem., Vol. 276, p 6591-6604; Chappel et al, 1993 J. Biol. Chem., Vol 268, p 25124-25131; Lazar et al, 2006 PNAS, 103; 4005-4010.

For example, the Fc region of tiragolumab, vibostolimab, etigilimab, ociperlimab, EOS-448, SEA-TGT, AGEN1777, AGEN1327, or JS006 may be engineered to contain a mutation that results in reduced Fc effector function. Substitution of any or all of positions 234, 235, 236 and/or 237 reduces affinity for Fcγ receptors, particularly FcγRI receptor (see, e.g., U.S. Pat. No. 6,624,821). Alanine is a preferred residue for substitution and L234A/L235A is a preferred dual mutation to reduce Fc effector function. Other combinations of mutations with reduced Fe effector functions include L234A/L235A/G237A, E233P/L234V/L235A/AG236, A327G/A330S/P331S, K322A, L234A and L235A, L234F/L235E/P331S. Optionally, positions 234, 236 and/or 237 in human IgG2 are substituted with alanine and position 235 with glutamine. (see, e.g., U.S. Pat. No. 5,624,821.) Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 of IgG2 residues at positions 233-236 and IgG4 residues at positions 327, 330 and 331 greatly reduces ADCC and CDC (see, for example, Armour KL. Et al., 1999 Eur J Immunol. 29(8):2613-24; and Shields R L. et al., 2001. J Biol Chem. 276(9):6591-604). N297A, N297Q, or N297G (Eu numbering) mutations reduce glycosylation and thereby Fc effector functions. Other substitutions can also be made in the constant regions of antibodies of the present disclosure to reduce Fc effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA, 103:4005, 2006).

Anti-TIGIT antibodies suitable for the disclosed methods may be antibodies from which one or several amino acids at the amino or carboxyl terminus of the light and/or heavy chain, such as the C-terminal lysine of the heavy chain, may be missing or derivatized in a proportion or all of the molecules. Substitutions can be made in the constant regions of antibodies of the present disclosure to prolong half-life in humans (see, e.g., Hinton et al., J. Biol. Chem. 279:6213, 2004). Exemplary substitutions include a Gln at position 250 and/or a Leu at position 428 (Eu numbering) for increasing the half-life of an antibody.

In some embodiments, the antibody is a monoclonal antibody. In some embodiments of any of the embodiments disclosed herein, the antibody is chimeric, humanized, or veneered. In some embodiments, the antibody is a humanized antibody. In some embodiments, the antibody is a human antibody.

Variants of domvanalimab and suitable therapeutic antibodies may be monoclonal or polyclonal antibodies, though are preferably monoclonal. Such monoclonal antibodies having TIGIT-binding and/or neutralizing activity can be obtained, for example, by the following procedure: anti-TIGIT monoclonal antibodies can be prepared by using as an antigen TIGIT or a fragment thereof that is derived from a mammal, such as human, by known methods, and then antibodies having TIGIT-binding and/or neutralizing activity are selected from the thus obtained anti-TIGIT monoclonal antibodies. Specifically, a desired antigen or cells expressing the desired antigen are used as a sensitizing antigen for immunization according to conventional immunization methods. Anti-TIGIT monoclonal antibodies can be prepared by fusing the obtained immune cells with known parental cells using conventional cell fusion methods, and screening them for monoclonal antibody-producing cells (hybridomas) by conventional screening methods. Animals to be immunized include, for example, mammals such as mice, rats, rabbits, sheep, monkeys, goats, donkeys, cows, horses, and pigs. The antigen can be prepared using the known TIGIT gene sequence according to known methods, for example, by methods using baculovirus (for example, WO 98/46777). Variants of domvanalimab and suitable therapeutic antibodies may also include intrabodies, peptibodies, nanobodies, single domain antibodies, multi-specific antibodies (e.g., bispecific antibodies, diabodies, triabodies, tetrabodies, tandem di-scFV, tandem tri-scFv), darpins, heavy chain monomers, heavy chain dimers, or single-domain antibodies (i.e., a V_{H}H fragment or a “camelid-like” antibody), any of which may be derived from the sequence and/or binding domain of domvanalimab.

Hybridomas can be prepared, for example, according to the method of Milstein et al. (Kohler, G. And Milstein, C., Methods Enzymol. (1981) 73: 3-46). When the immunogenicity of an antigen is low, immunization may be performed after linking the antigen with a macromolecule having immunogenicity, such as albumin. Antigens used to prepare monoclonal antibodies that have a binding and/or neutralizing activity against human TIGIT are not particularly limited, as long as they enable preparation of antibodies that have a binding and/or neutralizing activity against human TIGIT. For example, it is known that there are a number of variants of human TIGIT, and any variant may be used as an immunogen as long as it enables preparation of antibodies that have a binding and/or neutralizing activity against human TIGIT. Alternatively, under the same condition, a peptide fragment of TIGIT or a protein in which artificial mutations have been introduced into the natural TIGIT sequence may be used as an immunogen. Suitable immunogens that may be used in preparing antibodies that have an activity of binding and/or neutralizing TIGIT in the present disclosure are described in Example 2 of U.S. Pat. No. 10,537,633.

The TIGIT-binding activity of therapeutic antibodies can be determined by methods known to those skilled in the art. Methods for determining the antigen-binding activity of an antibody include, for example, ELISA (enzyme-linked immunosorbent assay), EIA (enzyme immunoassay), RIA (radioimmunoassay), and fluorescent antibody method. For example, when enzyme immunoassay is used, antibody-containing samples, such as purified antibodies and culture supernatants of antibody-producing cells, are added to antigen-coated plates. A secondary antibody labeled with an enzyme, such as alkaline phosphatase, is added and the plates are incubated. After washing, an enzyme substrate, such as p-nitrophenyl phosphate, is added, and the absorbance is measured to evaluate the antigen-binding activity. The binding and/or neutralizing activity of a therapeutic antibody against TIGIT can be measured, for example, by observing the effect of suppressing the growth of the TIGIT-dependent cell line. Other methods for determining TIGIT-binding activity include receptor occupancy (RO) assays. For example, RO may be assessed by flow cytometry using pre- and post-dose whole blood samples. A commercially available anti-TIGIT antibody that is competitive (i.e., a competitive antibody) with the anti-TIGIT antibody of interest (e.g., domvanalimab) may be used to determine a TIGIT RO, measured by a decrease in the detectable anti-TIGIT antibody signal that occurs following dosing of the antibody of interest (e.g., domvanalimab). A competitive antibody may be any antibody that competes for binding of an epitope targeted by the antibody of interest (e.g., domvanalimab).

### IV. Combinations with a Disclosed Anti-TIGIT Antibody

The present disclosure contemplates the use of anti-TIGIT antibodies with reduced or abolished Fc effector function (e.g., domvanalimab and other anti-TIGIT antibodies of Section III) alone or in combination with one or more additional therapy. Each additional therapy can be a therapeutic agent or another treatment modality. In embodiments comprising one or more additional therapeutic agents, each agent may target a different, but complementary, mechanism of action. The additional therapeutic agents can be small chemical molecules; macromolecules such as proteins, antibodies, peptibodies, peptides, DNA, RNA or fragments of such macromolecules; or cellular or gene therapies. Non-limiting examples of additional treatment modalities include surgical resection of a tumor, bone marrow transplant, radiation therapy, and photodynamic therapy. The use of anti-TIGIT antibodies with reduced or abolished Fc effector function (e.g., domvanalimab) in combination with one or more additional therapy may have a synergistic or additive therapeutic or a prophylactic effect on the underlying disease, disorder, or condition. In addition or alternatively, the combination therapy may allow for a dose reduction of one or more of the therapies, thereby ameliorating, reducing or eliminating adverse effects associated with one or more of the therapies.

In embodiments comprising one or more additional treatment modality, the anti-TIGIT antibodies with reduced or abolished Fc effector function can be administered before, after or during treatment with the additional treatment modality. In embodiments comprising one or more additional therapeutic agent, the therapeutic agent(s) used in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a patient's course of therapy. If the combination is formulated as separate compositions, in some embodiments, the separate compositions are provided together in a kit.

In some embodiments, one or more of the additional therapeutic agents is a chemotherapeutic agent. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, pomalidomide, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU): folic acid analogs such as denopterin, methotrexate, pemetrexed, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab paclitaxel, and docetaxel; chlorambucil; gemcitabine; 6-thioguanine; mercaptopurine; methotrexate; platinum and platinum coordination complexes such as cisplatin, carboplatin and oxaliplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT 11; topoisomerase inhibitors such as irinotecan, topotecan, etoposide, mitoxantrone, teniposide; difluoromethylornithine (DMFO); retinoic acid; esperamicins; capecitabine; anthracyclines and pharmaceutically acceptable salts, acids or derivatives of any of the above. In certain embodiments, combination therapy comprises a chemotherapy regimen that includes one or more chemotherapeutic agents. In one embodiment, combination therapy comprises a chemotherapeutic regimen comprising FOLFOX (folinic acid, fluorouracil, and oxaliplatin), FOLFIRI (folinic acid, fluorouracil, and irinotecan), a taxane (e.g., docetaxel, paclitaxel, nab-paclitaxel, etc.), CAPOX (capecitabine and oxaliplatin), XELOX (capecitabine and oxaliplatin), irinotecan, a fluoropyrimidine-containing chemotherapy (e.g., fluorouracil, capecitabine, floxuridine), a platinum-based chemotherapeutic agent, or gemcitabine.

In some embodiments, one or more of the additional therapeutic agents is a radiopharmaceutical. A radiopharmaceutical is a form of internal radiation therapy in which a source of radiation (i.e., one or more radionuclide) is put inside a subject's body. The radiation source can be in solid or liquid form. Non-limiting examples of radiopharmaceuticals include sodium iodide 1-131, radium-223 dichloride, lobenguane iodine-131, radioiodinated vesicles (e.g., saposin C-dioleoylphosphatidylserine (SapC-DOPS) nanovesicles), various forms of brachytherapy, and various forms of targeted radionuclides. Targeted radionuclides comprise a radionuclide associated (e.g., by covalent or ionic interactions) with a molecule (“a targeting agent”) that specifically binds to a target on a cell, typically a cancer cell or an immune cell. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen (i.e., an antigen enriched but not specific to a cancer cell), a tumor-specific antigen (i.e., an antigen with minimal to no expression in normal tissue), or a neo-antigen (i.e., an antigen specific to the genome of a cancer cell generated by non-synonymous mutations in the tumor cell genome). Non-limiting examples of targeted radionuclides include radionuclides attached to: somatostatin or peptide analogs thereof (e.g., 177Lu-Dotatate, etc.); prostate specific membrane antigen or peptide analogs thereof (e.g., 177Lu-PSMA-617, 225Ac-PSMA-617, 177Lu-PSMA-I&T, 177Lu-MIP-1095, etc.): a receptor's cognate ligand, peptide derived from the ligand, or variants thereof (e.g., 188Re-labeled VEGF₁₂₅₋₁₃₆ or variants thereof with higher affinity to VEGF receptor, etc.); antibodies targeting tumor antigens (e.g., 131I-tositumomab, 90Y-ibritumomab tiuxetan, CAM-H2-I131 (Precirix NV), 1131-omburtamab, etc.).

In some embodiments, one or more of the additional therapeutic agents is a hormone therapy. Hormone therapies act to regulate or inhibit hormonal action on tumors. Examples of hormone therapies include, but are not limited to: selective estrogen receptor degraders such as fulvestrant, GDC-9545, SAR439859, RG6171, AZD9833, rintodestrant, ZN-c5, LSZ102, D-0502, LY3484356, SHR9549; selective estrogen receptor modulators such as tamoxifen, raloxifene, 4-hydroxytamoxifen, trioxifene, keoxifene, toremifene; aromatase inhibitors such as anastrozole, exemestane, letrozole and other aromatase inhibiting 4(5)-imidazoles; gonadotropin-releasing hormone agonists such as nafarelin, triptorelin, goserelin; gonadotropin-releasing hormone antagonists such as degarelix; antiandrogens such as abiraterone, enzalutamide, apalutamide, darolutamide, flutamide, nilutamide, bicalutamide, leuprolide; 5a-reductase inhibitors such as finasteride, dutasteride; and the like. In certain embodiments, combination therapy comprises administration of a hormone or related hormonal agent. In one embodiment, combination therapy comprises administration of enzalutamide.

In some embodiments, one or more of the additional therapeutic agents is an epigenetic modulator. An epigenetic modulator alters an epigenetic mechanism controlling gene expression, and may be, for example, an inhibitor or activator of an epigenetic enzyme. Non-limiting examples of epigenetic modulators include DNA methyltransferase (DNMT) inhibitors, hypomethylating agents, and histone deacetylase (HDAC) inhibitors. In one or more embodiments, anti-TIGIT antibodies with reduced or abolished Fc effector function (e.g., domvanalimab) can be combined with DNA methyltransferase (DNMT) inhibitors or hypomethylating agents. Exemplary DNMT inhibitors include decitabine, zebularine and azacitadine. In one or more embodiments, combinations of anti-TIGIT antibodies with reduced or abolished Fc effector function (e.g., domvanalimab) with a histone deacetylase (HDAC) inhibitor is also contemplated. Exemplary HDAC inhibitors include vorinostat, givinostat, abexinostat, panobinostat, belinostat and trichostatin A.

In some embodiments, one or more of the additional therapeutic agents is an ATP-adenosine axis-targeting agent. ATP-adenosine axis-targeting agents alter signaling mediated by adenine nucleosides and nucleotides (e.g., adenosine, AMP, ADP, ATP), for example by modulating the level of adenosine or targeting adenosine receptors. Adenosine and ATP, acting at different classes of receptors, often have opposite effects on inflammation, cell proliferation and cell death. For instance, ATP and other adenine nucleotides have antitumor effects via activation of the PS2Y1 receptor subtype, while accumulation of adenosine in the tumor microenvironment has been shown to inhibit the antitumor function of various immune cells and to augment the immunosuppressive activity of myeloid and regulatory T cells by binding to cell surface adenosine receptors. In certain embodiments, an ATP-adenosine axis-targeting agent is an inhibitor of an ectonucleotidase involved in the conversion of ATP to adenosine or an antagonist of adenosine receptor. Ectonucleotidases involved in the conversion of ATP to adenosine include the ectonucleoside triphosphate diphosphohydrolase 1 (ENTPD1, also known as CD39 or Cluster of Differentiation 39) and the ecto-5′-nucleotidase (NT5E or 5NT, also known as CD73 or Cluster of Differentiation 73). Exemplary small molecule CD73 inhibitors include CB-708, ORIC-533, LY3475070 and AB680. Exemplary anti-CD39 and anti-CD73 antibodies include ES002, TTX-030, IPH-5201, SRF-617, CPI-006, oleclumab (MEDI9447), NZV930, IPH5301, uliledlimab (TJD5, TJ004309), and BMS-986179. In one embodiment, the present disclosure contemplates combination of anti-TIGIT antibodies with reduced or abolished Fc effector function (e.g., domvanalimab) described herein with a CD73 inhibitor such as those described in WO 2017/120508, WO 2018/067424, WO 2018/094148, and WO 2020/046813. In further embodiments, the CD73 inhibitor is quemliclustat. Adenosine can bind to and activate four different G-protein coupled receptors: A₁R, A₂ₐR, A_{2b}R, and A₃R. A₂R antagonists include etrumadenant, inupadenant, taminadenant, caffeine citrate, NUV-1182, TT-702, DZD-2269, INCB-106385, EVOEXS-21546, AZD-4635, imaradenant, RVU-330, ciforadenant, PBF-509, PBF-999, PBF-1129, and CS-3005. In some embodiments, the present disclosure contemplates the combination of anti-TIGIT antibodies with reduced or abolished Fc effector function (e.g., domvanalimab) described herein with an A₂ₐR antagonist, an A_{2b}R antagonist, or an antagonist of A₂ₐR and A_{2b}R. In some embodiments, the present disclosure contemplates the combination of the anti-TIGIT antibodies with reduced or abolished Fc effector function (e.g., domvanalimab) described herein with the adenosine receptor antagonists described in WO 2018/136700, WO 2018/204661, WO 2018/213377, or WO 2020/023846, WO 2020/102646. In one embodiment, the adenosine receptor antagonist is etrumadenant.

In some embodiments, one or more of the additional therapeutic agents is a targeted therapy. In one aspect, a targeted therapy may comprise a chemotherapeutic agent, a radionuclide, a hormone therapy, or another small molecule drug attached to a targeting agent. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations or gene fusions in the tumor cell genome). In some embodiments, the targeting agent is an antibody and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations or gene fusions in the tumor cell genome). In other aspects, a targeted therapy may inhibit or interfere with a specific protein that helps a tumor, survive, grow and/or spread. Non-limiting examples of such targeted therapies include signal transduction inhibitors, RAS signaling inhibitors, inhibitors of oncogenic transcription factors, activators of oncogenic transcription factor repressors, angiogenesis inhibitors, immunotherapeutic agents, ATP-adenosine axis-targeting agents, AXL inhibitors, PARP inhibitors, PAK4 inhibitors, PI3K inhibitors, HIF2α inhibitors, CD39 inhibitors, CD73 inhibitors, A₂R antagonists, TIGIT antagonists, and PD-1 antagonists. ATP-adenosine axis-targeting agents are described above, while other agents are described in further detail below.

In some embodiments, one or more of the additional therapeutic agents is a signal transduction inhibitor. Signal transduction inhibitors are agents that selectively inhibit one or more steps in a signaling pathway. Signal transduction inhibitors (STIs) contemplated by the present disclosure include but are not limited to: (i) BCR-ABL kinase inhibitors (e.g., imatinib); (ii) epidermal growth factor receptor tyrosine kinase inhibitors (EGFR TKIs), including small molecule inhibitors (e.g., gefitinib, erlotinib, afatinib, icotinib, and osimertinib), and anti-EGFR antibodies; (iii) inhibitors of the human epidermal growth factor (HER) family of transmembrane tyrosine kinases, e.g., HER-2/neu receptor inhibitors (e.g., trastuzumab and HER-3 receptor inhibitors; (iv) vascular endothelial growth factor receptor (VEGFR) inhibitors including small molecule inhibitors (e.g., axitinib, sunitinib and sorafenib), VEGF kinase inhibitors (e.g., lenvatinib, cabozantinib, pazopanib, tivozanib, XL092, etc.), anti-VEGF antibodies (e.g., bevacizumab), and anti-VEGFR antibodies (e.g., ramucirumab, etc,); (v) inhibitors of AKT family kinases or the AKT pathway (e.g., rapamycin); (vi) inhibitors of serine/threonine-protein kinase B-Raf (BRAF), such as, for example, vemurafenib, dabrafenib and encorafenib; (vii) inhibitors of rearranged during transfection (RET), including, for example, selpercatinib and pralsetinib; (viii) tyrosine-protein kinase Met (MET) inhibitors (e.g., tepotinib, tivantinib, cabozantinib and crizotinib); (ix) anaplastic lymphoma kinase (ALK) inhibitors (e.g., ensartinib, ceritinib, lorlatinib, crizotinib, and brigatinib); (x) inhibitors of the RAS signaling pathway (e.g., inhibitors of KRAS, HRAS, RAF, MEK, ERK) as described elsewhere herein; (xi) FLT-3 inhibitors (e.g., gilteritinib); (xii) inhibitors of Trop-2; (xiii) inhibitors of the JAK/STAT pathway, e.g., JAK inhibitors including tofacitinib and ruxolitinib, or STAT inhibitors such as napabucasin; (xiv) inhibitors of NF-kB; (xv) cell cycle kinase inhibitors (e.g., flavopiridol); (xvi) phosphatidyl inositol kinase (PI3K) inhibitors; and (xix) protein kinase B (AKT) inhibitors (e.g., capivasertib, miransertib). In one or more embodiments, the additional therapeutic agent comprises an inhibitor of EGFR, VEGFR, HER-2, HER-3, BRAF, RET, MET, ALK, RAS (e.g., KRAS, MEK, ERK), FLT-3, JAK, STAT, NF-kB, PI3K, AKT, or any combinations thereof.

In some embodiments, one or more of the additional therapeutic agents is a RAS signaling inhibitor. Oncogenic mutations in the RAS family of genes, e.g., HRAS, KRAS, and NRAS, are associated with a variety of cancers. For example, mutations of G12C, G12D, G12V, G12A, G13D, Q61H, G13C and G12S, among others, in the KRAS family of genes have been observed in multiple tumor types. Direct and indirect inhibition strategies have been investigated for the inhibition of mutant RAS signaling. Indirect inhibitors target effectors other than RAS in the RAS signaling pathway, and include, but are not limited to, inhibitors of RAF, MEK, ERK, PI3K, PTEN, SOS (e.g., SOS1), mTORC1, SHP2 (PTPN11), and AKT. Non-limiting examples of indirect inhibitors under development include RMC-4630, RMC-5845, RMC-6291, RMC-6236, JAB-3068, JAB-3312, TNO155, RLY-1971, B11701963. Direct inhibitors of RAS mutants have also been explored, and generally target the KRAS-GTP complex or the KRAS-GDP complex. Exemplary direct RAS inhibitors under development include, but are not limited to, sotorasib (AMG510), MRTX849, mRNA-5671 and ARS1620. In some embodiments, the one or more RAS signaling inhibitors are selected from the group consisting of RAF inhibitors, MEK inhibitors, ERK inhibitors, PI3K inhibitors, PTEN inhibitors, SOS1 inhibitors, mTORC1 inhibitors, SHP2 inhibitors, and AKT inhibitors. In other embodiments the one or more RAS signaling inhibitors directly inhibit RAS mutants.

In some embodiments one or more of the additional therapeutic agents is an inhibitor of a phosphatidylinositol 3-kinase (PI3K), particularly an inhibitor of the PI3Kγ isoform. PI3Kγ inhibitors can stimulate an anti-cancer immune response through the modulation of myeloid cells, such as by inhibiting suppressive myeloid cells, dampening immune-suppressive tumor-infiltrating macrophages or by stimulating macrophages and dendritic cells to make cytokines that contribute to effective T cell responses thereby decreasing cancer development and spread. Exemplary PI3Kγ inhibitors include copanlisib, duvelisib, AT-104, ZX-101, tenalisib, eganelisib, SF-1126, AZD3458, and pictilisib. In some embodiments, the anti-TIGIT antibodies with reduced or abolished Fc effector function (e.g., domvanalimab) can be combined with one or more PI3Kγ inhibitors described in WO 2020/0247496A1.

In some embodiments, one or more of the additional therapeutic agents is an inhibitor of arginase. Arginase has been shown to be either responsible for or participate in inflammation-triggered immune dysfunction, tumor immune escape, immunosuppression and immunopathology of infectious disease. Exemplary arginase compounds include CB-1158 and OAT-1746. In some embodiments, the anti-TIGIT antibodies with reduced or abolished Fe effector function (e.g., domvanalimab) can be combined with one or more arginase inhibitors described in WO/2019/173188 and WO 2020/102646.

In some embodiments, one or more of the additional therapeutic agents is an inhibitor of an oncogenic transcription factor or an activator of an oncogenic transcription factor repressor. Suitable agents may act at the expression level (e.g., RNAi, siRNA, etc.), through physical degradation, at the protein/protein level, at the protein/DNA level, or by binding in an activation/inhibition pocket. Non-limiting examples include inhibitors of one or more subunit of the MLL complex (e.g., HDAC, DOT1L, BRD4, Menin, LEDGF, WDR5, KDM4C (JMJD2C) and PRMT1), inhibitors of hypoxia-inducible factor (HIF) transcription factor, and the like.

In some embodiments, one or more of the additional therapeutic agents is an inhibitor of a hypoxia-inducible factor (HIF) transcription factor, particularly HIF-2α. Exemplary HIF-2α inhibitors include belzutifan, ARO-HIF2, PT-2385, AB521, and those described in WO 2021113436 and WO 2021188769. In some embodiments, the anti-TIGIT antibodies with reduced or abolished Fc effector function (e.g., domvanalimab) can be combined with one or more HIF-2α inhibitors described in WO 2021188769.

In some embodiments, one or more of the additional therapeutic agents is an inhibitor of anexelekto (AXL). The AXL signaling pathway is associated with tumor growth and metastasis, and is believed to mediate resistance to a variety of cancer therapies. There are a variety of AXL inhibitors under development that also inhibit other kinases in the TAM family (i.e., TYRO3, MERTK), as well as other receptor tyrosine kinases including MET, FLT3, RON and AURORA, among others. Exemplary multikinase inhibitors include sitravatinib, rebastinib, glesatinib, gilteritinib, merestinib, cabozantinib, foretinib, BMS777607, LY2801653, S49076, GSK1363089, and RXDX-106. AXL specific inhibitors have also been developed, e.g., small molecule inhibitors including DS-1205, SGI-7079, SLC-391, TP-0903 (i.e., dubermatinib), BGB324 (i.e., bemcentinib) and DP3975; anti-AXL antibodies such as ADCT-601; and antibody drug conjugates (ADCs) such as BA3011. Another strategy to inhibit AXL signaling involves targeting AXL's ligand, GAS6. For example, AVB-500 is under development as is a Fe fusion protein that binds the GAS6 ligand thereby inhibiting AXL signaling.

In some embodiments, one or more of the additional therapeutic agents is an inhibitor of p21-activated kinase 4 (PAK4). PAK4 overexpression has been shown across a variety of cancer types, notably including those resistant to PD-1 therapies. While no PAK4 inhibitors have been approved, some are in development, and exhibit dual PAK4/NAMPT inhibitor activity, e.g., ATG-019 and KPT-9274. In some embodiments, the compounds according to this disclosure are combined with a PAK4 selective inhibitor. In some embodiments, the compounds according to this disclosure are combined with a PAK4/NAMPT dual inhibitor, e.g., ATG-019 or KPT-9274.

In some embodiments, one or more of the additional therapeutic agents is (i) an agent that inhibits the enzyme poly (ADP-ribose) polymerase (e.g., olaparib, niraparib and rucaparib, etc.); (ii) an inhibitor of the Bcl-2 family of proteins (e.g., venetoclax, navitoclax, etc.); (iii) an inhibitor of MCL-1; (iv) an inhibitor of the CD47-SIRPα pathway (e.g., an anti-CD47 antibody); (v) an isocitrate dehydrogenase (IDH) inhibitor, e.g., IDH-1 or IDH-2 inhibitor (e.g., ivosidenib, enasidenib, etc.).

In some embodiments, one or more of the additional therapeutic agents is an immunotherapeutic agent. Immunotherapeutic agents useful in the treatment of cancers typically elicit or amplify an immune response to cancer cells. Non-limiting examples of suitable immunotherapeutic agents include: immunomodulators; cellular immunotherapies; vaccines; gene therapies; ATP-adenosine axis-targeting agents; immune checkpoint modulators. ATP-adenosine axis-targeting agents are described above. Immunomodulators, cellular immunotherapies, vaccines, gene therapies, and immune checkpoint modulators are described further below.

In some embodiments, one or more of the additional therapeutic agents is an immunotherapeutic agent, more specifically a cytokine or chemokine, such as, IL1, IL2, IL12, IL18, ELC/CCL19, SLC/CCL21, MCP-1, IL-4, IL-18, TNF, IL-15, MDC, IFNa/b, M-CSF, IL-3, GM-CSF, IL-13, and anti-IL-10; bacterial lipopolysaccharides (LPS); an organic or inorganic adjuvant that activates antigen-presenting cells and promote the presentation of antigen epitopes on major histocompatibility complex molecules including, but not limited to Toll-like receptor (TLR) agonists, antagonists of the mevalonate pathway, agonists of STING; indoleamine 2,3-dioxygenase 1 (IDO1) inhibitors and immune-stimulatory oligonucleotides, as well as other T cell adjuvants.

In some embodiments, one or more of the additional therapeutic agents is an immunotherapeutic agent, more specifically a cellular therapy. Cellular therapies are a form of treatment in which viable cells are administered to a subject. In certain embodiments, one or more of the additional therapeutic agents is a cellular immunotherapy that activates or suppresses the immune system. Cellular immunotherapies useful in the treatment of cancers typically elicit or amplify an immune response. The cells can be autologous or allogenic immune cells (e.g., monocytes, macrophages, dendritic cells, NK cells, T cells, etc.) collected from one or more subject. Alternatively, the cells can be “(re)programmed” allogenic immune cells produced from immune precursor cells (e.g., lymphoid progenitor cells, myeloid progenitor cells, common dendritic cell precursor cells, stem cells, induced pluripotent stem cells, etc.). In some embodiments, such cells may be an expanded subset of cells with distinct effector functions and/or maturation markers (e.g., adaptive memory NK cells, tumor infiltrating lymphocytes, immature dendritic cells, monocyte-derived dendritic cells, plasmacytoid dendritic cells, conventional dendritic cells (sometimes referred to as classical dendritic cells), M1 macrophages, M2 macrophages, etc.), may be genetically modified to target the cells to a specific antigen and/or enhance the cells' anti-tumor effects (e.g., engineered T cell receptor (TCR) cellular therapies, chimeric antigen receptor (CAR) cellular therapies, lymph node homing of antigen-loaded dendritic cells, etc.), may be engineered to express of have increased expression of a tumor-associated antigen, or may be any combination thereof. Non-limiting types of cellular therapies include CAR-T cell therapy, CAR-NK cell therapy, TCR therapy, and dendritic cell vaccines. Exemplary cellular immunotherapies include sipuleucel-T, tisagenlecleucel, lisocabtagene maraleucel, idecabtagene vicleucel, brexucabtagene autoleucel, and axicabtagene ciloleucel, as well as CTX110, JCAR015, JCAR017, MB-CART19.1, MB-CART20.1, MB-CART2019.1, UniCAR02-T-CD123, BMCA-CAR-T, JNJ-68284528, BNT211, and NK-92/5.28.z.

In some embodiments, one or more of the additional therapeutic agents is an immunotherapeutic agent, more specifically a gene therapy. Gene therapies comprise recombinant nucleic acids administered to a subject or to a subject's cells ex vivo in order to modify the expression of an endogenous gene or to result in heterologous expression of a protein (e.g., small interfering RNA (siRNA) agents, double-stranded RNA (dsRNA) agents, micro RNA (miRNA) agents, viral or bacterial gene delivery, etc.), as well as gene editing therapies that may or may not comprise a nucleic acid component (e.g., meganucleases, zinc finger nucleases, TAL nucleases, CRISPR/Cas nucleases, etc.), oncolytic viruses, and the like. Non-limiting examples of gene therapies that may be useful in cancer treatment include Gendicine® (rAd-p53), Oncorine® (rAD5-H101), talimogene laherparepvec, Mx-dnG1, ARO-HIF2 (Arrowhead), CTX110 (CRISPR Therapeutics), CTX120 (CRISPR Therapeutics), and CTX130 (CRISPR Therapeutics).

In some embodiments, one or more of the additional therapeutic agent is an immunotherapeutic agent, more specifically an agent that modulates an immune checkpoint. Immune checkpoints are a set of inhibitory and stimulatory pathways that directly affect the function of immune cells (e.g., B cells, T cells, NK cells, etc.). Immune checkpoints engage when proteins on the surface of immune cells recognize and bind to their cognate ligands. The present invention contemplates the use of anti-TIGIT antibodies with reduced or abolished Fc effector function (e.g., domvanalimab) described herein in combination with agonists of stimulatory or co-stimulatory pathways and/or antagonists of inhibitory pathways. Agonists of stimulatory or co-stimulatory pathways and antagonists of inhibitory pathways may have utility as agents to overcome distinct immune suppressive pathways within the tumor microenvironment, inhibit T regulatory cells, reverse/prevent T cell anergy or exhaustion, trigger innate immune activation and/or inflammation at tumor sites, or combinations thereof.

In some embodiments, one or more of the additional therapeutic agents is an immune checkpoint inhibitor. As used herein, the term “immune checkpoint inhibitor” refers to an antagonist of an inhibitory or co-inhibitory immune checkpoint. The terms “immune checkpoint inhibitor”, “checkpoint inhibitor” and “CPI” may be used herein interchangeably. Immune checkpoint inhibitors may antagonize an inhibitory or co-inhibitory immune checkpoint by interfering with receptor-ligand binding and/or altering receptor signaling. Examples of immune checkpoints (ligands and receptors), some of which are selectively upregulated in various types of cancer cells, that can be antagonized include PD-1 (programmed cell death protein 1); PD-L1 (PD1 ligand); BTLA (B and T lymphocyte attenuator); CTLA-4 (cytotoxic T-lymphocyte associated antigen 4); TIM-3 (T cell immunoglobulin and mucin domain-containing protein 3); LAG-3 (lymphocyte activation gene 3); TIGIT (T cell immunoreceptor with Ig and ITIM domains); CD276 (B7-H3), PD-L2, Galectin 9, CEACAM-1, BTLA, CD69, Galectin-1, CD113, GPR56, VISTA, 2B4, CD48, GARP, PD1H, LAIR1, TIM-1, and TIM-4, and Killer Inhibitory Receptors, which can be divided into two classes based on their structural features: i) killer cell immunoglobulin-like receptors (KIRs), and ii)C-type lectin receptors (members of the type II transmembrane receptor family). Also contemplated are other less well-defined immune checkpoints that have been described in the literature, including both receptors (e.g., the 2B4 (also known as CD244) receptor) and ligands (e.g., certain B7 family inhibitory ligands such B7-H3 (also known as CD276) and B7-H4 (also known as B7-S1, B7x and VCTN1)). [See Pardoll, (April 2012) Nature Rev. Cancer 12:252-64].

In some embodiments, an immune checkpoint inhibitor is a CTLA-4 antagonist. In further embodiments, the CTLA-4 antagonist can be an antagonistic CTLA-4 antibody. Suitable antagonistic CTLA-4 antibodies include, for example, monospecific antibodies such as ipilimumab or tremelimumab or zalifrelimab, as well as bispecific antibodies such as MED15752 and KN046.

In some embodiments, an immune checkpoint inhibitor is a PD-1 antagonist. In further embodiments, the PD-1 antagonist can be an antagonistic PD-1 antibody. Suitable antagonistic PD-1 antibodies include, for example, monospecific antibodies such as budigalimab, camrelizumab, cosibelimab, dostarlimab, cemiplimab, ezabenlimab (BI-754091), MEDI-0680 (AMP-514; WO2012/145493), nivolumab, pembrolizumab, pidilizumab (CT-011), pimivalimab, retifanlimab, sasanlimab, spartalizumab, sintilimab, tislelizumab, toripalimab, and zimberelimab; as well as bispecific antibodies such as LY3434172. In still further embodiments, the PD-1 antagonist can be a recombinant protein composed of the extracellular domain of PD-L2 (B7-DC) fused to the Fc portion of IgG1 (AMP-224). In certain embodiments, an immune checkpoint inhibitor is zimberelimab.

In some embodiments, an immune checkpoint inhibitor is a PD-L1 antagonist. In further embodiments, the PD-I antagonist can be an antagonistic PD-L1 antibody small molecule or peptide. Suitable antagonistic PD-L1 antibodies include, for example, monospecific antibodies such as avelumab, atezolizumab, balstilimab, durvalumab, BMS-936559, and envafolimab as well as bispecific antibodies such as LY3434172 and KN046.

In some embodiments, one or more of the additional therapeutic agents activates a stimulatory or co-stimulatory immune checkpoint. Examples of stimulatory or co-stimulatory immune checkpoints (ligands and receptors) include B7-1, B7-2, CD28, 4-1BB (CD137), 4-1BBL, ICOS, ICOS-L, OX40, OX40L, GITR, GITRL, CD70, CD27, CD40, DR3 and CD2.

In some embodiments, an agent that activates a stimulatory or co-stimulatory immune checkpoint is a CD137 (4-1BB) agonist. In further embodiments, the CD137 agonist can be an agonistic CD137 antibody. Suitable CD137 antibodies include, for example, urelumab and PF-05082566 (WO12/32433). In some embodiments, an agent that activates a stimulatory or co-stimulatory immune checkpoint is a GITR agonist. In further embodiments, the GITR agonist can be an agonistic GITR antibody. Suitable GITR antibodies include, for example, BMS-986153, BMS-986156, TRX-518 (WO06/105021, WO09/009116) and MK-4166 (WO11/028683). In some embodiments, an agent that activates a stimulatory or co-stimulatory immune checkpoint is an OX40 agonist. In further embodiments, the OX40 agonist can be an agonistic OX40 antibody. Suitable OX40 antibodies include, for example, MEDI-6383, MEDI-6469, MEDI-0562, PF-04518600, GSK3174998, BMS-986178, and MOXR0916. In some embodiments, an agent that activates a stimulatory or co-stimulatory immune checkpoint is a CD40 agonist. In further embodiments, the CD40 agonist can be an agonistic CD40 antibody, such as dacetuzumab, selicrelumab, APX005M, ADC-1013, or CDX-1140. In some embodiments, an agent that activates a stimulatory or co-stimulatory immune checkpoint is a CD27 agonist. In further embodiments, the CD27 agonist can be an agonistic CD27 antibody. Suitable CD27 antibodies include, for example, varlilumab.

In some embodiments, one or more of the additional therapies is an immunotherapeutic agent, more specifically a signal transduction inhibitor. Intracellular signaling molecules that influence immune cell functions may also be suitable targets for improving antitumor immunity. For example, one or more of the additional therapies may be an inhibitor of an intracellular signaling molecule. is an inhibitor of hematopoietic progenitor kinase 1 (HPK1). HPK1 is serine/threonine kinase that functions as a negative regulator of activation signals generated by the T cell antigen receptor. As another example, one or more of the additional therapies may be an inhibitor of Cbl-b, an E3 ubiquitin ligase involved in the regulation of TCR signaling (e.g., AP401). As another example, one or more of the additional therapies may be an inhibitor of diacylglycerol kinase (DGK). In some embodiments, the inhibitor is a small molecule. Non-limiting examples of small molecule HKP1 inhibitors in clinical development include CFI-402411 and BGB-15025; non-limiting examples of Cbl-b inhibitors in clinical development include AP401.

In some embodiments, one or more of the additional therapeutic agents is an agent that inhibits or depletes immune-suppressive immune cells. For example, to inhibit or deplete immunosuppressive macrophages or monocytes the agent may be CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264), or an antibody disclosed in WO14/036357.

In some embodiments, each additional therapeutic agent can independently be a chemotherapeutic agent, a radiopharmaceutical, a hormone therapy, an epigenetic modulator, a targeted agent, an immunotherapeutic agent, a cellular therapy, or a gene therapy. For example, in one embodiment, the present disclosure contemplates the use of anti-TIGIT antibodies with reduced or abolished Fc effector function (e.g., domvanalimab) in combination with one or more chemotherapeutic agent and optionally one or more additional therapeutic agents, wherein each additional therapeutic agent is independently a radiopharmaceutical, a hormone therapy, a targeted agent, an immunotherapeutic agent, a cellular therapy, or a gene therapy. In another embodiment, the present disclosure contemplates the use anti-TIGIT antibodies with reduced or abolished Fc effector function (e.g., domvanalimab) of the present disclosure in combination with one or more chemotherapeutic agent and optionally one or more additional therapeutic agents, wherein each additional therapeutic agent is independently a targeted agent, an immunotherapeutic agent, or a cellular therapy. In another embodiment, the present disclosure contemplates the use of anti-TIGIT antibodies with reduced or abolished Fc effector function (e.g., domvanalimab) of the present disclosure in combination with one or more chemotherapeutic agent and one or more tyrosine kinase inhibitor, and optionally one or more additional therapeutic agents, wherein each additional therapeutic agent is independently a targeted agent, an immunotherapeutic agent, or a cellular therapy. In another embodiment, the present disclosure contemplates the use of anti-TIGIT antibodies with reduced or abolished Fc effector function (e.g., domvanalimab) of the present disclosure in combination with one or more chemotherapeutic agent and one or more inhibitor independently selected from (i) BCR-ABL kinase inhibitor; (ii) an EGFR inhibitor (e.g., EGFR TKI or anti-EGFR antibody); (iii) HER-2/neu receptor inhibitor; (iv) an anti-angiogenic agent (e.g., anti-VEGF antibody, VEGFR TKI, VEGF kinase inhibitors, etc.); (v) AKT inhibitor; (vi) BRAF inhibitor; (vii) RET inhibitor; (viii) MET inhibitor; and (ix) ALK inhibitor, and optionally one or more additional therapeutic agents, wherein each additional therapeutic agent is independently a targeted agent, an immunotherapeutic agent, or a cellular therapy. In another embodiment, the present disclosure contemplates the use of the anti-TIGIT antibodies with reduced or abolished Fc effector function (e.g., domvanalimab) of the present disclosure in combination with one or more immunotherapeutic agent and optionally one or more additional therapeutic agent, wherein each additional therapeutic agent is independently a radiopharmaceutical, a hormone therapy, a targeted agent, a chemotherapeutic agent, a cellular therapy, or a gene therapy. In another embodiment, the present disclosure contemplates the use of the anti-TIGIT antibodies with reduced or abolished Fc effector function (e.g., domvanalimab) of the present disclosure in combination with one or more immunotherapeutic agents and one or more chemotherapeutic agent, and optionally one or more additional therapeutic agent, wherein each additional therapeutic agent is independently a radiopharmaceutical, a hormone therapy, a targeted agent, a cellular therapy, or a gene therapy. In another embodiment, the present disclosure contemplates the use of the anti-TIGIT antibodies with reduced or abolished Fc effector function (e.g., domvanalimab) of the present disclosure in combination with one or more immunotherapeutic agents and optionally one or more additional therapeutic agents, wherein each additional therapeutic agent is independently a chemotherapeutic agent, a targeted agent, or a cellular therapy. In another embodiment, the present disclosure contemplates the use of the anti-TIGIT antibodies with reduced or abolished Fc effector function (e.g., domvanalimab) of the present disclosure in combination with one or more immune checkpoint inhibitors and/or one or more ATP-adenosine axis-targeting agents, and optionally one or more additional therapeutic agents, wherein each additional therapeutic agent is independently a chemotherapeutic agent, a targeted agent, an immunotherapeutic agent, or a cellular therapy. In another embodiment, the present disclosure contemplates the use of the anti-TIGIT antibodies with reduced or abolished Fc effector function (e.g., domvanalimab) of the present disclosure in combination with one or more immune checkpoint inhibitors and/or one or more ATP-adenosine axis-targeting agents and/or one or more chemotherapeutic agents, and optionally one or more additional therapeutic agents, wherein each additional therapeutic agent is independently a targeted agent, an immunotherapeutic agent, or a cellular therapy. In another embodiment, the present disclosure contemplates the use of the anti-TIGIT antibodies with reduced or abolished Fc effector function (e.g., domvanalimab) of the present disclosure in combination with one or more immune checkpoint inhibitors and/or one or more ATP-adenosine axis-targeting agents, and one or more inhibitor independently selected from (i) BCR-ABL kinase inhibitor; (ii) an EGFR inhibitor (e.g., EGFR TKI or anti-EGFR antibody); (iii) HER-2/neu receptor inhibitor; (iv) an anti-angiogenic agent (e.g., anti-VEGF antibody, VEGFR TKI, VEGF kinase inhibitors, etc.); (v) AKT inhibitor; (vi) BRAF inhibitor; (vii) RET inhibitor; (viii) MET inhibitor; and (ix) ALK inhibitor. In further embodiments of the above (a) the targeted agent can be a PI3K inhibitor, an arginase inhibitor, a HIF2a inhibitor, an AXL inhibitor, a PAK4 inhibitor, or an anti-angiogenic agent; (b) the immunotherapeutic agent is an ATP-adenosine axis-targeting agent, cytokine therapy, an immune checkpoint inhibitor, or a combination thereof; (c) the ATP-adenosine axis-targeting agent is an A₂ₐR and/or A_{2b}R antagonist, a CD73 inhibitor, or a CD39 inhibitor; (d) the ATP-adenosine axis-targeting agent is etrumadenant or quemliclustat; (e) the immunotherapeutic agent is an anti-PD-1 antagonist antibody or an anti-PD-1 antagonist antibody, optionally selected from the group consisting of budigalimab, camrelizumab, cosibelimab, dostarlimab, cemiplimab, ezabenlimab, nivolumab, pembrolizumab, pidilizumab, pimivalimab, retifanlimab, sasanlimab, spartalizumab, sintilimab, tislelizumab, toripalimab, zimberelimab, LY3434172, avelumab, atezolizumab, balstilimab, durvalumab, envafolimab, LY3434172 and KN046; (f) the immunotherapeutic agent is zimberelimab; (g), the anti-angiogenic agent is pazopanib, sorafenib, sunitinib, bevacizumab, axitinib, lenvatinib, tivozanib, or cabozantinib; or (h) any combination thereof. In still further embodiments of the above, the present disclosure contemplates the use of the anti-TIGIT antibodies with reduced or abolished Fc effector function (e.g., domvanalimab) of the present disclosure in combination with etrumadenant, quemliclustat, zimberelimab, or any combination thereof. In still any of the foregoing embodiments of the above, the anti-TIGIT antibody can be domvanalimab.

In a specific embodiment, the disclosure contemplates the use of domvanalimab in combination with an A₂ₐR antagonist, an A_{2b}R antagonist, an antagonist of A₂ₐR and A_{2b}R, a CD73 inhibitor, a CD39 inhibitor, a HIF2a inhibitor, an AXL inhibitor, an HPK1 inhibitor, a PI3K inhibitor, an immune checkpoint inhibitor, or a combination thereof.

In a specific embodiment, the disclosure contemplates the use of domvanalimab in combination with an A₂ₐR antagonist, an A_{2b}R antagonist, an antagonist of A₂ₐR and A_{2b}R, a CD73 inhibitor, a CD39 inhibitor, a HIF2a inhibitor, a PD-1 antagonist, a PD-L1 antagonist, or a combination thereof.

In a specific embodiment, the disclosure contemplates the use of domvanalimab in combination with an A₂ₐR antagonist, an A_{2b}R antagonist, an antagonist of A₂ₐR and A_{2b}R, a CD73 inhibitor, a CD39 inhibitor, a HIF2a inhibitor, a PD-1 antagonist, a PD-L1 antagonist, or a combination thereof.

In a specific embodiment, the disclosure contemplates the use of domvanalimab in combination with a PD-1 antagonist or a PD-L1 antagonist.

In a specific embodiment, the disclosure contemplates the use of domvanalimab in combination with a CTLA-4 antagonist.

Selection of the additional therapeutic agent(s) may be informed by current standard of care for a particular cancer and/or mutational status of a subject's cancer and/or stage of disease. Detailed standard of care guidelines are published, for example, by National Comprehensive Cancer Network (NCCN). See, for instance, NCCN Colon Cancer v3.2021, NCCN Hepatobiliary Cancer v5.2021, NCCN Kidney Cancer, v3.2022, NCCN NSCLC v7.2021, NCCN Pancreatic Adenocarcinoma v2.2021, NCCN Esophageal and Esophagogastric Junction Cancers v4.2021, NCCN Gastric Cancer v5.2021, Cervical Cancer v1.2022, Ovarian Cancer/Fallopian Tube Cancer/Primary Peritoneal Cancer v3.2021.

### V. Pharmaceutical Compositions

Provided herein are pharmaceutical compositions for use in the treatment or prevention of cancer and other diseases. The pharmaceutical compositions comprise an anti-TIGIT antibody (e.g., domvanalimab or another antibody of Section III) with a reduced capacity to bind FcγRs or an inability to bind FcγRs, particularly activating FcγRs.

The phrase “comprise(s) domvanalimab or a fragment or variant thereof as an active ingredient” means comprising domvanalimab or a fragment or variant thereof as at least one of the active ingredients, and does not limit the proportion of the antibody. Similarly, the phrase “comprise(s) an anti-TIGIT antibody with reduced or abolished Fc effector function” and similar descriptions of the disclosed antibodies means comprising an anti-TIGIT antibody or a fragment or variant thereof as at least one of the active ingredients, and does not limit the proportion of the antibody. In addition, the pharmaceutical compositions of the present disclosure may also comprise, in combination with an anti-TIGIT antibody (e.g., domvanalimab) or a fragment or variant thereof or an equivalent thereof, other ingredients that enhance the treatment or prevention of cancer. Non-limiting examples include, but are not limited to, checkpoint inhibitors (CPIs), such as a CTLA-4 antagonist, a PD-1 antagonist, a PD-L1 antagonist, or a combination thereof. Specific CPIs that may be combined with the disclosed anti-TIGIT antibodies (e.g., domvanalimab) include, but are not limited to ipilimumab (YERVOY®), nivolumab (OPDIVO®), pembrolizumab (KEYTRUDA®), cemiplimab (LIBTAYO®), avelumab (BAVENCIO®), durvalumab (IMFINZI®), atezolizumab (TECENTRIQ®), and zimberelimab (AB122).

Pharmaceutical compositions of an anti-TIGIT antibody (e.g., domvanalimab or a fragment or variant thereof or an equivalent thereof) of the present disclosure can be prepared as formulations (see, for example, Remington's Pharmaceutical Science, Mark Publishing Company, Easton, USA). The pharmaceutical compositions generally comprise a carrier and/or additive in addition to the antibody. For example, in some embodiments, the pharmaceutical composition comprises one or more surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA, pentetic acid), suspending agents, isotonizing agents, binders, disintegrators, lubricants, fluidity promoters, corrigents, light anhydrous silicic acid, lactose, crystalline cellulose, mannitol, starch, carmelose calcium, carmelose sodium, hydroxypropylcellulose, hydroxypropylmethylcellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, and inorganic salt. In some embodiments, the pharmaceutical composition comprises one or more other low-molecular-weight polypeptides, proteins such as serum albumin, gelatin, and immunoglobulin. In some embodiments, the pharmaceutical composition comprises one or more amino acids such as glycine, glutamine, histidine, asparagine, arginine, and lysine.

An anti-TIGIT antibody (e.g., domvanalimab or a fragment or variant thereof) may be prepared as an aqueous solution for injection, in which the anti-TIGIT antibody (e.g., domvanalimab or a fragment or variant thereof) may be dissolved in an isotonic solution containing, for example, physiological saline, dextrose, or other excipients or tonifiers (i.e., tonicity agents). The tonifier may include, for example, D-sorbitol, D-mannose, D-mannitol, trehalose, sodium chloride, or combinations thereof. In addition, appropriate buffers (Tris/Tris-HCl, histidine/histidine HCL, etc.), chelators (e.g., EDTA, pentetic acid, etc.), preservatives, and solubilizing agents, for example, alcohols (for example, ethanol), polyalcohols (for example, propylene glycols, PEGs, etc.), non-ionic detergents (polysorbate 80, HCO-50, etc.) may be used concomitantly. In one embodiment, an anti-TIGIT antibody may be formulated for dilution as an aqueous solution comprising about 10 mg/mL to about 100 mg/mL antibody or about 20 mg/mL to about 60 mg/mL antibody, a buffer containing about 10 mM to about 30 mM or about 15 to about 30 mM histidine/histidine-HCl, about 1% to about 10% or about 4% to about 10% (weight/volume) of an excipient selected from the group consisting of sucrose, dextrose, trehalose, sorbitol and mannitol, about 0 mg/mL to about 10 mg/mL NaCl, and about 0.05 mg/mL to about 0.6 mg/mL polysorbate 80. In one embodiment, an anti-TIGIT antibody may be formulated for injection as an aqueous solution comprising about 10 mg/mL to about 100 mg/mL antibody, a buffer containing about 10 to about 25 mM or about 15 to about 25 mM His/His-Cl, about 3% to about 10% (weight/volume) stabilizer (e.g., sucrose, dextrose, mannitol, etc.), about 0 mg/mL to about 10 mg/mL tonicity agent (e.g., NaCl, etc.), and about 0.1 mg/mL to about 0.3 mg/mL non-ionic detergent (e.g., polysorbate 80). In one embodiment, an anti-TIGIT antibody may be formulated for injection as an aqueous solution comprising about 10 mg/mL to about 100 mg/mL antibody, a buffer containing about 10 to about 25 mM or about 15 to about 25 mM His/His-Cl, about 5% to about 10% (weight/volume) of an excipient selected from the group consisting of sucrose, dextrose, and mannitol, about 0 mg/mL to about 10 mg/mL NaCl, and about 0.1 mg/mL to about 0.3 mg/mL polysorbate 80. In one embodiment, an anti-TIGIT antibody may be formulated for injection as an aqueous solution comprising about 20 mg/mL to about 60 mg/mL antibody, a buffer containing about 10 to about 25 mM or about 15 to about 25 mM His/His-Cl, about 5% to about 10% (weight/volume) of an excipient selected from the group consisting of sucrose, dextrose, and mannitol, about 0 mg/mL to about 10 mg/mL NaCl, and about 0.1 mg/mL to about 0.3 mg/mL polysorbate 80. In one embodiment, an anti-TIGIT antibody may be formulated for injection as an aqueous solution comprising or consisting of about 20 mg/mL to about 60 mg/mL antibody, a buffer containing about 15 to about 20 mM or about 20 mM His/His-Cl, about 5% to about 10% (weight/volume) of an excipient selected from the group consisting of sucrose, dextrose, and mannitol, and about 0.1 mg/mL to about 0.2 mg/mL polysorbate 80. In one embodiment, an anti-TIGIT antibody may be formulated for injection as an aqueous solution comprising or consisting of about 20 mg/mL to about 60 mg/mL antibody, a buffer containing about 15 to about 20 mM or about 20 mM His/His-Cl, about 5% to about 10% (weight/volume) sucrose, and about 0.1 mg/mL to about 0.2 mg/mL polysorbate 80. In one embodiment, an anti-TIGIT antibody may be formulated for injection as an aqueous solution comprising or consisting of about 20 mg/mL to about 60 mg/mL antibody, a buffer containing about 15 to about 20 mM or about 20 mM His/His-Cl, about 8% (weight/volume) sucrose, and about 0.2 mg/mL polysorbate 80. In one embodiment, an anti-TIGIT antibody may be formulated for injection as an aqueous solution comprising or consisting of 20 mg/mL to 60 mg/mL antibody, a buffer containing about 15 to about 20 mM or 20 mM His/His-Cl, 8% (weight/volume) sucrose, and 0.2 mg/mL polysorbate 80. In the foregoing embodiments, the pH of the aqueous solution is preferably about pH 5.0 to about pH 6.0, about pH 5.3 to about pH 6.0, about pH 5.5 to about pH 6.0, about pH 5.5 to about pH 5.8, or about pH 5.8. In some embodiments of the foregoing, the anti-TIGIT antibody is domvanalimab, or antigen binding fragment of domvanalimab or a variant of domvanalimab.

The pharmaceutical compositions of the present disclosure can be administered either orally or parenterally, but are preferably administered parenterally. Specifically, the pharmaceutical compositions are administered to patients by injection or percutaneous administration. Injections include, for example, intravenous injections, intravenous infusions, intramuscular injections, and subcutaneous injections, for systemic or local administration. For the purposes of this disclosure, injectable pharmaceutical compositions can include undiluted formulations (e.g., formulations to be used “as-is”) or formulations that are to be diluted with a physiological solution, such as saline (0.9% sodium chloride), dextrose in water (e.g., 5% dextrose) prior to administration, and the like.

In some embodiments, the anti-TIGIT antibody is domvanalimab, or an antigen-binding fragment of domvanalimab or a variant of domvanalimab, formulated as an aqueous solution for dilution prior to intravenous administration, the aqueous solution comprising or consisting of about 10 mg/mL to about 100 mg/mL antibody or about 20 mg/mL to about 60 mg/mL antibody, a buffer containing about 10 mM to about 30 mM histidine/histidine-HCl, about 1% to about 10% (weight/volume) of an excipient selected from the group consisting of sucrose, trehalose, sorbitol and mannitol, about 0 mg/mL to about 10 mg/mL NaCl, and about 0.05 mg/mL to about 0.6 mg/mL polysorbate 80 or about 0.1 mg/mL to about 0.3 mg/mL polysorbate 80.

In some embodiments, the anti-TIGIT antibody is domvanalimab, or an antigen-binding fragment of domvanalimab or a variant of domvanalimab, formulated as an aqueous solution for dilution prior to intravenous administration, the aqueous solution comprising or consisting of about 10 mg/mL to about 100 mg/mL antibody or about 20 mg/mL to about 60 mg/mL antibody, a buffer containing about 15 mM to about 30 mM histidine/histidine-HCl, about 1% to about 10% (weight/volume) of an excipient selected from the group consisting of sucrose, trehalose, sorbitol and mannitol, about 0 mg/mL to about 10 mg/mL NaCl, and about 0.05 mg/mL to about 0.6 mg/mL polysorbate 80 or about 0.1 mg/mL to about 0.3 mg/mL polysorbate 80.

In some embodiments, the anti-TIGIT antibody is domvanalimab, or an antigen-binding fragment of domvanalimab or a variant of domvanalimab, formulated as an aqueous solution for dilution prior to intravenous administration, the aqueous solution comprising or consisting of about 10 mg/mL to about 100 mg/mL antibody or about 20 mg/mL to about 60 mg/mL antibody, a buffer containing about 15 mM to about 20 mM histidine/histidine-HCl, about 1% to about 10% (weight/volume) of an excipient selected from the group consisting of sucrose, trehalose, sorbitol and mannitol, about 0 mg/mL to about 10 mg/mL NaCl, and about 0.05 mg/mL to about 0.6 mg/mL polysorbate 80 or about 0.1 mg/mL to about 0.3 mg/mL polysorbate 80.

In some embodiments, the anti-TIGIT antibody is domvanalimab, or an antigen-binding fragment of domvanalimab or a variant of domvanalimab, formulated as an aqueous solution for dilution prior to intravenous administration, the aqueous solution comprising or consisting of about 20 mg/mL to about 60 mg/mL antibody, a buffer containing about 15 mM to about 20 mM histidine/histidine-HCl, about 8% (weight/volume) of an excipient selected from sucrose, about 0 mg/mL to about 10 mg/mL NaCl, and about 0.05 mg/mL to about 0.6 mg/mL polysorbate 80 or about 0.1 mg/mL to about 0.3 mg/mL polysorbate 80.

In some embodiments, the anti-TIGIT antibody is domvanalimab, or an antigen-binding fragment of domvanalimab or a variant of domvanalimab, formulated as an aqueous solution for dilution prior to intravenous administration, the aqueous solution comprising or consisting of about 20 mg/mL to about 60 mg/mL antibody, a buffer containing about 20 mM histidine/histidine-HCl, about 8% (weight/volume) sucrose, and about 0.2 mg/mL polysorbate 80. In some embodiments, the pH of the solution is pH 5.5 to pH 6.0, or about pH 5.8.

The administration methods can be properly selected according to the patient's age, weight, and condition. The single-administration dose can be selected, for example, from within the range of 0.0001 to 100 mg of an antibody (e.g., domvanalimab, or another antibody described herein) per kg body weight. For example, when the antibody is administered to human patients intravenously, the dose of the antibody can be selected from within the range of 0.01 to 100 mg/kg body weight, preferably 0.05 to 100 mg/kg body weight, or more preferably 0.1 to 100 mg/kg. In some embodiments, the antibody is administered at a dose of, for example, about 0.01 to about 50 mg/kg, about 0.05 mg/kg to about 50 mg/kg, about 0.1 mg/kg to about 50 mg/kg, about 0.1 mg/kg to about 40 mg/kg, about 0.1 mg/kg to 30 mg/kg, about 0.1 mg/kg to about 20 mg/kg, about 0.3 mg/kg to 50 mg/kg, about 0.3 mg/kg to about 40 mg/kg, about 0.3 mg/kg to 30 mg/kg, about 0.3 mg/kg to about 20 mg/kg, about 0.4 mg/kg to 50 mg/kg, about 0.4 mg/kg to about 40 mg/kg, about 0.4 mg/kg to 30 mg/kg, about 0.4 mg/kg to about 20 mg/kg, about 0.5 mg/kg to 50 mg/kg, about 0.5 mg/kg to about 40 mg/kg, about 0.5 mg/kg to 30 mg/kg, about 0.5 mg/kg to about 20 mg/kg body weight of an antibody (e.g., domvanalimab, or another antibody described herein). In preferred embodiments, the antibody may be administered at a dose in the range of 0.1 mg/kg to 30 mg/kg, 0.5 mg/kg to 20 mg/kg, 0.5 mg/kg to 10 mg/kg, 1.0 mg/kg to 10 mg/kg, 10 mg/kg to 20 mg/kg, 10 mg/kg to 15 mg/kg, or 15 mg/kg to 20 mg/kg. Accordingly, the administered dose may be 0.1 mg/kg, 0.5 mg/kg, 1 mg/kg, 2 mg/lg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg or any dose in between the foregoing doses. In particular embodiments, the effective amount of domvanalimab or a fragment or variant thereof is about 0.1 mg/kg, about 0.5 mg/kg, about 1 mg/kg, about 1.5 mg/kg, about 2 mg/kg, about 2.5 mg/kg, about 5.0 mg/kg, about 10.0 mg/kg, about 15.0 mg/kg, or about 20.0 mg/kg. For the purposes of the forgoing doses, these amounts may be administered as appropriate, such as weekly, once every other week (Q2W), once every three weeks (Q3W), or once every four weeks (Q4W). In some embodiments, for example, the antibody may be administered at a dose of 10 mg/kg Q2W, Q3W, or Q4W. In some embodiments, the antibody may be administered at a dose of 15 mg/kg Q2W, Q3W, or Q4W. In some embodiments, the antibody may be administered at a dose of 20 mg/kg Q2W, Q3W, or Q4W.

In some embodiments, a dose of an antibody (e.g., domvanalimab, or another antibody described herein) can be selected irrespective of a subject's body weight. For example, in some embodiments, a dose of an antibody can be selected from within the range of 500 mg to 2000 mg of the antibody (e.g., domvanalimab, or another antibody described herein), irrespective of a subject's body weight. In some embodiments, a dose of an antibody can be selected from within the range of 700 mg to 1800 mg. In some embodiments, a dose of an antibody can be selected from within the range of 700 mg to 1500 mg, or 700 mg to 1400 mg. In some embodiments, a dose of an antibody can be selected from within the range of 1000 mg to 1500 mg. In some embodiments, a dose of an antibody can be selected from within the range of 1200 mg to 1500 mg. In some embodiments, the dose of the antibody (e.g., domvanalimab, or another antibody described herein) may be about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, or about 1800 mg.

For the purposes of any of the foregoing methods of treatment, a dose cycle may comprise, for example, dosing twice a week, once a week, once every 2 weeks, once every 3 weeks, once every 4 weeks, once every 5 weeks, once every 6 weeks, once every 7 weeks, or once every 8 weeks. Subjects may be administered 1, 2, 3, 4, 5, 6, 7, 8, 9, 10 or more dosing cycles. For example, a subject may be administered a plurality of dosing cycles until a suitable clinical endpoint is reached, for example disease progression, remission, or other clinical endpoint suitable based on the subject's cancer or intolerance.

In particular embodiments, wherein the anti-TIGIT antibody is domvanalimab or antigen-binding fragment thereof, a dose cycle may comprise about 10 mg/kg to about 20 mg/kg of antibody administered Q2W, Q3W or Q4W. In a specific example, a dose cycle may comprise about 10 mg/kg of antibody administered Q2W. In another specific example, a dose cycle may comprise about 15 mg/kg of antibody administered Q2W. In another specific example, a dose cycle may comprise about 15 mg/kg of antibody administered Q3W. In another specific example, a dose cycle may comprise about 20 mg/kg of antibody administered Q3W. In another specific example, a dose cycle may comprise about 20 mg/kg of antibody administered Q4W. In some embodiments, a dose cycle may comprise about 500 mg to about 2000 mg of antibody administered Q2W, Q3W, or Q4W. In some embodiments, a dose cycle may comprise about 600 mg to about 1600 mg of antibody administered Q2W, Q3W, or Q4W. In some embodiments, a dose cycle may comprise about 1000 mg to about 1500 mg of antibody administered Q2W, Q3W, or Q4W. In a specific example, a dose cycle may comprise about 600 mg to about 800 mg of antibody administered Q2W, Q3W, or Q4W. In a specific example, a dose cycle may comprise about 900 mg to about 1200 mg of antibody administered Q2W, Q3W, or Q4W. In a specific example, a dose cycle may comprise about 1200 mg to about 1600 mg of antibody administered Q2W, Q3W, or Q4W. In a specific example, a dose cycle may comprise about 600 mg to about 800 mg of antibody administered Q2W. In a specific example, a dose cycle may comprise about 900 mg to about 1200 mg of antibody administered Q3W. In a specific example, a dose cycle may comprise about 1200 mg to about 1600 mg of antibody administered Q4W. In a specific example, a dose cycle may comprise about 1000 mg of antibody administered Q2W. In another specific example, a dose cycle may comprise about 1200 mg of antibody administered Q2W. In another specific example, a dose cycle may comprise about 1200 mg of antibody administered Q3W. In another specific example, a dose cycle may comprise about 1500 mg of antibody administered Q3W. In another specific example, a dose cycle may comprise about 1500 mg of antibody administered Q4W. In one or more of the foregoing embodiments, the antibody may be administered intravenously, for example by an IV infusion.

In some embodiments, administration methods may comprise administering the anti-TIGIT antibody (e.g., domvanalimab) or a fragment or variant thereof along with at least one other antibody. For example, in some embodiments, anti-TIGIT antibody (e.g., domvanalimab) or a fragment or variant thereof may be co-administered with an antagonist antibody that binds to another checkpoint target, such as CTLA-4, PD-1, or PD-L1. In some embodiments, the anti-TIGIT antibody (e.g., domvanalimab) or a fragment or variant thereof and the at least one other antibody may be co-administered at the same dose. In some embodiments, the anti-TIGIT antibody (e.g., domvanalimab) or a fragment or variant thereof and the at least one other antibody may be co-administered at different doses. In some embodiments, the anti-TIGIT antibody (e.g., domvanalimab) or a fragment or variant thereof and the at least one other antibody may be administered in the same formulation or in separate formulations that are administered independently. In those embodiments in which the anti-TIGIT antibody (e.g., domvanalimab) or a fragment or variant thereof and the at least one other antibody are in separate formulations that are administered independently, the antibodies may be administered at the same time (i.e., concurrently), at about the same time, or in sequence (e.g., wherein the anti-TIGIT antibody (e.g., domvanalimab) or a fragment or variant thereof is administered first and the other antibody is administered sometime thereafter, or wherein the other antibody is administered first and the anti-TIGIT antibody (e.g., domvanalimab) or a fragment or variant thereof is administered sometime thereafter).

Any of the pharmaceutical compositions disclosed herein, including the pharmaceutical compositions comprising domvanalimab and fragments or variants thereof, can be used for treating and/or preventing cancer and achieving the disclosed therapeutic endpoints. Optimal doses and routes of administration may vary.

### VI. Treatment and Prevention of Cancer

The present disclosure provides treatments and preventions for cancer using anti-TIGIT antibodies with reduced or absent Fc effector function (e.g., domvanalimab and other antibodies of Section III). The methods are characterized by the unexpected advantages described herein. In some embodiments, the improved safety allows for patient selection, dosing and combination regimens. The disclosed anti-TIGIT antibodies with reduced or absent Fc effector function (e.g., domvanalimab and other antibodies of Section III) and compositions comprising the same can be used alone or in combination with one or more additional therapy to treat or prevent cancer and achieve certain biological endpoints as explained in more detail herein.

In general, the disclosed treatment methods comprise administering to a subject with cancer an anti-TIGIT antibody that has reduced binding to FcγR as compared to WT IgG1. Administration of the anti-TIGIT antibody (e.g., domvanalimab or other antibody of Section III) may comprise one or more (e.g., one, two, or three or more) dosing cycles. The anti-TIGIT antibody (e.g., domvanalimab) may be used in combination with one or more additional therapy. For example, in some embodiments the anti-TIGIT antibody may be administered along with an additional therapeutic agent such as, for example, an immunotherapeutic agent or a chemotherapeutic agent.

Treatment may be demonstrated by clinical or non-clinical efficacy. Non-limiting examples of clinical efficacy may include a reduction in tumor size, a reduction in tumor number, a reduction in metastasis, achievement of stable disease (SD), achievement of a partial response (PR), achievement of a complete response (CR), increased overall survival (OS), increased progression-free survival (PFS), increased time to progression, increased disease free survival, increased duration of response, an increase in duration of clinical benefit, an increase in time to treatment failure, a decrease in time to initial response, or any combination thereof. Non-limiting examples of non-clinical efficacy may include an increase in immune cell (e.g., T cell and/or NK cell) activation and/or proliferation in the periphery and/or in the tumor microenvironment, an increase in memory and antigen experienced T cells (e.g., CD39+CD103+CD8+ T cells) in the periphery, an increase in pro-inflammatory cytokines and/or chemokines in blood, plasma or serum, a molecular response by ctDNA dynamics, TCR receptor dynamics, a change in gene expression in cells from the tumor microenvironment, or any combination thereof. Measurements of an increase or decrease, as appropriate, may be made against baseline (i.e., prior to treatment), SOC, or other CPIs.

Unexpectedly, these treatments comprising an anti-TIGIT antibody that has reduced binding to FcγR as compared to WT IgG1 may be better tolerated compared to similar treatments with Fc-enabled anti-TIGIT antibodies (i.e., antibodies that bind to activating FcγRs substantially similar to WT IgG1 or with enhanced binding to WT IgG1), as demonstrated by fewer and/or less severe adverse events, fewer dose reductions, fewer temporary treatment disruptions (e.g., drug holidays or delays in treatment cycles), fewer treatment discontinuations, or any combination thereof. In particular, the disclosed treatments may have fewer and/or less severe treatment emergent adverse events as compared to similar treatments comprising an Fe-enabled anti-TIGIT antibody, fewer and/or less severe treatment emergent adverse events (TEAEs) as compared to similar treatments comprising an Fc-enabled anti-TIGIT antibody, fewer and/or less severe immune-related adverse events as compared to similar treatments comprising an Fc-enabled anti-TIGIT antibody, or fewer and/or less severe treatment-related immune-related adverse events as compared to similar treatments comprising an Fc-enabled anti-TIGIT antibody.

In one aspect, the present disclosure provides a method for treating cancer in a human subject in need thereof, comprising administering to the subject an anti-TIGIT antibody that has reduced binding to one or more activating human FcγRs as compared to wild type (WT) human IgG1, wherein treatment results in a reduction of one or more adverse event as compared to a similar treatment comprising an Fc-enabled anti-TIGIT antibody. In some embodiments, the subject does not experience an adverse event.

In another aspect, the present disclosure provides a method for treating cancer in a human subject in need thereof, comprising administering to the subject an anti-TIGIT antibody that has reduced binding to one or more activating human FcγRs as compared to wild type (WT) human IgG1, wherein the subject has a reduced likelihood of experiencing one or more adverse event as compared to a similar treatment comprising an Fc-enabled anti-TIGIT antibody.

In another aspect, the present disclosure provides a method for treating cancer in a human subject in need thereof without significantly increasing the likelihood of one or more immune-related adverse event, comprising administering to the subject an anti-TIGIT antibody that has reduced binding to one or more activating human FcγRs as compared to wild type (WT) human IgG1 and one or more additional therapy. Assessment of whether there is an increased likelihood of experiencing an adverse event after administration of a treatment comprising an anti-TIGIT antibody that has reduced binding to one or more activating human FcγRs may be made in comparison to the treatment lacking the anti-TIGIT antibody.

In another aspect, the present disclosure provides a method for reducing one or more adverse event experienced by a human subject being treated for cancer with an anti-TIGIT antibody, comprising administering to the subject an anti-TIGIT antibody that has reduced binding to one or more activating human FcγRs as compared to wild type (WT) human IgG1.

In another aspect, the present disclosure provides a method for reducing one or more adverse event experienced by a human subject being treated for cancer with an anti-TIGIT antibody and an additional immunotherapeutic agent, comprising administering to the subject an anti-TIGIT antibody that has reduced binding to one or more activating human FcγRs as compared to wild type (WT) human IgG1 and an additional immunotherapeutic agent.

In another aspect, the present disclosure provides a method for reducing dose reductions, temporary treatment disruptions, or treatment discontinuations experienced by a human subject being treated for cancer with an anti-TIGIT antibody, comprising administering to the subject an anti-TIGIT antibody that has reduced binding to one or more activating human FcγRs as compared to wild type (WT) human IgG1.

In some embodiments of the foregoing methods, the adverse event is a TEAE, optionally a treatment-related TEAE. In further embodiments of the foregoing methods, the adverse event is an immune-related AE (irAE), optionally a treatment-related irAE.

While treatment settings vary, for the purposes of the present disclosure, the assessment of whether a treatment comprising an anti-TIGIT antibody that has reduced binding to one or more activating human FcγRs results in a reduction of one or more adverse event, in dose reductions, in temporary treatment disruptions, or in treatment discontinuations, as compared to a similar treatment comprising an Fc-enabled anti-TIGIT antibody, may be made by comparing the occurrence of the event (e.g., adverse event, dose reduction, temporary treatment disruption, treatment discontinuation) between two populations. In view of the disclosure and the examples set forth herein, a person of skill in the art can determine populations that are appropriate for comparison.

The present disclosure also provides methods of blocking or preventing the binding of TIGIT to CD155 in a human subject without a significant reduction in Tregs, CD8+ T cells, CD4+ T cells, or a combination thereof, comprising administering to the subject an anti-TIGIT antibody that has reduced binding to FcγR as compared to WT IgG1, wherein any reduction in reduction in Tregs, CD8+ T cells, CD4+ T cells, or a combination thereof differs from a baseline measure prior to administration of the anti-TIGIT antibody by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the subject has cancer. In some embodiments of this method, the subject may undergo 1, 2, 3, or 4 or more dose cycles, which may comprise dosing once a week, once every 2 weeks, once every 3 weeks, once every 4 weeks, once every 5 weeks, once every 6 weeks, once every 7 weeks, or once every 8 weeks. In some embodiments, in some embodiments, any potential reduction in Tregs, CD8+ T cells, CD4+ T cells, or a combination thereof is assessed 1 week, 2 weeks, 3 weeks, 4 weeks, 5 weeks, 6 weeks, 7 weeks, or 8 weeks, after administration of the anti-TIGIT antibody. In some embodiments, administration of the anti-TIGIT antibody does not result in an immune-related adverse event. In some embodiments, this method may further comprise administering to the subject an immunotherapeutic agent, such as a checkpoint inhibitor (e.g., ipilimumab (YERVOY®), nivolumab (OPDIVO®), pembrolizumab (KEYTRUDA®), cemiplimab (LIBTAYO®), avelumab (BAVENCIO®), durvalumab (IMFINZI®), atezolizumab (TECENTRIQ®), or zimberelimab (AB 122).

a. Adverse Events

As used herein, the term “adverse event” or “AE” refers to any untoward medical occurrence in a subject administered a pharmaceutical product regardless of causal attribution. An AE can therefore be any of the following: (i) Any unfavorable and unintended sign (including an abnormal laboratory finding), symptom, or disease temporally associated with the use of a medicinal product, whether or not considered related to the medicinal product, (ii) Any new disease or exacerbation of an existing disease (a worsening in the character, frequency, or severity of a known condition), (iii) Recurrence of an intermittent medical condition (e.g., headache) not present at baseline, (iv) Any deterioration in a laboratory value or other clinical test (e.g., ECG, X-ray) that is associated with symptoms or leads to a change in study treatment or concomitant treatment or discontinuation from study treatment. An event which is part of the natural course of the disease under study (i.e., disease progression, death due to disease progression) should not be considered an AE. Instead, signs and symptoms of clinical sequelae resulting from disease progression should be considered only if they fulfill the AE definition.

The absence or presence of an adverse event, as well as its severity, can be assessed by physical examination and/or laboratory assessments. Clinically significant abnormal laboratory findings are those that are not associated with the underlying disease unless judged by the clinician to be more severe than expected for the subject's condition. Information on adverse events can be collected and coded to a standard term appropriate for the disease being treated, for example, according to the National Cancer Institute (NCI) Common Terminology Criteria for Adverse Events (CTCAE), or the Medical Dictionary for Regulatory Activities (MedDRA). Recommendations for testing and diagnosis are known in the art; see, for example, Schneider et al., “Management of Immune-Related Adverse Events in Patients Treated with Immune Checkpoint Inhibitor Therapy: ASCO Guideline Update,” Journal of Clinical Oncology, 2021, Vol. 39, No. 36.

The terms “severe adverse event” and “serious adverse event” are not synonymous. Severity and seriousness can be independently assessed for each AE. A serious adverse event is any adverse event that meets any of the following criteria: (i) Is fatal (i.e., the adverse event actually causes or leads to death), (ii) Is life threatening (i.e., the adverse event, in the view of the clinician, places the subject at immediate risk of death), (iii) Requires or prolongs inpatient hospitalization, (iv) Results in persistent or significant disability/incapacity (i.e., the adverse event results in substantial disruption of the subject's ability to conduct normal life functions), (v) Is a congenital anomaly/birth defect, (vi) Is a significant medical event in the clinician's judgment (e.g., may jeopardize the subject or may require medical/surgical intervention to prevent one of the outcomes listed above). Deaths that occur during the treatment period that are attributed by the clinician solely to progression of the disease under treatment should not be recorded as a serious adverse event.

Severity refers to the intensity of an adverse event. A severe adverse event may be of relatively minor medical significance (such as severe headache without any further findings). An assessment of severity grade can be made by a clinician, for example according to the NCI CTCAE (version 5.0). The table below can be used for assessing severity for AEs that are not specifically listed in the NCJ CTCAE. Not all grades may be appropriate for all AEs.

| Grade | Severity |
|---|---|
| 1 | Mild; asymptomatic or mild symptoms; clinical or diagnostic |
| | observations only; or intervention not indicated |
| 2 | Moderate; minimal, local, or non-invasive intervention indicated; |
| | or limiting age-appropriate instrumental activities of daily living^{a} |
| 3 | Severe or medically significant, but not immediately |
| | life-threatening; hospitalization or prolongation of hospitalization |
| | indicated; disabling; or limiting self-care activities of daily |
| | living^{b, c} |
| 4 | Life-threatening consequences or urgent intervention indicated^{d} |
| 5 | Death related to adverse event^{d} |

^{a}Instrumental activities of daily living refer to preparing meals, shopping for groceries or clothes, using the telephone, managing money, etc.
^{b}Examples of self-care activities of daily living include bathing, dressing and undressing, feeding oneself, using the toilet, and taking medications, as performed by patients who are not bedridden.
^{c}If an event is assessed as a “significant medical event,” it must be reported as a serious adverse event, per the definition of serious adverse event.
^{d}Grade 4 and 5 events must be reported as serious adverse events should the event meet the definition of serious adverse event.

A treatment-emergent adverse event (TEAE) is defined as any event not present prior to the initiation of the treatments or any event already present that worsens in either intensity or frequency following exposure to the treatments. Clinicians and medical professionals can use their knowledge of a subject, the circumstances surrounding the event, and an evaluation of potential alternative causes to determine whether an adverse event is considered to be related to treatment. A treatment-related adverse event means that the event is at least possibly related to the treatment. For example, there may be a plausible temporal relationship between the onset of the adverse event and administration of study treatment, and the adverse event cannot be readily explained by the participant's clinical state, intercurrent illness, or concomitant therapies; and/or the adverse event follows a known pattern of response to study treatment; and/or the adverse event abates or resolves upon discontinuation of study treatment or dose reduction and, if applicable, reappears upon re-challenge. An unrelated adverse event means the event is unlikely related to treatment. For example, evidence exists that the adverse event has an etiology other than treatment (e.g., preexisting medical condition, underlying disease, intercurrent illness, or concomitant medication); and/or the adverse event has no plausible temporal relationship to administration of the treatment (e.g., a cancer diagnosed 2 days after first dose of study treatment).

CPI therapy can be associated with a spectrum of side effects (i.e., adverse events) that are related to the mechanism of action, and which may be different from other systemic therapies such as cytotoxic chemotherapy or radiation therapy. The side effects may involve any organ or system of the body. Side effect associated with CPI therapy include but are not limited to immune-related adverse events. An immune-related adverse event may be defined as a treatment-emergent adverse event with Standardized Medical Queries (SMQs) of Hypersensitivity, Immune-mediated disorders, and autoimmune disorders. Common immune-related adverse events associated with CPI therapy include immune-mediated cutaneous and subcutaneous, gastrointestinal (GI), pulmonary, endocrine, musculoskeletal, renal, hematological, neurological, cardiovascular, and ocular adverse events, as well as infusion-related reactions. As noted above, it was expected that in order for anti-TIGIT antibodies to have efficacy, and in particular clinical efficacy, the antibody would need to be Fc-enabled. Incidences of adverse events, including immune-related adverse events, in humans resulting from treatment with regimens comprising CPIs are known in the art. In regards to those reported for Fc-enabled anti-TIGIT antibodies, see, for example, Mettu et al. 2022 Clin Cancer Res DOI:10.1158/1078-0432.CCR-21-2780 (Etigilimab); Van den Mooter et al. 2021 AACR (Poster CT118); Niu et al. 2022 Annals of Oncology DOI:10.1016/j.annonc.2021.11.002; Cho et al. 2021 Annals of Oncology 32 (suppl_7): S1428-S1457; and R. Shapira-Frommer et al. 2022 AACR (Poster CT508).

In some embodiments of the disclosed methods, the immune-related adverse event is a skin or subcutaneous tissue disorder, a GI disorder, a respiratory, thoracic or mediastinal disorder, an endocrine disorder, a musculoskeletal or connective tissue disorder, renal or urinary disorder, a hepatobiliary disorder, a blood or lymphatic system disorder, a nervous system disorder, a cardiac disorder, an infection or infestation, an injury, poisoning or procedural complication, or an eye disorder, as the terms are characterized in the NCI CTCAE v5. In some embodiments, the immune-related adverse event is a skin or subcutaneous tissue disorder, a GI disorder, a hepatobiliary disorder, an endocrine disorder, or a respiratory, thoracic or mediastinal disorder. In some embodiments, the immune-related adverse event may be Grade 1, Grade 2, Grade 3, or Grade 4. In some embodiments, the immune-related adverse event may be Grade 3 or higher. In certain embodiments of the above, the immune-related adverse event may be a severe adverse event.

In some embodiments of the disclosed methods, one or more immune-related adverse event is an immune-mediated skin or subcutaneous tissue disorder. Non-limiting examples of immune-related skin or subcutaneous disorders reported with CPIs include but are not limited to bullous dermatitis, dry skin, eczema, erythema multiforme, erythroderma, inflammatory dermatoses, maculo-papular rash, pain of skin, palmar-plantar erythrodysesthesia syndrome, pruritus, rash, Stevens-Johnson syndrome, and vitiligo. In some embodiments, one or more immune-related adverse event is an immune-mediated skin or subcutaneous tissue disorder adverse event, optionally selected from rash, maculo-papular rash, psoriasis, pruritis, and vitiligo. In some embodiments, one or more immune-related adverse event is an immune-mediated skin or subcutaneous tissue disorder selected from rash, maculo-papular rash, psoriasis, and pruritis. Despite the potential for lower grade presentation, immune-mediated skin toxicities may cause increased symptom burden, affect health-related quality of life (QoL) among patients treated with CPIs, and/or lead to treatment dose-reduction, disruption, or discontinuation.

In some embodiments of the disclosed methods, one or more immune-related adverse event is an immune-related GT disorder. Non-limiting examples of immune-related GT disorders reported with CPIs include but are not limited to colitis, diarrhea, enterocolitis, gastritis, hepatitis, oral mucositis, dry mouth, and pancreatitis. In some embodiments, one or more immune-related adverse event is an immune-related GI disorder selected from colitis, diarrhea, enterocolitis, gastritis, hepatitis, oral mucositis, dry mouth, and pancreatitis. In some embodiments, one or more immune-related adverse event is hepatitis.

In some embodiments of the disclosed methods, one or more immune-related adverse event is an immune-related respiratory, thoracic or mediastinal disorder. Immune-related respiratory, thoracic or mediastinal disorders reported with CPIs include but are not limited to dyspnea and pneumonitis. In some embodiments, one or more immune-related adverse event is pneumonitis.

In some embodiments of the disclosed methods, one or more immune-related adverse event is an immune-related endocrine disorder. Immune-related endocrine disorders reported with CPIs include but are not limited to hypothyroidism, hyperthyroidism, thyrotoxicosis, primary adrenal insufficiency, hypophysitis, and diabetes. In some embodiments, one or more immune-related adverse event is an immune-mediate endocrine adverse event selected from hypothyroidism, hyperthyroidism, adrenal insufficiency, and diabetes.

In some embodiments of the disclosed methods, one or more immune-related adverse event is an immune-related musculoskeletal or connective tissue disorder. Immune-related musculoskeletal or connective tissue disorders reported with CPIs include but are not limited to arthralgia, arthritis, myalgia, myositis, and polymyalgia-like syndrome. In some embodiments, one or more immune-related adverse event is an immune-related musculoskeletal or connective tissue selected from arthralgia, arthritis, myalgia myositis, and polymyalgia-like syndrome.

In some embodiments of the disclosed methods, one or more immune-related adverse event is an immune-related renal or urinary disorder. Non-limiting examples of immune-related renal or urinary disorders reported with CPIs include nephritis or acute kidney injury (AKI). In some embodiments, one or more immune-related adverse event is an immune-related renal or urinary disorder selected from nephritis and AKI.

In some embodiments of the disclosed methods, one or more immune-related adverse event is an immune-related nervous system disorder. Immune-related nervous system disorders reported with CPIs include but are not limited to myasthenia gravis or myasthenic syndrome, myasthenia gravis with myositis overlap, aseptic meningitis, encephalitis, posterior reversible encephalopathy syndrome, Guillain-Barre syndrome or Guillain-Barre-like syndrome, enteric neuropathy, transverse myelitis, and a variety of other peripheral neuropathy phenotypes and demyelinating disorders (e.g., multiple sclerosis, acute-disseminated encephalomyelitis, optic neuritis, neuromyelitis optica, etc.). In some embodiments, one or more immune-related adverse event is a neurologic immune-related adverse event, optionally selected from myasthenia gravis, myasthenic syndrome, aseptic meningitis, encephalitis, posterior reversible encephalopathy syndrome, Guillain-Barre syndrome, Guillain-Barre-like syndrome, enteric neuropathy, and transverse myelitis.

In some embodiments of the disclosed methods, one or more immune-related adverse event is an immune-related blood or lymphatic disorder. Immune-related blood or lymphatic disorders reported with CPIs include but are not limited to cryoglobulinemia, anemia, thrombotic thrombocytopenic purpura (TTP), hemolytic uremic syndrome, decreased lymphocyte count, immune thrombocytopenia (ITP), and hemophilia A. In some embodiments, one or more immune-related adverse event is an related blood or lymphatic disorder selected from cryoglobulinemia, hemolytic anemia, acquired TTP, hemolytic uremic syndrome, aplastic anemia, lymphopenia, ITP, and acquired hemophilia A.

In some embodiments of the disclosed methods, one or more immune-related adverse event is an immune-related cardiac disorder. Immune-related cardiac disorders reported with CPIs include but are not limited to myocarditis, pericarditis, airhythmias, impaired ventricular function with heart failure, vasculitis, and venous thromboembolism. In some embodiments, one or more immune-related adverse event is an immune-related cardiac disorder selected from myocarditis, pericarditis, arrhythmias, impaired ventricular function with heart failure, vasculitis, and venous thromboembolism.

In some embodiments of the disclosed methods, one or more immune-related adverse event is an immune-related eye disorder. Immune-related eye disorders reported with CPIs include but are not limited to conjunctivitis, orbital inflammation, uveitis, iritis, and episcleritis.

In some embodiments, one or more immune-related adverse event is an immune-related ocular disorder selected from conjunctivitis, orbital inflammation, uveitis, iritis, and episcleritis.

In some embodiments of the disclosed methods, one or more immune-related adverse event is an immune-related is a general disorder, injury, or procedural complication. Non-limiting examples reported with CPIs include chills, swelling of the face (edema face), fatigue, pyrexia, and infusion related reaction. In some embodiments, one or more immune-related adverse event is selected from chills, swelling of the face (edema face), fatigue, pyrexia, and infusion related reaction.

In some embodiments of the disclosed methods, one or more immune-related adverse event is selected from rash, oral mucositis, dry mouth, colitis/diarrhea, hepatitis, pneumonitis, hypophysitis, hypothyroidism, hyperthyroidism, adrenal insufficiency, diabetes, nephritis, pancreatitis, myositis, arthritis, Guillain-Barre syndrome, myasthenia gravis, posterior reversible encephalopathy syndrome, aseptic meningitis, enteric neuropathy, transverse myelitis, autoimmune encephalitis, cardiotoxicity (e.g., myocarditis and conduction abnormalities), hematologic toxicity (e.g., red cell aplasia, neutropenia, thrombocytopenia, acquired hemophilia A, and cryoglobulinemia), and eye inflammation (e.g., episcleritis, conjunctivitis, uveitis or orbital inflammation). In some embodiments, one or more immune-related adverse event is selected from rash, oral mucositis, dry mouth, colitis/diarrhea, hepatitis, pneumonitis, hypophysitis, hypothyroidism, hyperthyroidism, adrenal insufficiency, and diabetes. In some embodiments, one or more immune-related adverse event is selected from arthritis, hepatitis, hypothyroidism, hyperthyroidism, infusion-related reactions, maculo-papular rash, pneumonitis, pruritis, psoriasis, rash, and swelling Face. In some embodiments, one or more immune-related adverse event is selected from infusion-related reactions, maculo-papular rash, pruritis, psoriasis, and rash.

In one or more of the foregoing embodiments, the occurrence of an immune-related adverse event may be decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, or more. In one or more of the foregoing embodiments, the occurrence of immune-mediated adverse event may occur in about 50% or less of the population treated, about 45% or less of the population treated, about 40% or less of the population treated, about 35% or less of the population treated, about 30% or less of the population treated, about 25% or less of the population treated, about 20% or less of the population treated, about 15% or less of the population treated, about 10% or less of the population treated, about 5% or less of the population treated, less than 5% of the population treated.

In a specific example, the occurrence of pruritus in a population treated may be ≤40%, <35%, ≤30%, ≤25%, ≤20%, ≤20%, ≤15%, ≤10%, ≤5%, ≤4%, ≤3%, ≤2%, or ≤1%. In a specific example, the occurrence of rash in a population treated may be 40%, ≤35%, ≤30%, <25%, ≤20%, ≤20%, ≤15%, ≤10%, ≤5%, ≤4%, ≤3%, ≤2%, or ≤1%. In a specific example, the occurrence of maculo-papular rash in a population treated may be ≤40%, ≤35%, ≤30%, <25%, ≤20%, ≤20%, ≤15%, ≤10%, ≤5%, ≤4%, ≤3%, ≤2%, or ≤1%. In a specific example, the occurrence of infusion-related reactions in a population treated may be ≤40%, ≤35%, <30%, ≤25%, ≤20%, ≤20%, ≤15%, ≤10%, ≤5%, ≤4%, ≤3%, ≤2%, or ≤1%. In a specific example, the occurrence of immune-mediated hepatitis in a population treated may be ≤40%, <35%, ≤30%, ≤25%, ≤20%, ≤20%, ≤15%, ≤10%, ≤5%, ≤4%, ≤3%, ≤2%, or ≤1%. In a specific example, the occurrence of pneumonitis in a population treated may be ≤40%, ≤35%, <30%, ≤25%, ≤20%, ≤20%, ≤15%, ≤10%, ≤5%, ≤4%, ≤3%, ≤2%, or ≤1%. In a specific example, the occurrence of arthritis in a population treated may be ≤40%, ≤35%, ≤30%, <25%, ≤20%, ≤20%, ≤15%, ≤10%, ≤5%, ≤4%, ≤3%, 2%, or ≤1%. In a specific example, the occurrence of hyperthyroidism in a population treated may be ≤40%, ≤35%, ≤30%, ≤25%, 20%, ≤20%, ≤15%, ≤10%, ≤5%, ≤4%, ≤3%, ≤2%, or ≤1%. In a specific example, the occurrence of hypothyroidism in a population treated may be ≤40%, ≤35%, ≤30%, ≤25%, <20%, ≤20%, ≤15%, ≤10%, ≤5%, ≤4%, ≤3%, ≤2%, or ≤1%. In a specific example, the occurrence of psoriasis in a population treated may be ≤40%, ≤35%, ≤30%, ≤25%, ≤20%, <20%, ≤15%, ≤10%, ≤5%, ≤4%, ≤3%, ≤2%, or ≤1%. In a specific example, the occurrence of swelling face in a population treated may be ≤40%, ≤35%, ≤30%, ≤25%, ≤20%, ≤20%, <15%, ≤10%, ≤5%, ≤4%, ≤3%, ≤2%, or ≤1%. In a specific example, the occurrence of a skin or subcutaneous tissue disorder in a population treated may be 40%, ≤35%, ≤30%, ≤25%, <20%, ≤20%, ≤15%, ≤10%, ≤5%, ≤4%, ≤3%, 2%, or ≤1%. In a specific example, the occurrence of a gastrointestinal disorder in a population treated may be 40%, ≤35%, ≤30%, <25%, ≤20%, ≤20%, ≤15%, ≤10%, ≤5%, ≤4%, ≤3%, ≤2%, or ≤1%.

Without being bound by theory, the reduction in number, frequency, and/or severity of immune-related adverse events may be a result of the sustained maintenance of one or several immunological parameters for a duration after administration and/or across multiple dosing cycles. For example, the present disclosure shows that the number of T cells, in particular Treg and CD8+ T cells, measured in a sample obtained from a subject treated with anti-TIGIT antibodies that have reduced binding to FcγR as compared to WT IgG1 is more than a corresponding measurement in a population of subjects treated with an Fc-enabled anti-TIGIT antibody. Moreover, the present disclosure shows that anti-TIGIT antibodies that have reduced binding to FcγR as compared to WT IgG1 do not reduce the total number of CD8+ T cells or Treg cells after treatment, or if there is a reduction the difference is less than a corresponding measurement in a population of subjects treated with an Fc-enabled anti-TIGIT antibody and/or is within the range seen for healthy subjects. As used herein, the term “corresponding measurement in a population of subjects” refers to an average measurement for the population of subjects, which is typically comprised of patients with similar disease (e.g., type and stage of cancer), that is obtained using the same assay, on the same sample type (e.g., blood, tumor biopsy), on the same day post-administration. This maintenance of CD8+ T cells and Treg cells may be preserved through multiple dosing cycles (e.g., 2, 3, 4, or more dosing cycles) and for days (e.g., 1, 2, 3, 4, 5, 6, or 7) or even weeks (e.g., 1, 2, 3, 4, 5, 6, 7, or 8 or more) following administration of the anti-TIGIT antibody with reduced or absent Fc effector function. Because absolute cell numbers vary from subject to subject, it may be preferred to reference a percent change from baseline or a fold change from baseline, which can be calculated using a measurement from a first sample at baseline and a measurement from second sample obtained an amount of time after the administration of the anti-TIGIT antibody.

b. Efficacy

For the purposes of any of the foregoing methods of treatment, treatment may be demonstrated by clinical efficacy or non-clinical efficacy. Non-limiting examples of clinical efficacy include a reduction in tumor size, a reduction in tumor number, a reduction in metastasis, achievement of stable disease (SD), achievement of a partial response (PR), achievement of a complete response (CR), increased overall survival (OS), increased progression-free survival (PFS), increased time to progression, increased disease free survival, increased duration of response, an increase in duration of clinical benefit, an increase in time to treatment failure, or any combination thereof. Non-limiting examples of non-clinical efficacy may include an increase in immune cell (e.g., T cell and/or NK cell) activation and/or proliferation in the periphery and/or in the tumor microenvironment, an increase in memory and antigen experienced T cells (e.g., CD39+CD103+CD8+ T cells) in the periphery, an increase in pro-inflammatory cytokines and/or chemokines in blood, plasma or serum, a molecular response by ctDNA dynamics, TCR receptor dynamics, a change in gene expression in cells from the tumor microenvironment, or any combination thereof.

In some embodiments, treatment results in a reduction in tumor size, a reduction in tumor number, a reduction in metastasis, or a combination thereof. In some embodiments, treatment results in stable disease (SD), a partial response (PR), a complete response (CR), or a combination thereof. A person of skill in the art can determine the proper criteria for determining SD, PR, and CR depending upon the type of cancer. For example, SD, PR, and CR may be defined for solid tumors based on the percent change from baseline for measurable target lesions per RECIST 1.1 over time. For example, in some embodiments, PR may refer to a decrease of 30% (inclusive) up to 100%, complete response may refer to when all lesions have disappeared (i.e., 100% decrease), and stable disease typically may refer to an increase of less than 20% to a decrease of 30% (not inclusive).

In some embodiments, treatment results in an improvement in overall survival (OS), progression-free survival (PFS), disease control rate (DCR), overall response rate (ORR), or a combination thereof compared to placebo or a current standard of care. In some embodiments, treatment results in an improvement in overall survival (OS), progression-free survival (PFS), disease control rate (DCR), overall response rate (ORR), or a combination thereof compared to treatment with an Fc-enabled anti-TIGIT antibody, such as AB308, BMS-986207, tiragolumab, vibostolimab, etigilimab, ociperlimab, ralzapastotug, EOS-448, SEA-TGT, AGEN1777, AGEN1327, and JS006. In some embodiments, treatment results in an improvement in overall survival (OS), progression-free survival (PFS), disease control rate, overall response rate, or a combination thereof compared to treatment with a CPI (e.g., ipilimumab, nivolumab, pembrolizumab, cemiplimab, avelumab, durvalumab, atezolizumab, and zimberelimab alone. In particular embodiments of the above, the improvement may be statistically significant.

In some embodiments, treatment results in non-inferior overall survival (OS), progression-free survival (PFS), disease control rate (DCR), overall response rate (ORR), or a combination thereof, as compared to a current standard of care. In some embodiments, treatment results in non-inferior overall survival (OS), progression-free survival (PFS), disease control rate (DCR), overall response rate (ORR), or a combination thereof, as compared to treatment with an Fc-enabled anti-TIGIT antibody, such as AB308, BMS-986207, ralzapastotug, tiragolumab, vibostolimab, etigilimab, ociperlimab, EOS-448, SEA-TGT, AGEN1777, AGEN1327, and JS006. In some embodiments, treatment results in non-inferior overall survival (OS), progression-free survival (PFS), disease control rate (DCR), overall response rate (ORR), or a combination thereof, as compared to treatment with a CPI (e.g., ipilimumab, nivolumab, pembrolizumab, cemiplimab, avelumab, durvalumab, atezolizumab, and zimberelimab alone).

The present disclosure also shows that the percent change from baseline for the number of CD8+ T cells and/or Tregs, measured in blood, or tumor biopsy samples obtained 1, 2, 3, 4, 5, 6, or 7 days, or 1, 2, 3, 4, 5, 6, 7, or 8 or more weeks after administration of the anti-TIGIT antibody differs by less than a corresponding measurement in a population of subjects treated with an Fc-enabled anti-TIGIT antibody and/or is within the range seen for healthy subjects. In some subjects, the percent change from baseline for the number of CD8+ T cells and/or Tregs may not significantly differ from a baseline number of CD8+ T cells or Tregs prior to administration. Similarly, the number of CD8+ T cells or Tregs measured in blood, or tumor biopsy samples obtained 1, 2, 3, 4, 5, 6, or 7 days, or 1, 2, 3, 4, 5, 6, 7, or 8 or more weeks after administration of the anti-TIGIT antibody may differ by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% compared to a baseline number of CD8+ T cells or Tregs, respectively, prior to administration. Additionally or alternatively, the number of CD8+ T cells or Tregs measured in blood, or tumor biopsy samples obtained after 1, 2, 3, or 4 dosing cycles of the anti-TIGIT antibody may not significantly differ from a baseline number of CD8+ T cells or Tregs, respectively, prior to commencing treatment. Additionally or alternatively, the number of CD8+ T cells or Tregs measured in blood, or tumor biopsy samples obtained after 1, 2, 3, or 4 dosing cycles of the anti-TIGIT antibody may differ by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% compared to a baseline number of CD8+ T cells or Tregs, respectively, prior to commencing treatment.

As a result, the ratio of CD8+ T cell to Tregs may not significantly change through the course of treatment. In other words, an analysis of CD8+ T cells and Treg cells, measured in a blood sample or in a tumor sample, 1, 2, 3, 4, 5, 6, 7, or 8 or more weeks after administration of the anti-TIGIT antibody may show that the ratio of CD8+ T cell to Tregs differs by less than a corresponding measurement in a population of subjects treated with an Fc-enabled anti-TIGIT antibody and/or is within the range seen for healthy subjects. In some examples, the ratio of CD8+ T cell to Tregs differs by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% compared to a baseline ratio of CD8+ T cells to Tregs prior to commencing treatment. Similarly, an analysis of CD8+ T cells and Treg cells, measured in blood, or tumor biopsy, 1, 2, 3, 4, 5, 6, 7, or 8 or more weeks after administration of the anti-TIGIT antibody may show that the ratio of CD8+ T cell to TIGIT+Tregs differs by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% compared to a baseline ratio of CD8+ T cells to Tregs prior to commencing treatment. Additionally or alternatively, an analysis of CD8+ T cells and Treg cells, measured in blood, or tumor biopsy, after 1, 2, 3, or 4 dosing cycles of the anti-TIGIT antibody may show that the ratio of CD8+ T cell to Tregs differs by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% compared to a baseline ratio of CD8+ T cells to Tregs prior to commencing treatment. Additionally or alternatively, an analysis of CD8+ T cells and Treg cells, measured in blood, or tumor biopsy, after 1, 2, 3, or 4 dosing cycles of the anti-TIGIT antibody may show that the ratio of CD8+ T cell to TIGIT+ Tregs differs by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% compared to a baseline ratio of CD8+ T cells to Tregs prior to commencing treatment.

Additionally, after administration of an anti-TIGIT antibody that has reduced binding to FcγR as compared to WT IgG1, there may be (i) an increase in the subject's CD8+ T cell and/or NK cell proliferation, (ii) an increase in the subject's CD8+ T cell function, (iii) a decrease in CD8+ T cell exhaustion, or (iv) a combination thereof, as compared to the subject's baseline measurement. In fact, the subject may experience myeloid cell proliferation, lymphoid cell proliferation, or a combination thereof. Indeed, administration of the disclosed anti-TIGIT antibodies may result in monocyte activation, lymphocyte activation, or both. Thus, in some embodiments, 1, 2, 3, 4, 5, 6, or 7 days, or 1, 2, 3, 4, 5, 6, 7, or 8 or more weeks after administration of the anti-TIGIT antibody there may be (i) an increase in the subject's CD8+ T cell and/or NK cell expansion or proliferation, (ii) an increase in the subject's CD8+ T cell function, (iii) a decrease in CD8+ T cell exhaustion, or (iv) a combination thereof. In some embodiments, 1, 2, 3, 4, 5, 6, or 7 days, or 1, 2, 3, 4, 5, 6, 7, or 8 or more weeks after administration of the anti-TIGIT antibody the subject may exhibit myeloid and/or lymphoid expansion or proliferation or activation. In some embodiments, after 1, 2, 3, or 4 dosing cycles of the anti-TIGIT antibody there may be (i) an increase in the subject's CD8+ T cell and/or NK cell expansion or proliferation, (ii) an increase in the subject's CD8+ T cell function, (iii) a decrease in CD8+ T cell exhaustion, or (iv) a combination thereof. In some embodiments, after 1, 2, 3, or 4 dosing cycles of the anti-TIGIT antibody the subject may exhibit myeloid and/or lymphoid expansion or proliferation or activation. Further, concurrently with the foregoing expansions/proliferations/activations, the decrease in total Tregs or TIGIT+Tregs may not significantly differ from or may differ by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30 compared to a baseline measure taken prior to administration or commencement of treatment.

Myeloid and lymphoid cell numbers in tissue or blood may be quantified (absolute numbers or relative numbers) by immunophenotyping, i.e., a process of using antibodies (or other antigen-specific reagent) to detect and quantify cell-associated antigens. Lymphoid cell markers may include but are not limited to CD3, CD4, CD8, CD16, CD25, CD39, CD45, CD56, CD103, CD127, and FOXP3. CD4 and CD8 can distinguish T cell with different effector functions (e.g., CD4⁺ T cells and CD8⁺ T cells). Co-expression of different cell markers can further distinguish sub-groups. For example, co-expression of CD39 and CD103 can differentiate tumor-specific T cells (CD8⁺CD39⁺CD103⁺ T cells) from bystander T cells in the tumor microenvironment (TME), while co-expression of CD4, CD25, and FOXP3 can distinguish a population of Treg cells (CD4⁺CD25⁺FOXP3⁺Tregs). For myeloid cells, suitable markers may include but are not limited to CD14, CD68, CD80, CD83, CD86, CD163, and CD206. Ki67 is a non-limiting example of a suitable marker of cell proliferation, such that an increase in Ki67 positive cells (e.g., CD4⁺ T cells, CD8⁺ T cells, CD163⁺, CD206⁺ macrophages, etc.) as compared to a reference sample indicate cell proliferation. IFN Q, Ki-67, and GranB are non-limiting examples of suitable markers of CD8⁺ T cell function, such that an increase in IFN Q, Ki-67⁺ and/or GranB⁺ CD8⁺ T cells indicates an increase in CD8⁺ T cell function in patients. A primary feature of exhausted T cells is the sustained coexpression of multiple inhibitory surface receptors, referred to commonly as immune checkpoints, including but not limited to CTLA4, PD-1, TIM3, TIGIT, and SLAMF6. Recent evidence suggests that the exhausted phenotype in CD8+ T cells is not homogeneous and includes various subgroups, such as “progenitor” and “terminally-differentiated”, with varied capacities for effector function and proliferation dispersed among the subgroups. See, for example, McLane et al., “CD8 T Cell Exhaustion During Chronic Viral Infection and Cancer,” Annual Review of Immunology, 2019, 37: 457-495, the disclosures of which are incorporated herein by reference. For instance, terminally exhausted CD8+ T cells are TIM3⁺ and characterized by high levels of PD-1 (PD-1^{hi}) on their surface.

It is also believed that the disclosed methods and treatments with anti-TIGIT antibodies with reduced or abolished binding to FcγRs do not cause (or cause minimal) disruptions to T cell receptor (TCR) diversity among various T cell populations. For example, the disclosed methods and treatments with anti-TIGIT antibodies with reduced or abolished binding to FcγRs may result in an on-treatment increase in T cell receptor diversity, as measured in, for example, a blood sample. Newly activated T cells clones may also make their way to the tumor site, resulting in an increase TCR diversity in a tumor sample. In some embodiments, 1, 2, 3, 4, 5, 6, or 7 days, or 1, 2, 3, 4, 5, 6, 7, or 8 or more weeks after administration of the anti-TIGIT antibody the subject may maintain or increase TCR diversity compared to a baseline established prior to administration. In some embodiments, after 1, 2, 3, or 4 dosing cycles of the anti-TIGIT antibody the subject may maintain or increase TCR diversity compared to a baseline established prior to commencement of treatment. TCR diversity may be measured by methods known in the art, including, for example, by RNAseq. See, for example, Chiffelle et al., “T-cell repertoire analysis and metrics of diversity and clonality,” Current Opinion in Biotechnology, 2020, 65: 284-295, the disclosures of which are incorporated herein by reference.

Taken together, the disclosure provides that administration of or treatment with an anti-TIGIT antibody with reduced or absent Fc effector functions resulting from a reduced capacity or inability to bind to FcγRs (e.g., FcγRT, FcγRIIA, FcγRIIA), a subject may not experience perturbations of the immune system that have commonly accompanied Fc-enabled anti-TIGIT antibodies, and thus making the disclosed methods unexpectedly safe.

Accordingly, the present disclosure provides—in addition to treatments with fewer or less severe side effects and immune-related adverse events—methods for treating cancer in a human subject in need thereof, comprising administering to the subject an anti-TIGIT antibody that has reduced binding to FcγR as compared to WT IgG1, wherein the subject's ratio of CD8+ T cells to Tregs measured in a first sample at baseline and in a second sample 1 or more day, or 1 or more weeks, after a first administration of the anti-TIGIT antibody differs by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the administering can comprise one or more dosing cycles (e.g., 1, 2, 3, or 4 or more), in which the anti-TIGIT antibody is administered and then re-administered at a later time (e.g., 1, 2, 3, 4, 5, 6, 7, or 8 or more weeks after the prior administration). For example, the one or more dosing cycles may comprise administering the anti-TIGIT antibody to the subject once every 2 weeks, once every 3 weeks, once every 4 weeks, once every 5 weeks, once every 6 weeks, once every 7 weeks, or once every 8 weeks. In some embodiments, the one or more dosing cycles may comprise administering the anti-TIGIT antibody to the subject at a dose of about 10 mg/kg to about 20 mg/kg. In some embodiments, the one or more dosing cycles may comprise administering the anti-TIGIT antibody to the subject at a dose in a range of about 500 mg to about 2000 mg, about 600 mg to about 800 mg, about 900 mg to about 1200 mg, about 1200 mg to about 1600 mg, or about 1200 mg to about 1500 mg. In some embodiments, the first sample and second sample are selected from a blood sample, a tumor sample, and a combination thereof. In some embodiments, the second sample is obtained 1, 2, 3, 4, 5, 6, or 7 days, or 1, 2, 3, 4, 5, or 6 weeks after a first administration of the anti-TIGIT antibody. In some embodiments, the ratio of CD8+ T cells to Tregs differs by less than a corresponding ratio at a corresponding time in a patient treated with a Fc-enabled anti-TIGIT antibody, such as AB308, BMS-986207, tiragolumab, vibostolimab, etigilimab, ociperlimab, ralzapastotug, EOS-448, SEA-TGT, AGEN1777, AGEN1327, and JS006.

Additionally or alternatively, the present disclosure provides methods for treating cancer in a human subject in need thereof, comprising administering to the subject an anti-TIGIT antibody that has reduced binding to FcγR as compared to WT IgG1, wherein the subject's ratio of CD8+ T cells to TIGIT+Tregs measured in a first sample at baseline and in a second sample 1 or more days, or 1 or more weeks after a first administration of administering the anti-TIGIT antibody differs by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the administering can comprise one or more dosing cycles (e.g., 1, 2, 3, or 4 or more), in which the anti-TIGIT antibody is administered and then re-administered at a later time (e.g., 1, 2, 3, 4, 5, 6, 7, or 8 or more weeks after the prior administration). For example, the one or more dosing cycles may comprise administering the anti-TIGIT antibody to the subject once every 2 weeks, once every 3 weeks, once every 4 weeks, once every 5 weeks, once every 6 weeks, once every 7 weeks, or once every 8 weeks. In some embodiments, the one or more dosing cycles may comprise administering the anti-TIGIT antibody to the subject at a dose of about 10 mg/kg to about 20 mg/kg. In some embodiments, the one or more dosing cycles may comprise administering the anti-TIGIT antibody to the subject at a dose in a range of about 500 mg to about 2000 mg, about 600 mg to about 800 mg, about 900 mg to about 1200 mg, about 1200 mg to about 1600 mg, or about 1200 mg to about 1500 mg. In some embodiments, the first sample and second sample are selected from a blood sample, a tumor sample, and a combination thereof. In some embodiments, the second sample is obtained 1, 2, 3, 4, 5, 6, or 7 days, or 1, 2, 3, 4, 5, or 6 weeks after a first administration of the anti-TIGIT antibody. In some embodiments, the ratio of CD8+ T cells to TIGIT+Tregs differs by less than a corresponding ratio at a corresponding time in a patient treated with a Fe-enabled anti-TIGIT antibody, such as AB308, BMS-986207, tiragolumab, vibostolimab, etigilimab, ociperlimab, ralzapastotug, EOS-448, SEA-TGT, AGEN1777, AGEN1327, and JS006.

Additionally or alternatively, the present disclosure provides methods for treating cancer in a human subject in need thereof, comprising administering to the subject an anti-TIGIT antibody that has reduced binding to FcγR as compared to WT IgG1, wherein the subject's Tregs, TIGIT+Tregs, or a combination thereof measured in a first sample at baseline and in a second sample 1 or more day, or 1 or more weeks, after a first administration of administering the anti-TIGIT antibody differs by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the administering can comprise one or more dosing cycles (e.g., 1, 2, 3, or 4 or more), in which the anti-TIGIT antibody is administered and then re-administered at a later time (e.g., 1, 2, 3, 4, 5, 6, 7, or 8 or more weeks after the prior administration). For example, the one or more dosing cycles may comprise administering the anti-TIGIT antibody to the subject once every 2 weeks, once every 3 weeks, once every 4 weeks, once every 5 weeks, once every 6 weeks, once every 7 weeks, or once every 8 weeks. In some embodiments, the one or more dosing cycles may comprise administering the anti-TIGIT antibody to the subject at a dose of about 10 mg/kg to about 20 mg/kg. In some embodiments, the one or more dosing cycles may comprise administering the anti-TIGIT antibody to the subject at a dose in a range of about 500 mg to about 2000 mg, about 600 mg to about 800 mg, about 900 mg to about 1200 mg, about 1200 mg to about 1600 mg, or about 1200 mg to about 1500 mg. In some embodiments, the first sample and second sample are selected from a blood sample, a tumor sample, and a combination thereof. In some embodiments, the second sample is obtained 1, 2, 3, 4, 5, 6, or 7 days, or 1, 2, 3, 4, 5, or 6 weeks after a first administration of the anti-TIGIT antibody. In some embodiments, the amount of Tregs or TIGIT+Tregs differs by less than a corresponding amount at a corresponding time in a patient treated with a Fc-enabled anti-TIGIT antibody, such as AB308, BMS-986207, tiragolumab, vibostolimab, etigilimab, ociperlimab, EOS-448, SEA-TGT, AGEN1777, AGEN1327, ralzapastotug, and JS006.

Additionally or alternatively, the present disclosure provides methods for treating cancer in a human subject in need thereof, comprising administering to the subject an anti-TIGIT antibody that has reduced binding to FcγR as compared to WT IgG1, wherein the subject's Tregs, TIGIT+Tregs, or a combination thereof differs by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% during treatment with the anti-TIGIT antibody. In some embodiments, the administering can comprise one or more dosing cycles (e.g., 1, 2, 3, or 4 or more), in which the anti-TIGIT antibody is administered and then re-administered at a later time (e.g., 1, 2, 3, 4, 5, 6, 7, or 8 or more weeks after the prior administration). For example, the one or more dosing cycles may comprise administering the anti-TIGIT antibody to the subject once every 2 weeks, once every 3 weeks, once every 4 weeks, once every 5 weeks, once every 6 weeks, once every 7 weeks, or once every 8 weeks. In some embodiments, the one or more dosing cycles may comprise administering the anti-TIGIT antibody to the subject at a dose of about 10 mg/kg to about 20 mg/kg. In some embodiments, the one or more dosing cycles may comprise administering the anti-TIGIT antibody to the subject at a dose in a range of about 500 mg to about 2000 mg, about 600 mg to about 800 mg, about 900 mg to about 1200 mg, about 1200 mg to about 1600 mg, or about 1200 mg to about 1500 mg. In some embodiments, the lack of a significant difference in Tregs, TIGIT+Tregs, or a combination thereof may be observed at least 1 day, at least 1 week, at least 2 weeks, at least 3 weeks, or at least 4 weeks after administration of the anti-TIGIT antibody. In some embodiments, the lack of a significant difference in Tregs, TIGIT+Tregs, or a combination thereof may be observed after 1, 2, 3, or 4 or more dosing cycles. In some embodiments, the amount of Tregs or TIGIT+Tregs differs by less than a corresponding amount at a corresponding time in a patient treated with a Fc-enabled anti-TIGIT antibody, such as AB308, BMS-986207, tiragolumab, vibostolimab, etigilimab, ociperlimab, ralzapastotug, EOS-448, SEA-TGT, AGEN1777, AGEN1327, and JS006.

Additionally or alternatively, the present disclosure provides methods for treating cancer in a human subject in need thereof, comprising administering to the subject an anti-TIGIT antibody that has reduced binding to FcγR as compared to WT IgG1, wherein after administration of the anti-TIGIT antibody, there is (i) an increase in the subject's CD8+ T cell expansion or proliferation, (ii) an increase in the subject's CD8+ T cell function, (iii) a decrease in CD8+ T cell exhaustion, or (iv) a combination thereof. In some embodiments, any decrease in Tregs or TIGIT+Tregs is no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the administering can comprise one or more dosing cycles (e.g., 1, 2, 3, or 4 or more), in which the anti-TIGIT antibody is administered and then re-administered at a later time (e.g., 1, 2, 3, 4, 5, 6, 7, or 8 or more weeks after the prior administration). For example, the one or more dosing cycles may comprise administering the anti-TIGIT antibody to the subject once every 2 weeks, once every 3 weeks, once every 4 weeks, once every 5 weeks, once every 6 weeks, once every 7 weeks, or once every 8 weeks. In some embodiments, the one or more dosing cycles may comprise administering the anti-TIGIT antibody to the subject at a dose of about 15 mg/kg. In some embodiments, the one or more dosing cycles may comprise administering the anti-TIGIT antibody to the subject at a dose in a range of about 500 mg to about 2000 mg, about 600 mg to about 800 mg, about 900 mg to about 1200 mg, about 1200 mg to about 1600 mg, or about 1200 mg to about 1500 mg. In some embodiments, the (i) increase in the subject's CD8+ T cell expansion or proliferation, (ii) increase in the subject's CD8+ T cell function, (iii) decrease in CD8+ T cell exhaustion, or (iv) a combination thereof may be observed at least 1 week, at least 2 weeks, at least 3 weeks, or at least 4 weeks after administration of the anti-TIGIT antibody. In some embodiments, the (i) increase in the subject's CD8+ T cell expansion or proliferation, (ii) increase in the subject's CD8+ T cell function, (iii) decrease in CD8+ T cell exhaustion, or (iv) a combination thereof may be observed after 1, 2, 3, or 4 or more dosing cycles. In some embodiments, the (i) increase in the subject's CD8+ T cell expansion or proliferation, (ii) increase in the subject's CD8+ T cell function, (iii) decrease in CD8+ T cell exhaustion, or (iv) a combination thereof is greater than a corresponding measure at a corresponding time in a patient treated with a Fc-enabled anti-TIGIT antibody, such as AB308, BMS-986207, tiragolumab, vibostolimab, etigilimab, ociperlimab, EOS-448, SEA-TGT, AGEN1777, AGEN1327, ralzapastotug, and JS006.

Additionally or alternatively, the present disclosure provides methods for treating cancer in a human subject in need thereof, comprising administering to the subject an anti-TIGIT antibody that has reduced binding to FcγR as compared to WT IgG1, wherein after a first administration of the anti-TIGIT antibody, there is an expansion or proliferation or activation of myeloid cells, lymphoid cells, or a combination thereof. In some embodiments, any decrease in Tregs or TIGIT+Tregs is no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the administering can comprise one or more dosing cycles (e.g., 1, 2, 3, or 4 or more), in which the anti-TIGIT antibody is administered and then re-administered at a later time (e.g., 1, 2, 3, 4, 5, 6, 7, or 8 or more weeks after the prior administration). For example, the one or more dosing cycles may comprise administering the anti-TIGIT antibody to the subject once every 2 weeks, once every 3 weeks, once every 4 weeks, once every 5 weeks, once every 6 weeks, once every 7 weeks, or once every 8 weeks. In some embodiments, the one or more dosing cycles may comprise administering the anti-TIGIT antibody to the subject at a dose of about 10 mg/kg to about 20 mg/kg. In some embodiments, the one or more dosing cycles may comprise administering the anti-TIGIT antibody to the subject at a dose in a range of about 500 mg to about 2000 mg, about 600 mg to about 800 mg, about 900 mg to about 1200 mg, about 1200 mg to about 1600 mg, or about 1200 mg to about 1500 mg. In some embodiments, the expansion or proliferation or activation of myeloid cells, lymphoid cells, or a combination thereof may be observed at least one day, at least 1 week, at least 2 weeks, at least 3 weeks, or at least 4 weeks after administration of the anti-TIGIT antibody. In some embodiments, the expansion or proliferation or activation of myeloid cells, lymphoid cells, or a combination thereof may be observed after 1, 2, 3, or 4 or more dosing cycles. In some embodiments, the expansion or proliferation or activation of myeloid cells, lymphoid cells, or a combination thereof is greater than a corresponding measure at a corresponding time in a patient treated with a Fc-enabled anti-TIGIT antibody, such as AB308, BMS-986207, tiragolumab, vibostolimab, etigilimab, ociperlimab, ralzapastotug, EOS-448, SEA-TGT, AGEN1777, AGEN1327, and JS006.

c. One or More Additional Therapy

For the purposes of any of the forgoing methods, the methods of treatment can further comprise administration of one or more additional therapy. Suitable additional therapies are described in Section IV.

In some of the foregoing methods, the additional therapy may be an immunotherapeutic agent. Immunotherapeutic agents that may be suitably combined with the disclosed anti-TIGIT antibodies having reduced or abolished Fc effector function include but are not limited to checkpoint inhibitors, such as antagonists of CTLA-4, PD-1, and PD-L1, as well as ATP-adenosine targeting agents, such as antagonists of A₂ₐR and/or A_{2b}R, inhibitors of CD39, and inhibitors of CD73. Specific CPIs that may be combined with the disclosed an anti-TIGIT antibodies (e.g., domvanalimab) include, but are not limited to, ipilimumab (YERVOY®), nivolumab (OPDIVO®), pembrolizumab (KEYTRUDA®), cemiplimab (LIBTAYO®), avelumab (BAVENCIO®), durvalumab (IMFINZI®), atezolizumab (TECENTRIQ®), and zimberelimab (AB122). Treatment-related adverse events, including immune-related adverse events, have been observed with many immune checkpoint inhibitors CPIs, such as CTLA-4 antagonists and PD-1/PD-L1 antagonists. For example, known PD-(L)1 class risk effects include identified risks such as Stevens-Johnson syndrome, myocarditis, pneumonitis, immune-mediated lung disease, and interstitial lung disease; potential risks such as infusion related reactions, immune-mediated hepatitis, and immune-mediated enterocolitis. For the purposes of the present methods of treatment, it is believed that combining an anti-TIGIT antibody having reduced or abolished Fc effector function (e.g., domvanalimab) with a CPI (such as those listed above) will not result in an increase in the likelihood, overall occurrence, or severity of one or more treatment-related adverse event (e.g., immune-related adverse events, etc.) that may occur when a given CPI is administered alone. It is also believed the disclosed methods of administration of an anti-TIGIT antibody having reduced or abolished Fc effector function (e.g., domvanalimab) will not result in a further loss, decrease, or inactivation of T cells (e.g., CD8+ T cells), Tregs, and/or other myeloid or lymphoid beyond what would be expected for the given CPI.

In some of the foregoing methods, the additional therapy may be one or more chemotherapeutic agents. One of skill in the art may choose a suitable chemotherapeutic regimen, the decision of which may be informed by current standard of care for a particular cancer and/or mutational status of a subject's cancer and/or stage of disease. Detailed standard of care guidelines are published, for example, by National Comprehensive Cancer Network (NCCN). It is understood in the art that many chemotherapeutic agents cause a decrease in T cells and other immune cells. For the purposes of the disclosed methods, administration of an anti-TIGIT antibody having reduced or abolished Fc effector function (e.g., domvanalimab) will not result in a further loss, decrease, or inactivation of T cells (e.g., CD8+ T cells), Tregs, and/or other myeloid or lymphoid beyond what would be expected for the given chemotherapeutic agent.

In some embodiments, the one or more additional therapeutic agent comprises etrumadenant. In some embodiments, a therapeutically effective amount of etrumadenant is about 50 mg to about 250 mg per day administered orally, about 50 mg to about 225 mg per day administered orally, about 50 mg to about 150 mg per day administered orally, or about 100 mg to about 250 mg per day administered orally. In some embodiments, a therapeutically effective amount of etrumadenant is about 50 mg, about 75 mg, about 100 mg, about 150 mg, about 175 mg, about 200 mg, about 225 mg, or about 250 mg per day administered orally.

In some embodiments, the one or more additional therapeutic agent comprises zimberelimab and etrumadenant. In some embodiments, a therapeutically effective amount of zimberelimab is about 360 mg intravenously administered every three weeks, or about 480 mg intravenously administered every four weeks, and a therapeutically effective amount of etrumadenant is about 50 mg to about 250 mg per day administered orally or about 50 mg to about 150 mg per day administered orally. In some embodiments, a therapeutically effective amount of zimberelimab is about 720 mg intravenously administered every six weeks, about 760 mg intravenously administered every six weeks, about 960 mg intravenously administered every six weeks, or about 760 mg to about 960 mg intravenously administered every six weeks, and a therapeutically effective amount of etrumadenant is about 50 mg to about 250 mg per day administered orally or about 50 mg to about 150 mg per day administered orally.

In some embodiments, the one or more additional therapeutic agent comprises quemliclustat. In some embodiments, a therapeutically effective amount of quemliclustat is about 100 mg to about 200 mg intravenously administered every two weeks, or about 300 mg intravenously administered every three weeks.

In some embodiments, the one or more additional therapeutic agent comprises zimberelimab and quemliclustat. In some embodiments, a therapeutically effective amount of zimberelimab is about 360 mg intravenously administered every three weeks, or about 480 mg intravenously administered every four weeks, and a therapeutically effective amount of quemliclustat is about 300 mg intravenously administered every three weeks. In some embodiments, a therapeutically effective amount of zimberelimab is about 720 mg intravenously administered every six weeks, about 760 mg intravenously administered every six weeks, about 960 mg intravenously administered every six weeks, or about 760 mg to about 960 mg intravenously administered every six weeks, and a therapeutically effective amount of quemliclustat is about 300 mg intravenously administered every three weeks.

d. Dose Cycle

For the purposes of any of the foregoing methods of treatment, a dose cycle may comprise, for example, dosing twice a week, once a week, once every 2 weeks, once every 3 weeks, once every 4 weeks, once every 5 weeks, once every 6 weeks, once every 7 weeks, or once every 8 weeks. In some embodiments, a dose cycle may comprise about 10 mg/kg to about 20 mg/kg of antibody of the disclosure (e.g., domvanalimab) administered Q2W, Q3W or Q4W. In a specific example, a dose cycle may comprise about 10 mg/kg of antibody administered Q2W. In another specific example, a dose cycle may comprise about 15 mg/kg of antibody administered Q2W. In another specific example, a dose cycle may comprise about 15 mg/kg of antibody administered Q3W. In another specific example, a dose cycle may comprise about 20 mg/kg of antibody administered Q3W. In another specific example, a dose cycle may comprise about 20 mg/kg of antibody administered Q4W. In some embodiments, a dose cycle may comprise about 500 mg to about 2000 mg of antibody administered Q2W, Q3W, or Q4W. In some embodiments, a dose cycle may comprise about 600 mg to about 1600 mg of antibody administered Q2W, Q3W, or Q4W. In some embodiments, a dose cycle may comprise about 1000 mg to about 1500 mg of antibody administered Q2W, Q3W, or Q4W. In a specific example, a dose cycle may comprise about 600 mg to about 800 mg of antibody administered Q2W, Q3W, or Q4W. In a specific example, a dose cycle may comprise about 900 mg to about 1200 mg of antibody administered Q2W, Q3W, or Q4W. In a specific example, a dose cycle may comprise about 1200 mg to about 1600 mg of antibody administered Q2W, Q3W, or Q4W. In a specific example, a dose cycle may comprise about 600 mg to about 800 mg of antibody administered Q2W. In a specific example, a dose cycle may comprise about 900 mg to about 1200 mg of antibody administered Q3W. In a specific example, a dose cycle may comprise about 1200 mg to about 1600 mg of antibody administered Q4W. In a specific example, a dose cycle may comprise about 1000 mg of antibody administered Q2W. In another specific example, a dose cycle may comprise about 1200 mg of antibody administered Q2W. In another specific example, a dose cycle may comprise about 1200 mg of antibody administered Q3W. In another specific example, a dose cycle may comprise about 1500 mg of antibody administered Q3W. In another specific example, a dose cycle may comprise about 1500 mg of antibody administered Q4W.

e. Anti-TIGIT Antibody

Suitable anti-TIGIT antibodies of the foregoing methods are described in Section III. In some embodiments of the foregoing methods, the anti-TIGIT antibody is an IgG4 or an IgG1 with reduced ability to bind FcγR. In some embodiments, an anti-TIGIT antibody is an IgG4 or an IgG1 that does not bind to FcγR. Various modifications to IgG1s that can reduce or abolish FcγR binding are described above.

In some embodiments of the foregoing methods, the anti-TIGIT antibody is domvanalimab or an antigen-binding fragment of domvanalimab. In some embodiments, the anti-TIGIT antibody competitively inhibits binding of domvanalimab to human TIGIT by at least 50% (e.g., at least 50%, at least 55%, at least 60%, at least 65%, at least 70%, at least 80%, at least 85%, at least 90%, at least 95%) as described in Section 111. In some embodiments, the anti-TIGIT antibody binds the same epitope of TIGIT as domvanalimab. In some embodiments of the foregoing methods, the anti-TIGIT antibody is a variant domvanalimab. In some embodiments, an anti-TIGIT antibody comprises a heavy chain CDR1 comprising the amino acid sequence with at least 90%, at least 91%, at least 92%, at least 93%, at least 94%, at least 95%, at least 96%, at least 97%, at least 98%, at least 99%, or 100% sequence identity to SEQ ID NO: 2, a heavy chain CDR2 comprising the amino acid sequence with at least 90%, at least 91%, at least 92%, at least 93%, at least 94%, at least 95%, at least 96%, at least 97%, at least 98%, at least 99%, or 100% sequence identity to SEQ ID NO: 3, a heavy chain CDR3 comprising the amino acid sequence with at least 90%, at least 91%, at least 92%, at least 93%, at least 94%, at least 95%, at least 96%, at least 97%, at least 98%, at least 99%, or 100% sequence identity to SEQ ID NO: 4, a light chain CDR1 comprising the amino acid sequence with at least 90%, at least 91%, at least 92%, at least 93%, at least 94%, at least 95%, at least 96%, at least 97%, at least 98%, at least 99%, or 100% sequence identity to SEQ ID NO: 5, a light chain CDR2 comprising the amino acid sequence with at least 90%, at least 91%, at least 92%, at least 93%, at least 94%, at least 95%, at least 96%, at least 97%, at least 98%, at least 99%, or 100% sequence identity to SEQ ID NO: 6, and a light chain CDR3 comprising the amino acid sequence with at least 90%, at least 91%, at least 92%, at least 93%, at least 94%, at least 95%, at least 96%, at least 97%, at least 98%, at least 99%, or 100% sequence identity to SEQ ID NO: 7.

In some embodiments of the foregoing methods, an anti-TIGIT antibody comprises a heavy chain variable region with at least 90%, at least 91%, at least 92%, at least 93%, at least 94%, at least 95%, at least 96%, at least 97%, at least 98%, at least 99%, or 100% sequence identity to SEQ ID NO: 8 or 10 and a light chain variable region with at least 90%, at least 91%, at least 92%, at least 93%, at least 94%, at least 95%, at least 96%, at least 97%, at least 98%, at least 99%, or 100% sequence identity to SEQ ID NO: 9 or 11. In some embodiments, an anti-TIGIT antibody comprises a heavy chain with at least 90%, at least 91%, at least 92%, at least 93%, at least 94%, at least 95%, at least 96%, at least 97%, at least 98%, at least 99%, or 100% sequence identity to SEQ ID NO: 12 and a light chain with at least 90%, at least 91%, at least 92%, at least 93%, at least 94%, at least 95%, at least 96%, at least 97%, at least 98%, at least 99%, or 100% sequence identity to SEQ ID NO: 13.

In some embodiments of the foregoing methods, the anti-TIGIT antibody may be formulated for injection as an aqueous solution comprising about 10 mg/mL to about 100 mg/mL antibody or about 20 mg/mL to about 60 mg/mL antibody, a buffer containing about 15 to about 25 mM His/His-Cl, about 5% to about 10% (weight/volume) of an excipient selected from the group consisting of sucrose, dextrose, and mannitol, about 0 mg/mL to about 10 mg/mL NaCl, and about 0.1 mg/mL to about 0.3 mg/mL polysorbate 80. Further embodiments are described in Section V.

f. Subject

A patient (or subject) in need of the treatments, prophylaxis, or methods described herein may be a human subject with cancer. In some embodiments, the cancer or a tumor biopsy thereof (which may comprise both tumor cells and immune cells) may be PD-1 positive or overexpressing. In some embodiments, the cancer may be PD-L1 positive or overexpressing. In some embodiments, the cancer may be CTLA positive or overexpressing. In some embodiments, the cancer may be CD73 positive or overexpressing. In some embodiments, the cancer may be DNAM-1 positive or overexpressing. In some embodiments, the cancer may be PVR positive or overexpressing. In some embodiments, the cancer may be TIGIT positive or overexpressing. In some embodiments, the cancer may be Ki67 positive or overexpressing, optionally CD8 positive and Ki67 positive or overexpressing. In some embodiments, the cancer may have a high tumor mutation burden (TMB). In some embodiments, the cancer may not have a genomic mutation for which a targeted therapy has received marketing approval by a regulatory authority. In some embodiments, the cancer (i) may be positive or overexpressing or down-regulating a biomarker selected from the group consisting of CD73, DNAM-1, PVR, TIGIT, PD-L1, PD-1, CTLA, and CD8-Ki67, or any combination thereof; (ii) may have a high TMB; (iii) may not have a genomic mutation for which a targeted therapy has received marketing approval by a regulatory authority; or (iv) may have any combination of (i) through (iii). Identification of cancers positive for and/or overexpressing a biomarker may occur using a suitable immunohistochemistry (IHC) assay and sample of a tumor biopsy. Criteria for biomarker “positive” and biomarker “overexpressing” or “high” cancers may be influenced by tumor site and/or type, antibody used, and assay conditions. Suitable antibodies for evaluating PD-L1 expression include antibody clones 22C3, 28-8, SP142, SP263 and 73-10. PD-L1 expression may be reported as a tumor proportion score (TPS), combined positive score (CPS), percentage of tumor cells with any PD-L1 membrane staining above background (% TC), proportion of tumor area occupied by PD-L1 expressing tumor-infiltrating cells (% IC), or tumor area positivity (TAP) score. TPS is the percentage of viable tumor cells showing partial or complete membrane staining at any intensity. TPS or % TC are typically used for assessing PD-L1 expression in NSCLC, although other measures may be used. When TPS or % TC is used to assess PD-L1 expression, a PD-L1 positive tumor may have a TPS or % TC score of ≥1%, ≥5%, ≥10%, or ≥50%, where a score of ≥50% typically is referred to as PD-L1 high. CPS and TAP are among the approaches used to quantify PD-L1 expression in tumors other than NSCLC (e.g., HNSCC, melanoma, UBC, TNBD, cervical, esophageal, gastric, GEJ, RCC, HCC, etc.) CPS is the number of PD-L1 staining cells (tumor, lymphocytes or macrophages) divided by the number of viable tumor cells, multiplied by 100. TAP score is defined as the total percentage of the tumor area covered by tumor cells with PD-L1 membrane staining at any intensity and tumor-associated immune cells with PD-L1 staining at any intensity. A PD-L1 positive tumor may have a CPS of ≥1, ≥5, ≥10 or ≥20, or a TAP of ≥1%, ≥5%, ≥10% or ≥20%. Suitable antibodies for evaluating DNAM-1 include antibody clone R102 (Sino Biologicals). Suitable antibodies for evaluating PVR include antibody clone D3G7H (Cell Signaling Technology). Suitable antibodies for evaluating TIGIT include antibody clone SP410 (Spring Biosciences). Suitable antibodies for evaluating Ki67 include antibody clone MIB-1. Tumor mutational burden (TMB) is defined as the number of mutations per DNA megabases. TMB has historically been assessed with whole genome sequencing (WGS) or whole exome sequencing (WES) of tumor tissue, however, good correlation between targeted next generation sequencing (NGS) and WES has been reported. NGS also can concomitantly be used to detect specific genetic alterations. Although not as established as measuring TMB from a biopsy sample of tumor tissue, TMB and genetic alterations may also be identified from circulating tumor DNA in the plasma. In some embodiments, identification of a biomarker positive, overexpressing or high cancer and/or evaluation of TMB and/or genetic alterations may occur using an FDA-approved test, such as PD-L1 IHC 22C3 (pharmDx), PD-L1 IHC 28-8 (pharmDx), PD-L1 SP142 Assay (Ventana), PD-L1 SP263 Assay (Ventana), FoundationOne CDx (to characterize individual's genomic profiles, including TMB status), and MSK-IMPACT tumor profiling (to characterize individual's genomic profiles, including TMB status).

The antibodies described herein are useful in the treatment and/or prophylaxis of cancer (e.g., carcinomas, sarcomas, leukemias, lymphomas and myelomas). In some embodiments, the present disclosure provides methods for the treatment and/or prophylaxis of cancer with an antibody of the present disclosure and at least one additional therapeutic agent, examples of which are set forth elsewhere herein. In certain embodiments, the present disclosure provides methods for the treatment and/or prophylaxis of cancer with an antibody of the present disclosure and at least one additional therapeutic agent that is an immuno-oncology dug, optionally with one or more chemotherapeutic agent. In further embodiments, the immuno-oncology drug is an immune checkpoint inhibitor or an agent that targets the ATP-adenosine axis. In still further embodiments, the ATP-adenosine axis-targeting agent is an A₂ₐR antagonist, an A_{2b}R antagonist, an antagonist of A₂ₐR and A_{2b}R, a CD73 inhibitor, or a CD39 inhibitor, and the immune checkpoint inhibitor blocks the activity of at least one of PD-1, PD-L1, BTLA, LAG3, a B7 family member, TIM3, or CTLA-4. In still further embodiments, the ATP-adenosine axis-targeting agent is an A₂ₐR antagonist, an A_{2b}R antagonist, an antagonist of A₂ₐR and A_{2b}R, a CD73 inhibitor, or a CD39 inhibitor, and the immune checkpoint inhibitor blocks the activity of at least one of PD-1, PD-Li, or CTLA-4. In still further embodiments, the ATP-adenosine axis-targeting agent is an A₂ₐR antagonist, an A_{2b}R antagonist, an antagonist of A₂ₐR and A_{2b}R, a CD73 inhibitor, or a CD39 inhibitor, and the immune checkpoint inhibitor blocks the activity of at least one of PD-1 or PD-L1. To achieve treatment and/or prophylaxis, an antibody of the present disclosure, and additional therapeutic agent(s), are administered in a “therapeutically effective amount”.

The methods of the present disclosure may be practiced in an adjuvant setting. “Adjuvant setting” refers to a clinical setting in which a subject has a history of a proliferative disease, particularly cancer, and generally (but not necessarily) has been responsive to therapy, which includes, but is not limited to, surgery, radiotherapy, and/or chemotherapy. However, because of a history of the proliferative disease, these subjects are considered to be at risk of relapse and/or disease progression. Treatment or administration in the “adjuvant setting” refers to a subsequent mode of treatment. Generally, adjuvant therapy is given in addition to the primary treatment to decrease the risk of the disease or condition recurring. In some embodiments, provided herein is a method for treating or effecting prophylaxis of cancer including administering to a subject having or at risk of cancer a therapeutically effective amount of any of the antibodies disclosed herein in an adjuvant setting.

The methods provided herein may also be practiced in a “neoadjuvant setting,” that is, the method may be carried out before the primary therapy. In some aspects, the subject has previously been treated. In other aspects, the subject has not previously been treated. In some aspects, the primary treatment is a first line therapy. In some embodiments, provided herein is a method for treating or effecting prophylaxis of cancer including administering to a subject having or at risk of having cancer a therapeutically effective amount of any of the antibodies disclosed herein in a neoadjuvant setting.

The methods provided herein may also be indicated as first line, second line, third line, or greater treatment.

In some embodiments, the cancer may be locally advanced and/or unresectable, metastatic, or at risk of becoming metastatic. Alternatively, or in addition, the cancer may be recurrent or no longer responding to a treatment, such as a standard of care. In a specific embodiment, the cancer may be (i) locally advanced, unresectable, locally advanced unresectable, or metastatic, (ii) no longer responding to a treatment, such as a standard of care, a CPI, or more specifically, a PD-1 or PD-L1 antagonist, or (iii) a combination of (i) and (ii). Determination of whether a subject is “no longer responding to treatment” can be made the subject's medical provider, for example, based on a plateauing of a response or disease progression.

Exemplary types of cancer contemplated by this disclosure include cancer of the genitourinary tract (e.g., bladder, kidney, renal cell, penile, prostate, testicular, Von Hippel-Lindau disease, etc.), uterus, cervix, ovary, breast, gastrointestinal tract (e.g., esophagus, oropharynx, stomach, small or large intestines, colon, or rectum), bone, bone marrow, skin (e.g., melanoma), head and neck, liver, gall bladder, bile ducts, heart, lung, pancreas, salivary gland, adrenal gland, thyroid, brain (e.g., gliomas), ganglia, central nervous system (CNS), peripheral nervous system (PNS), the hematopoietic system (i.e., hematological malignancies), and the immune system (e.g., spleen or thymus).

In some embodiments, the antibodies according to this disclosure are useful in the treatment and/or prophylaxis of solid tumors. The solid tumor may be an ovarian cancer, endometrial cancer, breast cancer, lung cancer (small cell or non-small cell), colorectal cancer, prostate cancer, cervical cancer, biliary cancer, pancreatic cancer (e.g., pancreatic neuroendocrine tumors (pNET)), gastric cancer, esophageal cancer, liver cancer (e.g., hepatocellular carcinoma), kidney cancer (e.g., renal cell carcinoma), head-and-neck tumors, mesothelioma, skin cancer (e.g., melanoma, Merkel cell carcinoma), sarcomas, central nervous system (CNS) hemangioblastomas, and brain tumors (e.g., gliomas, such as astrocytoma, oligodendroglioma and glioblastomas). The solid tumor may also be a locally advanced and/or unresectable solid tumor, a metastatic solid tumor, a recurrent solid tumor, a solid tumor no longer responding to a treatment, such as a standard of care, or a combination thereof.

In some embodiments, the antibodies according to this disclosure are useful in the treatment and/or prophylaxis of lung cancer, genitourinary cancer, gastrointestinal cancer, cervical cancer, ovarian cancer, or a combination thereof, optionally wherein the tumor is a locally advanced and/or unresectable tumor, a metastatic tumor, a recurrent tumor, a tumor no longer responding to a treatment, such as a standard of care, or any combination thereof.

In some embodiments, the cancer is lung cancer. In further embodiments, the lung cancer is non-small cell lung cancer (NSCLC), optionally wherein the cancer is (i) locally advanced, unresectable, locally advanced unresectable, or metastatic, (ii) no longer responding to a treatment, such as a standard of care, or (iii) a combination of (i) and (ii). In still further embodiments, the NSCLC may be lung squamous cell carcinoma or lung adenocarcinoma. For instance, in one example, the lung cancer is locally advanced, unresectable NSCLC where the cancer has not progressed following definitive platinum-based concurrent chemoradiation therapy. In another example, the lung cancer is locally advanced or metastatic, squamous or non-squamous NSCLC that is treatment naïve with respect to locally advanced or metastatic disease.

In some embodiments, a patient in need of the treatments, prophylaxis, or methods described herein may be a human subject with NSCLC (squamous or non-squamous disease), and in certain embodiments a patient with unresectable locally advanced disease (Stage IIIA, IIIB, IIIC) or metastatic disease (Stage IV). In some embodiments, the NSCLC may be PD-L1 high, corresponding to TPS ≥50% or TC ≥50% as measured by a clinically validated PD-L1 IHC assay or FDA-approved test, such as PharmDx 22C3, 28-8 pharmDx (Dako). a SP263 assay (Ventana), or SP142 assay (Ventana). In some embodiments, the NSCLC may be PD-L1 expressing, corresponding to TPS <50% or TC<50% as measured by a clinically validated PD-L1 IHC assay or FDA-approved test, for example, about 1%, about 5%, about 10%, about 15%, about 20%, about 25%, about 30%, about 35%, about 40%, about 45%, about 46%, about 47%, about 48% about 49%, or ranges thereof such as about 1-10%, about 10%-20%, about 20-30%, about 30-40%, about 40-49%, about 1-49%, about 1-25%, or about 25-49%. In some embodiments, the NSCLC may be PD-L1 expressing, corresponding to TPS ≥20%, ≥25%, >30%, ≥35%, ≥40%, or ≥45%, or TC ≥20%, ≥25%, ≥30%, ≥35%, ≥40%, or ≥45%, as measured by a clinically validated PD-L1 IHC assay or FDA-approved test. In some embodiments, the cancer may be tumor mutational burden-high (TMB-H; ≥10 mutations/megabase (mut/Mb), as determined by an FDA-approved test). The cancer may or may not have a genomic mutation for which a targeted therapy has received marketing approval by a regulatory authority, non-limiting examples of genes with such mutation include ALK fusion oncogene, EGFR, ROS, BRAF, and NTRK. In the foregoing embodiments, the cancer may be positive or overexpressing or down-regulating a biomarker selected from the group consisting of CD73, DNAM-1, PVR, TIGIT, and CD8-Ki67, or any combination thereof.

In some embodiments, the cancer is a gastrointestinal cancer. In various embodiments, the gastrointestinal cancer may be esophageal cancer, gastric cancer, colorectal cancer, pancreatic cancer or liver cancer, optionally wherein the cancer is (i) locally advanced, unresectable, locally advanced unresectable, or metastatic and/or (ii) no longer responding to a treatment, such as a standard of care.

In some embodiments, the cancer is an upper GI cancer, such as esophageal or gastric cancer, optionally wherein the upper GI cancer is (i) locally advanced, unresectable, locally advanced unresectable, or metastatic and/or (ii) no longer responding to a treatment, such as a standard of care. In further embodiments, the upper GI cancer is an adenocarcinoma, a squamous cell carcinoma, or any combination thereof. In still further embodiments, the upper GI cancer is esophageal adenocarcinoma (EAC), esophageal squamous cell carcinoma (ESCC), gastroesophageal junction adenocarcinoma (GEJ), gastric adenocarcinoma (also referred to herein as “gastric cancer”) or any combination thereof, optionally wherein the cancer is (i) locally advanced, unresectable, locally advanced unresectable, or metastatic and/or (ii) no longer responding to a treatment, such as a standard of care. In still further embodiments, the upper GI cancer is esophageal adenocarcinoma (EAC), gastroesophageal junction adenocarcinoma (GEJ), gastric adenocarcinoma (GA) or any combination thereof, optionally wherein the cancer is (i) locally advanced, unresectable, locally advanced unresectable, or metastatic and/or (ii) no longer responding to a treatment, such as a standard of care.

In some embodiments, a patient in need of the treatments, prophylaxis, or methods described herein may be a human subject with gastrointestinal cancer, optionally an (i) upper GI cancer, (ii) a GI cancer selected from the group consisting of GA, GEJ, ESCC, EAC, and any combination thereof, (iii) a GI cancer selected from the group consisting of GA, GEJ, EAC, and any combination thereof; and in certain embodiments a patient with locally advanced unresectable or metastatic disease. The patient may or may not have had treatment with a prior systemic treatment and may be checkpoint inhibitor (CPI) naïve or experienced. In some embodiments, the cancer may PD-L1 expressing (e.g., TAP (tumor area positivity) <1%, ≥1%, ≥5%, ≥10%, 1% to <5%, 5% to <10%, or >10%, measured by the Ventana PD-L1 (SP263) Assay, or an equivalent value measured by another clinically validated PD-Li IHC assay.) In some embodiments, the cancer may be tumor mutational burden-high (TMB-H; ≥10 mutations/megabase (mut/Mb), as determined by a clinically validated or an FDA-approved test). The cancer may or may not have a genomic mutation for which a targeted therapy has received marketing approval by a regulatory authority, non-limiting examples of genes with such mutation include ALK fusion oncogene, EGFR, ROS, BRAF, and NTRK. In the foregoing embodiments, the cancer may be positive or overexpressing or down-regulating a biomarker selected from the group consisting of CD73, DNAM-1, PVR, TIGIT, and CD8-Ki67, or any combination thereof.

In some embodiments, the antibodies according to this disclosure are useful in the treatment and/or prophylaxis of NSCLC, head and neck squamous cell carcinoma (HNSCC), renal cell carcinoma (RCC), breast cancer, colorectal cancer (CRC), melanoma, bladder cancer, ovarian cancer, endometrial cancer, Merkel Cell, and gastroesophageal cancer.

The present disclosure also provides methods of treating or preventing other cancer-related diseases, disorders or conditions. The use of the term(s) cancer-related diseases, disorders and conditions is meant to refer broadly to conditions that are associated, directly or indirectly, with cancer, and includes, e.g., angiogenesis, precancerous conditions such as dysplasia, and non-cancerous proliferative diseases disorders or conditions, such as benign proliferative breast disease and papillomas. For clarity, the term(s) cancer-related disease, disorder and condition do not include cancer per se. In some embodiments, the present disclosure provides methods for treating cancer-related diseases, disorders or conditions with an antibody of the present disclosure and at least one additional therapeutic agent, examples of which are set forth elsewhere herein.

The following embodiments are given to illustrate the present disclosure. It should be understood that the invention is not to be limited to the embodiments that follow.

Embodiment 1: A method for treating cancer in a human subject in need thereof, comprising administering to the subject an anti-TIGIT antibody that has reduced binding to one or more activating human FcγRs as compared to WT human IgG1, wherein the subject's percent change from baseline for a measurement of T cells differs by less than a corresponding measurement in a population of subjects treated with an Fc-enabled anti-TIGIT antibody or is not statistically different than a corresponding measurement in a population of healthy subjects; and wherein the measurement of CD8+ T cells is (i) a ratio of CD8+ T cells to Tregs measured in a first sample at baseline and in a second sample obtained 1 or more days, or 1 or more weeks, after the administration of the anti-TIGIT antibody; or (ii) an absolute count of Tregs, CD8+ T cells, or a combination thereof measured in a first sample at baseline and in a second sample obtained 1 or more days, or 1 or more weeks, after the administration of the anti-TIGIT antibody.

Embodiment 2: The method of embodiment 1, wherein (i) the subject's ratio of total CD8+ T cells to Tregs measured in a first sample at baseline and in a second sample 1 or more weeks after a first administration of administering the anti-TIGIT antibody differs by no more than 1%, 2%. 3%, 4%. 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%; or (ii) the subject's Tregs, CD8+ T cells, or a combination thereof measured in a first sample at baseline and in a second sample 1 or more weeks after a first administration of administering the anti-TIGIT antibody differs by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

Embodiment 3: The method of embodiment 1 or 2, wherein the administering comprises one or more dosing cycles.

Embodiment 4: The method of embodiment 3, wherein a dosing cycle comprises administering the anti-TIGIT antibody to the subject once every 2 weeks, once every 3 weeks, or once every 4 weeks.

Embodiment 5: The method of any one of embodiments 1-4, wherein anti-TIGIT antibody is administered to the subject at a dose of about 5 mg/kg, about 10 mg/kg, about 15 mg/kg, about 20 mg/kg, or about 25 mg/kg.

Embodiment 6: The method of any one of embodiments 1-5, wherein anti-TIGIT antibody is administered to the subject at a dose in a range of about 500 mg to about 2000 mg, about 600 mg to about 800 mg, about 900 mg to about 1200 mg, about 1200 mg to about 1600 mg, or about 1200 mg to about 1500 mg.

Embodiment 7: The method of any one of embodiments 1-6 further comprising administration of one or more additional therapeutic agent, wherein the one or more additional therapeutic agent is, optionally, an immunotherapeutic agent, a chemotherapeutic agent, a chemotherapeutic regimen, or a combination thereof.

Embodiment 8: The method of any one of embodiments 1-7, wherein the first sample and second sample are selected from a blood sample, a tumor sample, and a combination thereof.

Embodiment 9: The method of any one of embodiments 1-8, wherein the second sample is obtained 2, 3, 4, 5, or 6 weeks after a first administration of the anti-TIGIT antibody.

Embodiment 10: The method of any one of embodiments 1-9, wherein the Fc-enabled anti-TIGIT antibody is selected from AB308, BMS-986207, tiragolumab, vibostolimab, etigilimab, ociperlimab, ralzapastotug, EOS-448, SEA-TGT, AGEN1777, AGEN1327, and JS006.

Embodiment 11: The method of any one of embodiments 1-10, wherein the subject does not experience an immune-related adverse event.

Embodiment 12: The method of any one of embodiments 1-10, wherein the subject is less likely to experience an immune-related adverse event, or optionally a >Grade 3 immune-related adverse event, than a subject being treated with an Fc-enabled anti-TIGIT antibody.

Embodiment 13: The method of embodiment 1 or 12, wherein the immune-related adverse event is selected from: (i) a skin or subcutaneous tissue disorder, a gastrointestinal disorder, a hepatobiliary disorder, an endocrine disorder, or a respiratory, thoracic or mediastinal disorder; (ii) a skin or subcutaneous tissue disorder; (iii) rash, oral mucositis, dry mouth, colitis, diarrhea, hepatitis, pneumonitis, endocrinopathies, hypophysitis, hypothyroidism, hyperthyroidism, adrenal insufficiency, diabetes, or a combination thereof; (iv) arthritis, hepatitis, hypothyroidism, hyperthyroidism, infusion-related reaction, maculo-papular rash, pneumonitis, pruritis, psoriasis, rash, swelling face, or a combination thereof; or (v) infusion-related reaction, maculo-papular rash, pruritis, psoriasis, rash; or a combination thereof.

Embodiment 14: A method for treating cancer in a human subject in need thereof, comprising administering to the subject an anti-TIGIT antibody that has reduced binding to FcγR as compared to WT IgG1, wherein after a first administration of the anti-TIGIT antibody, there is (i) an increase in the subject's CD8+ T cell proliferation, (ii) an increase in the subject's CD8+ T cell function, (iii) a decrease in CD8+ T cell exhaustion, or (iv) a combination thereof, and wherein any decrease in Tregs or TIGIT+Tregs is less than a corresponding measurement in a population of similar patients treated with an Fc-enabled anti-TIGIT antibody or is not statistically different than a corresponding measurement in a population of healthy subjects.

Embodiment 15: The method of embodiment 14, wherein the increase in the subject's CD8+ T cell proliferation, the increase in the subject's CD8+ T cell function, the decrease in CD8+ T cell exhaustion, and any decrease in total Tregs or TIGIT+Tregs are determined by measurements of a first sample obtained at baseline and a second sample obtained at least one week after the first administration of the anti-TIGIT antibody.

Embodiment 16: The method of embodiment 15, wherein the first sample and second sample are selected from a blood sample, a tumor sample, and a combination thereof.

Embodiment 17: The method of embodiment 15 or 16, wherein the second sample is obtained 2, 3, 4, 5, or 6 weeks after a first administration of the anti-TIGIT antibody.

Embodiment 18: The method of any one of embodiments 14-17 further comprising comprises two or more dosing cycles of the anti-TIGIT antibody.

Embodiment 19: The method of embodiment 18, wherein a dosing cycle comprises administering the anti-TIGIT antibody to the subject once every 2 weeks, once every 3 weeks, or once every 4 weeks.

Embodiment 20: The method of any one of embodiments 14-19, wherein anti-TIGIT antibody is administered to the subject at a dose of about 5 mg/kg, about 10 mg/kg, about 15 mg/kg, about 20 mg/kg, or about 25 mg/kg.

Embodiment 21: The method of any one of embodiments 14-20, wherein anti-TIGIT antibody is administered to the subject at a dose in a range of about 500 mg to about 2000 mg, about 600 mg to about 800 mg, about 900 mg to about 1200 mg, about 1200 mg to about 1600 mg, or about 1200 mg to about 1500 mg.

Embodiment 22: The method of any one of embodiments 14-21, wherein any decrease in total Tregs or TIGIT+Tregs is no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% throughout treatment with the anti-TIGIT antibody.

Embodiment 23: The method of any one of embodiments 14-22 further comprising administration of one or more additional therapeutic agent, wherein the one or more additional therapeutic agent is, optionally, an immunotherapeutic agent, a chemotherapeutic agent, a chemotherapeutic regimen, or a combination thereof.

Embodiment 24: The method of any one of embodiments 14-23, wherein the ratio of CD8+ T cells to Tregs differs by less than a corresponding ratio at a corresponding time in a patient treated with a Fc-enabled anti-TIGIT antibody.

Embodiment 25: The method of embodiment 14, wherein the Fc-enabled anti-TIGIT antibody is selected from AB308, BMS-986207, tiragolumab, vibostolimab, etigilimab, ociperlimab, ralzapastotug, EOS-448, SEA-TGT, AGEN1777, AGEN1327, and JS006.

Embodiment 26: The method of any one of embodiments 14-25, wherein the subject does not experience an immune-related adverse event.

Embodiment 27: The method of any one of embodiments 14-25, wherein the subject is less likely to experience an immune-related adverse event, or optionally a ≥Grade 3 immune-related adverse event, than a subject being treated with an Fc-enabled anti-TIGIT antibody.

Embodiment 28: The method of embodiment 26 or 27, wherein the immune-related adverse event is selected from: (i) a skin or subcutaneous tissue disorder, a gastrointestinal disorder, a hepatobiliary disorder, an endocrine disorder, or a respiratory, thoracic or mediastinal disorder; (ii) a skin or subcutaneous tissue disorder; (iii) arthritis, hepatitis, hypothyroidism, hyperthyroidism, infusion-related reaction, maculo-papular rash, pneumonitis, pruritis, psoriasis, rash, swelling face, or a combination thereof; or (iv) infusion-related reaction, maculo-papular rash, pruritis, psoriasis, rash; or a combination thereof.

Embodiment 29: The method of any one of embodiments 1-28, wherein treatment results in a reduction in tumor size, a reduction in tumor number, a reduction in metastasis, stable disease (SD), partial response (PR), complete response (CR), or a combination thereof.

Embodiment 30: The method of any one of embodiments 1-29, wherein the treatment results in an improvement in overall survival (OS), progression-free survival (PFS), disease control rate (DCR), overall response rate (ORR), or a combination thereof compared to placebo or a standard of care.

Embodiment 31: A method of blocking or preventing the binding of TIGIT to CD155 in a human subject without a significant reduction in Tregs, CD8+ T cells, CD4+ T cells, or a combination thereof, comprising administering to the subject an anti-TIGIT antibody that has reduced binding to FcγR as compared to WT IgG1.

Embodiment 32: The method of embodiment 31, wherein any reduction in reduction in Tregs, CD8+ T cells, CD4+ T cells, or a combination thereof differs from a baseline measure prior to administration of the anti-TIGIT antibody by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

Embodiment 33: The method of embodiment 31 or 32, any reduction in Tregs, CD8+ T cells, CD4+ T cells, or a combination thereof is assessed 1 week, 2 weeks, 3 weeks, 4 weeks, 5 weeks, 6 weeks, 7 weeks, or 8 weeks, after administration of the anti-TIGIT antibody.

Embodiment 34: The method of any one of embodiments 31-33, wherein the subject has cancer.

Embodiment 35: The method of any one of embodiments 31-34, where the anti-TIGIT antibody is administered for 1, 2, 3, or 4 dosing cycles.

Embodiment 36: The method of embodiment 35, wherein a dosing cycle comprises administering the anti-TIGIT antibody to the subject once every 2 weeks, once every 3 weeks, or once every 4 weeks.

Embodiment 37: The method of any one of embodiments 31-36, wherein anti-TIGIT antibody is administered to the subject at a dose of about 5 mg/kg, about 10 mg/kg, about 15 mg/kg, about 20 mg/kg, or about 25 mg/kg.

Embodiment 38: The method of any one of embodiments 31-37, wherein anti-TIGIT antibody is administered to the subject at a dose in a range of about 500 mg to about 2000 mg, about 600 mg to about 800 mg, about 900 mg to about 1200 mg, about 1200 mg to about 1600 mg, or about 1200 mg to about 1500 mg.

Embodiment 39: The method of any one of embodiments 31-38, the method does not result in an immune-related adverse event.

Embodiment 40: The method of any one of embodiments 31-38, wherein the subject is less likely to experience an immune-related adverse event, or optionally a >Grade 3 immune-related adverse event, than a subject being treated with an Fc-enabled anti-TIGIT antibody.

Embodiment 41: The method of embodiment 39 or 40, wherein the immune-related adverse event is selected from (i) a skin or subcutaneous tissue disorder, a gastrointestinal disorder, a hepatobiliary disorder, an endocrine disorder, or a respiratory, thoracic or mediastinal disorder; (ii) a skin or subcutaneous tissue disorder; (iii) arthritis, hepatitis, hypothyroidism, hyperthyroidism, infusion-related reaction, maculo-papular rash, pneumonitis, pruritis, psoriasis, rash, swelling face, or a combination thereof; or (iv) infusion-related reaction, maculo-papular rash, pruritis, psoriasis, rash; or a combination thereof.

Embodiment 42: The method of any one of embodiments 31-41 further comprising administering to the subject of one or more additional therapeutic agent, wherein the one or more additional therapeutic agent is, optionally, an immunotherapeutic agent, a chemotherapeutic agent, a chemotherapeutic regimen, or a combination thereof.

Embodiment 43: The method of any one of embodiments 31-42, wherein any decrease in Tregs is no more than 1%, 2%, 3%, 4%, 5%, 6%, 7%, 8%, 9%, 10%, 11%, 12%, 13%, 14%, or 15% throughout treatment with the anti-TIGIT antibody.

Embodiment 44: The method of any one of embodiments 31-43, wherein after the first administration of the anti-TIGIT antibody there is (i) an increase in the subject's CD8+ T cell proliferation, (ii) an increase in the subject's CD8+ T cell function, (iii) a decrease in CD8+ T cell exhaustion, or (iv) a combination thereof.

Embodiment 45: The method of any one of embodiments 31-44, wherein (i) the subject's ratio of total CD8+ T cells to Tregs measured in a first sample at baseline and in a second sample 1 or more weeks after the first administration of the anti-TIGIT antibody differs by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%; (ii) the amount of CD8+ T cells, Tregs, or a combination thereof measured in a first sample at baseline and in a second sample 1 or more weeks after the first administration of the anti-TIGIT antibody differs by no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30; or (iii) a combination thereof.

Embodiment 46: The method of any one of embodiments 1-45, wherein the anti-TIGIT antibody that has reduced binding to one or more activating human FcγR comprises an Fc region with reduced ability to bind one or more activating human FcγR, and is optionally an IgG4 or an IgG1.

Embodiment 47: The method of any one of embodiments 1-46, wherein the anti-TIGIT that has reduced binding to one or more activating human FcγR does not bind to one or more activating human FcγR.

Embodiment 48: The method of any one of embodiments 1-47, wherein the anti-TIGIT antibody comprises a heavy chain CDR1 comprising the amino acid sequence of SEQ ID NO: 2, a heavy chain CDR2 comprising the amino acid sequence of SEQ ID NO: 3, a heavy chain CDR3 comprising the amino acid sequence of SEQ ID NO: 4, a light chain CDR1 comprising the amino acid sequence of SEQ ID NO: 5, a light chain CDR2 comprising the amino acid sequence of SEQ ID NO: 6, and a light chain CDR3 comprising the amino acid sequence of SEQ ID NO: 7.

Embodiment 49: The method of any one of embodiments 1-48, wherein the anti-TIGIT antibody comprises a heavy chain variable region with at least 90% sequence identity to SEQ ID NO: 8 or 10 and a light chain variable region with at least 90% sequence identity to SEQ ID NO: 9 or 11.

Embodiment 50: The method of any one of embodiments 1-49, wherein the anti-TIGIT antibody comprises a heavy chain with at least 90% sequence identity to SEQ ID NO: 12 and a light chain with at least 90% sequence identity to SEQ ID NO: 13.

Embodiment 51: The method of any one of embodiments 1-50, wherein the anti-TIGIT antibody binds to the same epitope of TIGIT as domvanalimab or competes for binding to TIGIT with domvanalimab.

Embodiment 52: The method of any one of the preceding embodiments, wherein the cancer is a solid tumor optionally wherein the tumor is a locally advanced and/or unresectable tumor; a metastatic tumor; a recurrent tumor; a tumor no longer responding to a treatment, optionally wherein the treatment is a standard of care, a checkpoint inhibitor, a PD-1 antagonist, or a PD-L1 antagonist; or any combination thereof.

Embodiment 53: The method of embodiment 52, wherein the cancer is lung cancer, genitourinary cancer, gastrointestinal cancer.

Embodiment 54: The method of embodiment 53, wherein the cancer is lung cancer.

Embodiment 55: The method of embodiment 54, wherein, the lung cancer is non-small cell lung cancer (NSCLC), optionally wherein the cancer is (i) locally advanced, unresectable, locally advanced unresectable, or metastatic, (ii) no longer responding to a treatment optionally wherein the treatment is a standard of care, a checkpoint inhibitor, a PD-1 antagonist, or a PD-L1 antagonist, or (iii) a combination of (i) and (ii).

Embodiment 56: The method of embodiment 54, wherein the lung cancer is squamous or non-squamous, unresectable locally advanced disease or metastatic disease.

Embodiment 57: The method of embodiment 53, wherein the cancer is gastrointestinal cancer.

Embodiment 58: The method of embodiment 57, wherein the gastrointestinal cancer is esophageal cancer, gastric cancer, colorectal cancer, pancreatic cancer or liver cancer, optionally wherein the cancer is (i) locally advanced, unresectable, locally advanced unresectable, or metastatic and/or (ii) no longer responding to a treatment, such as a standard of care.

Embodiment 59: The method of embodiment 57, wherein the gastrointestinal cancer is esophageal cancer or gastric cancer, optionally wherein the cancer is (i) locally advanced, unresectable, locally advanced unresectable, or metastatic and/or (ii) no longer responding to a treatment, such as a standard of care, a checkpoint inhibitor, a PD-1 antagonist, or a PD-L1 antagonist.

Embodiment 60: The method of embodiment 57, wherein the gastrointestinal cancer is esophageal adenocarcinoma, esophageal squamous cell carcinoma, gastroesophageal junction adenocarcinoma, or gastric adenocarcinoma, optionally wherein the cancer is (i) locally advanced, unresectable, locally advanced unresectable, or metastatic and/or (ii) no longer responding to a treatment, such as a standard of care.

Embodiment 61: The method of embodiment 52, wherein the cancer is NSCLC, head and neck squamous cell carcinoma (HNSCC), renal cell carcinoma (RCC), breast cancer, colorectal cancer (CRC), melanoma, bladder cancer, ovarian cancer, endometrial cancer, Merkel Cell, or gastroesophageal cancer.

Embodiment 62: The method of any one of the preceding embodiments, wherein PD-L1 expression of the cancer is TPS ≥50%, as measured by a clinically validated PD-L1 IHC assay or FDA-approved test.

Embodiment 63: The method of any one of embodiments 1 to 61, wherein PD-L1 expression of the cancer is TPS ≤50% as measured by a clinically validated PD-L1 IHC assay or FDA-approved test.

Embodiment 64: The method of embodiment 63, wherein PD-L1 expression is about 1-10%, about 10%-20%, about 20-30%, about 30-40%, about 40-49%, about 1-49%, about 1-25%, or about 25-49%, as measured by a clinically validated PD-L1 IHC assay or FDA-approved test.

Embodiment 65: The method of any one of the preceding embodiments, wherein the cancer is tumor mutational burden-high (TMB-H; ≥10 mutations/megabase (mut/Mb), as determined by an FDA-approved test).

Embodiment 66: The method of any one of embodiments 1 to 64, wherein the cancer is not TMB-H.

Embodiment 67: The method of any one of the preceding embodiments, wherein the cancer does not have an actionably oncogenic mutation in ALK, EGFR, ROS, BRAF, or NTRK and/or has wildtype ALK, EGFR, ROS, BRAF, and/or NTRK.

Embodiment 68: The method of any one of the preceding embodiments, wherein the cancer expresses or overexpresses one or more biomarker selected CD73, DNAM-1, PVR, TIGIT, and CD8-Ki67.

Embodiment 71: The method of any one of embodiments 7, 23, or 42, wherein the immunotherapeutic agent is a checkpoint inhibitor, optionally selected from ipilimumab, nivolumab, pembrolizumab, cemiplimab, avelumab, durvalumab, atezolizumab, and zimberelimab; or an ATP-adenosine axis-targeting agent, optionally selected from an A₂ₐR antagonist, an A_{2b}R antagonist, an A₂ₐR and A_{2b}R antagonist, a CD73 inhibitor, and a CD39 inhibitor.

Embodiment 72: The method of embodiment 71, wherein the chemotherapeutic regimen is a fluoropyrimidine-containing chemotherapy or a platinum-containing chemotherapy.

Embodiment 73: The method of embodiment 72, wherein the fluoropyrimidine is fluorouracil-containing chemotherapy and the platinum-containing chemotherapy is carboplatin, cisplatin, or oxaliplatin.

Embodiment 74: The method of embodiment 72, wherein the chemotherapeutic regimen is FOLFOX, CAPOX, cisplatin and pemetrexed, carboplatin and pemetrexed, carboplatin and paclitaxel, or carboplatin and nab-paclitaxel.

Embodiment 75: A method for treating cancer in a human subject in need thereof, comprising administering to the subject an anti-TIGIT antibody that has reduced binding to one or more activating human FcγRs as compared to wild type (WT) human IgG1, wherein treatment results in a reduction of one or more adverse event as compared to a similar treatment comprising an Fc-enabled anti-TIGIT antibody.

Embodiment 76: A method for treating cancer in a human subject in need thereof, comprising administering to the subject an anti-TIGIT antibody that has reduced binding to one or more activating human FcγRs as compared to wild type (WT) human IgG1, wherein the subject has a reduced likelihood of experiencing one or more adverse event as compared to treatment with an Fc-enabled anti-TIGIT antibody.

Embodiment 77: The method of embodiment 75 or 76, wherein the Fc-enabled anti-TIGIT antibody is selected from AB308, BMS-986207, tiragolumab, vibostolimab, etigilimab, ociperlimab, ralzapastotug, EOS-448, SEA-TGT, AGEN1777, AGEN1327, JS006, and an Fc-enabled version of domvanalimab comprising a wild-type IgG1 Fc region.

Embodiment 78: A method for treating cancer in a human subject in need thereof without significantly increasing the likelihood of adverse event as compared to the standard of care, comprising administering to the subject an anti-TIGIT antibody that has reduced binding to one or more activating human FcγRs as compared to wild type (WT) human IgG1 in combination with one or more additional therapy.

Embodiment 79: A method for reducing one or more adverse event experienced by a human subject being treated for cancer with an anti-TIGIT antibody, comprising administering to the subject an anti-TIGIT antibody that has reduced binding to one or more activating human FcγRs as compared to wild type (WT) human IgG1.

Embodiment 80: A method for reducing one or more adverse event experienced by a human subject being treated for cancer with an anti-TIGIT antibody and an additional immunotherapeutic agent, comprising administering to the subject an anti-TIGIT antibody that has reduced binding to one or more activating human FcγRs as compared to wild type (WT) human IgG1 and an additional immunotherapeutic agent.

Embodiment 81: The method of embodiment 80, wherein the immunotherapeutic agent is a checkpoint inhibitor, optionally selected from ipilimumab, nivolumab, pembrolizumab, cemiplimab, avelumab, durvalumab, atezolizumab, and zimberelimab.

Embodiment 82: The method of any one of embodiments 75 to 81, wherein the adverse event is a treatment-related adverse event, a treatment-emergent adverse event, an immune-related adverse event, a treatment-related immune-related adverse event, a treatment-related adverse event leading to treatment discontinuation, a treatment-related adverse event leading to treatment disruption, a serious adverse event leading to treatment discontinuation, a serious adverse event leading to treatment disruption, a serious adverse event, a grade 3 or higher serious adverse event, or a grade 3 or higher treatment-related adverse event.

Embodiment 83: The method of embodiment 82, wherein the immune-related adverse event is selected from:
(i) a skin or subcutaneous tissue disorder, a gastrointestinal disorder, a hepatobiliary disorder, an endocrine disorder, or a respiratory, thoracic or mediastinal disorder;
(ii) a skin or subcutaneous tissue disorder;
(iii) rash, oral mucositis, dry mouth, colitis, diarrhea, hepatitis, pneumonitis, endocrinopathies, hypophysitis, hypothyroidism, hyperthyroidism, adrenal insufficiency, diabetes, or a combination thereof;
(iv) arthritis, hepatitis, hypothyroidism, hyperthyroidism, infusion-related reaction, maculo-papular rash, pneumonitis, pruritis, psoriasis, rash, swelling face, or a combination thereof; or
(v) infusion-related reaction, maculo-papular rash, pruritis, psoriasis, rash, or a combination thereof.

Embodiment 84: A method for reducing one or more dose reduction, temporary treatment disruption, or treatment discontinuation experienced by a human subject being treated for cancer with an anti-TIGIT antibody, comprising administering to the subject an anti-TIGIT antibody that has reduced binding to one or more activating human FcγRs as compared to wild type (WT) human IgG1.

Embodiment 85: A method for reducing one or more dose reduction, temporary treatment disruption, or treatment discontinuation experienced by a human subject being treated for cancer with an anti-TIGIT antibody and an additional immunotherapeutic agent, comprising administering to the subject an anti-TIGIT antibody that has reduced binding to one or more activating human FcγRs as compared to wild type (WT) human IgG1.

Embodiment 86: The method of embodiment 87, wherein the immunotherapeutic agent is a checkpoint inhibitor, optionally selected from ipilimumab, nivolumab, pembrolizumab, cemiplimab, avelumab, durvalumab, atezolizumab, and zimberelimab.

Embodiment 87: The method of any one of embodiments 84-86, the method further comprising a reduction in one or more immune-related adverse event, as compared to a similar treatment comprising an Fc-enabled anti-TIGIT antibody.

Embodiment 88: The method of embodiment 87, wherein the immune-related adverse event is selected from:
(i) a skin or subcutaneous tissue disorder, a gastrointestinal disorder, a hepatobiliary disorder, an endocrine disorder, or a respiratory, thoracic or mediastinal disorder;
(ii) a skin or subcutaneous tissue disorder;
(iii) rash, oral mucositis, dry mouth, colitis, diarrhea, hepatitis, pneumonitis, endocrinopathies, hypophysitis, hypothyroidism, hyperthyroidism, adrenal insufficiency, diabetes, or a combination thereof;
(iv) arthritis, hepatitis, hypothyroidism, hyperthyroidism, infusion-related reactions, maculo-papular rash, pneumonitis, pruritis, psoriasis, rash, swelling face, or a combination thereof; or
(v) infusion-related reactions, maculo-papular rash, pruritis, psoriasis, rash; or a combination thereof.

Embodiment 89: The method of any one of embodiments 75-88, wherein administering comprises one or more dosing cycles.

Embodiment 90: The method of embodiment 89, wherein a dosing cycle comprises administering the anti-TIGIT antibody to the subject once every 2 weeks, once every 3 weeks, or once every 4 weeks.

Embodiment 91: The method of any one of embodiments 75-90, wherein the anti-TIGIT antibody is administered to the subject at a dose of about 5 mg/kg, about 10 mg/kg, about 15 mg/kg, about 20 mg/kg, or about 25 mg/kg.

Embodiment 92: The method of any one of embodiments 75-90, wherein the anti-TIGIT antibody is administered to the subject at a dose of about 500 mg to about 2000 mg, about 600 mg to about 800 mg, about 900 mg to about 1200 mg, about 1200 mg to about 1600 mg, or about 1200 mg to about 1500 mg.

Embodiment 93: The method of embodiment 89, wherein a dosing cycle comprises administering the anti-TIGIT antibody to the subject at a dose of about 1200 mg once every three weeks or at a dose of about 1600 mg once every four weeks.

Embodiment 94: The method of any one of embodiments 75-93, further comprising administration of one or more additional therapeutic agent, wherein the one or more additional therapeutic agent is, optionally, an immunotherapeutic agent, a chemotherapeutic agent, a chemotherapeutic regimen, or a combination thereof.

Embodiment 95: The method of embodiment 94, wherein the immunotherapeutic agent is
a checkpoint inhibitor, optionally selected from ipilimumab, nivolumab, pembrolizumab, cemiplimab, avelumab, durvalumab, atezolizumab, and zimberelimab; or
an ATP-adenosine axis-targeting agent, optionally selected from an A₂ₐR antagonist, an A_{2b}R antagonist, an A₂ₐR and A_{2b}R antagonist, a CD73 inhibitor, and a CD39 inhibitor.

Embodiment 96: The method of embodiment 95, wherein the chemotherapeutic regimen is a fluoropyrimidine-containing chemotherapy or a platinum-containing chemotherapy.

Embodiment 97: The method of embodiment 96, wherein the fluoropyrimidine-containing chemotherapy is fluorouracil and the platinum-containing chemotherapy is carboplatin, cisplatin, or oxaliplatin.

Embodiment 98: The method of embodiment 96, wherein the chemotherapeutic regimen is FOLFOX, CAPOX, cisplatin and pemetrexed, carboplatin and pemetrexed, carboplatin and paclitaxel, or carboplatin and nab-paclitaxel.

Embodiment 99: The method of any one of embodiments 75-98, wherein the anti-TIGIT antibody that has reduced binding to one or more activating human FcγR is a human IgG4 or a human IgG1 with reduced ability to bind one or more activating human FcγR.

Embodiment 100: The method of any one of embodiments 75-99, wherein the anti-TIGIT that has reduced binding to one or more activating human FcγR does not bind to one or more activating human FcγR.

Embodiment 101: The method of any one of embodiments 75-100, wherein the anti-TIGIT antibody comprises a heavy chain CDR1 comprising the amino acid sequence of SEQ ID NO: 2, a heavy chain CDR2 comprising the amino acid sequence of SEQ ID NO: 3, a heavy chain CDR3 comprising the amino acid sequence of SEQ ID NO: 4, a light chain CDR1 comprising the amino acid sequence of SEQ ID NO: 5, a light chain CDR2 comprising the amino acid sequence of SEQ ID NO: 6, and a light chain CDR3 comprising the amino acid sequence of SEQ ID NO: 7.

Embodiment 102: The method of any one of embodiments 75-101, wherein the anti-TIGIT antibody comprises a heavy chain variable region with at least 90% sequence identity to SEQ ID NO: 8 or 10 and a light chain variable region with at least 90% sequence identity to SEQ ID NO: 9 or 11.

Embodiment 103: The method of any one of embodiments 75-102, wherein the anti-TIGIT antibody comprises a heavy chain with at least 90% sequence identity to SEQ ID NO: 12 and a light chain with at least 90% sequence identity to SEQ ID NO: 13.

Embodiment 104: The method of any one of embodiments 75-103, wherein the anti-TIGIT antibody binds to the same epitope of TIGIT as domvanalimab or the anti-TIGIT antibody competitively inhibits binding of domvanalimab to human TIGIT by at least 50%.

Embodiment 105: The method of any one of embodiments 75-104. wherein the cancer is a solid tumor, optionally wherein the tumor is a locally advanced and/or unresectable tumor; a metastatic tumor; a recurrent tumor; a tumor no longer responding to a treatment optionally wherein the treatment is a standard of care, a checkpoint inhibitor, a PD-1 antagonist, or a PD-L1 antagonist; or any combination thereof.

Embodiment 106: The method of embodiments 105, wherein the cancer is lung cancer, genitourinary cancer, or gastrointestinal cancer.

Embodiment 107: The method of embodiments 106, wherein the cancer is lung cancer.

Embodiment 108: The method of embodiments 107, wherein the lung cancer is non-small cell lung cancer (NSCLC), optionally wherein the cancer is (i) locally advanced, unresectable, locally advanced unresectable, or metastatic, (ii) no longer responding to a treatment, optionally wherein the treatment is a standard of care, a checkpoint inhibitor, a PD-1 antagonist, or a PD-L1 antagonist, or (iii) a combination of (i) and (ii).

Embodiment 109: The method of embodiments 108, wherein the lung cancer is squamous or non-squamous, unresectable locally advanced disease or metastatic disease.

Embodiment 110: The method of any one of embodiments 107 to 109, wherein the subject is checkpoint inhibitor naïve.

Embodiment 111: The method of any one of embodiments 107 to 109, wherein the subject is checkpoint inhibitor experienced.

Embodiment 112: The method of embodiment 106, wherein the cancer is gastrointestinal cancer.

Embodiment 113: The method of embodiment 107, wherein the gastrointestinal cancer is esophageal cancer, gastric cancer, colorectal cancer, pancreatic cancer or liver cancer, optionally wherein the cancer is (i) locally advanced, unresectable, locally advanced unresectable, or metastatic and/or (ii) no longer responding to a treatment, such as a standard of care, a checkpoint inhibitor, a PD-1 antagonist, or a PD-L1 antagonist.

Embodiment 114: The method of embodiment 113, wherein the gastrointestinal cancer is esophageal cancer or gastric cancer, optionally wherein the cancer is (i) locally advanced, unresectable, locally advanced unresectable, or metastatic and/or (ii) no longer responding to a treatment, such as a standard of care.

Embodiment 115: The method of embodiment 114, wherein the gastrointestinal cancer is esophageal adenocarcinoma, esophageal squamous cell carcinoma, gastroesophageal junction adenocarcinoma, or gastric adenocarcinoma, optionally wherein the cancer is (i) locally advanced, unresectable, locally advanced unresectable, or metastatic and/or (ii) no longer responding to a treatment, such as a standard of care.

Embodiment 116: The method of embodiment 115, wherein the cancer is NSCLC, head and neck squamous cell carcinoma (HNSCC), renal cell carcinoma (RCC), breast cancer, colorectal cancer (CRC), melanoma, bladder cancer, ovarian cancer, endometrial cancer, Merkel Cell, or gastroesophageal cancer.

Embodiment 117: The method of any one of embodiments 75-116, wherein PD-L1 expression of the cancer is TPS ≥50%, as measured by a clinically validated PD-L1 THC assay or FDA-approved test.

Embodiment 118: The method of any one of embodiments 75 to 116, wherein the PD-Li, corresponding to TPS ≤50% as measured by a clinically validated PD-L1 IHC assay or FDA-approved test.

Embodiment 119: The method of embodiment 118, wherein PD-L1 expression of the cancer is about 1-10%, about 10%-20%, about 20-30%, about 30-40%, about 40-49%, about 1-49%, about 1-25%, or about 25-49%, as measured by a clinically validated PD-L1 IHC assay or FDA-approved test.

Embodiment 120: The method of embodiment 118, wherein PD-L1 expression of the cancer is ≤1%, as measured by a clinically validated PD-L1 IHC assay or FDA-approved test.

Embodiment 121: The method of any one of embodiments 75-120, wherein the cancer is tumor mutational burden-high (TMB-H; ≥10 mutations/megabase (mut/Mb), as determined by an FDA-approved test.

Embodiment 122: The method of any one of embodiments 75-120, wherein the cancer is not TMB-H.

Embodiment 123: The method of any one of embodiments 75-122, wherein the cancer does not have an actionable oncogenic mutation in ALK, EGFR, ROS, BRAF, or NTRK and/or has wildtype ALK, EGFR, ROS, BRAF, and/or NTRK.

Embodiment 124: The method of any one of embodiments 75-123, wherein the cancer expresses or overexpresses one or more biomarker selected CD73, DNAM-1, PVR, TIGIT, and CD8-Ki67.

Embodiment 125: The method of any one of embodiments 75, 76, 77, 79, and 84, wherein the anti-TIGIT antibody that has reduced binding to one or more activating human FcγRs as compared to wild type (WT) human IgG1 is domvanalimab and the subject is also administered a therapeutically effective amount of PD-1 antagonist or a PD-L1 antagonist.

Embodiment 126: The method of any one of embodiment 78, 80, or 85, wherein the anti-TIGIT antibody that has reduced binding to one or more activating human FcγRs as compared to wild type (WT) human IgG1 is domvanalimab and an additional agent is a PD-1 antagonist or a PD-L1 antagonist.

Embodiment 127: The method of embodiment 125 or 126, wherein the cancer is locally advanced, metastatic, or unresectable non-small cell lung cancer (NSCLC) or locally advanced and unresectable NSCLC, optionally wherein domvanalimab is administered at a dose of about 1200 mg once every three weeks or at a dose of about 1600 mg once every four weeks.

Embodiment 128: The method of embodiment 127, wherein the cancer is metastatic NSCLC, and domvanalimab and the PD-1 antagonist or PD-L1 antagonist are administered in combination with a chemotherapeutic agent or a chemotherapeutic regimen as a first-line treatment.

Embodiment 129: The method of embodiment 128, wherein the cancer is
metastatic non-squamous NSCLC and domvanalimab and the PD-1 antagonist or PD-L1 antagonist are administered in combination with a chemotherapeutic regimen, optionally where the chemotherapeutic regimen is pemetrexed and a platinum-containing chemotherapy; or
metastatic squamous NSCLC and domvanalimab and the PD-1 antagonist or PD-L1 antagonist are administered in combination with a chemotherapeutic regimen, optionally where the chemotherapeutic regimen is a taxane and a platinum-containing chemotherapy.

Embodiment 130: The method of embodiment 127, wherein the cancer is locally advanced or metastatic NSCLC, and domvanalimab and the PD-1 antagonist or PD-L1 antagonist are administered as a first-line treatment without additional therapeutic agents.

Embodiment 131: The method of embodiment 127, wherein the cancer is locally advanced or metastatic NSCLC, and domvanalimab and the PD-1 antagonist or PD-L1 antagonist are administered in combination with an A₂ₐR and/or A_{2b}R antagonist or a CD73 inhibitor as a first-line treatment.

Embodiment 132: The method of embodiment 127, wherein the cancer is metastatic NSCLC, and domvanalimab and the PD-1 antagonist or PD-L1 antagonist are administered as a second-line or greater treatment, optionally in combination with one or more additional therapeutic agent.

Embodiment 133: The method of embodiment 132, wherein the subject has disease progression on or after treatment with a platinum-containing chemotherapy, a checkpoint inhibitor optionally wherein the checkpoint inhibitor is a PD-1 antagonist or a PD-L1 antagonist, or a targeted therapy optionally wherein the targeted therapy is a tyrosine kinase inhibitor.

Embodiment 134: The method of embodiment 132 or 133, wherein domvanalimab and the PD-1 antagonist or PD-L1 antagonist are administered without additional therapeutic agents,

Embodiment 135: The method of embodiment 132 or 133, wherein the subject is administered an additional therapeutic agent in combination with domvanalimab and the PD-1 antagonist or PD-L1 antagonist, optionally wherein each additional therapeutic agent is selected from an A₂ₐR antagonist, an A_{2b}R antagonist, an A₂ₐR/A_{2b}R antagonist, a CD73 inhibitor, a chemotherapeutic agent, or a chemotherapeutic regimen.

Embodiment 136: The method of embodiment 127, wherein the cancer is locally advanced, unresectable NSCLC and the subject's disease has not progressed following chemoradiation therapy, optionally wherein domvanalimab and the PD-1 antagonist or PD-L1 antagonist are administered without additional therapeutic agents.

Embodiment 137: The method of embodiment 125 or 126, wherein the cancer is Stage IB, Stage II, or Stage III NSCLC, and domvanalimab and the PD-i antagonist or PD-Li antagonist are administered as an adjuvant treatment after complete surgical resection, optionally wherein domvanalimab is administered at a dose of about 1200 mg once every three weeks or at a dose of about 1600 mg once every four weeks.

Embodiment 138: The method of Embodiment 125 or 126, wherein the cancer is resectable NSCLC, and domvanalimab and the PD-1 antagonist or PD-L1 antagonist are administered in the neoadjuvant setting, in combination with a chemotherapeutic regimen comprising a platinum-containing chemotherapy.

Embodiment 139: The method of any one of embodiments 127 to 128, wherein the cancer is: without an actionable oncogenic mutation in epidermal growth factor (EGFR) or anaplastic lymphoma kinase (ALK); and/or PD-L1 expression of the cancer as measured by a clinically validated PD-L1 IHC assay or FDA-approved test is ≥1%, ≥5%, ≥10%, or ≥50%.

Embodiment 140: The method of embodiment 125 or 126, wherein the cancer is esophageal, gastroesophageal. junction (GEJ), or gastric adenocarcinoma (GA), optionally wherein domvanalimab is administered at a dose of about 1200 mg once every three weeks or at a dose of about 1600 mg once every four weeks.

Embodiment 141: The method of embodiment 140, wherein the esophageal, GEJ, or GA cancer is locally advanced unresectable or metastatic, and domvanalimab and the PD-1 antagonist or PD-L1 antagonist are administered in combination with a chemotherapeutic agent or a chemotherapeutic regimen as a first-line treatment.

Embodiment 142: The method of embodiment 141, wherein domvanalimab and the PD-1 antagonist or PD-L1 antagonist are administered in combination with a chemotherapeutic regimen comprising a fluoropyrimidine-containing and platinum-containing chemotherapy

Embodiment 143: The method of embodiment 140, wherein the esophageal, GEJ, or GA cancer is locally advanced, locally advanced unresectable or metastatic, and domvanalimab and the PD-1 antagonist or PD-L1 antagonist are administered as a second-line or greater treatment, optionally without additional therapeutic agents.

Embodiment 144: The method of embodiment 143, wherein the subject has disease progression on or after one or more line of therapy comprising a platinum-containing chemotherapy, a fluoropyrimidine-containing chemotherapy, a checkpoint inhibitor optionally wherein the checkpoint inhibitor is a PD-1 antagonist or a PD-L1 antagonist, a targeted agent optionally wherein the targeted agent is a HER2/neu-targeted therapy, or any combination thereof.

Embodiment 145: The method of embodiment 140, wherein the subject has completely resected esophageal or GEJ cancer with residual pathologic disease and has received neoadjuvant chemoradiotherapy.

Embodiment 146: The method of any one of embodiments 140-145, wherein PD-L1 expression of the cancer as measured by a clinically validated PD-L1 IHC assay or FDA-approved test is ≥1%, ≥5%, ≥10%, or ≥50%.

Embodiment 147: The method of any one of embodiments 75-146, wherein treatment results in a reduction in tumor size, a reduction in tumor number, a reduction in metastasis, stable disease, partial response, complete response, or a combination thereof.

Embodiment 148: The method of any one of embodiments 75-147, wherein the treatment results in an improvement in overall survival, progression-free survival, disease control rate, overall response rate, or a combination thereof compared to placebo or a standard of care.

Embodiment 149: The method of any one of embodiments 75-148, wherein the treatment results in an increased time to progression, increased disease-free survival, increased duration of response, an increase in duration of clinical benefit, an increase in time to treatment failure, or any combination thereof, compared to placebo or a standard of care.

Embodiment 150: The method of any one of embodiments 75-149, wherein the anti-TIGIT antibody that has reduced binding to one or more activating human FcγRs as compared to wild type (WT) human IgG1 is formulated for dilution as an aqueous solution comprising about 10 mg/mL to about 100 mg/mL antibody or about 20 mg/mL to about 60 mg/mL antibody, a buffer containing about 15 to about 30 mM histidine/histidine-Cl, about 5% to about 10% (weight/volume) of an excipient selected from the group consisting of sucrose, dextrose, trehalose, sorbitol, and mannitol, about 0 mg/mL to about 10 mg/mL NaCl, and about 0.05 mg/mL to about 0.6 mg/mL polysorbate 80.

### EXAMPLES

The following examples are given to illustrate the present disclosure. It should be understood that the invention is not to be limited to the specific conditions or details described in these examples.

### Example 1: Reduced Binding of Domvanalimab to FcγR as Compared to WT IgG1

The present example demonstrates the reduced binding of domvanalimab (also referred to as “AB154” or “dom”) to FcγR and reduced Fc effector function.

Antibody-dependent cell mediated cytotoxicity (ADCC) is mediated by the antibody Fc domain binding to a FcγR. Domvanalimab was tested by enzyme-linked immunosorbent assay (ELISA) for binding to FcγR isotypes I, IIA, IIB, IIIA, and IIIB. Human IgG1 was used as a positive control and buffer containing no antibody was used as a negative control. A: human FcγR1, B: human FcγR2A, C: human FcγR2B, D: human FcγR3A, and E: human FcγR3B were coated in wells of an ELISA plate, and binding of domvanalimab was detected using HRP-conjugated anti-human IgG secondary antibody.

Briefly, ELISA plate coating solution was prepared by diluting human Fc-gamma receptors in PBS to a final concentration of 1 μg/mL (FcγR1) or 4 μg/mL (Fcγ R2A, 2B, 3A and 3B). 30 μL of coating solution was added to each well of the test plates, and the ELISA plates were then incubated overnight at 4° C. to facilitate receptor binding. The following day, ELISA plates were washed four times with 80 μL wash buffer (1×PBS+0.05% Tween-20) per well, prior to blocking with 80 μL blocking buffer (1×PBS+2% BSA) per well at room temperature for 2 hours. A serial dilution of domvanalimab was prepared in antibody buffer (1×PBS with 0.5% BSA) covering a range of 0-1000 nM. Wells were washed four times with 80 μL wash buffer to remove blocking buffer prior to addition of 30 μL domvanalimab at various concentrations in antibody buffer.

Plates were then incubated for an hour prior to removal of well contents by aspiration followed by four washes with wash buffer. 30 μL of anti-human IgG HRP conjugate at 1:2500 dilution in antibody buffer was added to each of the washed wells. Plates were further incubated at room temperature for an additional 30 minutes. Contents of all wells were again removed by aspiration and plates washed 4 times with wash buffer followed by incubation with 30 μL of freshly prepared chemiluminescent substrate per well. Chemiluminescent substrate solution was prepared by combining equal volumes of component A and component B from the SUPERSIGNAL™ ELISA Pico Chemiluminescent Substrate following the manufacturers recommended procedure. Luminescent signals were captured using an Envision Multimode Reader (Perkin Elmer) system approximately 10 minutes after substrate addition.

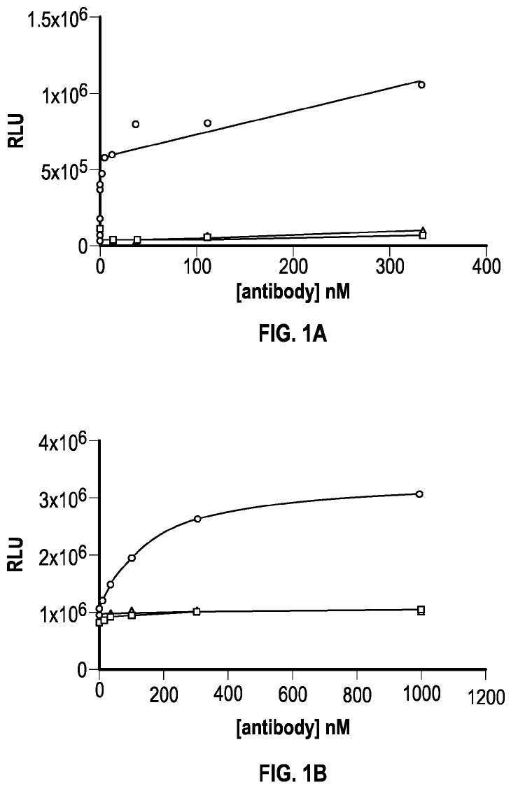

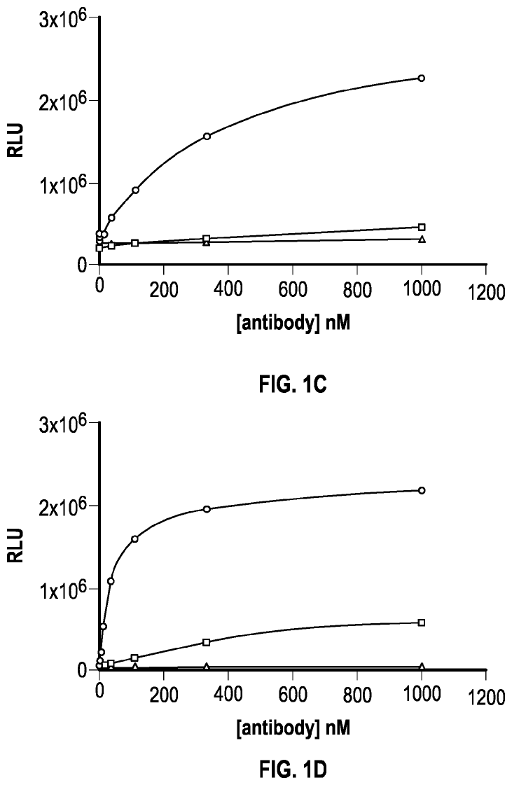

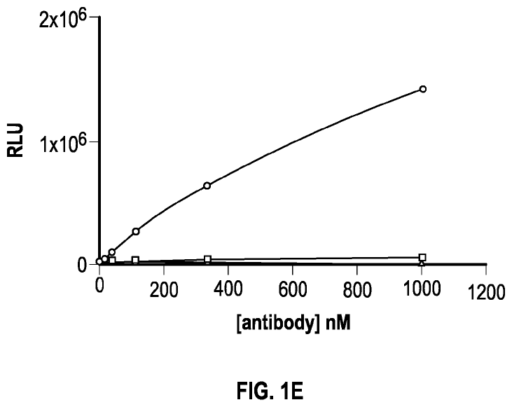

FcγRs 1, 2A, 2B and 3B showed no binding to domvanalimab in the ELISA assay. Fc-gamma receptor 3A showed modest binding to domvanalimab in the ELISA assay (˜20% of the binding exhibited between human IgG1 and Fc-gamma receptor 3). In sum, when compared with a wild-type IgG1 control antibody, no significant binding was observed with domvanalimab for any FcγR isotype, tested to a maximum concentration of 1 μM. Domvanalimab was also tested in an FcγR-IIIA (V158 high-affinity variant) effector reporter bioassay and found to be inactive at concentrations up to 1 μM. Results of these experiments are shown in FIGS. 1A-1E.

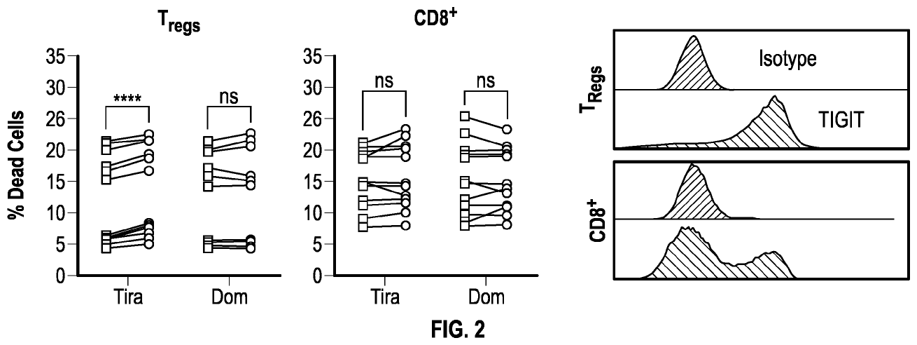

NK-mediated ADCC against Treg or CD8+ target cells isolated from peripheral blood mononuclear cells suspensions was evaluated for domvanalimab and also for tiragolumab, an Fc-enabled anti-TIGIT antibody. Tiragolumab was produced using sequences disclosed in the WHO Drug Information proposed INN publication (List 117; Vol 31 No. 2, 2017). Briefly, target cells (Treg or CD8+ T cells isolated from buffy coats or leukoreduction system chambers using Stem Cell Treg or CD8+ T cell isolation kits, respectively) were incubated with 3 g/mL of anti-TIGIT antibodies for 1 hour at 37° C. Freshly isolated NK cells (from buffy coats or leukoreduction system chambers using Stem Cell NK isolation kit) were then added at a 5:1 ratio of NK to target cells and co-cultured for −20 hours at 37° C. Co-cultures were then transferred to 96-well staining plates, washed with FACS buffer (HBSS, no Ca²⁺/Mg²⁺, 0.5% FBS, 1 mM EDTA) and resuspended in 25 μL/well of Fixable Live-Dead Aqua for 30 minutes at 4° C. Cells were then washed with FACS buffer and resuspended in 120 μL/well of 1% PFA in PBS before acquiring on the LSR Fortessa. Data was analyzed in Flowjo (Treestar) and statistical analyses completed in Graphpad Prism. Statistical analysis: Two-way ANOVA with Sidak's multiple comparisons test comparing anti-TIGIT antibody with appropriate isotype control. Results of these experiments are shown in FIG. 2.

A CDC assay was performed with human complement and a Jurkat cell line stably overexpressing human TIGIT in the presence of domvanalimab or a positive control antibody at increasing concentrations up to 33 nM. Briefly, 50,000 Jurkat-TIGIT cells in RPMI 1640 medium (Invitrogen) containing 10% fetal bovine serum (FBS) were seeded into a 96-well round bottom plates (Nunc). Test antibodies were added starting at a top concentration of 50 μg/ml followed by a three-fold dilution series and allowed to incubate with cells for 1 hour at 37° C. in a 5% CO₂ incubator. After this 1-hour incubation, human complement was added and allowed to incubate for an additional 3 hours at 37° C. in a 5% CO₂ incubator. Following the completion of this 3-hour incubation, 5 g/ml of propidium iodide (ThermoFisher) was added and samples were analyzed by flow cytometry (BD FACSCalibur) to determine the percentages of propidium iodide positivity as a readout for cell death. No cytotoxicity was seen for domvanalimab at any concentration tested.

### Example 2: Pharmacokinetics of Domvanalimab in Humans

The present example illustrates the pharmacokinetics of domvanalimab in human subjects. 43 human subjects (n=10 monotherapy; n=33 combination therapy) were administered Q2W doses of domvanalimab in the range of 0.5-10 mg/kg, Q3W doses of 10 or 15 mg/kg, and Q4W doses of 15 or 20 mg/kg either as a monotherapy or in combination with zimberelimab (AB122). Subjects had ≥1 measurable lesion per RECIST v1.1 and ECOG performance status score 0-1.

In the first dosing cycle, after Q2W dosing, the mean area under the concentration time curve over the first dosage interval, concentration at end of infusion (Ceoinf), and concentration at the end of the dosing interval (Ctrough) increased by 17-fold, 15-fold, and 27-fold over the 20-fold dose range of 0.5-10 mg/kg, respectively, indicating that the PK was roughly dose proportional over the range studied after Q2W administration. Over the entire dose range, the exposure also increased roughly linearly after first dose. The PK of domvanalimab after monotherapy and in combination with zimberelimab at 1 mg/kg and 3 mg/kg were broadly similar. At doses of 10 mg/kg Q2W, 15 mg/kg Q3W, and 20 mg/kg Q4W, the steady-state Ctrough was ≥10 g/mL, the effective concentration for domvanalimab. Examination of the mean PK plots indicated similar PK across the dosing regimens. Accumulation of domvanalimab exposure was observed following multiple dose administration—the accumulation ratio ranged from 1.62-2.22-fold based on mean AUCtau between the first and third doses across all dosing regimens.

All available PK data from this study were combined in a population PK model. In this study, domvanalimab was administered IV as a monotherapy at doses in the range 0.5 to 3 mg/kg, and in combination with zimberelimab over the dose range 3 to 20 mg/kg at schedules of Q2W, Q3W, and Q4W regimen. The population PK model confirmed that the PK of domvanalimab was dose-linear over the dose range and consistent across the dosing regimens.

The terminal half-life was estimated based on the model to be 19.9 days, and the volume of distribution approximated plasma volume, typical of other monoclonal antibodies. Simulations were run with the model of n=500 patients each at 10 mg/kg Q2W, 15 mg/kg Q3W, and 20 mg/kg Q4W dosing regimens for 24 weeks. Simulations indicated that all 3 dosing regimens would maintain domvanalimab concentrations above the effective level of 10 μg/mL in a vast majority of patients.

### Example 3: Pharmacodynamics of Domvanalimab in Human Subjects

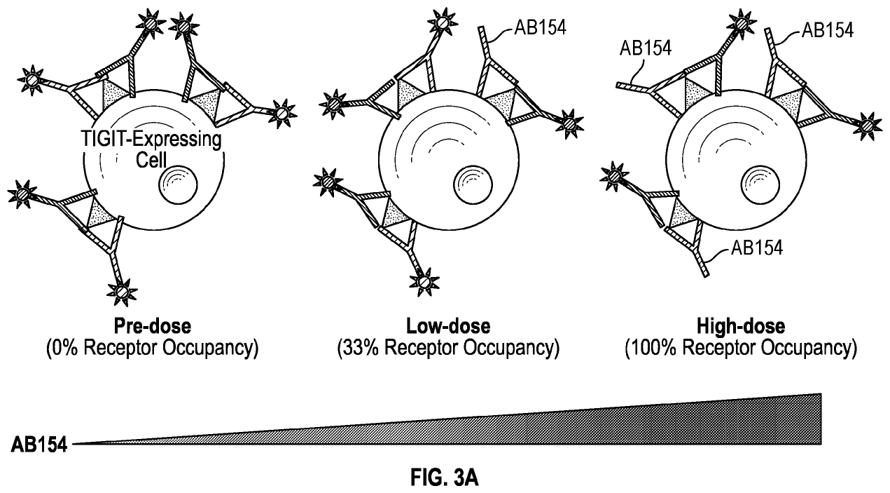

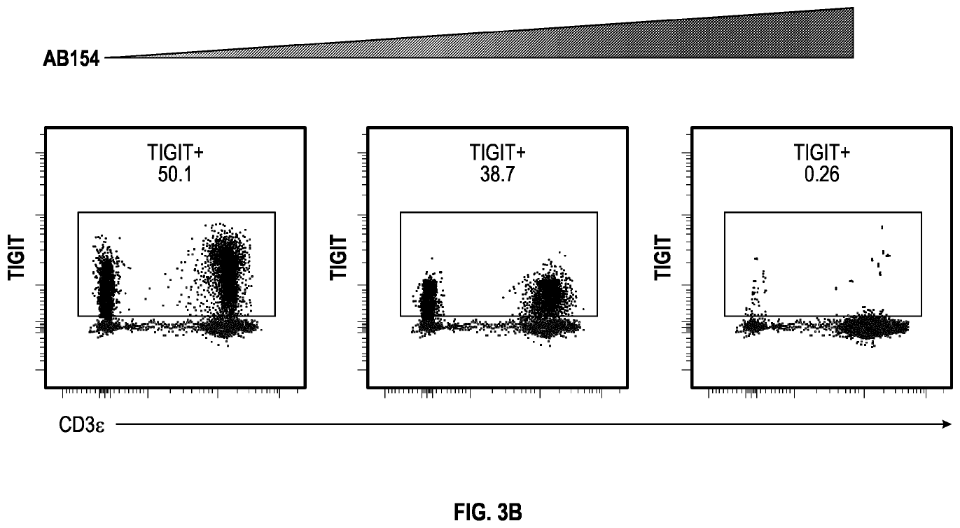

The present example shows receptor occupancy (RO) data from 19 human subjects administered domvanalimab (n=10 monotherapy; n=9 combination therapy with zimberelimab). The RO assay is depicted in FIG. 3A and FIG. 3B.

The RO of domvanalimab on select peripheral lymphocyte populations in whole blood collected from patients enrolled in a clinical study was assessed by flow cytometry using pre- and post-dose whole blood samples. A commercially available anti-TIGIT antibody (MBSA43) that is competitive with domvanalimab was used to determine TIGIT RO, measured by a decrease in the detectable anti-TIGIT antibody signal that occurs following domvanalimab dosing. RO was determined on various immune cell types using antibodies specific for surface markers CD3, CD4, CD8, CD56, FoxP3, and Ki67. RO was calculated as follows:

$$RO ( \% ) = 100 - \frac{\% TIGIT on surface detected by MBSA 43 in ABI 54 - dosed sample}{\% TIGIT on surface detected by MBSA 43 in ABI 54 - free ( 0 nM ) sample} \times 100$$

When dosed Q2W, RO ≥98% was achieved in both monotherapy and combination therapy cohorts on all cell types 1 hour after the end of infusion of the first dose. This complete RO was maintained on Day 15 prior to the following dose, and at all subsequent PD time points assessed prior to the following dose at Day 29, Day 57 and Day 85. Domvanalimab RO was also evaluated in Q3W and Q4W dosing regimens in combination with zimberelimab. Overall, a total of 24 participants have demonstrated maximal RO across all dose groups evaluated (0.5 mg/kg Q2W, 1.0 mg/kg Q2W, 3 mg/kg Q2W, 10 mg/kg Q2W, 10 mg/kg Q3W, 15 mg/kg Q3W, 15 mg/kg Q4W, 20 mg/kg Q4W), at all post-dose time points.

### Example 4: PD-1, TIGIT, and CD226 are Expressed on Various Tumor Infiltrating T Cells Representing Various Phenotypic Profiles and Differentiation States

Cell subsets from frozen dissociated gastric/esophageal (G/E) and lung (NSCLC) cancer patient tumors (sourced from Discovery Life Sciences) were phenotyped using flow cytometry.

The cell subsets selected included those that TIGIT blockade may impact. Briefly, frozen dissociated tumor samples were thawed, counted, and added at 0.2-1×10⁶ cells per well in a 96-well round-bottom staining plate. Cells were washed with FACS buffer (HBSS, no Ca²⁺/Mg²⁺/0.5% FBS, 1 mM EDTA) and resuspended in 50 μL/well of Fixable Live-Dead Aqua/Fc block for 15 minutes at room temperature. The cells were then stained with fluorophore-conjugated antibodies against extracellular targets (minimally including CD45, CD3, CD8, CD16, CD14, CD56, CD155, PD-L1, CD226, PD-1, TIGIT, CD103, CD39) for 30 minutes at 4° C. Cells were washed with FACS buffer and resuspended in fixation buffer (reagents from Invitrogen FoxP3 transcription factor staining buffer set) for 45 minutes at room temperature. Cells were then washed in 1× permeabilization buffer (Invitrogen FoxP3 transcription factor staining buffer set) and incubated for 15 minutes at room temperature in permeabilization buffer containing 20% normal mouse and rat serum to prevent non-specific intracellular antibody binding. Intracellular antibodies (fluorphore-conjugated anti-FoxP3) were then added and incubated for 30 minutes at 4° C. Cells were washed in 1× permeabilization buffer, resuspended in 120 L/well of FACS buffer, acquired on the LSR Fortessa, and analyzed on Flowjo (Treestar).

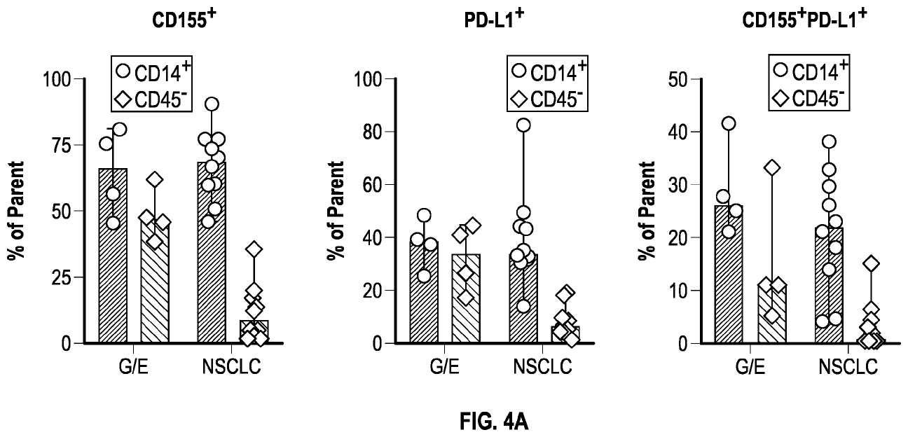

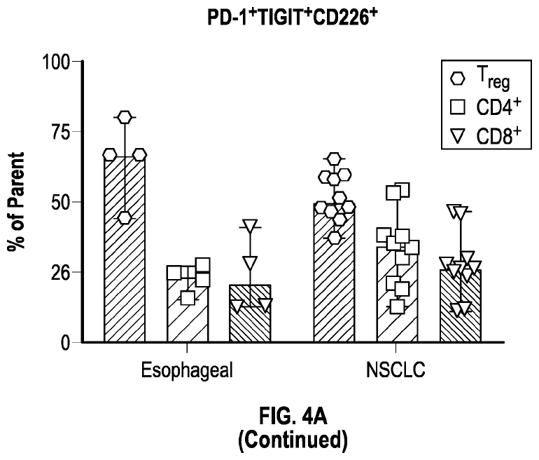

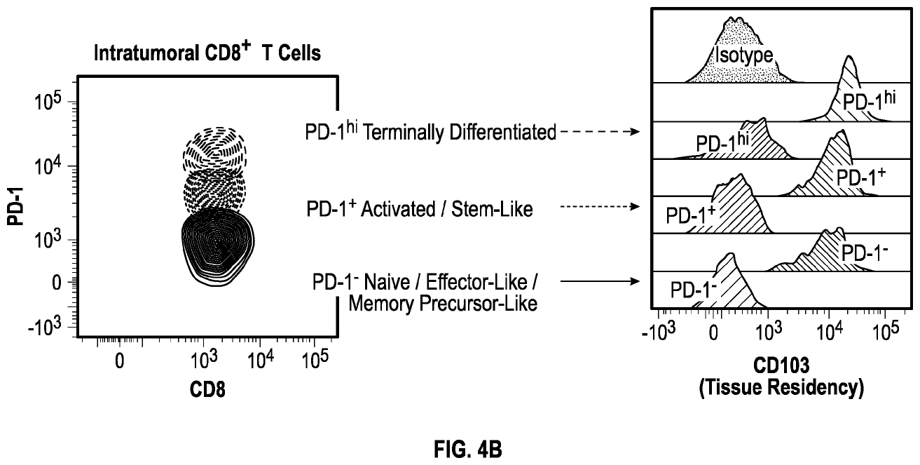

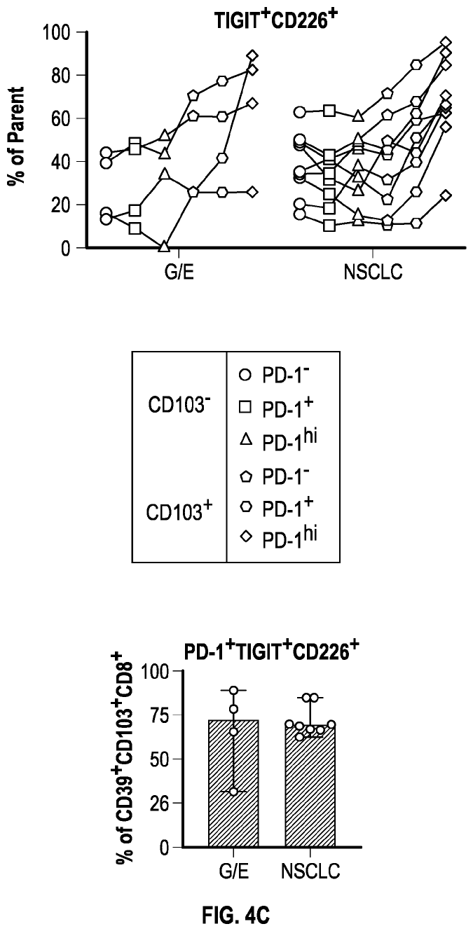

FIG. 4A shows CD155 and PD-L1 ligand expression on CD14⁺ monocytes (CD45⁺CD3⁻ CD16⁻CD56⁻CD14⁺) and cancer/stromal cells (CD45⁻) and co-expression of PD-1, TIGIT, and CD226 on broad T cell populations. FIG. 4B shows a contour plot (NSCLC subject) of PD-1 expression on CD8⁺ T cells encompassing various states of activation and differentiation. Each population was further characterized into tissue resident (CD103⁺) and circulating (CD103⁻) CD8⁺ populations, resulting in six subsets. FIG. 4C shows co-expression of TIGIT and CD226 on the six CD8⁺ T cell populations from FIG. 4B and co-expression of PD-1, TIGIT, and CD226 on probable tumor-specific CD39⁺CD103⁺CD8⁺ T cells. Lines connect populations from the same subject. Each symbol represents a unique subject while bars and error denote median ±range. PD-1^{-/+/hi} population descriptions based on published literature (Kurtulus et al., Immunity, 2019, 50(1):181-194; Dolina et al. 2021 Front Immunol DOI:10.3389/fimmu.2021.715234).

The above data show human tumor-infiltrating lymphocytes from a variety of cancer types expressed appreciable levels of TIGIT on relevant immune populations, including T_{regs} and CD8⁺ T cells with tumor reactive or pre-dysfunctional stem-like phenotypes.

### Example 5: Phase 1 Study of Domvanalimab Alone or in Combination with Zimberelimab in Participants with Pathologically Confirmed Solid Tumors

This example relates to an ongoing Phase 1, dose-escalation study with domvanalimab escalated alone or in combination with zimberelimab in participants with pathologically confirmed solid tumors (≥1 measurable lesion per RECIST v1.1) and ECOG performance status score 0-1. Overall, a total of 75 participants have been administered domvanalimab at doses ranging from 0.5 mg/kg to 20 mg/kg (weight-based dose) or 1200 mg to 1500 mg (flat dose). The number of participants exposed to domvanalimab by treatment cohort and arm is summarized in Table 1. In all instances, domvanalimab and zimberelimab were administered intravenously.

TABLE 1
Summary of human patient exposure to domvanalimab.

| | | No. of |
|---|---|---|
| Treatment | | Participants |
| Arm | Dosing Regimen | Exposed |
| Arm A | AB154 (0.5, 1, 3 mg/kg) Q2W | 10 |
| Arm B | AB154 (0.5, 1, 3 mg/kg) Q2W + AB122 | 9 |
| | (240 mg) Q2W | |
| Arm C | AB154 (10, 15 mg/kg) Q2W + AB122 | 13 |
| | (360 mg) Q2W | |
| Arm D | AB154 (15, 20 mg/kg) Q4W + AB122 | 12 |
| | (480 mg) Q4W | |
| Arm E | AB154 (15 mg/kg) Q3W + AB122 (720 mg) | 6 |
| | Q4W | |
| | AB154 (15 mg/kg) Q3W + AB122 (960 mg) | 6 |
| | Q6W | |
| Arm F | AB154 (1200 mg) Q3W + AB122 (360 mg) | 6 |
| | Q3W | |
| | AB154 (1500 mg) Q4W + AB122 (480 mg) | 7 |
| | Q4W | |
| | AB154 (1200 mg) Q3W + AB122 (720 mg) | 6 |
| | Q4W | |

AB154 = domvanalimab;
AB122 = zimberelimab

Cumulative safety data, as of an initial data cutoff, is as follows.

In the monotherapy cohort (N=10), all 10 participants (100%) had at least 1 treatment-emergent adverse event (TEAE), 3 (30%) of whom had domvanalimab-related TEAEs. The most common (>10%) TEAE regardless of causality was dyspnea (N=3; 30%). All other TEAEs occurred in no more than 1 participant (10%) each. All TEAEs considered related to domvanalimab were Grade 1: fatigue, hyperglycemia, nausea, and pruritus (N=1 each; 10%). Four (40%) participants experienced a total of 5 serious adverse events (SAEs), none of which were considered related to domvanalimab. The SAEs were decreased appetite, dyspnea, hyponatremia, peritonitis bacterial, and pleural effusion (N=1 each; 10%). Dose delays/interruptions due to a TEAE occurred in 1 (10%) participant and 3 (30%) participants discontinued domvanalimab due to a TEAE.

In the pooled combination therapy arms (N=52), 43 (82.7%) participants had at least 1 TEAE. The most common (≥10%) TEAEs regardless of causality were fatigue (N=8; 15.4%), nausea (N=8; 15.4%), and headache (N=6; 11.5%). A total of 29 (55.8%) participants experienced 81 domvanalimab-related TEAEs: all but 5 events were Grade 1 or 2.

A total of 15 (28.8%) participants in the pooled combination arms experienced a total of 22 SAEs. The most common (≥2%) SAEs were small intestinal obstruction (N=4; 7.7%) and immune-mediated hepatitis (N=2; 3.8%). Three (5.8%) participants experienced 1 SAE each deemed related to domvanalimab. These related SAEs were immune-mediated hepatitis (N=2; 3.8%) and liver function test increased (N=1; 1.9%), all of which were Grade 3. Dose delays/interruptions due to a TEAE occurred in 8 (15.4%) participants and 5 (9.6%) participants discontinued treatment due to a TEAE. No deaths have occurred due to treatment-related AEs in this study.

As of a data cutoff of about ten months after the initial cutoff, a total of 56 participants have received doses of domvanalimab ≥10 mg/kg (equivalent to 700-1400 mg) on a Q2W-Q4W schedule in combination with zimberelimab. A profile of immune-related TEAEs for these participants is shown in Table 2. All events are Grade 1 or 2.

TABLE 2
Exemplary immune-related TEAEs (irTEAEs)
in participants who received doses of
domvanalimab ≥10 mg/kg (n = number of participants)

| | dom + zim |
|---|---|
| irTEAE | n (%) |
| Hypothyroidism | 5 (8.9%) |
| Pruritis | 4 (7.1%) |
| Rash | 4 (7.1%) |
| Maculo-papular rash | 3 (5.4%) |
| Infusion related reaction | 3 (5.4%) |
| Immune-mediated hepatitis | 2 (3.6%) |
| Pneumonitis | 2 (3.6%) |
| Arthritis | 2 (3.6%) |
| Hyperthyroidism | 2 (3.6%) |
| Psoriasis | 1 (1.8%) |
| Swelling face | 1 (1.8%) |

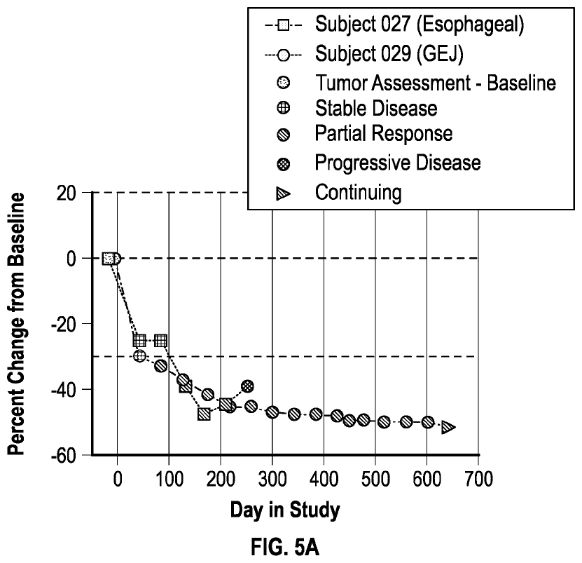

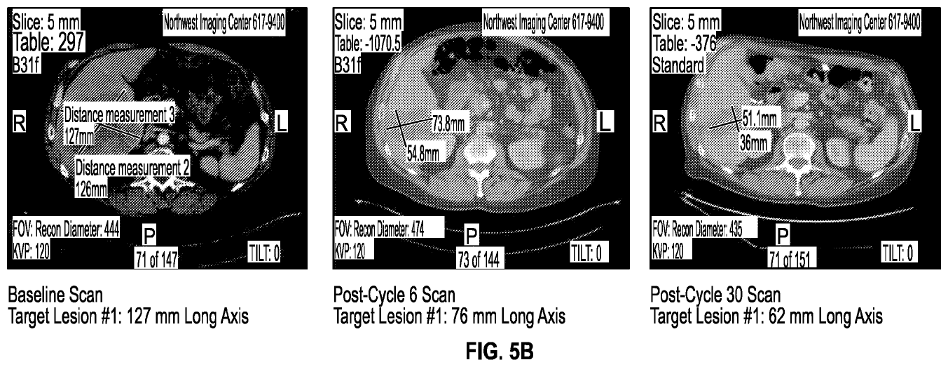

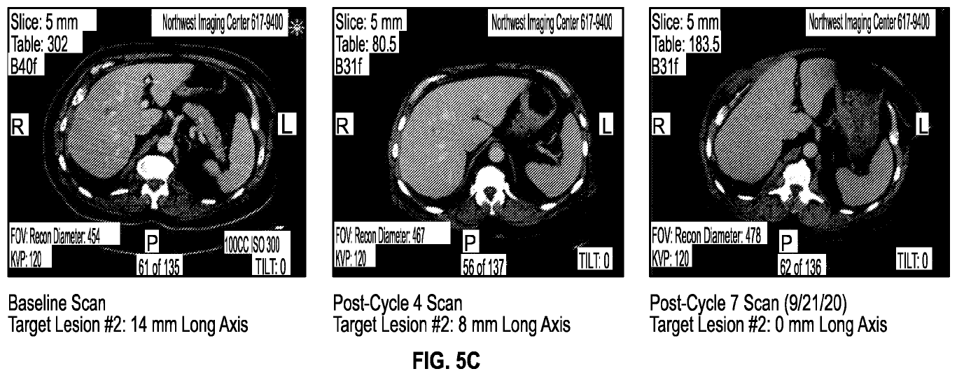

Although the primary objective of the study is to evaluate safety and tolerability of domvanalimab in combination with zimberelimab in a heterogenous patient population representing multiple advanced malignancies, subjects are evaluated for a clinical response. FIG. 5A depicts percent change from baseline for measurable target lesions per RECIST 1.1 over time for two study participants. Partial response (PR) is defined as a decrease of 30% (inclusive) up to 100%. Progressive disease is defined as an increase of 20% or greater. Subject 027 is a 68 year-old male, with Stage IV esophageal adenocarcinoma (PD-L1 (CPS) ˜2%) that had received 3 prior lines of treatment: FOLFOX, carboplatin/paclitaxel, and pembrolizumab. Subject 029 is a 69 year-old male, with Stage IVb gastroesophageal cancer (PD-L1 status unknown) that had received 1 prior line of treatment: FOLFOX. Both participants received 10 mg/kg domvanalimab Q3W and 360 mg zimberelimab Q3W. FIG. 5B and FIG. 5C are scans demonstrating a decrease in target lesion size in the two participants. Scans show one of two target lesions with each patient having lesions not documented in the scans shown here.

### Example 6: Phase 2 Study of Zimberelimab, Etrumadenant, and Domvanalimab Combination Therapy in Front-Line, Non-Small Cell Lung Cancer

This example summarizes data from an ongoing Phase 2, open-label, randomized, 3-arm study evaluating the safety and efficacy of domvanalimab plus zimberelimab vs. zimberelimab alone vs. domvanalimab plus zimberelimab and etrumadenant in 150 people as a first-line treatment for high PD-L1 expression (Tumor Proportion Score (TPS) ≥50% by PharmDx 22C3 or tumor cell membrane ≥50% by Ventana SP263) and EGFR/ALK wild-type, metastatic (Stage IV) squamous or non-squamous NSCLC. Participants were randomized 1:1:1 across the three study arms and treated until disease progression or loss of clinical benefit. Arm 1 received zimberelimab 360 mg IV Q3W (Arm 1=Z). Arm 2 received domvanalimab 15 mg/kg IV Q3W and zimberelimab 360 mg IV Q3W (Arm 2=DZ). Arm 3 received domvanalimab 15 mg/kg IV Q3W, zimberelimab 360 mg IV Q3W, and etrumadenant 150 mg PO QD (Arm 3=EDZ). Patients in the zimberelimab monotherapy arm with confirmed progression had the option to cross over to the triplet arm. Co-primary endpoints were objective response rate (ORR) and progression-free survival (PFS). Secondary endpoints included safety, duration of response and disease control rates. Additional assessments of response/on-treatment effects may include measures of immune cell activation, immune cell suppression, immune cell proliferation, T cell receptor dynamics, and/or increases in memory and antigen experienced T cells in periphery or tumor microenvironment, as well as molecular response assessment by ctDNA dynamics and/or changes in tumor cell markers or gene expression.

As of an initial data cutoff, a total of 74 participants were exposed to domvanalimab. Cumulative domvanalimab safety data are provided from all domvanalimab containing treatment arms. Overall, 69 (93.2%) participants had at least 1 TEAE and 42 (56.7%) of those participants had a treatment related AE related to domvanalimab. Thirty-four (45.9%) participants had a TEAE Grade ≥3 and 8 (10.8%) participants had treatment related TEAEs Grade ≥3 related to domvanalimab. Serious adverse events occurred in 19 (25.6%) participants, 3 (8.3%) of whom had treatment related SAEs. TEAE leading to a domvanalimab dose modification/interruption occurred in 20 (27.0%) participants and 3 (4.0%) participants discontinued domvanalimab due to a TEAE. There were two fatal cases which were considered related to study treatment—1 (1.3%) fatal case of pneumonitis in a participant who received domvanalimah, zimberelimab, and etrumadenant combination treatment, and 1 (1.3%) fatal case of myocarditis in a participant who received domvanalimab and zimberelimab combination treatment. Additional safety data are provided in Tables 3-7. Immune-related adverse events are defined as a treatment-emergent adverse event with Standardized Medical Queries (SMQs) of Hypersensitivity, Immune-mediated disorders, and autoimmune disorders.

Further data were obtained from an interim analysis with a data cutoff of Aug. 31, 2022. A total of 150 patients were randomized with a median follow-up of 11.8 months (range: 0.03-23.5). One patient was improperly randomized to Arm 2 (DZ) and subsequently deemed ineligible for treatment. This patient is included in the intent to treat (ITT) populations, but not the safety population, the latter being defined as patients who received at least one dose of study drug.149 patients received at least one dose of study treatment.

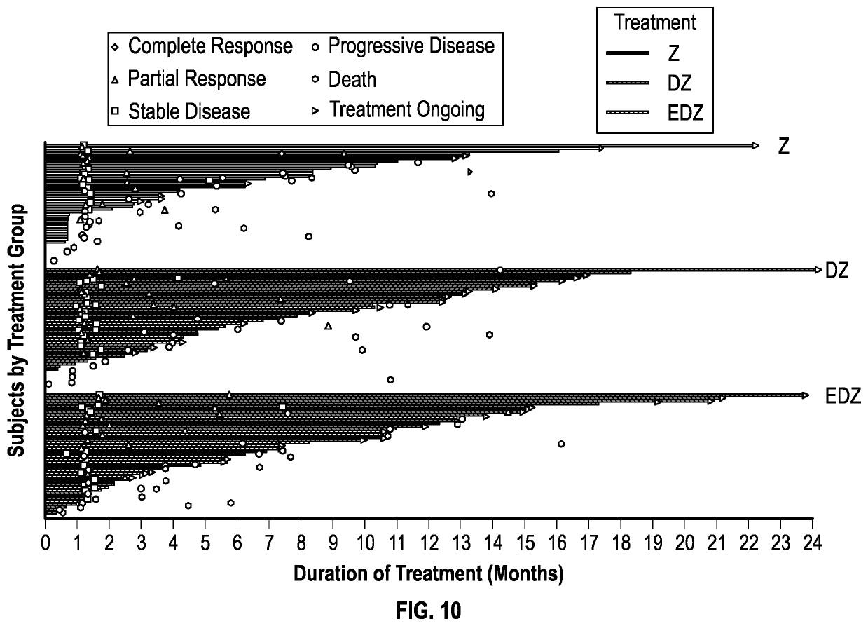

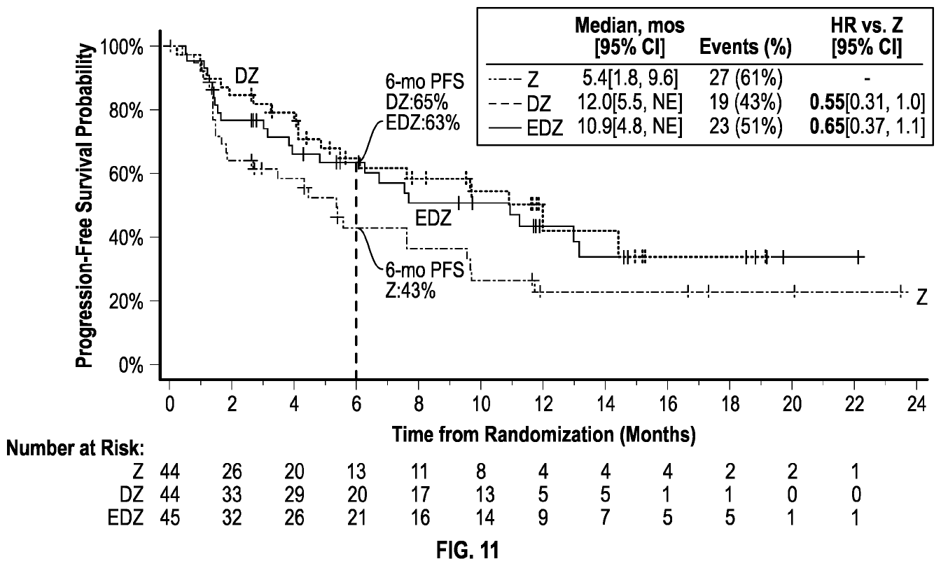

Efficacy for this interim analysis included 133 patients randomized at least 13 weeks prior to data cut-off (ITT-13), allowing for ≥2 post-baseline scans for potential response confirmation. With median follow-up of 11.8 months, domvanalimab-containing arms demonstrated improved ORR and PFS compared to zimberelimab. Improved response rate was seen in domvanalimab-containing arms across multiple subgroup analyses, including PD-L1 status, tobacco history, disease histology, race, etc. (data not shown). Approximately half of patients in domvanalimab-containing arms remain on study treatment compared to only 28% of patients with zimberelimab monotherapy. Patients remaining on treatment include 31 patients with partial response (Z: n=5; DZ: n=13); EDZ: n=13) and 14 patients with stable disease (Z: n=3 DZ: n=6; EDZ: n=5). Time to initial response ranged from 1.2 to 14.6 months across all arms. Median duration of response was not yet reached in any treatment arm (range in months: 1/3+, 17.8+). See FIG. 10 and FIG. 11.

In the safety population (149 patients), grade 3 treatment-emergent adverse events occurred in 58% (Z), 47% (DZ), and 52% (EDZ). Most common TEAEs (≥15% overall) were fatigue, nausea, constipation, dyspnea, decreased appetite and pneumonia. Grade 3 events occurring in ≥5% of patients were pneumonia (8.7%) and anemia (5.4%). Grade 5 TEAEs related to study treatment (per physician assessment) were interstitial lung disease (Z), myocarditis (DZ), pneumonitis (EDZ), and congestive heart failure (EDZ). The majority of pneumonitis events reported were primarily Grade 1-2. There was no clear increase in rates of pneumonitis in domvanalimab-containing arms compared to zimberelimab alone. All cases of rash were grade 1-2, manageable with topical corticosteroids, and more common in domvanalimab+zimberelimab+etrumadenant. There were no discontinuations resulting from rash or infusion-related reactions. A profile of immune-related TEAEs for these participants is shown in Table 8. Immune-related adverse events were again defined as a treatment-emergent adverse event with Standardized Medical Queries (SMQs) of Hypersensitivity, Immune-mediated disorders, and autoimmune disorders. Additional data are shown in Tables 9-11.

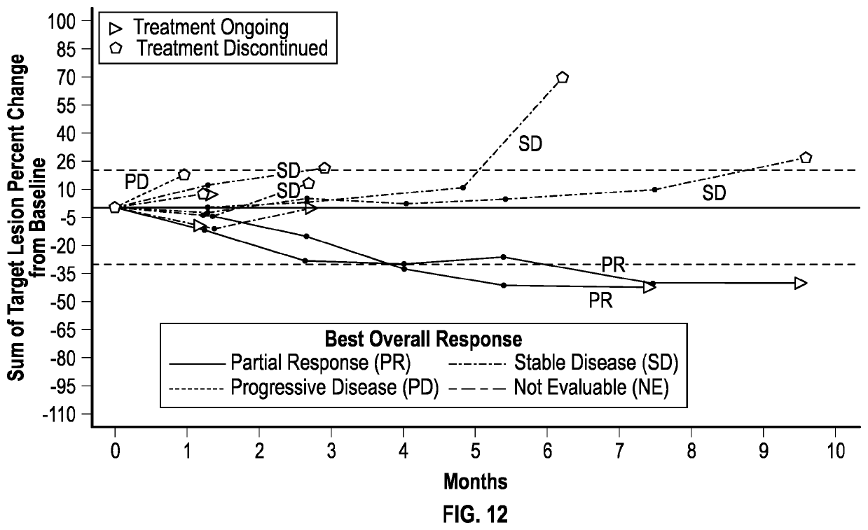

At the time of data-cut, a total of 12 patients received crossover treatment with EDZ, and 5 patients remain on crossover treatment. See FIG. 12. Crossover safety was generally consistent with first-line EDZ safety profile. Three Grade 3-4 TEAEs (no Grade 5) and 2 serious TEAEs were reported.

Overall, both domvanalimab containing arms demonstrated clinically meaningful improvement in ORR and PFS compared to zimberelimab. Treatment with zimberelimab, domvanalimab+zimberelimab, and etrumadenant+domvanalimab+zimberelimab was well tolerated, and the safety profiles of domvanalimab-containing arms were similar to zimberelimab.

TABLE 3
Overall safety data from the initial cutoff date.

| | | | etruma + |
|---|---|---|---|
| Safety evaluable | zim | dom + zim | dom + zim |
| population, n (%)* | (N = 38) | (N = 38) | (N = 36) |
| Any TEAEs (# of subjects) | 36 (95) | 34 (90) | 35 (97) |
| Any Grade ≥3 TEAEs | 20 (53) | 17 (45) | 17 (47) |
| Any SAEs | 18 (47) | 13 (34) | 16 (44) |
| Any Grade ≥3 SAEs | 17 (45) | 13 (34) | 25 (69) |

*n = number of subjects reporting at least one event of type specified. Percentage (%) based on number of subjects in the row category/number of subjects (N) withing the column treatment arm category.

TABLE 4
Treatment modifications, interruptions or discontinuations
as an initial data cutoff date.

| | | | dom + | etruma + |
|---|---|---|---|---|
| | | zim | zim | dom + zim |
| Safety evaluable population, n (%)* | | (N = 38) | (N = 38) | (N = 36) |
| TEAEs | Leading to zim | 4 (11) | 10 (26) | 10 (28) |
| leading to | mod./int. | | | |
| study drug | Leading to | — | 10 (26) | 10 (28) |
| modification/ | dom mod./int.. | | | |
| interruption** | Leading to etruma | — | — | 3 (8) |
| | mod./int.. | | | |
| TEAEs | Leading to | 10 (26) | 7 (18) | 6 (17) |
| leading to | zim discon. | | | |
| study drug | Leading to | — | 7 (18) | 6 (17) |
| discon. | dom discon. | | | |
| | Leading to etruma | — | — | 6 (17) |
| | discon. | | | |

| | | | |
|---|---|---|---|
| Any SAEs Leading to DC | 8 (21) | 4 (11) | 5 (14) |
| Any TEAEs Leading to Death | 3 (8) | 4 (11) | 4 (11) |

*n = number of subjects reporting at least one event of type specified. Percentage (%) based on number of subjects in the row category/number of subjects (N) withing the column treatment arm category.
**Drug modification/interruption also denotes delays in treatment cycles.

TABLE 5
Most common TEAEs (≥10% overall) as an initial data cutoff date.

| | | | | | AB122 + AB154 + | | | |
|---|---|---|---|---|---|---|---|---|
| | AB122 | | AB122 + AB154 | | AB928 | | | |
| | Monotherapy | | Combination | | Combination | | | |
| | (N = 38) | | Therapy (N = 38) | | Therapy (N = 36) | | Overall (N = 112) | |
| Preferred | Subjects | Events | Subjects | Events | Subjects | Events | Subjects | Events |
| Term | n (%) | n | n (%) | n | n (%) | n | n (%) | n |
| Any TEAEs | 36 (94.7) | 271 | 34 (89.5) | 215 | 35 (97.2) | 281 | 105 (93.8) | 767 |
| Fatiguc | 11 (28.9) | 11 | 5 (13.2) | 6 | 7 (19.4) | 7 | 23 (20.5) | 24 |
| Nausea | 7 (18.4) | 7 | 6 (15.8) | 6 | 9 (25.0) | 12 | 22 (19.6) | 25 |
| Constipation | 4 (10.5) | 6 | 9 (23.7) | 9 | 8 (22.2) | 9 | 21 (18.8) | 24 |
| Dyspnoea | 7 (18.4) | 9 | 4 (10.5) | 6 | 6 (16.7) | 12 | 17 (15.2) | 27 |
| Decreased | 6 (15.8) | 6 | 3 (7.9) | 3 | 7 (19.4) | 7 | 16 (14.3) | 16 |
| appetite | | | | | | | | |
| Diarrhoea | 6 (15.8) | 7 | 2 (5.3) | 3 | 7 (19.4) | 8 | 15 (13.4) | 18 |
| Pyrexia | 4 (10.5) | 4 | 3 (7.9) | 3 | 6 (16.7) | 6 | 13 (11.6) | 13 |
| Pneumonia | 2 (5.3) | 2 | 5 (13.2) | 6 | 5 (13.9) | 6 | 12 (10.7) | 14 |
| Pruritus | 6 (15.8) | 6 | 2 (5.3) | 2 | 4 (11.1) | 4 | 12 (10.7) | 12 |
| Rash | 3 (7.9) | 8 | 2 (5.3) | 2 | 7 (19.4) | 9 | 12 (10.7) | 19 |
| Pneumonitis | 6 (15.8) | 7 | 2 (5.3) | 2 | 3 (8.3) | 4 | 11 (9.8) | 13 |
| Vomiting | 3 (7.9) | 3 | 3 (7.9) | 3 | 5 (13.9) | 7 | 11 (9.8) | 13 |

TABLE 6
Most common Grade ≥3 TEAEs (>1 subject) as an initial data cutoff date.

| | | | | | AB122 + AB154 + | | | |
|---|---|---|---|---|---|---|---|---|
| | AB122 | | AB122 + AB154 | | AB928 | | | |
| | Monotherapy | | Combination | | Combination | | | |
| | (N = 38) | | Therapy (N = 38) | | Therapy (N = 36) | | Overall (N = 112) | |
| Preferred | Subjects | Events | Subjects | Events | Subjects | Events | Subjects | Events |
| Term | n (%) | n | n (%) | n | n (%) | n | n (%) | n |
| Any TEAEs of | 20 (52.6) | 41 | 17 (44.7) | 47 | 17 (47.2) | 39 | 54 (48.2) | 127 |
| Grade 3 or | | | | | | | | |
| Higher | | | | | | | | |
| Pneumonia | 2 (5.3) | 2 | 3 (7.9) | 4 | 4 (11.1) | 4 | 9 (8.0) | 10 |
| Dyspnoea | 3 (7.9) | 3 | 2 (5.3) | 2 | 2 (5.6) | 2 | 7 (6.3) | 7 |
| Anaemia | 2 (5.3) | 2 | 3 (7.9) | 3 | 1 (2.8) | 1 | 6 (5.4) | 6 |
| Pulmonary | 3 (7.9) | 3 | 2 (5.3) | 4 | 0 | 0 | 5 (4.5) | 7 |
| embolism | | | | | | | | |
| Pneumonitis | 2 (5.3) | 2 | 0 | 0 | 2 (5.6) | 3 | 4 (3.6) | 5 |
| Fatigue | 0 | 0 | 2 (5.3) | 2 | 1 (2.8) | 1 | 3 (2.7) | 3 |
| Hyperglycaemia | 2 (5.3) | 2 | 0 | 0 | 1 (2.8) | 1 | 3 (2.7) | 3 |
| Hypokalaemia | 3 (7.9) | 3 | 0 | 0 | 0 | 0 | 3 (2.7) | 3 |
| Pleural effusion | 2 (5.3) | 2 | 0 | 0 | 1 (2.8) | 1 | 3 (2.7) | 3 |
| Acute | 0 | 0 | 1 (2.6) | 1 | 1 (2.8) | 1 | 2 (1.8) | 2 |
| respiratory | | | | | | | | |
| failure | | | | | | | | |
| Asthenia | 2 (5.3) | 2 | 0 | 0 | 0 | 0 | 2 (1.8) | 2 |
| Chronic | 0 | 0 | 2 (5.3) | 2 | 0 | 0 | 2 (1.8) | 2 |
| obstructive | | | | | | | | |
| pulmonary | | | | | | | | |
| disease | | | | | | | | |
| Hyperkalaemia | 0 | 0 | 0 | 0 | 2 (5.6) | 2 | 2 (1.8) | 2 |
| Hypertension | 0 | 0 | 1 (2.6) | 1 | 1 (2.8) | 3 | 2 (1.8) | 4 |
| Hypoxia | 1 (2.6) | 1 | 0 | 0 | 1 (2.8) | 1 | 2 (1.8) | 2 |
| Interstitial lung | 2 (5.3) | 3 | 0 | 0 | 0 | 0 | 2 (1.8) | 3 |
| disease | | | | | | | | |
| Myocardial | 1 (2.6) | 1 | 1 (2.6) | 1 | 0 | 0 | 2 (1.8) | 2 |
| infarction | | | | | | | | |
| Pericardial | 1 (2.6) | 1 | 1 (2.6) | 1 | 0 | 0 | 2 (1.8) | 2 |
| effusion | | | | | | | | |
| Respiratory | 0 | 0 | 1 (2.6) | 1 | 1 (2.8) | 1 | 2 (1.8) | 2 |
| failure | | | | | | | | |
| Sepsis | 0 | 0 | 0 | 0 | 2 (5.6) | 2 | 2 (1.8) | 2 |
| Septic shock | 1 (2.6) | 1 | 1 (2.6) | 1 | 0 | 0 | 2 (1.8) | 2 |
| Spinal | 2 (5.3) | 2 | 0 | 0 | 0 | 0 | 2 (1.8) | 2 |
| compression | | | | | | | | |
| fracture | | | | | | | | |

TABLE 7
Most common SAE (>1 subject) as an initial data cutoff date.

| | | | | | AB122 + AB154 + | | | |
|---|---|---|---|---|---|---|---|---|
| | AB122 | | AB122 + AB154 | | AB928 | | | |
| | Monotherapy | | Combination | | Combination | | | |
| | (N = 38) | | Therapy (N = 38) | | Therapy (N = 36) | | Overall (N = 112) | |
| Preferred | Subjects | Events | Subjects | Events | Subjects | Events | Subjects | Events |
| Term | n (%) | n | n (%) | n | n (%) | n | n (%) | n |
| Any SAEs | 18 (47.4) | 30 | 13 (34.2) | 30 | 16 (44.4) | 24 | 47 (42.0) | 84 |
| Pneumonia | 2 (5.3) | 2 | 3 (7.9) | 4 | 1 (2.8) | 1 | 6 (5.4) | 7 |
| Pulmonary | 3 (7.9) | 3 | 2 (5.3) | 4 | 0 | 0 | 5 (4.5) | 7 |
| embolism | | | | | | | | |
| Pleural | 2 (5.3) | 3 | 1 (2.6) | 1 | 1 (2.8) | 1 | 4 (3.6) | 5 |
| effusion | | | | | | | | |
| Pneumonitis | 1 (2.6) | 1 | 0 | 0 | 3 (8.3) | 4 | 4 (3.6) | 5 |
| Anaemia | 0 | 0 | 1 (2.6) | 1 | 1 (2.8) | 1 | 2 (1.8) | 2 |
| Asthenia | 2 (5.3) | 2 | 0 | 0 | 0 | 0 | 2 (1.8) | 2 |
| COVID-19 | 0 | 0 | 0 | 0 | 2 (5.6) | 2 | 2 (1.8) | 2 |
| Chronic | 0 | 0 | 2 (5.3) | 3 | 0 | 0 | 2 (1.8) | 3 |
| obstructive | | | | | | | | |
| pulmonary | | | | | | | | |
| disease | | | | | | | | |
| Interstitial | 2 (5.3) | 3 | 0 | 0 | 0 | 0 | 2 (1.8) | 3 |
| lung disease | | | | | | | | |
| Myocardial | 1 (2.6) | 1 | 1 (2.6) | 1 | 0 | 0 | 2 (1.8) | 2 |
| infarction | | | | | | | | |
| Septic | 1 (2.6) | 1 | 1 (2.6) | 1 | 0 | 0 | 2 (1.8) | 2 |
| shock | | | | | | | | |
| Spinal | 2 (5.3) | 2 | 0 | 0 | 0 | 0 | 2 (1.8) | 2 |
| compression | | | | | | | | |
| fracture | | | | | | | | |

TABLE 8
Summary of Immune-related TEAEs occurring during first-line
treatment from as data cutoff of about 7 months later.

| | Subjects n (%) | | | |
|---|---|---|---|---|
| | Z | DZ | EDZ | Total |
| Any TEAEs that are | 24 (48.0) | 23 (46.9) | 30 (60.0) | 77 (51.7) |
| immune-related | | | | |
| Rash | 6 (12.0) | 5 (10.2) | 9 (18.0) | 20 (13.4) |
| Pneumonitis | 7 (14.0) | 4 (8.2) | 5 (10.0) | 16 (10.7) |
| Grade ≥3 | 3 (6) | 1 (2) | 2 (4) | 6 (4.0) |
| Pruritus | 8 (16.0) | 3 (6.1) | 5 (10.0) | 16 (10.7) |
| Rash maculo-papular | 1 (2.0) | 2 (4.1) | 7 (14.0) | 10 (6.7) |
| Hypothyroidism | 4 (8.0) | 3 (6.1) | 0 | 7 (4.7) |
| Rhinitis allergic | 0 | 1 (2.0) | 4 (8.0) | 5 (3.4) |
| Hyperthyroidism | 1 (2.0) | 1 (2.0) | 2 (4.0) | 4 (2.7) |
| Flushing | 1 (2.0) | 1 (2.0) | 1 (2.0) | 3 (2.0) |
| Interstitial lung disease | 3 (6.0) | 0 | 0 | 3 (2.0) |
| Mouth ulceration | 0 | 0 | 3 (6.0) | 3 (2.0) |
| Stomatitis | 0 | 2 (4.1) | 1 (2.0) | 3 (2.0) |
| Seasonal allergy | 0 | 0 | 2 (4.0) | 2 (1.3) |
| Tracheal obstruction | 0 | 1 (2.0) | 1 (2.0) | 2 (1.3) |
| Wheezing | 0 | 1 (2.0) | 1 (2.0) | 2 (1.3) |
| Acute respiratory failure | 0 | 1 (2.0) | 0 | 1 (0.7) |
| Bronchospasm | 0 | 1 (2.0) | 0 | 1 (0.7) |
| Bulbar palsy | 0 | 1 (2.0) | 0 | 1 (0.7) |
| Colitis | 0 | 1 (2.0) | 0 | 1 (0.7) |
| Contrast media reaction | 1 (2.0) | 0 | 0 | 1 (0.7) |
| Diabetes mellitus | 1 (2.0) | 0 | 0 | 1 (0.7) |
| Diabetic ketoacidosis | 1 (2.0) | 0 | 0 | 1 (0.7) |
| Drug eruption | 1 (2.0) | 0 | 0 | 1 (0.7) |
| Erythema | 1 (2.0) | 0 | 0 | 1 (0.7) |
| Eyelid oedema | 0 | 0 | 1 (2.0) | 1 (0.7) |
| Generalised oedema | 0 | 0 | 1 (2.0) | 1 (0.7) |
| Idiopathic urticaria | 0 | 0 | 1 (2.0) | 1 (0.7) |
| Immune-mediated | 1 (2.0) | 0 | 0 | 1 (0.7) |
| enterocolitis | | | | |
| Infusion related reaction | 0 | 1 (2.0) | 0 | 1 (0.7) |
| Lichen planus | 0 | 0 | 1 (2.0) | 1 (0.7) |
| Myocarditis | 0 | 1 (2.0) | 0 | 1 (0.7) |
| Myositis | 1 (2.0) | 0 | 0 | 1 (0.7) |
| Pancreatitis | 0 | 0 | 1 (2.0) | 1 (0.7) |
| Rash pustular | 0) | 0 | 1 (2.0) | 1 (0.7) |
| Respiratory failure | 1 (2.0) | 0 | 0 | 1 (0.7) |
| Rheumatoid arthritis | 0 | 0 | 1 (2.0) | 1 (0.7) |
| Thyroiditis | 0 | 1 (2.0) | 0 | 1 (0.7) |
| Type I diabetes mellitus | 1 (2.0) | 0 | 0 | 1 (0.7) |
| Urticaria | 0 | 1 (2.0) | 0 | 1 (0.7) |

TABLE 9
Overall safety data as of the later data cutoff.

| | | | etruma + |
|---|---|---|---|
| Safety evaluable | zim | dom + zim | dom + zim |
| population, n (%) | (n = 50) | (n = 49) | (n = 50) |
| Any TEAEs (# of subjects) | 50 (100) | 47 (95.9) | 48 (96.0) |
| Any Grade ≥3 TEAEs | 29 (58.0) | 23 (46.9) | 26 (52.0) |
| Any SAEs | 27 (54.0) | 16 (32.7) | 26 (52.0) |
| Any Grade ≥3 SAEs | 23 (46.0) | 16 (32.7) | 19 (38.0) |

TABLE 10
Treatment modifications, interruptions or discontinuations
as of the later data cutoff date.

| | | | dom + | etruma + |
|---|---|---|---|---|
| | | zim | zim | dom + zim |
| Safety evaluable population, n (%)* | | (N = 50) | (N = 49) | (N = 50) |
| TEAEs | Leading to study | 12 (24.0) | 12 (24.5) | 22 (44.0) |
| leading to | drug mod. int. | | | |
| study drug | Related to study | 7 (14.0) | 2 (4.1) | 10 (20.0) |
| modification/ | treatment | | | |
| interruption*** | Leading to zim | 12 (24) | 12 (24.5) | 16 (32.0) |
| | mod./int. | | | |
| | Related to zim | 7 (14.0) | 2 (4.1) | 5 (10.0) |
| | Leading to | — | 12 (24.5) | 16 (32.0) |
| | dom mod./int.. | | | |
| | Related to dom | | 2 (4.1) | 6 (12.0) |
| | Leading to etruma | — | | 21 (42.0) |
| | mod./int.. | | | |
| | Related to etruma | | | 7 (14.0) |
| TEAEs | Leading to zim | 14 (28) | 8 (16.3) | 10 (20.0) |
| leading to | discon. | | | |
| study drug | Related to zim | 9 (18.0) | 3 (6.1) | 3 (6.0) |
| discon. | Leading to | — | 8 (16.3) | 10 (20) |
| | dom discon. | | | |
| | Related to dom | | 2 (4.1) | 3 (6.0) |
| | Leading to ctruma | — | — | 10 (20) |
| | discon. | | | |
| | Related to etruma | | | 3 (6.0) |

| | | | |
|---|---|---|---|
| Any SAEs Leading to Study Drug | 9 (18.0) | 5 (10.2) | 8 (16.0) |
| Discontinuation | | | |
| Related to study treatment | | | |
| Any TEAEs Leading to Death | 3 (6.0) | 4 (8.2) | 7 (14.0) |
| Related to study treatment | 1 (2.0) | 1 (2.0) | 2 (4.0) |

*n = number of subjects reporting at least one event of type specified. Percentage (%) based on number of subjects in the row category number of subjects (N) withing the column treatment arm category.
**Drug modification/interruption also denotes delays in treatment cycles

TABLE 11
Efficacy and Safety Data as of the later data cutoff date.

| Efficacy Population | Z (n = 44) | DZ (n = 44) | EDZ (n = 45) |
|---|---|---|---|
| CORR n (%) [95% CI] | 12 (27) | 18 (41) | 18* (40) |
| | [15.0, 42.8] | [26.3, 56.8] | [25.7, 55.7] |
| Complete Response | 0 (0) | 0 (0) | 0 (0) |
| Partial Response | 12 (27) | 18 (41) | 18 (40) |
| Stable Disease | 14 (32) | 15 (34) | 16 (36) |
| Progressive Disease | 11 (25) | 2 (5) | 6 (13) |
| Not Evaluable | 7 (16) | 9 (21_ | 5 (11) |
| Median PFS (mo) | 5.4 | 12.0 | 10.9 |
| [95% CI] | [1.8, 9.6] | [5.5, NE] | [4.8, NE] |
| Hazard Ratio vs Z | — | 0.55 | 0.65 |
| [95% CI] | | [0.31, 1.0] | [0.37, 1.1] |
| 6-mo PFS % (95% CI) | 43 (27, 59) | 65 (49, 80) | 63 (48, 78) |
| Ongoing first-line | 14 | 25 | 24 |
| treatment | | | |
| Safety Population | Z (n = 50) | DZ (n = 49) | EDZ (n = 50) |
| Infusion-related reactions | 2 (4) | 2 (4) | 5 (10) |
| Rash | 6 (12) | 5 (10) | 9 (18) |
| Median Treatment | 9.8 (0, 97) | 21.0 (0, 105) | 24.3 (2, 107) |
| Duration, weeks | | | |
| (range) | | | |

cORR: confirmed Objective Response Rate, per RECIST v1.1
NE: Not Evaluable
Z: zimberelimab;
DZ: domvanalimab + zimberelimab;
EDZ: etrumadenant + domvanalimab + zimberelimab
Mo: month
*Three additional patients in Arm 3 (i.e., additional to the 18 patients with a cORR) had initial objective response and await confirmatory.

TABLE 12
Baseline Characteristics as of the later data cutoff date.

| ITT, n (%) | | Z (n = 50) | DZ (n = 50) | EDZ (n = 50) |
|---|---|---|---|---|
| Median Age, years (range) | | 66 (43, 84) | 69 (45, 92) | 69 (49, 83) |
| ≥65 years | | 28 (56) | 34 (68) | 35 (70) |
| Sex*: Male | | 34 (68) | 33 (66) | 34 (68) |
| Race | Asian | 25 (50) | 23 (46) | 27 (54) |
| | White | 20 (40) | 24 (48) | 21 (42) |

| | | | |
|---|---|---|---|
| Never Smokers | 7 (14) | 5 (10) | 5 (10) |
| ECOG Status*: 1 | 37 (74) | 35 (70) | 35 (70) |
| Squamous cell carcinoma | 9 (18) | 16 (32) | 16 (32) |
| Brain mets at baseline | 7 (14) | 7 (14) | 8 (16) |
| Liver mets at baseline | 9 (18) | 11 (22) | 4 (8) |
| Local PD-L1 scoring, % TPS | 80 (50, 100) | 70 (50, 100) | 78 (50, 100) |
| median (range) | | | |
| PD-L1: ≥75% | 30 (60) | 23 (46) | 29 (58) |

TABLE 13
Cross-Over Treatment as of the later data cutoff date

| | Crossover (EDZ) |
|---|---|
| Crossover, n (%) | (N = 12) |
| Confirmed ORR, [95% CI]* | 2 (17) [2.1, 48.4] |
| Complete Response | 0 (0) |
| Partial Response | 2 (17) |
| Stable Disease | 8 (67) |
| Progressive Disease | 1 (8) |
| Not evaluable | 1 (8) |
| Disease Control Rate, [95% CI] | 33% [9.9, 65.1] |

### Example 7: Phase 3 Study to Evaluate Zimberelimab Monotherapy Compared to Standard Chemotherapy or Zimberelimab Combined with Domvanalimab in Front-Line, PD-L1-Positive Locally Advanced or Metastatic Non-Small Cell Lung Cancer

This example summarizes an ongoing Phase 3, open-label, randomized study evaluating the safety and efficacy of zimberelimab plus domvanalimab in people with histologically confirmed, treatment naïve, PD-L1 high, squamous or non-squamous NSCLC that is locally advanced or metastatic (Stage IIIB-IV per American Joint Committee on Cancer, version 8) without actionable mutation. Actionable mutations include those mutations for which a targeted therapy is approved by local health authority and available at the time of enrollment (e.g., ALK fusion oncogene, certain EGFR, ROS, BRAF, NTRK mutations).

In Part 1 of the study, three arms were enrolled to evaluate the safety and efficacy of zimberelimab alone vs. zimberelimab plus domvanalimab vs. platinum doublet chemotherapy. Participants in Arm 1 received zimberelimab 360 mg IV Q3W until disease progression or intolerance. Participants in Arm 2 received domvanalimab 15 mg/kg IV Q3W and zimberelimab 360 mg IV Q3W until disease progression or intolerance. Participants in Arm 3 received carboplatin AUC 5 or 6 (maximum dose 900 mg) plus either paclitaxel 200 mg/m² IV Q3W or pemetrexed 500 mg/m² IV Q3W until disease progression, at which time they have the opportunity to crossover to Arm 1. PD-L1 high status (tumor proportion score (TPS); 50%) as determined by the 22C3 PharmDx assay was assessed and confirmed by central laboratories.

In Part 2 of the study, enrollment in Part 1 stopped and two new arms are enrolled to evaluate the safety and efficacy of zimberelimab plus domvanalimab vs. pembrolizumab. Participants in Arm 4 receive zimberelimab 360 mg by IV infusion Q3W plus domvanalimab 1200 mg by IV infusion Q3W on Day 1 of each 21-day cycle until disease progression or intolerance. Participants in Arm 5 receive pembrolizumab 200 mg by IV infusion Q3W on Day 1 of each 21-day cycle until disease progression or intolerance. Approximately 300 participants may be randomized to each of Arm 4 and Arm 5. Randomization is stratified based on Eastern Cooperative Oncology Group performance status (ECOG PS; 0 or 1), geographic region (Asia vs. non-Asia), and histology (squamous vs. non-squamous). PD-Li high status (tumor cell (TC) ≥50%) as determined by the Ventana SP263 IHC assay is assessed and confirmed by central laboratories. Participants already enrolled and assigned to Part 1 treatment arms may continue on study until disease progression, withdrawal of consent, or unacceptable toxicity. The total number ofparticipants in Part 1 is estimated to be approximately 125 with 50 participants each in Arm 1 and Arm 2, and 25 participants in Arm 3. In addition, crossover from Arm 3 to Arm 1 is still permitted.

Primary and secondary efficacy analyses are based on the intent to treat population, which comprises all randomized participants in Part 2 (i.e., a comparison of Arm 4 vs. Arm 5).

TABLE 14
OBJECTIVES AND ENDPOINTS

| Primary Objective | Primary Endpoint |
|---|---|
| To evaluate the efficacy of | OS |
| zimberelimab and domvanalimab | |
| combination therapy compared to | |
| pembrolizumab in OS (Arm 4 vs | |
| Arm 5) | |
| Secondary Objectives | Secondary Endpoints |
| To evaluate the efficacy of | PFS according to RECIST v1.1 as assessed |
| zimberelimab and domvanalimab | by BICR |
| combination therapy compared to | Confirmed ORR according to RECIST v1.1 |
| pembrolizumab in PFS and ORR | as assessed by BICR |
| (Arm 4 vs Arm 5) | |
| To assess the safety of zimberelimab | Presence of treatment-emergent adverse |
| and domvanalimab combination | events |
| therapy compared to pembrolizumab | Changes in vital signs measurements and |
| (Arm 4 vs Arm 5) | clinical laboratory parameters |
| To compare the effect of | Time to first symptom deterioration in |
| zimberelimab and domvanalimab | NSCLC-SAQ total score |
| relative to pembrolizumab (Arm 4 vs | |
| Arm 5) on health-related QOL using | |
| NSCLC-SAQ | |
| Exploratory Objectives | Exploratory Endpoints |
| To evaluate the efficacy of | PFS and ORR according to RECIST v1.1 as |
| zimberelimab and domvanalimab | assessed by the investigator |
| combination therapy compared to | DOR for confirmed response per RECIST |
| pembrolizumab in PFS, ORR, and | v1.1 |
| DOR (Arm 4 vs Arm 5) | |
| To assess disease-related symptoms | Mean change from baseline of the total score |
| and health related QOL between | and cach domain of NSCLC-SAQ |
| zimberelimab and domvanalimab | Mean change from baseline of total score of |
| versus pembrolizumab (Arm 4 vs | EORTC QLQ-C30, and of each domain |
| Arm 5) using NSCLC-SAQ; the | The proportion of participants with |
| EORTC QLQ-C30 v3; PGIS; and | meaningful change in each domain of |
| PGIC | NSCLC-SAQ and EORTC QLQ-C30 while |
| | on treatment |
| | Time to first improvement and time to first |
| | deterioration in each domain of NSCLC- |
| | SAQ and EORTC QLQ-C30 |
| To assess treatment-related | Frequency, severity, amount, interference, |
| symptoms between zimberelimab | and presence/absence of treatment-related |
| and domvanalimab versus | symptoms based on PRO-CTCAE |
| pembrolizumab (Arm 4 vs Arm 5) | |
| using the PRO-CTCAE | |
| To assess overall treatment | Mean change from baseline of FACT-Item |
| tolerability from a participant | GP5 score |
| perspective using FACT-Item GP5 | |
| To study exploratory biomarkers to | Exploratory biomarker analysis may include |
| understand the disease biology, | but are not limited to protein expression and |
| response to treatment, and to identify | NGS, RNAseq (immune context) and/or |
| surrogate markers of response | WES, change in ctDNA from baseline, on |
| | study, and at time of progression. |

BICR = blinded independent central review;
DOR = duration of response;
ctDNA = change in circulating tumor DNA;
EORTC QTQ-C30 v3 = European Organization for Research and Treatment of Cancer Quality of Life Questionnaire-Core 30 Version 3;
FACT-Item 3 = Functional Assessment of Cancer Therapy—Item GP5;
NGS = next-generation sequencing;
NSCLC-SAQ = non-small cell lung cancer Symptom Assessment Questionnaire;
ORR = objective response rate;
OS = overall survival;
PFS = progression-free survival;
PGIC = Patient Global Impression of Change;
PGIS = Patient Global Impression of Severity;
PRO-CTCAE = Patient-Reported Outcomes version of the Common Terminology Criteria for Adverse Events;
QOL = quality of life;
RECIST = Response Evaluation Criteria in Solid Tumors;
RNAseq = ribonucleic acid sequencing;
WES = whole exome sequencing.

Safety analyses will be based on the safety population, defined as all participants who receive at least 1 dose of investigational product. Adverse events (AEs) will be graded according to National Cancer Institute Common Terminology Criteria for Adverse Events (NCI CTCAE). Safety objectives include assessment of treatment-emergent adverse events and immune-related adverse events.

### Example 8-1: Phase 1B/2 Study of Domvanalimab and Zimberelimab in Patients with Advanced GI Malignancies

This example summarizes an ongoing Phase 1B/2 study evaluating the safety and efficacy of zimberelimab plus domvanalimab in 1L and ≥2L advanced GI malignancies. Arm 1 evaluates 1L treatment in CPI naïve patients without prior systemic therapy that have locally advanced or metastatic disease and measurable (RECIST 1.1) EAC, GEJ or gastric cancer. Patients receive (a) domvanalimab 20 mg/kg Q4W, zimberelimab 480 mg Q4W and FOLFOX (oxaliplatin 85 mg/m², leucovorin 400 mg/m², fluorouracil 400 mg/m² Day 1 and fluorouracil 1200 mg/m² D1-D2, Q2W) or (b) domvanalimab 15 mg/kg Q3W, zimberelimab 360 mg Q3W, and depending on region XELOX (capecitabine 1000 mg/m² BID D1-14 and oxaliplatin 130 mg/m² D1 Q3W). Arm 2 evaluates ≥2L treatment in CPI naïve patients with locally advanced or metastatic disease and measurable (RECIST 1.1) EAC, GEJ or gastric cancer. Patients receive domvanalimab 15 mg/kg Q3W and zimberelimab 260 mg Q3W. Arm 3 evaluates ≥2L treatment in CPI experienced patients with locally advanced or metastatic disease and measurable (RECIST 1.1) EAC, GEJ or gastric cancer. Patients receive domvanalimab 15 mg/kg Q3W and zimberelimab 260 mg Q3W.

Coprimary endpoints are safety and confirmed objective response rate (ORR) according to RECIST v1.1 as assessed by the investigator. At least 20 patients in each cohort are required to have TAP ≥5% measured by Ventana PD-L1 (SP263) Assay.

Additional assessments of response/on-treatment effects may include measures of immune cell activation, immune cell suppression, immune cell proliferation, T cell receptor dynamics, and/or increases in memory and antigen experienced T cells in periphery or tumor microenvironment, as well as molecular response assessment by ctDNA dynamics and/or changes in tumor cell markers or gene expression. Safety objectives include assessment of treatment-emergent adverse events.

### Example 8-2: Phase 2 Study of Domvanalimab and Zimberelimab in Patients with Advanced GI Malignancies

This example summarizes an ongoing Phase 2 study evaluating the safety and efficacy of zimberelimab plus domvanalimab in 1L and ≥2L advanced GI malignancies. Arm 1 evaluates 1L treatment in CPI naïve patients without prior systemic therapy that have locally advanced or metastatic disease and measurable (RECIST 1.1) EAC, GEJ or gastric cancer. Patients receive (a) domvanalimab 1600 mg Q4W, zimberelimab 480 mg Q4W and FOLFOX (oxaliplatin 85 mg/m², leucovorin 400 mg/m², fluorouracil 400 mg/m² Day 1 and fluorouracil 1200 mg/m² D1-D2, Q2W) or (b) zimberelimab 480 mg Q4W in addition to chemotherapy with FOLFOX. Arm 2 evaluates ≥2L treatment in CPI naïve patients with locally advanced or metastatic disease and measurable (RECIST 1.1) EAC, GEJ or gastric cancer. Patients receive domvanalimab 1200 mg Q3W and zimberelimab 360 mg Q3W. Arm 3 evaluates ≥2L treatment in CPI experienced patients with locally advanced or metastatic disease and measurable (RECIST 1.1) EAC, GEJ or gastric cancer. Patients receive domvanalimab 1200 mg Q3W and zimberelimab 360 mg Q3W.

Coprimary endpoints are safety and confirmed objective response rate (ORR), defined as the percentage of patients with measurable disease who have achieved a confirmed best response of complete response (CR) or partial response (PR) according to RECIST v1 0.1 as assessed by the investigator. PD-L1 expression will be monitored as participant accrual occurs and prospective testing of PD-L1 status may be required for participant selection if there is a significant imbalance between participants with high and low PD-L1 expression. Each cohort will enroll approximately 20 patients that have TAP ≥5% measured by Ventana PD-L1 (SP263) Assay or an equivalent level of PD-L1 expression measured by another clinically validated assay.

Additional assessments of response/on-treatment effects may include measures of immune cell activation, immune cell suppression, immune cell proliferation, T cell receptor dynamics, and/or increases in memory and antigen experienced T cells in periphery or tumor microenvironment, as well as molecular response assessment by ctDNA dynamics and/or changes in tumor cell markers or gene expression.

Safety objectives include assessment of treatment-emergent adverse events.

### Example 9: Phase 3 Study of Domvanalimab, Zimberelimab and Chemotherapy in Patients with Advanced EAC, GEJ, and Gastric Carcinoma

This example summarizes a planned Phase 3 study evaluating the safety and efficacy of zimberelimab plus domvanalimab plus chemotherapy (FOLFOX or CAPOX, investigator's choice) in patients with locally advanced unresectable or metastatic EAC, GEJ, and gastric carcinoma, without prior systemic treated, that have measurable disease (RECIST 1.1) EAC, GEJ or gastric cancer, regardless of PD-L1 expression. Patients will be randomized 1:1 to receive (a) domvanalimab (1200 mg Q3W or 1600 mg Q4W), zimberelimab (360 mg Q3W or 480 mg Q4W), and investigator's choice of chemotherapy or (b) nivolumab and investigator's choice of chemotherapy.

Coprimary endpoints are overall survival in the intent-to-treat (ITT) population and OS in TAP ≥5% measured by Ventana PD-L1 (SP263). Secondary endpoints include PFS per RECIST 1.1 in ITT population and PFS in TAP ≥5% measured by Ventana PD-L1 (SP263).

Additional assessments of response/on-treatment effects may include measures of immune cell activation, immune cell suppression, immune cell proliferation, T cell receptor dynamics, and/or increases in memory and antigen experienced T cells in periphery or tumor microenvironment, as well as molecular response assessment by ctDNA dynamics and/or changes in tumor cell markers or gene expression.

Safety objectives include assessment of treatment-emergent adverse events.

### Example 10: Clinical Safety of Domvanalimab

This example provides a summary of the clinical safety for 99 participants who received at least 1 dose of domvanalimab in the domvanalimab clinical program and who experienced a TEAE. Data are presented by study in Table 15 and by relationship to domvanalimab in Table 16. Table 15 summarizes very common (≥10%) TEAEs and Table 16 summarizes common (≥5%) TEAEs considered related to domvanalimab. Most SAEs occurred at a frequency of 1.0% or 2.0% by preferred term, with the exception of small bowel obstruction which occurred at a frequency of 4.0% (Table 15).

All TEAEs, related-TEAEs, and SAEs for these 99 participants (regardless of incidence) can be found in Table 18, Table 19, and Table 20, respectively (at the end of the Examples section).

TABLE 15
Summary of Treatment-emergent Adverse Events Occurring
in ≥10% in Domvanalimab-Treated Participants

| | | ARC-7 | Total |
|---|---|---|---|
| TEAE Preferred Term | (n = 62) | (n = 37) | (n = 99) |
| Any TEAEs | 53 (85.5%) | 31 (83.8%) | 84 (84.8%) |
| Nausea | 9 (14.5%) | 4 (10.8%) | 13 (13.1%) |
| Fatigue | 9 (14.5%) | 3 (8.1%) | 12 (12.1%) |
| Diarrhoea | 5 (8.1%) | 5 (13.5%) | 10 (10.1%) |
| Pruritus | 6 (9.7%) | 4 (10.8%) | 10 (10.1%) |

TABLE 16
Summary of All Domvanalimab-Related TEAEs Occurring
in >2 Participants in Domvanalimab-Treated Participants

| | | ARC-7 | Total |
|---|---|---|---|
| TEAE Preferred Term | (n = 62) | (n = 37) | (n = 99) |
| Any TEAEs | 32 (51.6%) | 18 (48.6%) | 50 (50.5%) |
| Pruritus | 5 (8.1%) | 4 (10.8%) | 9 (9.1%) |
| Fatigue | 4 (6.5%) | 2 (5.4%) | 6 (6.1%) |
| Nausea | 5 (8.1%) | 1 (2.7%) | 6 (6.1%) |
| Maculo-papular rash | 3 (4.8%) | 3 (8.1%) | 6 (6.1%) |
| Diarrhoea | 3 (4.8%) | 2 (5.4%) | 5 (5.1%) |
| Pyrexia | 3 (4.8%) | 2 (5.4%) | 5 (5.1%) |
| Arthralgia | 1 (1.6%) | 3 (8.1%) | 4 (4.0%) |
| Headache | 3 (4.8%) | 1 (2.7%) | 4 (4.0%) |
| Hypothyroidism | 4 (6.5%) | 0 | 4 (4.0%) |
| Rash | 1 (1.6%) | 3 (8.1%) | 4 (4.0%) |
| Decreased appetite | 1 (1.6%) | 2 (5.4%) | 3 (3.0%) |
| Hyperthyroidism | 2 (3.2%) | 1 (2.7%) | 3 (3.0%) |
| Tumour flare | 3 (4.8%) | 0 | 3 (3.0%) |

Based on clinical data to date, administration of domvanalimab in cancer patients has been well tolerated. The current safety profile for domvanalimab supports a favorable benefit/risk assessment and suggests a low incidence of immune-related adverse events.

### Example 11: Peripheral T Cells are not Depleted in Human Subjects Undergoing Domvanalimab+Zimberelimab Combination Therapy

Multiple clinical studies have longitudinally followed patients, or are following patients, being treated with domvanalimab alone or in combination with one or more additional therapy (e.g., zimberelimab, etrumadenant, chemotherapy, etc.). This example illustrates that treatment with domvanalimab does not deplete peripheral T cells in humans.

White blood cell counts for 11 patients have been followed for up to 85 days of treatment. See Table 17 for additional patient details. Cell counts were assessed in pre- and post-dose whole blood patient samples using commercially available Trucount tubes (BD Biosciences). CD45, CD3, CD4, CD8, CD25, and CD127 antibodies were used to detect peripheral blood T cells and Tregs using flow cytometry. The antibody cocktail and 100 D1 of whole blood was added to Trucount tubes, vortexed, and incubated for 15 minutes at room temperature. The stained whole blood was lysed using 900□□l of 1×BD FACs Lyse solution followed by vortexing, and incubation for 15 minutes at room temperature. Absolute counts were calculated as detailed in the Trucount technical data sheet. Briefly, the absolute count of a cell population (A) was obtained by dividing the number of positive cells events (X) by the number of bead events (Y), and then multiplying by the BD Trucount bead concentration (N/V, where N is the number of beads per test and V is the test volume). A=X/Y×N/V. Absolute cell count was then reported per μl of whole blood (WB).

Absolute cell counts were not measured in all patients due to limited sample collection. For some patients, white blood frequencies (not absolute cell counts) have been followed for up to 85 days of treatment. Frequencies of different populations were determined relative to a parent population and each timepoint was normalized to baseline (C1D1 predose) to compute fold change.

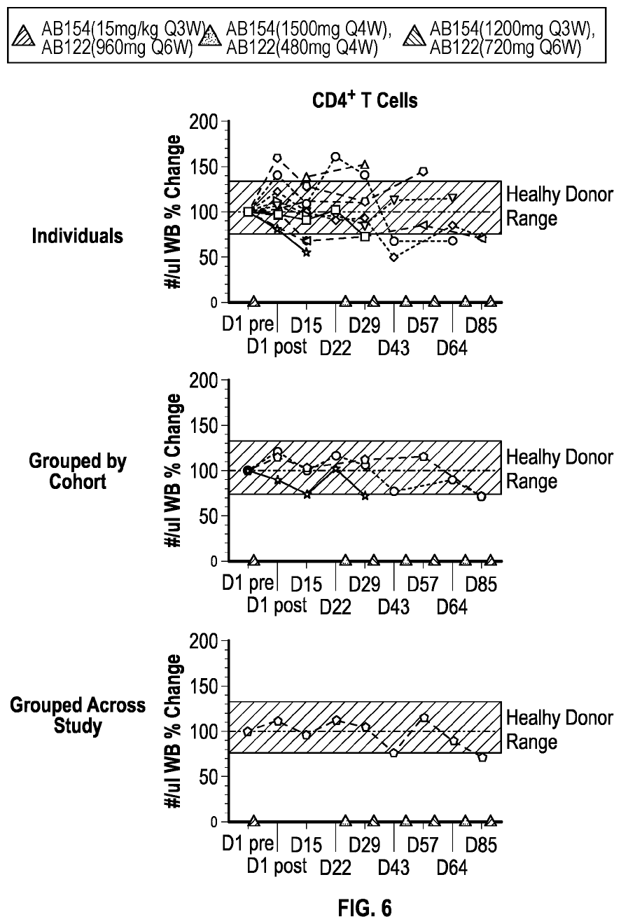

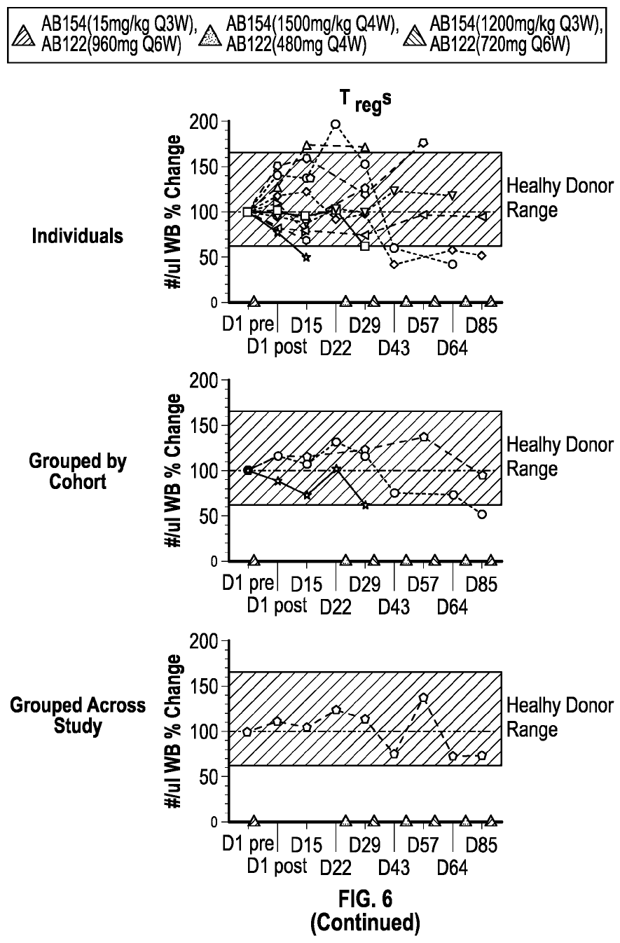

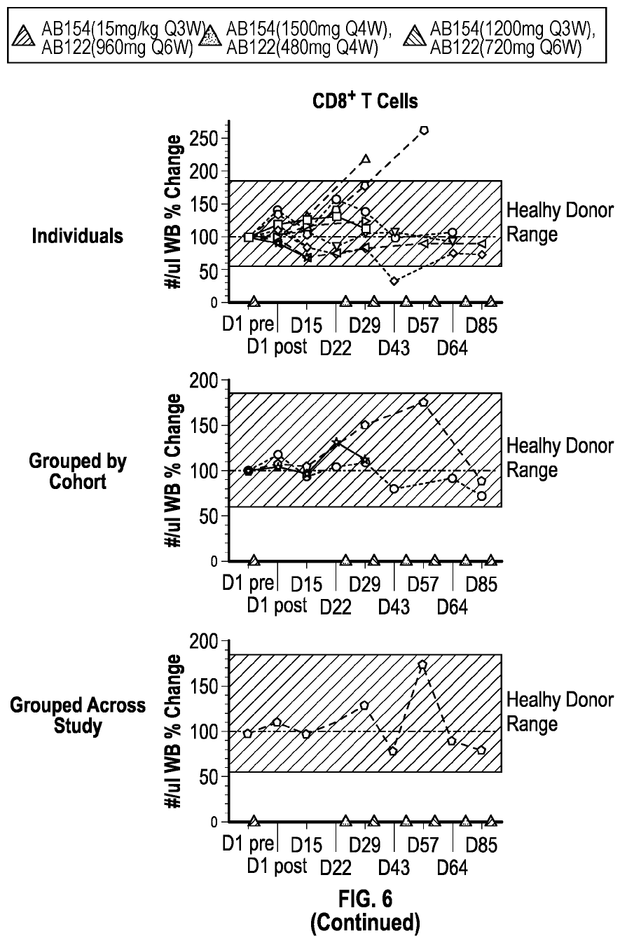

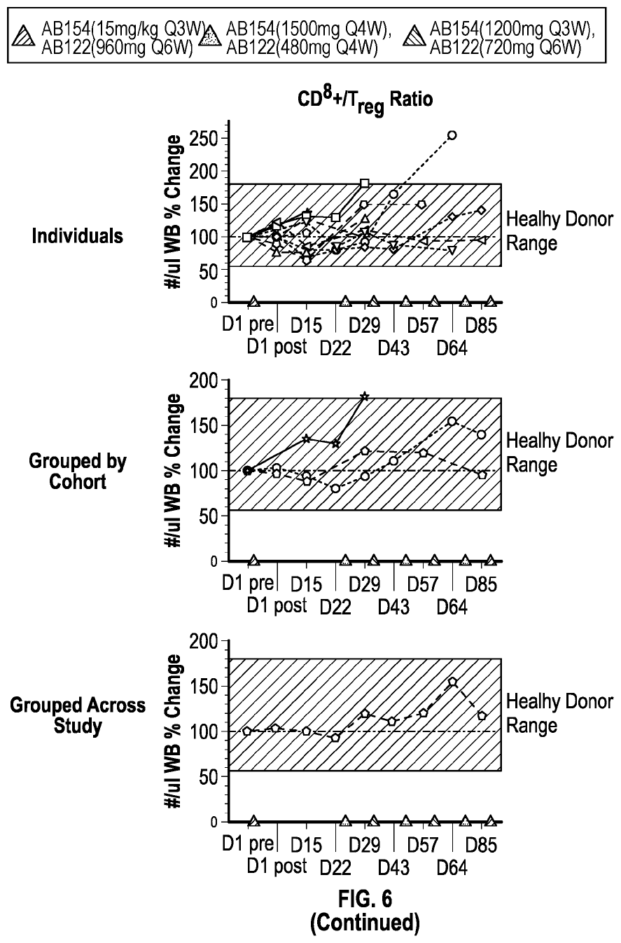

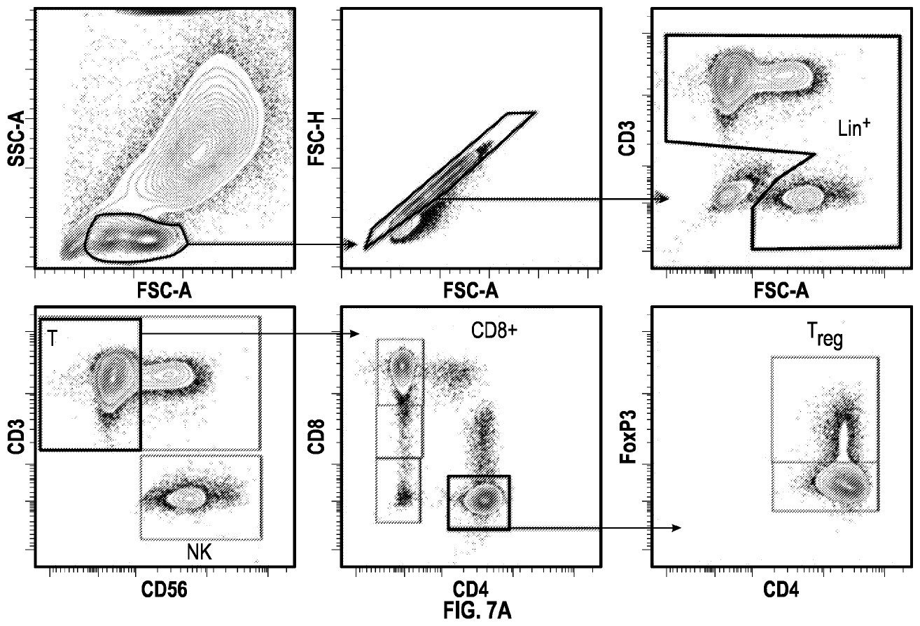

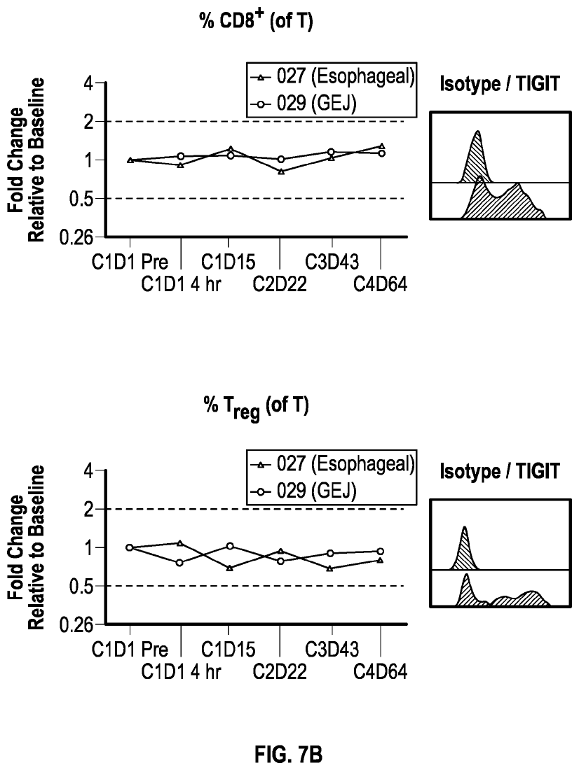

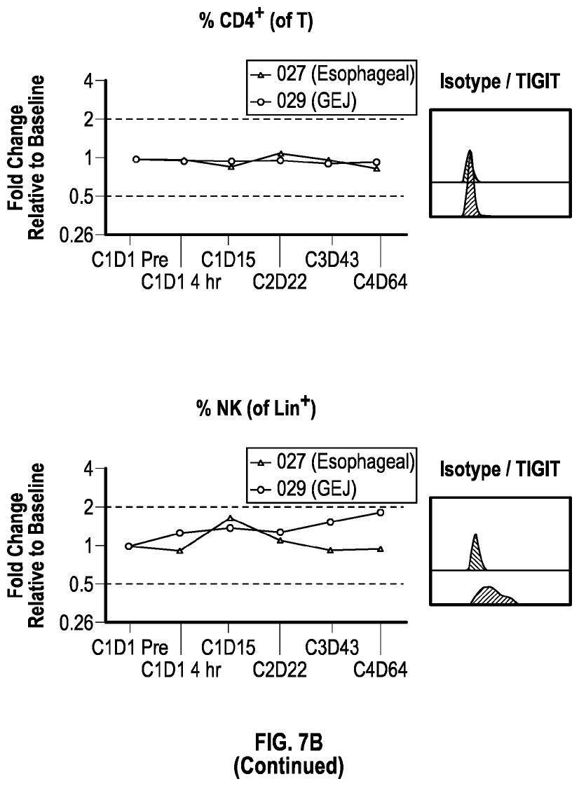

As shown in FIG. 6, absolute cell counts of CD4+ T cells, CD8+ T cells, and Tregs in patients that received zimberelimab and domvanalimab (n=11) remained largely unchanged compared to baseline with most changes remaining within or near the range observed for healthy donors (n=6, indicated by the grey shading). Cell frequencies were also stable over time, including in two partial responders treated with 15 mg/kg AB 154 Q3W+360 mg AB122 Q3W. See FIG. 7A and FIG. 7B. Additional information on these two responders is provided in Example 5.

TABLE 17

| Dosing Regimen | Indication |
|---|---|
| AB154 (15 mg/kg) Q3W + | Endometrial carcinoma |
| AB122 (960 mg) Q6W | Melanoma |
| AB154 (1500 mg) Q4W + | Unknown primary |
| AB122 (480 mg) Q4W | Endometrial |
| | Pancreatic |
| | Rectal cancer |
| | Fallopian Tube |
| AB154 (1200 mg) Q3W + | Duodenum Adenocarcinoma |
| AB122 (720 mg) Q6W | Colon Cancer |
| | Melanoma |
| | Pancreas Body Adenocarcincoma |

### Example 12: Domvanalimab Promotes CD8⁺ T Cell and NK Cell Engagement in Human Subjects

This example illustrates domvanalimab monotherapy and domvanalimab combination therapy has a measurable effect on CD8⁺ T cells and NK cells in the periphery.

In clinical trials evaluating domvanalimab, expression of Ki-67 has been used as a marker of CD8+ T cell and NK cell proliferation. Quantitation of Ki-67, a nuclear protein associated with cell cycle, is a common method to evaluate cell proliferation, especially in human samples ex vivo.

Immune cell profiling was conducted in whole blood by flow cytometry. For patients enrolled in the study described in Example 5 or Example 6, Ki67 changes on CD8⁺ T cells were evaluated in whole blood by similar methods. Extracellular staining cocktails which included (minimally) CD3, CD8, and CD56 antibodies were added to 500 ul of whole blood and incubated on ice for 30 minutes. These markers were used to identify CD8⁺ T cells (CD3⁺CD56⁻ CD8⁺) in both studies. For patients in the study of Example 6, extracellular cocktails also included CD45RA, CD39, and CD103 antibodies to identify an antigen-experienced memory CD8⁺ T cell population (CD3⁺CD56⁻CD8⁺CD45RA⁻CD39⁺CD103⁺). Following extracellular staining, all blood samples were fixed and lysed using 1×BD FACSLyse buffer for 20 minutes on ice and then stored at −80° C. for up to 6 weeks. Upon thawing, samples were centrifuged at 400×g for 5 min to remove the lysing solution, washed in PBS, and then fixed and permeabilized in 100 ul of 1×FoxP3 Fixation Buffer for 1 hour at room temperature. Permeabilized samples were washed in 1× permeabilization buffer, blocked using 25 ul of 20% normal mouse serum for 15 minutes at room temperature, then stained intracellularly using Ki67 antibody for 30 minutes on ice. Finally, samples were washed in 1× permeabilization buffer and resuspended in PBS prior to filtering and data acquisition on a BD FACSCanto cytometer. The maximum fold change observed with the first cycle of treatment (between C1D1 and C2D29) was also determined for Ki67 expression on CD8⁺ T cells.

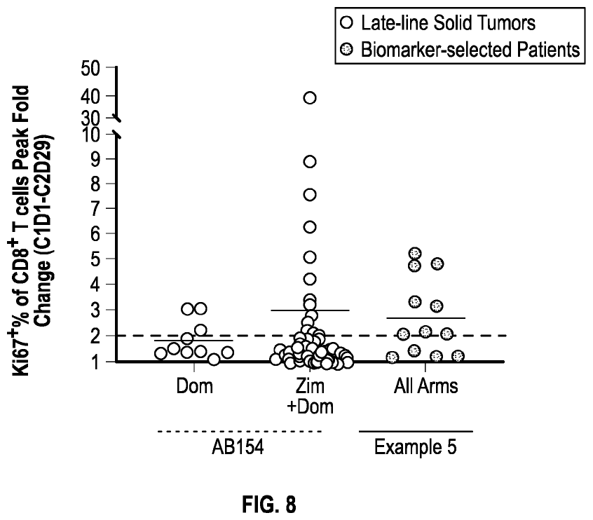

As shown in FIG. 8, patients enrolled in the study described in Example 5 often had increases in the proliferative marker (Ki67) on their circulating CD8⁺ T cells when domvanalimab was administered individually or in combination with zimberelimab. Increases in proliferating CD8⁺ T cells were also observed in patients enrolled in the study of Example 6 with PD-L1^{hi} (TPS ≥50%) non-small cell lung cancer (NSCLC) when zimberelimab was administered alone or in combination with domvanalimab or domvanalimab and etrumadenant.

In this data set, zimberelimab and domvanalimab monotherapy and zimberelimab combination therapy with domvanalimab showed similar frequencies of patients with Ki67 changes greater than 2-fold, though the magnitude of these responses varied.

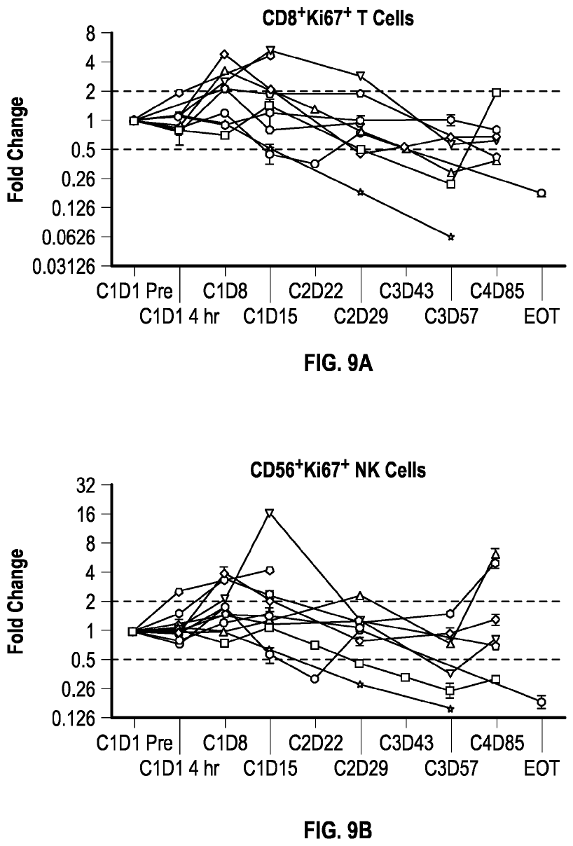

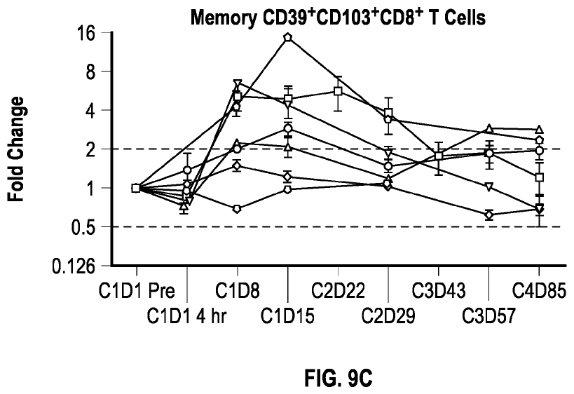

Ki67 expression on circulating NK cells was also evaluated in samples obtained from patients enrolled in the study of Example 6. Similar to CD8⁺ T cells, an increase in NK cell proliferation was observed within 1-2 weeks after the first dose (FIG. 9A and FIG. 9B).

Co-expression of CD39 and CD103 can differentiate tumor-specific T cells from bystander T cells in the tumor microenvironment (TME). At baseline they are typically present at low levels in the periphery. As shown in FIG. 9C, in samples obtained from subjects enrolled in the study described in Example 6, a transient increase in CD45RA−CD8+CD39+CD103+ memory T cells was observed in the periphery upon treatment in the 3 arms. Differences between the 3 study arms cannot be determined due to inadequate sample size.

### Example 13-1: Phase II, Open-Label, Platform Study, to Evaluate Immunotherapy-Based Combinations in Participants with Advanced Non-Small Cell Lung Cancer

A phase II open label platform study will be used to investigate the safety and efficacy of zimberelimab with other investigational products in participants with squamous or nonsquamous non-small cell lung cancer. Sub-study A will enroll treatment-naïve, PD-L1 high (TPS ≥50% by PharmDx 22C3 or 28-8 pharmDx (Dako) or % tumor cell (% TC) ≥50% by SP263 (Ventana)), metastatic NSCLC patients without actionable genomic aberrations (ALK fusion oncogene, actionable EGFR mutation, presence of any other tumor genomic aberration or driver mutation (e.g., ROS, BRAF, NTRK) for which a targeted therapy is approved by local health authority and available). Sub-study B will enroll treatment-naïve, metastatic, squamous or nonsquamous NSCLC patients without actionable genomic aberrations (see above), but without restriction to PD-L1 status. Sub-Study C will enroll metastatic, squamous or nonsquamous NSCLC patients who have documented disease progression on anti-PD-(L)1 and platinum-based chemotherapy in one or two lines of prior therapy and do not have known actionable genomic aberrations (see above). Arm A1: zimberelimab 360 mg Q3W+domvanalimab 5 mg/kg Q3W; Arm A2: zimberelimab 360 mg Q3W+domvanalimab 15 mg/kg Q3W; Arm A3: zimberelimab 480 mg Q4W+domvanalimab 1600 mg Q4W+quemliclustat 100 mg Q2W; Arm B1: zimberelimab 360 mg Q3W+quemliclustat 50 mg QW+platinum doublet chemotherapy; Arm B2: zimberelimab 360 mg Q3W+domvanalimab 1200 mg Q3W+platinum doublet chemotherapy; Arm B3: zimberelimab 360 mg Q3W+domvanalimab 1200 mg Q3W+quemliclustat 50 mg QW+platinum doublet chemotherapy; Arm C1: zimberelimab 360 mg Q3W+domvanalimab 1200 mg Q3W+docetaxel; Arm C2: zimberelimab 360 mg Q3W+quemliclustat 300 mg Q3W +docetaxel. Primary endpoints to be assessed include objective response rate, safety and tolerability. Secondary endpoints include progression-free survival, duration of response, overall survival, and PK.

### Example 13-2: Phase II, Open-label, Platform Study, to Evaluate Immunotherapy-based Combinations in Participants with Advanced Non-Small Cell Lung Cancer

A phase II open label platform study will be used to investigate the safety and efficacy of zimberelimab with other investigational products in participants with squamous or nonsquamous non-small cell lung cancer. Sub-study A will enroll treatment-naïve, PD-L1 high (TPS ≥50% by PharmDx 22C3 or 28-8 pharmDx (Dako) or % tumor cell (% TC) ≥50% by SP263 (Ventana)), metastatic NSCLC patients without actionable genomic aberrations (ALK fusion oncogene, actionable EGFR mutation, presence of any other tumor genomic aberration or driver mutation (e.g., ROS, BRAF, NTRK) for which a targeted therapy is approved by local health authority and available). Sub-study B will enroll treatment-naïve, metastatic, squamous or nonsquamous NSCLC patients without actionable genomic aberrations (see above), but without restriction to PD-L1 status. Sub-Study C will enroll metastatic, squamous or nonsquamous NSCLC patients who have documented disease progression on anti-PD-(L)1 and platinum-based chemotherapy in one or two lines of prior therapy and do not have known actionable genomic aberrations (see above). Arm A1: zimberelimab 360 mg Q3W+domvanalimab 800 mg Q3W; Arm A2: zimberelimab 360 mg Q3W+domvanalimab 1200 mg Q3W; Arm A3: zimberelimab 360 mg Q4W+quemliclustat 300 mg Q3W; Arm B1: zimberelimab 360 mg Q3W+quemliclustat 300 mg QW+platinum doublet chemotherapy; Arm B2: zimberelimab 360 mg Q3W+domvanalimab 1200 mg Q3W+platinum doublet chemotherapy; Arm B3: zimberelimab 360 mg Q3W+domvanalimab 1200 mg Q3W+quemliclustat 300 mg QW+platinum doublet chemotherapy; Arm Cl: zimberelimab 360 mg Q3W+domvanalimab 1200 mg Q3W+docetaxel; Arm C2: zimberelimab 360 mg Q3W+quemliclustat 300 mg Q3W+docetaxel. Primary endpoints to be assessed include objective response rate, safety and tolerability. Secondary endpoints include progression-free survival, duration of response, overall survival, and PK.

The primary efficacy endpoint is objective response rate, defined as the proportion of participants with a best overall response of complete or partial response as assessed by the investigator determined by RECIST v1.1. Secondary efficacy endpoints include disease control rate (percentage of measurable participants with measurable disease at baseline who achieve a best overall RECIST response of CR, PR, or SD), progression-free survival (time from treatment assignment until first documentation of progressive disease or death, whichever comes first), overall survival (time from treatment assignment until death due to any cause) and duration of response (defined as the time from first documentation of disease response (CR or PR) until first documentation of confirmed progressive disease or death).

TABLE 18
All Treatment-emergent Adverse Events
(Domvanalimab-Treated Pop. Only)

| Adverse | AB154CSP0001 | ARC-7 | Total |
|---|---|---|---|
| Event Preferred Term | (N = 62) | (N = 37) | (N = 99) |
| - Any AEs - | 53 ( 85.5%) | 31 ( 83.8%) | 84 ( 84.8%) |
| Nausea | 9 ( 14.5%) | 4 ( 10.8%) | 13 ( 13.1%) |
| Fatigue | 9 ( 14.5%) | 3 ( 8.1%) | 12 ( 12.1%) |
| Diarrhoea | 5 ( 8.1%) | 5 ( 13.5%) | 10 ( 10.1%) |
| Pruritus | 6 ( 9.7%) | 4 ( 10.8%) | 10 ( 10.1%) |
| Anaemia | 5 ( 8.1%) | 4 ( 10.8%) | 9 ( 9.1%) |
| Arthralgia | 4 ( 6.5%) | 4 ( 10.8%) | 8 ( 8.1%) |
| Back pain | 4 ( 6.5%) | 4 ( 10.8%) | 8 ( 8.1%) |
| Dyspnoea | 4 ( 6.5%) | 4 ( 10.8%) | 8 ( 8.1%) |
| Pyrexia | 5 ( 8.1%) | 3 ( 8.1%) | 8 ( 8.1%) |
| Constipation | 4 ( 6.5%) | 3 ( 8.1%) | 7 ( 7.1%) |
| Cough | 3 ( 4.8%) | 4 ( 10.8%) | 7 ( 7.1%) |
| Headache | 6 ( 9.7%) | 1 ( 2.7%) | 7 ( 7.1%) |
| Rash maculo-papular | 3 ( 4.8%) | 4 ( 10.8%) | 7 ( 7.1%) |
| Decreased appetite | 3 ( 4.8%) | 3 ( 8.1%) | 6 ( 6.1%) |
| Rash | 3 ( 4.8%) | 3 ( 8.1%) | 6 ( 6.1%) |
| Urinary tract infection | 5 ( 8.1%) | 1 ( 2.7%) | 6 ( 6.1%) |
| Abdominal pain | 5 ( 8.1%) | ( 0.0%) | 5 ( 5.1%) |
| Dehydration | 4 ( 6.5%) | 1 ( 2.7%) | 5 ( 5.1%) |
| Dizziness | 4 ( 6.5%) | 1 ( 2.7%) | 5 ( 5.1%) |
| Dysphagia | 3 ( 4.8%) | 2 ( 5.4%) | 5 ( 5.1%) |
| Hypothyroidism | 4 ( 6.5%) | 1 ( 2.7%) | 5 ( 5.1%) |
| Musculoskeletal pain | 3 ( 4.8%) | 2 ( 5.4%) | 5 ( 5.1%) |
| Vomiting | 2 ( 3.2%) | 3 ( 8.1%) | 5 ( 5.1%) |
| Blood creatinine | 3 ( 4.8%) | 1 ( 2.7%) | 4 ( 4.0%) |
| increased | | | |
| Hyperkalaemia | 2 ( 3.2%) | 2 ( 5.4%) | 4 ( 4.0%) |
| Hypotension | 1 ( 1.6%) | 3 ( 8.1%) | 4 ( 4.0%) |
| Oedema peripheral | 1 ( 1.6%) | 3 ( 8.1%) | 4 ( 4.0%) |
| Pleural effusion | 1 ( 1.6%) | 3 ( 8.1%) | 4 ( 4.0%) |
| Small intestinal | 4 ( 6.5%) | ( 0.0%) | 4 ( 4.0%) |
| obstruction | | | |
| Abdominal distension | 3 ( 4.8%) | ( 0.0%) | 3 ( 3.0%) |
| Ascites | 3 ( 4.8%) | ( 0.0%) | 3 ( 3.0%) |
| Asthenia | ( 0.0%) | 3 ( 8.1%) | 3 ( 3.0%) |
| Chronic obstructive | ( 0.0%) | 3 ( 8.1%) | 3 ( 3.0%) |
| pulmonary disease | | | |
| Hyperthyroidism | 2 ( 3.2%) | 1 ( 2.7%) | 3 ( 3.0%) |
| Infusion related reaction | 2 ( 3.2%) | 1 ( 2.7%) | 3 ( 3.0%) |
| Insomnia | 1 ( 1.6%) | 2 ( 5.4%) | 3 ( 3.0%) |
| Oral candidiasis | 2 ( 3.2%) | 1 ( 2.7%) | 3 ( 3.0%) |
| Pain | 2 ( 3.2%) | 1 ( 2.7%) | 3 ( 3.0%) |
| Tachycardia | 1 ( 1.6%) | 2 ( 5.4%) | 3 ( 3.0%) |
| Tumour flare | 3 ( 4.8%) | ( 0.0%) | 3 ( 3.0%) |
| Abdominal discomfort | 2 ( 3.2%) | ( 0.0%) | 2 ( 2.0%) |
| Acute kidney injury | ( 0.0%) | 2 ( 5.4%) | 2 ( 2.0%) |
| Alanine aminotransferase | 2 ( 3.2%) | ( 0.0%) | 2 ( 2.0%) |
| increased | | | |
| Arthritis | 2 ( 3.2%) | ( 0.0%) | 2 ( 2.0%) |
| Aspartate amino- | 2 ( 3.2%) | ( 0.0%) | 2 ( 2.0%) |
| transferase increased | | | |
| Blood alkaline | 2 ( 3.2%) | ( 0.0%) | 2 ( 2.0%) |
| phosphatase increased | | | |
| Blood bilirubin increased | 1 ( 1.6%) | 1 ( 2.7%) | 2 ( 2.0%) |
| Confusional state | 2 ( 3.2%) | ( 0.0%) | 2 ( 2.0%) |
| Delirium | ( 0.0%) | 2 ( 5.4%) | 2 ( 2.0%) |
| Dry mouth | ( 0.0%) | 2 ( 5.4%) | 2 ( 2.0%) |
| Dyspepsia | 1 ( 1.6%) | 1 ( 2.7%) | 2 ( 2.0%) |
| Flank pain | 2 ( 3.2%) | ( 0.0%) | 2 ( 2.0%) |
| Gamma-glutamyl- | 2 ( 3.2%) | ( 0.0%) | 2 ( 2.0%) |
| transferase increased | | | |
| Haemoptysis | 1 ( 1.6%) | 1 ( 2.7%) | 2 ( 2.0%) |
| Hyperglycemia | 2 ( 3.2%) | ( 0.0%) | 2 ( 2.0%) |
| Hypertension | ( 0.0%) | 2 ( 5.4%) | 2 ( 2.0%) |
| Hypoalbuminaemia | 1 ( 1.6%) | 1 ( 2.7%) | 2 ( 2.0%) |
| Hypokalaemia | 1 ( 1.6%) | 1 ( 2.7%) | 2 ( 2.0%) |
| Immune-mediated | 2 ( 3.2%) | ( 0.0%) | 2 ( 2.0%) |
| hepatitis | | | |
| Influenza like illness | 1 ( 1.6%) | 1 ( 2.7%) | 2 ( 2.0%) |
| Lymphopenia | 2 ( 3.2%) | ( 0.0%) | 2 ( 2.0%) |
| Malaise | ( 0.0%) | 2 ( 5.4%) | 2 ( 2.0%) |
| Muscle spasms | 1 ( 1.6%) | 1 ( 2.7%) | 2 ( 2.0%) |
| Paraesthesia | ( 0.0%) | 2 ( 5.4%) | 2 ( 2.0%) |
| Peripheral sensory | 2 ( 3.2%) | ( 0.0%) | 2 ( 2.0%) |
| neuropathy | | | |
| Pneumonitis | 1 ( 1.6%) | 1 ( 2.7%) | 2 ( 2.0%) |
| Pollakiuria | 2 ( 3.2%) | ( 0.0%) | 2 ( 2.0%) |
| Skin infection | 1 ( 1.6%) | 1 ( 2.7%) | 2 ( 2.0%) |
| Tinnitus | 1 ( 1.6%) | 1 ( 2.7%) | 2 ( 2.0%) |
| Upper respiratory tract | 1 ( 1.6%) | 1 ( 2.7%) | 2 ( 2.0%) |
| infection | | | |
| Weight decreased | ( 0.0%) | 2 ( 5.4%) | 2 ( 2.0%) |
| ATROPHIC CORDITIS | ( 0.0%) | 1 ( 2.7%) | 1 ( 1.0%) |
| (Verbatim) | | | |
| Acidosis | ( 0.0%) | 1 ( 2.7%) | 1 ( 1.0%) |
| Acute respiratory failure | ( 0.0%) | 1 ( 2.7%) | 1 ( 1.0%) |
| Adrenal insufficiency | ( 0.0%) | 1 ( 2.7%) | 1 ( 1.0%) |
| Alopecia | 1 ( 1.6%) | ( 0.0%) | 1 ( 1.0%) |
| Anxiety | ( 0.0%) | 1 ( 2.7%) | 1 ( 1.0%) |
| Aphasia | 1 ( 1.6%) | ( 0.0%) | 1 ( 1.0%) |
| Atrial fibrillation | ( 0.0%) | 1 ( 2.7%) | 1 ( 1.0%) |
| Atrioventricular block | 1 ( 1.6%) | ( 0.0%) | 1 ( 1.0%) |
| first degree | | | |
| Bile duct obstruction | 1 ( 1.6%) | ( 0.0%) | 1 ( 1.0%) |
| Bladder discomfort | ( 0.0%) | 1 ( 2.7%) | 1 ( 1.0%) |
| Blood cholesterol | 1 ( 1.6%) | ( 0.0%) | 1 ( 1.0%) |
| increased | | | |
| Blood lactate | ( 0.0%) | 1 ( 2.7%) | 1 ( 1.0%) |
| dehydrogenase increased | | | |
| Blood pressure increased | 1 ( 1.6%) | ( 0.0%) | 1 ( 1.0%) |
| Blood urea increased | 1 ( 1.6%) | ( 0.0%) | 1 ( 1.0%) |
| Bronchitis | ( 0.0%) | 1 ( 2.7%) | 1 ( 1.0%) |
| COVID-19 | ( 0.0%) | 1 ( 2.7%) | 1 ( 1.0%) |
| Cholangitis | ( 0.0%) | 1 ( 2.7%) | 1 ( 1.0%) |
| Coccydynia | ( 0.0%) | 1 ( 2.7%) | 1 ( 1.0%) |
| Cognitive disorder | 1 ( 1.6%) | ( 0.0%) | 1 ( 1.0%) |
| DEHYDRATION | 1 ( 1.6%) | ( 0.0%) | 1 ( 1.0%) |
| (Verbatim) | | | |
| Decp vein thrombosis | ( 0.0%) | 1 ( 2.7%) | 1 ( 1.0%) |
| Diabetes mellitus | 1 ( 1.6%) | ( 0.0%) | 1 ( 1.0%) |
| Dry eye | ( 0.0%) | 1 ( 2.7%) | 1 ( 1.0%) |
| Dry skin | ( 0.0%) | 1 ( 2.7%) | 1 ( 1.0%) |
| Dysaesthesia | 1 ( 1.6%) | ( 0.0%) | 1 ( 1.0%) |
| Dysarthria | ( 0.0%) | 1 ( 2.7%) | 1 ( 1.0%) |
| Dysphonia | ( 0.0%) | 1 ( 2.7%) | 1 ( 1.0%) |
| Dyspnoea exertional | ( 0.0%) | 1 ( 2.7%) | 1 ( 1.0%) |
| Early satiety | 1 ( 1.6%) | ( 0.0%) | 1 ( 1.0%) |
| Enterocolitis infectious | 1 ( 1.6%) | ( 0.0%) | 1 ( 1.0%) |
| Epigastric discomfort | ( 0.0%) | 1 ( 2.7%) | 1 ( 1.0%) |
| Eye infection | 1 ( 1.6%) | ( 0.0%) | 1 ( 1.0%) |
| Eye pruritus | ( 0.0%) | 1 ( 2.7%) | 1 ( 1.0%) |
| Fall | ( 0.0%) | 1 ( 2.7%) | 1 ( 1.0%) |
| Folliculitis | 1 ( 1.6%) | ( 0.0%) | 1 ( 1.0%) |
| Gastrointestinal wall | ( 0.0%) | 1 ( 2.7%) | 1 ( 1.0%) |
| thickening | | | |
| Gastrooesophageal | ( 0.0%) | 1 ( 2.7%) | 1 ( 1.0%) |
| reflux disease | | | |
| Generalised oedema | ( 0.0%) | 1 ( 2.7%) | 1 ( 1.0%) |
| Glaucoma | 1 ( 1.6%) | ( 0.0%) | 1 ( 1.0%) |
| Glomerular filtration rate | 1 ( 1.6%) | ( 0.0%) | 1 ( 1.0%) |
| decreased | | | |
| Glossopharyngeal nerve | 1 ( 1.6%) | ( 0.0%) | 1 ( 1.0%) |
| disorder | | | |
| Haematochezia | 1 ( 1.6%) | ( 0.0%) | 1 ( 1.0%) |
| Haematuria | 1 ( 1.6%) | ( 0.0%) | 1 ( 1.0%) |
| Haemorrhage intracranial | 1 ( 1.6%) | ( 0.0%) | 1 ( 1.0%) |
| Hemiparesis | 1 ( 1.6%) | ( 0.0%) | 1 ( 1.0%) |
| Herpes zoster | 1 ( 1.6%) | ( 0.0%) | 1 ( 1.0%) |
| Hiccups | ( 0.0%) | 1 ( 2.7%) | 1 ( 1.0%) |
| Hip fracture | ( 0.0%) | 1 ( 2.7%) | 1 ( 1.0%) |
| Hot flush | 1 ( 1.6%) | ( 0.0%) | 1 ( 1.0%) |
| Hyperuricaemia | 1 ( 1.6%) | ( 0.0%) | 1 ( 1.0%) |
| Hypoaesthesia | ( 0.0%) | 1 ( 2.7%) | 1 ( 1.0%) |
| Hypoglossal nerve | 1 ( 1.6%) | ( 0.0%) | 1 ( 1.0%) |
| disorder | | | |
| Hypomagnesaemia | 1 ( 1.6%) | ( 0.0%) | 1 ( 1.0%) |
| Hyponatraemia | 1 ( 1.6%) | ( 0.0%) | 1 ( 1.0%) |
| Infection | 1 ( 1.6%) | ( 0.0%) | 1 ( 1.0%) |
| Infusion site pain | ( 0.0%) | 1 ( 2.7%) | 1 ( 1.0%) |
| Inguinal hernia | 1 ( 1.6%) | ( 0.0%) | 1 ( 1.0%) |
| Inner ear disorder | 1 ( 1.6%) | ( 0.0%) | 1 ( 1.0%) |
| Intestinal obstruction | 1 ( 1.6%) | ( 0.0%) | 1 ( 1.0%) |
| Jaundice | 1 ( 1.6%) | ( 0.0%) | 1 ( 1.0%) |
| Joint swelling | ( 0.0%) | 1 ( 2.7%) | 1 ( 1.0%) |
| Lethargy | 1 ( 1.6%) | ( 0.0%) | 1 ( 1.0%) |
| Liver function test | 1 ( 1.6%) | ( 0.0%) | 1 ( 1.0%) |
| increased | | | |
| Localised infection | ( 0.0%) | 1 ( 2.7%) | 1 ( 1.0%) |
| Metabolic acidosis | ( 0.0%) | 1 ( 2.7%) | 1 ( 1.0%) |
| Mucosal inflammation | ( 0.0%) | 1 ( 2.7%) | 1 ( 1.0%) |
| Mucous stools | 1 ( 1.6%) | ( 0.0%) | 1 ( 1.0%) |
| Muscle strain | 1 ( 1.6%) | ( 0.0%) | 1 ( 1.0%) |
| Muscle tightness | 1 ( 1.6%) | ( 0.0%) | 1 ( 1.0%) |
| Muscular weakness | 1 ( 1.6%) | ( 0.0%) | 1 ( 1.0%) |
| Musculoskeletal | 1 ( 1.6%) | ( 0.0%) | 1 ( 1.0%) |
| chest pain | | | |
| Myalgia | ( 0.0%) | 1 ( 2.7%) | 1 ( 1.0%) |
| Nail disorder | 1 ( 1.6%) | ( 0.0%) | 1 ( 1.0%) |
| Nasal mucosal disorder | 1 ( 1.6%) | ( 0.0%) | 1 ( 1.0%) |
| Neck pain | 1 ( 1.6%) | ( 0.0%) | 1 ( 1.0%) |
| Neuralgia | 1 ( 1.6%) | ( 0.0%) | 1 ( 1.0%) |
| Non-cardiac chest pain | 1 ( 1.6%) | ( 0.0%) | 1 ( 1.0%) |
| Odynophagia | 1 ( 1.6%) | ( 0.0%) | 1 ( 1.0%) |
| Oral pain | 1 ( 1.6%) | ( 0.0%) | 1 ( 1.0%) |
| Oropharyngeal pain | ( 0.0%) | 1 ( 2.7%) | 1 ( 1.0%) |
| Pancreatitis | ( 0.0%) | 1 ( 2.7%) | 1 ( 1.0%) |
| Peritonitis bacterial | 1 ( 1.6%) | ( 0.0%) | 1 ( 1.0%) |
| Photodermatosis | 1 ( 1.6%) | ( 0.0%) | 1 ( 1.0%) |
| Pleuritic pain | 1 ( 1.6%) | ( 0.0%) | 1 ( 1.0%) |
| Pneumonia | ( 0.0%) | 1 ( 2.7%) | 1 ( 1.0%) |
| Productive cough | ( 0.0%) | 1 ( 2.7%) | 1 ( 1.0%) |
| Pruritus generalised | 1 ( 1.6%) | ( 0.0%) | 1 ( 1.0%) |
| Pulmonary pain | 1 ( 1.6%) | ( 0.0%) | 1 ( 1.0%) |
| Rash pruritic | 1 ( 1.6%) | ( 0.0%) | 1 ( 1.0%) |
| Respiratory failure | ( 0.0%) | 1 ( 2.7%) | 1 ( 1.0%) |
| Rhinitis allergic | ( 0.0%) | 1 ( 2.7%) | 1 ( 1.0%) |
| Rhinorrhoea | ( 0.0%) | 1 ( 2.7%) | 1 ( 1.0%) |
| Seasonal allergy | ( 0.0%) | 1 ( 2.7%) | 1 ( 1.0%) |
| Sensation of foreign body | ( 0.0%) | 1 ( 2.7%) | 1 ( 1.0%) |
| Septic shock | ( 0.0%) | 1 ( 2.7%) | 1 ( 1.0%) |
| Sinus bradycardia | ( 0.0%) | 1 ( 2.7%) | 1 ( 1.0%) |
| Sinus pain | ( 0.0%) | 1 ( 2.7%) | 1 ( 1.0%) |
| Sinusitis | 1 ( 1.6%) | ( 0.0%) | 1 ( 1.0%) |
| Sputum increased | ( 0.0%) | 1 ( 2.7%) | 1 ( 1.0%) |
| Subarachnoid | 1 ( 1.6%) | ( 0.0%) | 1 ( 1.0%) |
| haemorrhage | | | |
| Transaminases increased | 1 ( 1.6%) | ( 0.0%) | 1 ( 1.0%) |
| Tumour pain | 1 ( 1.6%) | ( 0.0%) | 1 ( 1.0%) |
| Ulna fracture | ( 0.0%) | 1 ( 2.7%) | 1 ( 1.0%) |
| Upper gastrointestinal | ( 0.0%) | 1 ( 2.7%) | 1 ( 1.0%) |
| haemorrhage | | | |
| Urticaria | ( 0.0%) | 1 ( 2.7%) | 1 ( 1.0%) |
| Vaginal infection | ( 0.0%) | 1 ( 2.7%) | 1 ( 1.0%) |
| Vascular compression | ( 0.0%) | 1 ( 2.7%) | 1 ( 1.0%) |
| Ventricular tachycardia | ( 0.0%) | 1 ( 2.7%) | 1 ( 1.0%) |

TABLE 19
All Domvanalimab-Related Adverse Events
(Domvanalimab-Treated Population Only)

| Adverse | AB154CSP0001 | ARC-7 | Total |
|---|---|---|---|
| Event Preferred Term | (N = 62) | (N = 37) | (N = 99) |
| - Any AEs - | 32 ( 51.6%) | 18 ( 48.6%) | 50 ( 50.5%) |
| Pruritus | 5 ( 8.1%) | 4 ( 10.8%) | 9 ( 9.1%) |
| Fatigue | 4 ( 6.5%) | 2 ( 5.4%) | 6 ( 6.1%) |
| Nausea | 5 ( 8.1%) | 1 ( 2.7%) | 6 ( 6.1%) |
| Rash maculo-papular | 3 ( 4.8%) | 3 ( 8.1%) | 6 ( 6.1%) |
| Diarrhoea | 3 ( 4.8%) | 2 ( 5.4%) | 5 ( 5.1%) |
| Pyrexia | 3 ( 4.8%) | 2 ( 5.4%) | 5 ( 5.1%) |
| Arthralgia | 1 ( 1.6%) | 3 ( 8.1%) | 4 ( 4.0%) |
| Headache | 3 ( 4.8%) | 1 ( 2.7%) | 4 ( 4.0%) |
| Hypothyroidism | 4 ( 6.5%) | ( 0.0%) | 4 ( 4.0%) |
| Rash | 1 ( 1.6%) | 3 ( 8.1%) | 4 ( 4.0%) |
| Decreased appetite | 1 ( 1.6%) | 2 ( 5.4%) | 3 ( 3.0%) |
| Hyperthyroidism | 2 ( 3.2%) | 1 ( 2.7%) | 3 ( 3.0%) |
| Tumour flare | 3 ( 4.8%) | ( 0.0%) | 3 ( 3.0%) |
| Back pain | 1 ( 1.6%) | 1 ( 2.7%) | 2 ( 2.0%) |
| Constipation | 1 ( 1.6%) | 1 ( 2.7%) | 2 ( 2.0%) |
| Immune-mediated | 2 ( 3.2%) | ( 0.0%) | 2 ( 2.0%) |
| hepatitis | | | |
| Infusion related reaction | 1 ( 1.6%) | 1 ( 2.7%) | 2 ( 2.0%) |
| Malaise | ( 0.0%) | 2 ( 5.4%) | 2 ( 2.0%) |
| Pain | 2 ( 3.2%) | ( 0.0%) | 2 ( 2.0%) |
| Pneumonitis | 1 ( 1.6%) | 1 ( 2.7%) | 2 ( 2.0%) |
| Abdominal distension | 1 ( 1.6%) | ( 0.0%) | 1 ( 1.0%) |
| Abdominal pain | 1 ( 1.6%) | ( 0.0%) | 1 ( 1.0%) |
| Adrenal insufficiency | ( 0.0%) | 1 ( 2.7%) | 1 ( 1.0%) |
| Alanine aminotransferase | 1 ( 1.6%) | ( 0.0%) | 1 ( 1.0%) |
| increased | | | |
| Anaemia | 1 ( 1.6%) | ( 0.0%) | 1 ( 1.0%) |
| Arthritis | 1 ( 1.6%) | ( 0.0%) | 1 ( 1.0%) |
| Aspartate amino- | 1 ( 1.6%) | ( 0.0%) | 1 ( 1.0%) |
| transferase increased | | | |
| Asthenia | ( 0.0%) | 1 ( 2.7%) | 1 ( 1.0%) |
| Cough | ( 0.0%) | 1 ( 2.7%) | 1 ( 1.0%) |
| Delirium | ( 0.0%) | 1 ( 2.7%) | 1 ( 1.0%) |
| Dry eye | ( 0.0%) | 1 ( 2.7%) | 1 ( 1.0%) |
| Dry mouth | ( 0.0%) | 1 ( 2.7%) | 1 ( 1.0%) |
| Dyspnoea exertional | ( 0.0%) | 1 ( 2.7%) | 1 ( 1.0%) |
| Flank pain | 1 ( 1.6%) | ( 0.0%) | 1 ( 1.0%) |
| Generalised oedema | ( 0.0%) | 1 ( 2.7%) | 1 ( 1.0%) |
| Hot flush | 1 ( 1.6%) | ( 0.0%) | 1 ( 1.0%) |
| Hyperglycaemia | 1 ( 1.6%) | ( 0.0%) | 1 ( 1.0%) |
| Hyperkalaemia | 1 ( 1.6%) | ( 0.0%) | 1 ( 1.0%) |
| Hypokalaemia | 1 ( 1.6%) | ( 0.0%) | 1 ( 1.0%) |
| Influenza like illness | 1 ( 1.6%) | ( 0.0%) | 1 ( 1.0%) |
| Infusion site pain | ( 0.0%) | 1 ( 2.7%) | 1 ( 1.0%) |
| Liver function test | 1 ( 1.6%) | ( 0.0%) | 1 ( 1.0%) |
| increased | | | |
| Lymphopenia | 1 ( 1.6%) | ( 0.0%) | 1 ( 1.0%) |
| Muscle spasms | 1 ( 1.6%) | ( 0.0%) | 1 ( 1.0%) |
| Musculoskeletal chest | 1 ( 1.6%) | ( 0.0%) | 1 ( 1.0%) |
| pain | | | |
| Myalgia | ( 0.0%) | 1 ( 2.7%) | 1 ( 1.0%) |
| Nail disorder | 1 ( 1.6%) | ( 0.0%) | 1 ( 1.0%) |
| Nasal mucosal disorder | 1 ( 1.6%) | ( 0.0%) | 1 ( 1.0%) |
| Oral pain | 1 ( 1.6%) | ( 0.0%) | 1 ( 1.0%) |
| Pancreatitis | ( 0.0%) | 1 ( 2.7%) | 1 ( 1.0%) |
| Paraesthesia | ( 0.0%) | 1 ( 2.7%) | 1 ( 1.0%) |
| Peripheral sensory | 1 ( 1.6%) | ( 0.0%) | 1 ( 1.0%) |
| neuropathy | | | |
| Pruritus generalised | 1 ( 1.6%) | ( 0.0%) | 1 ( 1.0%) |
| Pulmonary pain | 1 ( 1.6%) | ( 0.0%) | 1 ( 1.0%) |
| Rash pruritic | 1 ( 1.6%) | ( 0.0%) | 1 ( 1.0%) |
| Rhinorrhoea | ( 0.0%) | 1 ( 2.7%) | 1 ( 1.0%) |
| Transaminases increased | 1 ( 1.6%) | ( 0.0%) | 1 ( 1.0%) |
| Tumour pain | 1 ( 1.6%) | ( 0.0%) | 1 ( 1.0%) |
| Vomiting | ( 0.0%) | 1 ( 2.7%) | 1 ( 1.0%) |

TABLE 20
Summary of Treatment-emergent Serious Adverse Events (Domvanalimab-Treated Population Only)

| | Number of Subjects Exposure (N = 99) | | | | | | | | | |
|---|---|---|---|---|---|---|---|---|---|---|
| | SAEs n (%) | | | | | Related SAEs n (%) | | | | |
| System Organ Class | | Grade 1- | | | | | Grade 1- | | | |
| Preferred Term | Total | 2 | Grade 3 | Grade 4 | Grade 5 | Total | 2 | Grade 3 | Grade 4 | Grade 5 |
| - Any SAEs - | 31 (31.3%) | 6 (6.1%) | 24 (24.2%) | 2 (2.0%) | 3 (3.0%) | 6 (6.1%) | 1 (1.0%) | 5 (5.1%) | (0.0%) | 1 (1.0%) |
| Gastrointestinal disorders | 7 (7.1%) | 2 (2.0%) | 5 (5.1%) | (0.0%) | (0.0%) | (0.0%) | (0.0%) | (0.0%) | (0.0%) | (0.0%) |
| Small intestinal obstruction | 4 (4.0%) | 1 (1.0%) | 3 (3.0%) | (0.0%) | (0.0%) | (0.0%) | (0.0%) | (0.0%) | (0.0%) | (0.0%) |
| Abdominal pain | 1 (1.0%) | (0.0%) | 1 (1.0%) | (0.0%) | (0.0%) | (0.0%) | (0.0%) | (0.0%) | (0.0%) | (0.0%) |
| Ascites | 1 (1.0%) | (0.0%) | 1 (1.0%) | (0.0%) | (0.0%) | (0.0%) | (0.0%) | (0.0%) | (0.0%) | (0.0%) |
| Constipation | 1 (1.0%) | (0.0%) | 1 (1.0%) | (0.0%) | (0.0%) | (0.0%) | (0.0%) | (0.0%) | (0.0%) | (0.0%) |
| Nausea | 1 (1.0%) | (0.0%) | 1 (1.0%) | (0.0%) | (0.0%) | (0.0%) | (0.0%) | (0.0%) | (0.0%) | (0.0%) |
| Upper gastrointestinal | 1 (1.0%) | 1 (1.0%) | (0.0%) | (0.0%) | (0.0%) | (0.0%) | (0.0%) | (0.0%) | (0.0%) | (0.0%) |
| haemorrhage | | | | | | | | | | |
| Respiratory, thoracic and | 7 (7.1%) | 2 (2.0%) | 5 (5.1%) | 1 (1.0%) | 2 (2.0%) | 1 (1.0%) | (0.0%) | 1 (1.0%) | (0.0%) | 1 (1.0%) |
| mediastinal disorders | | | | | | | | | | |
| Chronic | 2 (2.0%) | 1 (1.0%) | 1 (1.0%) | (0.0%) | (0.0%) | (0.0%) | (0.0%) | (0.0%) | (0.0%) | (0.0%) |
| obstructive | | | | | | | | | | |
| pulmonary | | | | | | | | | | |
| disease | | | | | | | | | | |
| Dyspnoea | 2 (2.0%) | 1 (1.0%) | 1 (1.0%) | (0.0%) | (0.0%) | (0.0%) | (0.0%) | (0.0%) | (0.0%) | (0.0%) |
| Pleural effusion | 2 (2.0%) | (0.0%) | 2 (2.0%) | (0.0%) | (0.0%) | (0.0%) | (0.0%) | (0.0%) | (0.0%) | (0.0%) |
| Acute respiratory failure | 1 (1.0%) | (0.0%) | (0.0%) | 1 (1.0%) | (0.0%) | (0.0%) | (0.0%) | (0.0%) | (0.0%) | (0.0%) |
| Pneumonitis | 1 (1.0%) | (0.0%) | 1 (1.0%) | (0.0%) | 1 (1.0%) | 1 (1.0%) | (0.0%) | 1 (1.0%) | (0.0%) | 1 (1.0%) |
| Respiratory failure | 1 (1.0%) | (0.0%) | (0.0%) | (0.0%) | 1 (1.0%) | (0.0%) | (0.0%) | (0.0%) | (0.0%) | (0.0%) |
| General disorders and | 5 (5.1%) | 1 (1.0%) | 4 (4.0%) | (0.0%) | (0.0%) | 2 (2.0%) | 1 (1.0%) | 1 (1.0%) | (0.0%) | (0.0%) |
| administration | | | | | | | | | | |
| site conditions | | | | | | | | | | |
| Fatigue | 1 (1.0%) | (0.0%) | 1 (1.0%) | (0.0%) | (0.0%) | (0.0%) | (0.0%) | (0.0%) | (0.0%) | (0.0%) |
| Generalised oedema | 1 (1.0%) | (0.0%) | 1 (1.0%) | (0.0%) | (0.0%) | 1 (1.0%) | (0.0%) | 1 (1.0%) | (0.0%) | (0.0%) |
| Non-cardiac chest pain | 1 (1.0%) | (0.0%) | 1 (1.0%) | (0.0%) | (0.0%) | (0.0%) | (0.0%) | (0.0%) | (0.0%) | (0.0%) |
| Pain | 1 (1.0%) | (0.0%) | 1 (1.0%) | (0.0%) | (0.0%) | (0.0%) | (0.0%) | (0.0%) | (0.0%) | (0.0%) |
| Pyrexia | 1 (1.0%) | 1 (1.0%) | (0.0%) | (0.0%) | (0.0%) | 1 (1.0%) | 1 (1.0%) | (0.0%) | (0.0%) | (0.0%) |
| Infections and infestations | 5 (5.1%) | 1 (1.0%) | 5 (5.1%) | (0.0%) | (0.0%) | (0.0%) | (0.0%) | (0.0%) | (0.0%) | (0.0%) |
| COVID-19 | 1 (1.0%) | 1 (1.0%) | (0.0%) | (0.0%) | (0.0%) | (0.0%) | (0.0%) | (0.0%) | (0.0%) | (0.0%) |
| Localised infection | 1 (1.0%) | (0.0%) | 1 (1.0%) | (0.0%) | (0.0%) | (0.0%) | (0.0%) | (0.0%) | (0.0%) | (0.0%) |
| Peritonitis bacterial | 1 (1.0%) | (0.0%) | 1 (1.0%) | (0.0%) | (0.0%) | (0.0%) | (0.0%) | (0.0%) | (0.0%) | (0.0%) |
| Pneumonia | 1 (1.0%) | (0.0%) | 1 (1.0%) | (0.0%) | (0.0%) | (0.0%) | (0.0%) | (0.0%) | (0.0%) | (0.0%) |
| Septic shock | 1 (1.0%) | (0.0%) | 1 (1.0%) | (0.0%) | (0.0%) | (0.0%) | (0.0%) | (0.0%) | (0.0%) | (0.0%) |
| Skin infection | 1 (1.0%) | (0.0%) | 1 (1.0%) | (0.0%) | (0.0%) | (0.0%) | (0.0%) | (0.0%) | (0.0%) | (0.0%) |
| Urinary tract infection | 1 (1.0%) | (0.0%) | 1 (1.0%) | (0.0%) | (0.0%) | (0.0%) | (0.0%) | (0.0%) | (0.0%) | (0.0%) |
| Metabolism and nutrition | 4 (4.0%) | (0.0%) | 3 (3.0%) | (0.0%) | 1 (1.0%) | (0.0%) | (0.0%) | (0.0%) | (0.0%) | (0.0%) |
| disorders | | | | | | | | | | |
| Acidosis | 1 (1.0%) | (0.0%) | (0.0%) | (0.0%) | 1 (1.0%) | (0.0%) | (0.0%) | (0.0%) | (0.0%) | (0.0%) |
| Decreased appetite | 1 (1.0%) | (0.0%) | 1 (1.0%) | (0.0%) | (0.0%) | (0.0%) | (0.0%) | (0.0%) | (0.0%) | (0.0%) |
| Hyperkalaemia | 1 (1.0%) | (0.0%) | 1 (1.0%) | (0.0%) | (0.0%) | (0.0%) | (0.0%) | (0.0%) | (0.0%) | (0.0%) |
| Hyponatraemia | 1 (1.0%) | (0.0%) | 1 (1.0%) | (0.0%) | (0.0%) | (0.0%) | (0.0%) | (0.0%) | (0.0%) | (0.0%) |
| Hepatobiliary disorders | 3 (3.0%) | (0.0%) | 3 (3.0%) | (0.0%) | (0.0%) | 2 (2.0%) | (0.0%) | 2 (2.0%) | (0.0%) | (0.0%) |
| Immune-mediated hepatitis | 2 (2.0%) | (0.0%) | 2 (2.0%) | (0.0%) | (0.0%) | 2 (2.0%) | (0.0%) | 2 (2.0%) | (0.0%) | (0.0%) |
| Cholangitis | 1 (1.0%) | (0.0%) | 1 (1.0%) | (0.0%) | (0.0%) | (0.0%) | (0.0%) | (0.0%) | (0.0%) | (0.0%) |
| Injury, poisoning | 2 (2.0%) | (0.0%) | 1 (1.0%) | (0.0%) | 1 (1.0%) | (0.0%) | (0.0%) | (0.0%) | (0.0%) | (0.0%) |
| and procedural | | | | | | | | | | |
| complications | | | | | | | | | | |
| Fall | 1 (1.0%) | (0.0%) | 1 (1.0%) | (0.0%) | (0.0%) | (0.0%) | (0.0%) | (0.0%) | (0.0%) | (0.0%) |
| Subarachnoid haemorrhage | 1 (1.0%) | (0.0%) | (0.0%) | (0.0%) | 1 (1.0%) | (0.0%) | (0.0%) | (0.0%) | (0.0%) | (0.0%) |
| Musculoskeletal | 2 (2.0%) | (0.0%) | 2 (2.0%) | (0.0%) | (0.0%) | (0.0%) | (0.0%) | (0.0%) | (0.0%) | (0.0%) |
| and connective | | | | | | | | | | |
| tissue disorders | | | | | | | | | | |
| Back pain | 1 (1.0%) | (0.0%) | 1 (1.0%) | (0.0%) | (0.0%) | (0.0%) | (0.0%) | (0.0%) | (0.0%) | (0.0%) |
| Musculoskeletal pain | 1 (1.0%) | (0.0%) | 1 (1.0%) | (0.0%) | (0.0%) | (0.0%) | (0.0%) | (0.0%) | (0.0%) | (0.0%) |
| Nervous system disorders | 2 (2.0%) | (0.0%) | 2 (2.0%) | (0.0%) | (0.0%) | (0.0%) | (0.0%) | (0.0%) | (0.0%) | (0.0%) |
| Haemorrhage intracranial | 1 (1.0%) | (0.0%) | 1 (1.0%) | (0.0%) | (0.0%) | (0.0%) | (0.0%) | (0.0%) | (0.0%) | (0.0%) |
| Hemiparesis | 1 (1.0%) | (0.0%) | 1 (1.0%) | (0.0%) | (0.0%) | (0.0%) | (0.0%) | (0.0%) | (0.0%) | (0.0%) |
| Psychiatric disorders | 2 (2.0%) | 1 (1.0%) | 1 (1.0%) | (0.0%) | (0.0%) | 1 (1.0%) | (0.0%) | 1 (1.0%) | (0.0%) | (0.0%) |
| Confusional state | 1 (1.0%) | 1 (1.0%) | (0.0%) | (0.0%) | (0.0%) | (0.0%) | (0.0%) | (0.0%) | (0.0%) | (0.0%) |
| Delirium | 1 (1.0%) | (0.0%) | 1 (1.0%) | (0.0%) | (0.0%) | 1 (1.0%) | (0.0%) | 1 (1.0%) | (0.0%) | (0.0%) |
| Cardiac disorders | 1 (1.0%) | (0.0%) | (0.0%) | 1 (1.0%) | (0.0%) | (0.0%) | (0.0%) | (0.0%) | (0.0%) | (0.0%) |
| Ventricular tachycardia | 1 (1.0%) | (0.0%) | (0.0%) | 1 (1.0%) | (0.0%) | (0.0%) | (0.0%) | (0.0%) | (0.0%) | (0.0%) |
| Investigations | 1 (1.0%) | (0.0%) | 1 (1.0%) | (0.0%) | (0.0%) | 1 (1.0%) | (0.0%) | 1 (1.0%) | (0.0%) | (0.0%) |
| Liver function | 1 (1.0%) | (0.0%) | 1 (1.0%) | (0.0%) | (0.0%) | 1 (1.0%) | (0.0%) | 1 (1.0%) | (0.0%) | (0.0%) |
| test increased | | | | | | | | | | |

What is claimed:

1. A method for treating cancer in a human subject in need thereof, comprising administering to the subject an anti-TIGIT antibody that has reduced binding to one or more activating human FcγRs as compared to wild type (WT) human IgG1, wherein treatment results in a reduction of one or more adverse event as compared to a similar treatment comprising an Fc-enabled anti-TIGIT antibody.

2. A method for treating cancer in a human subject in need thereof, comprising administering to the subject an anti-TIGIT antibody that has reduced binding to one or more activating human FcγRs as compared to wild type (WT) human IgG1, wherein the subject has a reduced likelihood of experiencing one or more adverse event as compared to treatment with an Fc-enabled anti-TIGIT antibody.

3. The method of claim 1 or 2, wherein the Fc-enabled anti-TIGIT antibody is selected from AB308, BMS-986207, tiragolumab, vibostolimab, etigilimab, ociperlimab, EOS-448, SEA-TGT, AGEN1777, AGEN1327, JS006, ralzapastotug, and an Fc-enabled version of domvanalimab comprising a wild-type IgG1 Fc region.

4. A method for treating cancer in a human subject in need thereof without significantly increasing the likelihood of adverse event as compared to the standard of care, comprising administering to the subject an anti-TIGIT antibody that has reduced binding to one or more activating human FcγRs as compared to wild type (WT) human IgG1 in combination with one or more additional therapy.

5. A method for reducing one or more adverse event experienced by a human subject being treated for cancer with an anti-TIGIT antibody, comprising administering to the subject an anti-TIGIT antibody that has reduced binding to one or more activating human FcγRs as compared to wild type (WT) human IgG1.

6. A method for reducing one or more adverse event experienced by a human subject being treated for cancer with an anti-TIGIT antibody and an additional immunotherapeutic agent, comprising administering to the subject an anti-TIGIT antibody that has reduced binding to one or more activating human FcγRs as compared to wild type (WT) human IgG1 and an additional immunotherapeutic agent.

7. The method of claim 6, wherein the immunotherapeutic agent is a checkpoint inhibitor, optionally selected from ipilimumab, nivolumab, pembrolizumab, cemiplimab, avelumab, durvalumab, atezolizumab, and zimberelimab.

8. The method of any one of claims 1 to 7, wherein the adverse event is a treatment-related adverse event, a treatment-emergent adverse event, an immune-related adverse event, a treatment-related immune-related adverse event, a treatment-related adverse event leading to treatment discontinuation, a treatment-related adverse event leading to treatment disruption, a serious adverse event leading to treatment discontinuation, a serious adverse event leading to treatment disruption, a serious adverse event, a grade 3 or higher serious adverse event, or a grade 3 or higher treatment-related adverse event.

9. The method of 8, wherein the immune-related adverse event is selected from:
(i) a skin or subcutaneous tissue disorder, a gastrointestinal disorder, a hepatobiliary disorder, an endocrine disorder, or a respiratory, thoracic or mediastinal disorder;
(ii) a skin or subcutaneous tissue disorder;
(iii) rash, oral mucositis, dry mouth, colitis, diarrhea, hepatitis, pneumonitis, endocrinopathies, hypophysitis, hypothyroidism, hyperthyroidism, adrenal insufficiency, diabetes, or a combination thereof;
(iv) arthritis, hepatitis, hypothyroidism, hyperthyroidism, infusion-related reaction, maculo-papular rash, pneumonitis, pruritis, psoriasis, rash, swelling face, or a combination thereof; or
(v) infusion-related reaction, maculo-papular rash, pruritis, psoriasis, rash, or a combination thereof.

10. A method for reducing one or more dose reduction, temporary treatment disruption, or treatment discontinuation experienced by a human subject being treated for cancer with an anti-TIGIT antibody, comprising administering to the subject an anti-TIGIT antibody that has reduced binding to one or more activating human FcγRs as compared to wild type (WT) human IgG1.

11. A method for reducing one or more dose reduction, temporary treatment disruption, or treatment discontinuation experienced by a human subject being treated for cancer with an anti-TIGIT antibody and an additional immunotherapeutic agent, comprising administering to the subject an anti-TIGIT antibody that has reduced binding to one or more activating human FcγRs as compared to wild type (WT) human IgG1.

12. The method of claim 11, wherein the immunotherapeutic agent is a checkpoint inhibitor, optionally selected from ipilimumab, nivolumab, pembrolizumab, cemiplimab, avelumab, durvalumab, atezolizumab, and zimberelimab.

13. The method of any one of claims 10-12, the method further comprising a reduction in one or more immune-related adverse event, as compared to a similar treatment comprising an Fc-enabled anti-TIGIT antibody.

14. The method of 13, wherein the immune-related adverse event is selected from:
(i) a skin or subcutaneous tissue disorder, a gastrointestinal disorder, a hepatobiliary disorder, an endocrine disorder, or a respiratory, thoracic or mediastinal disorder;
(ii) a skin or subcutaneous tissue disorder;
(iii) rash, oral mucositis, dry mouth, colitis, diarrhea, hepatitis, pneumonitis, endocrinopathies, hypophysitis, hypothyroidism, hyperthyroidism, adrenal insufficiency, diabetes, or a combination thereof;
(iv) arthritis, hepatitis, hypothyroidism, hyperthyroidism, infusion-related reactions, maculo-papular rash, pneumonitis, pruritis, psoriasis, rash, swelling face, or a combination thereof; or
(v) infusion-related reactions, maculo-papular rash, pruritis, psoriasis, rash; or a combination thereof.

15. The method of any one of the preceding claims, wherein administering comprises one or more dosing cycles.

16. The method of claim 15, wherein a dosing cycle comprises administering the anti-TIGIT antibody to the subject once every 2 weeks, once every 3 weeks, or once every 4 weeks.

17. The method of any one of claims 1-16, wherein the anti-TIGIT antibody is administered to the subject at a dose of about 5 mg/kg, about 10 mg/kg, about 15 mg/kg, about 20 mg/kg, or about 25 mg/kg.

18. The method of any one of claims 1-16, wherein the anti-TIGIT antibody is administered to the subject at a dose of about 500 mg to about 2000 mg, about 600 mg to about 800 mg, about 900 mg to about 1200 mg, about 1200 mg to about 1600 mg, or about 1200 mg to about 1500 mg.

19. The method of claim 15, wherein a dosing cycle comprises administering the anti-TIGIT antibody to the subject at a dose of about 1200 mg once every three weeks or at a dose of about 1600 mg once every four weeks.

20. The method of any one of claims 1-19 further comprising administration of one or more additional therapeutic agent, wherein the one or more additional therapeutic agent is, optionally, an immunotherapeutic agent, a chemotherapeutic agent, a chemotherapeutic regimen, or a combination thereof.

21. The method of claim 20, wherein the immunotherapeutic agent is a checkpoint inhibitor, optionally selected from ipilimumab, nivolumab,
pembrolizumab, cemiplimab, avelumab, durvalumab, atezolizumab, and zimberelimab; or
an ATP-adenosine axis-targeting agent, optionally selected from an A₂ₐR antagonist, an A_{2b}R antagonist, an A₂ₐR and A_{2b}R antagonist, a CD73 inhibitor, and a CD39 inhibitor.

22. The method of claim 20, wherein the chemotherapeutic regimen is a fluoropyrimidine-containing chemotherapy or a platinum-containing chemotherapy.

23. The method of claim 22, wherein the fluoropyrimidine-containing chemotherapy is fluorouracil and the platinum-containing chemotherapy is carboplatin, cisplatin, or oxaliplatin.

24. The method of claim 22, wherein the chemotherapeutic regimen is FOLFOX, CAPOX, cisplatin and pemetrexed, carboplatin and pemetrexed, carboplatin and paclitaxel, or carboplatin and nab-paclitaxel.

25. The method of any one of the preceding claims, wherein the anti-TIGIT antibody that has reduced binding to one or more activating human FcγR is a human IgG4 or a human IgG1 with reduced ability to bind one or more activating human FcγR.

26. The method of any one of the preceding claims, wherein the anti-TIGIT that has reduced binding to one or more activating human FcγR does not bind to one or more activating human FcγR.

27. The method of any one of the preceding claims, wherein the anti-TIGIT antibody comprises a heavy chain CDR1 comprising the amino acid sequence of SEQ ID NO: 2, a heavy chain CDR2 comprising the amino acid sequence of SEQ ID NO: 3, a heavy chain CDR3 comprising the amino acid sequence of SEQ ID NO: 4, a light chain CDR1 comprising the amino acid sequence of SEQ ID NO: 5, a light chain CDR2 comprising the amino acid sequence of SEQ ID NO: 6, and a light chain CDR3 comprising the amino acid sequence of SEQ ID NO: 7.

28. The method of any one of the preceding claims, wherein the anti-TIGIT antibody comprises a heavy chain variable region with at least 90% sequence identity to SEQ ID NO: 8 or 10 and a light chain variable region with at least 90% sequence identity to SEQ ID NO: 9 or 11.

29. The method of any one of the preceding claims, wherein the anti-TIGIT antibody comprises a heavy chain with at least 90% sequence identity to SEQ ID NO: 12 and a light chain with at least 90% sequence identity to SEQ ID NO: 13.

30. The method of any one of the preceding claims, wherein the anti-TIGIT antibody binds to the same epitope of TIGIT as domvanalimab or the anti-TIGIT antibody competitively inhibits binding of domvanalimab to human TIGIT by at least 50%.

31. The method of any one of the preceding claims, wherein the cancer is a solid tumor, optionally wherein the tumor is a locally advanced and/or unresectable tumor; a metastatic tumor; a recurrent tumor; a tumor no longer responding to a treatment optionally wherein the treatment is a standard of care, a checkpoint inhibitor, a PD-1 antagonist, or a PD-L1 antagonist; or any combination thereof.

32. The method of claim 31, wherein the cancer is lung cancer, genitourinary cancer, or gastrointestinal cancer.

33. The method of claim 32, wherein the cancer is lung cancer.

34. The method of claim 33, wherein the lung cancer is non-small cell lung cancer (NSCLC), optionally wherein the cancer is (i) locally advanced, unresectable, locally advanced unresectable, or metastatic, (ii) no longer responding to a treatment, optionally wherein the treatment is a standard of care, a checkpoint inhibitor, a PD-1 antagonist, or a PD-L1 antagonist, or (iii) a combination of (i) and (ii).

35. The method of claim 34, wherein the lung cancer is squamous or non-squamous, unresectable locally advanced disease or metastatic disease.

36. The method of any one of claims 33 to 35, wherein the subject is checkpoint inhibitor naïve.

37. The method of any one of claims 33 to 35, wherein the subject is checkpoint inhibitor experienced.

38. The method of claim 32, wherein the cancer is gastrointestinal cancer.

39. The method of claim 38, wherein the gastrointestinal cancer is esophageal cancer, gastric cancer, colorectal cancer, pancreatic cancer or liver cancer, optionally wherein the cancer is (i) locally advanced, unresectable, locally advanced unresectable, or metastatic and/or (ii) no longer responding to a treatment, such as a standard of care, a checkpoint inhibitor, a PD-1 antagonist, or a PD-L1 antagonist.

40. The method of claim 39, wherein the gastrointestinal cancer is esophageal cancer or gastric cancer, optionally wherein the cancer is (i) locally advanced, unresectable, locally advanced unresectable, or metastatic and/or (ii) no longer responding to a treatment, such as a standard of care.

41. The method of claim 40, wherein the gastrointestinal cancer is esophageal adenocarcinoma, esophageal squamous cell carcinoma, gastroesophageal junction adenocarcinoma, or gastric adenocarcinoma, optionally wherein the cancer is (i) locally advanced, unresectable, locally advanced unresectable, or metastatic and/or (ii) no longer responding to a treatment, such as a standard of care.

42. The method of claim 41, wherein the cancer is NSCLC, head and neck squamous cell carcinoma (HNSCC), renal cell carcinoma (RCC), breast cancer, colorectal cancer (CRC), melanoma, bladder cancer, ovarian cancer, endometrial cancer, Merkel Cell, or gastroesophageal cancer.

43. The method of any one of the preceding claims, wherein PD-L1 expression of the cancer is TPS ≥50%, as measured by a clinically validated PD-L1 IHC assay or FDA-approved test.

44. The method of any one of claims 1 to 42, wherein the PD-L1, corresponding to TPS <50% as measured by a clinically validated PD-L1 IHC assay or FDA-approved test.

45. The method of claim 44, wherein PD-L1 expression of the cancer is about 1-10%, about 10%-20%, about 20-30%, about 30-40%, about 40-49%, about 1-49%, about 1-25%, or about 25-49%, as measured by a clinically validated PD-L1 IHC assay or FDA-approved test.

46. The method of claim 44, wherein PD-L1 expression of the cancer is ≤1%, as measured by a clinically validated PD-L1 IHC assay or FDA-approved test.

47. The method of any one of the preceding claims, wherein the cancer is tumor mutational burden-high (TMB-H; ≥10 mutations/megabase (mut/Mb), as determined by an FDA-approved test.

48. The method of any one of claims 1 to 46, wherein the cancer is not TMB-H.

49. The method of any one of the preceding claims, wherein the cancer does not have an actionable oncogenic mutation in ALK, EGFR, ROS, BRAF, or NTRK and/or has wildtype ALK, EGFR, ROS, BRAF, and/or NTRK.

50. The method of any one of the preceding claims, wherein the cancer expresses or overexpresses one or more biomarker selected CD73, DNAM-1, PVR, TIGIT, and CD8-Ki67.

51. The method of any one of claim 1, 2, 3, 5, or 10, wherein the anti-TIGIT antibody that has reduced binding to one or more activating human FcγRs as compared to wild type (WT) human IgG1 is domvanalimab and the subject is also administered a therapeutically effective amount of PD-1 antagonist or a PD-L1 antagonist.

52. The method of any one of claim 4, 6, or 11, wherein the anti-TIGIT antibody that has reduced binding to one or more activating human FcγRs as compared to wild type (WT) human IgG1 is domvanalimab and an additional agent is a PD-1 antagonist or a PD-L1 antagonist.

53. The method of claim 51 or 52, wherein the cancer is locally advanced, metastatic, or unresectable non-small cell lung cancer (NSCLC) or locally advanced and unresectable NSCLC, optionally wherein domvanalimab is administered at a dose of about 1200 mg once every three weeks or at a dose of about 1600 mg once every four weeks.

54. The method of claim 53, wherein the cancer is metastatic NSCLC, and domvanalimab and the PD-1 antagonist or PD-L1 antagonist are administered in combination with a chemotherapeutic agent or a chemotherapeutic regimen as a first-line treatment.

55. The method of claim 54, wherein the cancer is
metastatic non-squamous NSCLC and domvanalimab and the PD-1 antagonist or PD-L1 antagonist are administered in combination with a chemotherapeutic regimen, optionally where the chemotherapeutic regimen is pemetrexed and a platinum-containing chemotherapy; or
metastatic squamous NSCLC and domvanalimab and the PD-1 antagonist or PD-L1 antagonist are administered in combination with a chemotherapeutic regimen, optionally where the chemotherapeutic regimen is a taxane and a platinum-containing chemotherapy.

56. The method of claim 53, wherein the cancer is locally advanced or metastatic NSCLC, and domvanalimab and the PD-1 antagonist or PD-L1 antagonist are administered as a first-line treatment without additional therapeutic agents.

57. The method of claim 53, wherein the cancer is locally advanced or metastatic NSCLC, and domvanalimab and the PD-1 antagonist or PD-L1 antagonist are administered in combination with an A₂ₐR and/or A_{2b}R antagonist or a CD73 inhibitor as a first-line treatment.

58. The method of claim 53, wherein the cancer is metastatic NSCLC, and domvanalimab and the PD-1 antagonist or PD-L1 antagonist are administered as a second-line or greater treatment, optionally in combination with one or more additional therapeutic agent.

59. The method of claim 58, wherein the subject has disease progression on or after treatment with a platinum-containing chemotherapy, a checkpoint inhibitor optionally wherein the checkpoint inhibitor is a PD-1 antagonist or a PD-L1 antagonist, or a targeted therapy optionally wherein the targeted therapy is a tyrosine kinase inhibitor.

60. The method of claim 58 or 59, wherein domvanalimab and the PD-1 antagonist or PD-L1 antagonist are administered without additional therapeutic agents,

61. The method of claim 58 or 59, wherein the subject is administered an additional therapeutic agent in combination with domvanalimab and the PD-1 antagonist or PD-L1 antagonist, optionally wherein each additional therapeutic agent is selected from an A₂ₐR antagonist, an A_{2b}R antagonist, an A₂ₐR/A_{2b}R antagonist, a CD73 inhibitor, a chemotherapeutic agent, or a chemotherapeutic regimen.

62. The method of claim 53, wherein the cancer is locally advanced, unresectable NSCLC and the subject's disease has not progressed following chemoradiation therapy, optionally wherein domvanalimab and the PD-1 antagonist or PD-L1 antagonist are administered without additional therapeutic agents.

63. The method of claim 51 or 52, wherein the cancer is Stage IB, Stage II, or Stage III NSCLC, and domvanalimab and the PD-1 antagonist or PD-L1 antagonist are administered as an adjuvant treatment after complete surgical resection, optionally wherein domvanalimab is administered at a dose of about 1200 mg once every three weeks or at a dose of about 1600 mg once every four weeks.

64. The method of claim 51 or 52, wherein the cancer is resectable NSCLC, and domvanalimab and the PD-1 antagonist or PD-L1 antagonist are administered in the neoadjuvant setting, in combination with a chemotherapeutic regimen comprising a platinum-containing chemotherapy.

65. The method of any one of claims 53 to 64, wherein the cancer is: without an actionable oncogenic mutation in epidermal growth factor (EGFR) or anaplastic lymphoma kinase (ALK); and/or PD-L1 expression of the cancer as measured by a clinically validated PD-Li IHC assay or FDA-approved test is ≥1%, ≥5%, ≥10%, or ≥50%.

66. The method of claim 51 or 52, wherein the cancer is esophageal, gastroesophageal. junction (GEJ), or gastric adenocarcinoma (GA), optionally wherein domvanalimab is administered at a dose of about 1200 mg once every three weeks or at a dose of about 1600 mg once every four weeks.

67. The method of claim 66, wherein the esophageal, GEJ, or GA cancer is locally advanced unresectable or metastatic, and domvanalimab and the PD-1 antagonist or PD-L1 antagonist are administered in combination with a chemotherapeutic agent or a chemotherapeutic regimen as a first-line treatment.

68. The method of claim 67, wherein domvanalimab and the PD-1 antagonist or PD-L1 antagonist are administered in combination with a chemotherapeutic regimen comprising a fluoropyrimidine-containing and platinum-containing chemotherapy

69. The method of claim 66, wherein the esophageal, GEJ, or GA cancer is locally advanced, locally advanced unresectable or metastatic, and domvanalimab and the PD-1 antagonist or PD-L1 antagonist are administered as a second-line or greater treatment, optionally without additional therapeutic agents.

70. The method of claim 69, wherein the subject has disease progression on or after one or more line of therapy comprising a platinum-containing chemotherapy, a fluoropyrimidine-containing chemotherapy, a checkpoint inhibitor optionally wherein the checkpoint inhibitor is a PD-1 antagonist or a PD-L1 antagonist, a targeted agent optionally wherein the targeted agent is a HER2/neu-targeted therapy, or any combination thereof.

71. The method of claim 66, wherein the subject has completely resected esophageal or GEJ cancer with residual pathologic disease and has received neoadjuvant chemoradiotherapy.

72. The method of any one of claims 66-71, wherein PD-L1 expression of the cancer as measured by a clinically validated PD-L1 IHC assay or FDA-approved test is ≥1%, ≥5%, ≥10%, or ≥50%.

73. The method of any one of the preceding claims, wherein treatment results in a reduction in tumor size, a reduction in tumor number, a reduction in metastasis, stable disease, partial response, complete response, or a combination thereof.

74. The method of any one of the preceding claims, wherein the treatment results in an improvement in overall survival, progression-free survival, disease control rate, overall response rate, or a combination thereof compared to placebo or a standard of care.

75. The method of any one of the preceding claims, wherein the treatment results in an increased time to progression, increased disease-free survival, increased duration of response, an increase in duration of clinical benefit, an increase in time to treatment failure, or any combination thereof, compared to placebo or a standard of care.

76. The method of any one of the preceding claims, wherein the anti-TIGIT antibody that has reduced binding to one or more activating human FcγRs as compared to wild type (WT) human IgG1 is formulated for dilution as an aqueous solution comprising about 10 mg/mL to about 100 mg/mL antibody or about 20 mg/mL to about 60 mg/mL antibody, a buffer containing about 15 to about 30 mM histidine/histidine-C1, about 5% to about 10% (weight/volume) of an excipient selected from the group consisting of sucrose, dextrose, trehalose, sorbitol, and mannitol, about 0 mg/mL to about 10 mg/mL NaCl, and about 0.05 mg/mL to about 0.6 mg/mL polysorbate 80.
